(19) United States
(12) Patent Application Publication
(10) Pub. No.: US 2005/0026189 A1
(43) Pub. Date: Feb. 3, 2005

(54) Microbial operons

(76) Inventors: Liangsu Wang, San Diego, CA (US); Carlos Zamudio, La Jolla, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/857,625

(22) Filed: May 28, 2004

Related U.S. Application Data
(60) Provisional application No. 60/474,768, filed on May 29, 2003.

(51) Int. Cl. C12Q 1/68; G06F 19/00; G01N 33/48; G01N 33/50

(52) U.S. Cl. 435/6; 702/20

(57) ABSTRACT

Described herein is a method for predicting operons in prokaryotes. Also described herein are vectors comprising operons predicted using the this method as well as methods of using antisense nucleic acids complementary to at least a portion of a predicted proliferation-required operon to inhibit cellular proliferation. Methods of using such antisense nucleic acids to sensitize cells for use in assays to identify compounds which possess the ability to inhibit cellular proliferation are also described.

### RELATED APPLICATIONS

This application is a nonprovisional of and claims priority under 35 U.S.C. § 119(e) to U.S. Provisional Patent Application Ser. No. 60/474,768, entitled MICROBIAL OPERONS, filed May 29, 2003, the disclosure of which is incorporated herein by reference in its entirety.

### SEQUENCE LISTING

The present application is being filed along with duplicate copies of a CD-ROM marked “Copy 1” and “Copy 2” containing a Sequence Listing in electronic format. The duplicate copies of the CD-ROM each contain a file entitled ELITRA036A.ST25.txt created on May 27, 2004 which is 2,252,898 bytes in size. The information on these duplicate CD-ROMs is incorporated herein by reference in its entirety.

### TECHNICAL FIELD

The invention described herein relates to genomic organization and operon structure as well as their application to lead compound investigations. In particular, the invention relates to methods for predicting microbial operons and the use of operons comprising genes encoding products whose inhibition reduces proliferation as targets for identifying compounds that inhibit proliferation.

### BACKGROUND

Elucidation of operon structure and organization is becoming increasingly important to the newly emerging practices in drug discovery which are based on the identification of genes and gene products that are required for microbial proliferation. Although many proliferation-required genes are monocistronic, a large number of such genes are present in the cell as part of a polycistronic operon. When one or more proliferation-required genes are interspersed in an operon with nonessential genes, methods for determining which of the genes in the operon are essential can be complicated by polar effects. For example, the inhibition of a single nonessential gene in an operon may result in the inhibition of one or more proliferation-required genes that are present in the same operon. If it is unknown that the nonessential gene is part of the operon, then the nonessential gene may be misidentified as a proliferation-required gene. Thus, without the knowledge of the operon structure of an organism, many nonessential genes would remain misidentified as required for proliferation. Given the possibility that the genes encoding true drug targets can be masked by polar effects it would be useful to predict the structure of operons of targeted organisms.

With more and more genomic sequences becoming available, it becomes possible to elucidate operon structure yet numerous problems exist. For example, it remains extremely difficult to predict operons in organisms solely from their genome sequences or in the relative absence of extensive experimental data. For the methods which are currently used for predicting operons based on raw genome sequence, there remain serious concerns with the accuracy of the prediction.

Staphylococcus aureus is an example of a microbial pathogen for which there exists a wealth of genomic sequence data but limited experimental evidence by which to determine operons. This gram-positive bacterium is a major human pathogen causing both community-acquired and hospital-acquired infections. Antibiotic resistant strains of Staphylococcus aureus have recently been identified, including those that are now resistant to all available antibiotics, thereby severely limiting the options of care available to physicians. Accordingly, a method of accurate operon structure prediction, which would aid in the identification of novel drug targets thereby furthering the development of new antimicrobial compounds effective against multiple drug resistant microbes, such as Staphylococcus aureus, would be of enormous utility. Likewise, methods which permit the screening of test compounds to identify compounds which inhibit microbial proliferation and methods which permit identification of the specific gene which encodes the gene product that is the target of an antimicrobial compound would provide useful information for further drug development.

### SUMMARY OF THE INVENTION

Identification of operon structure and organization within a genome may desirably aid in understanding gene regulation and function in prokaryotic organisms. In various embodiments, the present teachings describe a consensus-based approach to operon identification that integrates a number of complimentary operon prediction methods. The disclosed methods may desirably improve the identification and association of co-transcribed genes within a prokaryotic organism's genome as compared to conventional systems employing singular prediction models.

In one aspect, operon prediction according to the present teachings may be used to score the likelihood that adjacent gene pairs within a prokaryotic organism's genome are co-transcribed. Gene pairs are identified and segregated into discrete bins indicative of distinct operons on the basis of a calculated score using the consensus operon prediction model. Operon boundaries are identified by comparing the score associated with a selected gene pair with a threshold. Using this approach, the predicted operons, the genes contained therein, and the associated operon boundaries may be mapped back to the prokaryotic organism's genome to generate an annotated genomic map for the selected prokaryotic organism. The genomic map may also be annotated at the intersections for each adjacent gene pair to indicate the probability that the genes of the gene pair are contained in the same operon.

An application of the present teachings is demonstrated for the prokaryotic organism, Staphylococcus aureus wherein over 90% of the identified gene pairs were associated with operon prediction scores indicating a high confidence of either being in distinct operons or in the same operon. Application of an empirically derived threshold for this organism, predicted over 1397 operons from the protein-encoding genes in the Staphylococcus aureus strain Mu50 genome. Of the identified operons, approximately 62% were predicted to be monocistronic and approximately 38% were predicted to be polycistronic. Comparison with experimentally determined values for Staphylococcus aureus operons from literature sources indicates that the disclosed methods successfully predicted operon boundaries and genes contained within the each operon interior with a high degree of accuracy.

As will be described in greater detail hereinbelow, operon prediction according to the present teachings provides a means to identify operons with a high degree of accuracy even when applied to less-well characterized genomes wherein limited experimental evidence for operon structure is available. Furthermore, the consensus-based approach to operon prediction may be readily modified by the inclusion or exclusion of various operon prediction approaches as determined by the investigator. This flexibility allows an investigator to adjust the stringency of the analysis and to make use of available data and information that may vary from one organism to the next. Additionally, the methods may be customized to include operon prediction methods other than those specifically disclosed to thereby accommodate other techniques and provide improved operon identification capabilities as compared using a singular approach alone.

Further aspects of the present invention are described in the numbered paragraphs below.

1. A method for predicting operons, the method comprising:
identifying consecutive genes within at least a portion of a target prokaryotic organism's genome;
determining each gene's orientation in the genome relative to its flanking genes;
segregating the genes into a plurality of bins on the basis of their orientation such that consecutive genes in the same orientation are grouped into the same bin;
performing a composite operon prediction analysis comprising pairing each gene within a selected bin with its respective flanking genes and associating a confidence score with each gene pair, wherein the confidence score reflects the likelihood that a selected gene pair resides in the same operon using the criteria of orientation and the application of at least one operon prediction method;
determining operon boundaries by identifying gene pairs having confidence scores that fall below a selected threshold; and
associating genes contained between operon boundaries as putative operons.

2. The method of Paragraph 1, wherein the target prokaryotic organism is Bacillus subtilis.

3. The method of Paragraph 1, wherein the target prokaryotic organism is Escherichia coli.

4. The method of Paragraph 1, wherein the target prokaryotic organism is selected from the group consisting of Aquifex aeolicus, Borrelia burgdorferi, Bacillus halodurans, Buchnera species, Clostridium acetobutylicum, Caulobacter crescentus, Campylobacter jejun, Chlamydia pneumoniae, Chlamydia trachomatis, Haemophilus influenzae, Helicobacter pylori, Listeria monocytogenes, Listeria innocua, Lactococcus lactis, Mycoplasma genitalium, Mycobacterium leprae, Mycoplasma pneumoniae, Mycoplasma pulmonis, Mycobacterium tuberculosis, Neisseria meningitides, Pseudomonas aeruginosa, Pasteurella multocida, Rickettsia conorii, Rickettsia prowazekii, Sinorhizobium meliloti, Streptococcus pneumoniae, Streptococcus pyogenes, Salmonella typhi, Salmonella typhimurium, Synechocystis species, Thermotoga maritime, Treponema pallidum, Ureaplasma urealyticum, Vibrio cholerae, Xylella fastidiosa, Yersinia pestis and Enterococcus facaelis.

5. The method of Paragraph 1, wherein the target prokaryotic organism is selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis and any species falling within the genera of any of the above species.

6. The method of Paragraph 1, wherein the target prokaryotic organism is a Gram positive bacterium.

7. The method of Paragraph 1, wherein the target prokaryotic organism is selected from the group consisting of Bacillus anthracis, Clostridium acetobutylicum, Clostridium botulinum, Clostridium difficile, Clostridium perfringens, Corynebacterium diptheriae, Enterococcus faecalis, Enterococcus faecium, Listeria monocytogenes, Mycobacterium avium, Mycobacterium bovis, Mycobacterium leprae, Mycobacterium tuberculosis, Nocardia asteroides, Staphylococcus aureus, Staphylococcus epidermidis, Staphylococcus haemolyticus, Streptococcus pneumoniae, Streptococcus mutans, Streptococcus pyogenes and any species falling within the genera of any of the above species.

8. The method of Paragraph 1, wherein the target prokaryotic organism is Staphylococcus aureus.

9. The method of Paragraph 8, wherein said Staphylococcus aureus is strain Mu50.

10. The method of Paragraph 1, wherein segregation of the genes into the plurality of bins further comprises identifying monocistronic operons by identifying genes where both the 5′ and 3′ flanking genes are oppositely oriented relative to the selected gene.

11. The method of Paragraph 10, wherein monocistronic operons are excluded from further analysis.

12. The method of Paragraph 1, wherein segregation of the genes into the plurality of bins further comprises identifying genes having at least one 5′ or 3′ flanking gene in the same orientation.

13. The method of Paragraph 12, wherein only putative polycistronic operons are subjected to composite operon prediction analysis.

14. The method of Paragraph 1, wherein the results from the at least one operon prediction method are evaluated with respect to gene pair orientation and according to a pre-selected scoring criteria to determine the confidence score value associated with each gene pair.

15. The method of Paragraph 1, wherein the at least one operon prediction method comprises an intergenic distance analysis.

16. The method of Paragraph 1, wherein the at least one operon prediction method comprises a pairwise assessment of gene conservation.

17. The method of Paragraph 16, wherein the pairwise assessment of gene conservation comprises identifying gene pairs associated with the target organism that are conserved with respect to at least one comparison organism.

18. The method of Paragraph 16, wherein the pairwise assessment of gene conservation further comprises identifying conserved gene pairs associated with the target organism that are adjacent and oriented in the same direction and further possess an adjacent gene pair homolog in at least one comparison organism having a similar orientation.

19. The method of Paragraph 18, further comprising ordering the plurality of conserved gene pairs to reflect their relative coordinates with respect to the target prokaryotic organism genome.

20. The method of Paragraph 1, wherein the at least one operon prediction method comprises a conserved gene cluster analysis.

21. The method of Paragraph 20, wherein the conserved gene cluster analysis further comprises the steps of:
(a) identifying gene pairs contained in the bins for the target organism having an intergenic distance below a selected intergenic distance threshold;
(b) comparing the sequence and location of identified target organism gene pairs with homologous gene pairs from at least one comparison organism to identify conserved gene pairs between the target organism and the comparison organism;
(c) repeating steps (a)-(b) for each remaining comparison organism; and
(d) evaluating the conserved gene pairs between the target organism and each comparison organism with respect to one another to identify conserved gene clusters.

22. The method of Paragraph 21, wherein the selected intergenic distance threshold is approximately 300 bp.

23. The method of Paragraph 21, wherein the conserved gene cluster analysis further comprises identifying orthologs representative of conserved order between genes in each gene cluster.

24. The method of Paragraph 21, wherein orthologs are identified using a BLASTP or BLASTN sequence evaluation application.

25. The method of Paragraph 20, wherein the conserved gene cluster analysis further comprises pairwise gene assessment in the target organism relative to homologous gene pairs in at least one comparison organism to identify gene pairs whose sequence and location are conserved across the genomes of the at least one comparison organisms.

26. The method of Paragraph 1, wherein the at least one operon prediction method comprises a transcriptional terminator analysis.

27. The method of Paragraph 26, wherein the transcriptional terminator analysis further comprises:
extracting a portion of the sequence on both sides of a stop codon associated with each gene;
evaluating the portion of the sequence on both sides of the stop codon of each gene to identify prospective transcriptional terminators; and
associating a transcriptional terminator value with each gene on the basis of identified prospective transcriptional terminators.

28. The method of Paragraph 27, wherein the portion of the sequence on both sides of the stop codon associated with each gene is evaluated to identify prospective prokaryotic factor-independent transcription terminators.

29. The method of Paragraph 28, wherein a region encompassing approximately −20 to approximately +200 bases around the stop codon associated with each gene is evaluated for transcriptional terminators.

30. The method of Paragraph 28, wherein the prokaryotic factor-independent transcriptional terminators are identified using a third-party software application.

31. The method of Paragraph 30, wherein the third-party software application used to identify the prokaryotic factor-independent transcriptional terminators is Terminator, part of the GCG Wisconsin Package of Programs.

32. The method of Paragraph 31, wherein putative terminators with an S-value greater than approximately 0 are identified as prokaryotic factor-independent transcriptional terminators.

33. The method of Paragraph 1, wherein the confidence score reflects a numerical assessment of the likelihood that the genes of the gene pair reside in the same operon.

34. The method of Paragraph 33, wherein the confidence score is used to further classify each gene pair as being part of distinct operons or being part of the same operon.

35. The method of Paragraph 1, wherein a numerical value is associated with the confidence score and reflects the likelihood of selected genes residing in the same operon, wherein the numerical value is selected from the group consisting of:
(a) a first numerical value which indicates that the genes of the gene pair are unlikely to be in the same operon if any of the following three criteria are met:
   (i) the selected genes are in different orientations;
   (ii) the intergenic distance between the selected genes is greater than approximately 300 bp; or
   (iii) the intergenic distance between the selected genes is greater than 100 bp and there are no conserved gene clusters;
(b) a second numerical value which indicates that the selected genes might be in the same operon but the confidence is low if:
   (i) the selected genes have an intergenic distance greater than approximately 60 bp,
   (ii) the selected genes are conserved over approximately five or fewer comparison organisms, and
   (iii) a predicted transcriptional terminator exists between the selected genes;
(c) a third numerical value which indicates that the selected genes are probably in the same operon if any of the following criteria are met:
   (i) the selected genes are conserved in at least approximately 10 comparison organisms;
   (ii) the intergenic distance between the selected genes is approximately less than or equal to 30 bp;
   or if at least two of the following requirements are met:
   (i) the intergenic distance between the selected genes is approximately less than or equal to 50 bp;
   (ii) no predicted transcriptional terminators exist; or
   (iii) the selected genes are conserved in approximately greater or equal to 5 comparison organisms but less than approximately 10 comparison organisms;
(d) a fourth numerical value which indicates that the selected genes are likely to reside in the same operon if the genes do not meet any of the above requirements.

36. A method for predicting operons comprising:
(a) identifying consecutive genes within at least a portion of the genome of a target organism;
(b) determining each gene's orientation in the genome relative to its flanking genes;
(c) segregating consecutive genes in the same orientation into the same bin;
(d) identifying the intergenic distance between each consecutive gene and its flanking genes;
(e) identifying orthologous genes in at least one comparison organism that are in the same order and orientation as the corresponding genes in the target organism;
(f) predicting transcriptional terminators in the sequence flanking the stop codon of each gene;
(g) predicting the likelihood that two consecutive genes in the same bin are in the same operon by performing at least one of steps (h)-(l) below:
(h) predicting that two selected consecutive genes in the same bin are unlikely to be in the same operon if any of the following three criteria are met:
   (i) the two consecutive genes are in different orientations;
   (ii) the intergenic distance between the two consecutive genes is greater than approximately 300 bp; or
   (iii) the intergenic distance between the two consecutive genes is greater than 100 bp and there are no conserved gene clusters;
(i) predicting that two consecutive genes in a multi-gene bin have a low likelihood of being in the same operon if:
   (i) the two consecutive genes have an intergenic distance greater than approximately 60 bp,
   (ii) the two consecutive genes are conserved over approximately 5 or less comparison organisms, and
   (iii) a predicted transcriptional terminator exists between the two consecutive genes;
(j) predicting that two consecutive genes are probably in the same operon if any of the following criteria are met:
   (i) the two consecutive genes are conserved in at least approximately 10 comparison organisms;
   (ii) the intergenic distance between the two consecutive genes is approximately less than or equal to 30 bp;
(k) predicting that two consecutive genes are probably in the same operon if at least two of the following requirements were met:
   (i) the intergenic distance between the two consecutive genes is approximately less than or equal to 50 bp;
   (ii) no predicted transcriptional terminators between the two consecutive genes exist; or
   (iii) the two consecutive genes are conserved in approximately greater than or equal to 5 comparison organisms but less than approximately 10 comparison organisms;
(l) predicting that two consecutive genes are probably in the same operon if the consecutive genes do not meet any of the above requirements.

37. A computer-based system for predicting operons within a target prokaryotic organism, the system comprising:
a database for storing information describing a plurality of genes relating to at least a portion of the target organism's genome;
a program which performs the operations of;
   identifying consecutive genes from the plurality of genes stored in the database;
   determining the orientation of each consecutive gene relative to its flanking genes;
   segregating the consecutive genes into a plurality of bins on the basis of their orientation such that consecutive genes in the same orientation are grouped into the same bin;
   performing a composite operon prediction analysis comprising pairing each consecutive gene within a selected bin with its respective flanking genes and associating a confidence score with each gene pair, wherein the confidence score reflects the likelihood that a selected gene pair resides in the same operon using the criteria of orientation and the application of at least one operon prediction method;
   determining operon boundaries by identifying gene pairs having confidence scores that fall below a selected threshold; and
   associating genes contained between operon boundaries as operons.

38. The system of Paragraph 37 wherein the target prokaryotic organism is Bacillus subtilis.

39. The system of Paragraph 37, wherein the target prokaryotic organism is Escherichia coli.

40. The system of Paragraph 37, wherein the target prokaryotic organism is selected from the group consisting of Aquifex aeolicus, Borrelia burgdorferi, Bacillus halodurans, Buchnera species, Clostridium acetobutylicum, Caulobacter crescentus, Campylobacter jejun, Chlamydia pneumoniae, Chlamydia trachomatis, Haemophilus influenzae, Helicobacter pylori, Listeria monocytogenes, Listeria innocua, Lactococcus lactis, Mycoplasma genitalium, Mycobacterium leprae, Mycoplasma pneumoniae, Mycoplasma pulmonis, Mycobacterium tuberculosis, Neisseria meningitides, Pseudomonas aeruginosa, Pasteurella multocida, Rickettsia conorii, Rickettsia prowazekii, Sinorhizobium meliloti, Streptococcus pneumoniae, Streptococcus pyogenes, Salmonella typhi, Salmonella typhimurium, Synechocystis species, Thermotoga maritime, Treponema pallidum, Ureaplasma urealyticum, Vibrio cholerae, Xylella fastidiosa, Yersinia pestis and Enterococcus facaelis.

41. The system of Paragraph 37, wherein the target prokaryotic organism is selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis and any species falling within the genera of any of the above species.

42. The system of Paragraph 37, wherein the target prokaryotic organism is a Gram positive bacterium.

43. The system of Paragraph 37, wherein the target prokaryotic organism is selected from the group consisting of Bacillus anthracis, Clostridium acetobutylicum, Clostridium botulinum, Clostridium difficile, Clostridium perfringens, Corynebacterium diptheriae, Enterococcus faecalis, Enterococcus faecium, Listeria monocytogenes, Mycobacterium avium, Mycobacterium bovis, Mycobacterium leprae, Mycobacterium tuberculosis, Nocardia asteroides, Staphylococcus aureus, Staphylococcus epidermidis, Staphylococcus haemolyticus, Streptococcus pneumoniae, Streptococcus mutans, Streptococcus pyogenes and any species falling within the genera of any of the above species.

44. The system of Paragraph 37, wherein the target prokaryotic organism is Staphylococcus aureus.

45. The system of Paragraph 44, wherein said Staphylococcus aureus is strain Mu50.

46. The system of Paragraph 37, wherein the program operations of segregating the consecutive genes into the plurality of bins further comprises identifying monocistronic operons by identifying genes where both the 5′ and 3′ flanking genes are oppositely oriented relative to the selected gene.

47. The system of Paragraph 46, wherein the program operations further comprise excluding monocistronic operons from further analysis.

48. The system of Paragraph 37, wherein the program operations of segregating the consecutive genes into the plurality of bins further comprises identifying genes having at least one 5′ or 3′ flanking gene in the same orientation.

49. The system of Paragraph 48, wherein the program further performs the operation of selectively subjecting putative polycistronic operons to composite operon prediction analysis.

50. The system of Paragraph 37, wherein the program operations further comprises evaluating the results from the at least one operon prediction method with respect to gene pair orientation and according to a pre-selected scoring criteria to determine the confidence score value associated with each gene pair.

51. The system of Paragraph 37, wherein the program operations of application of the at least one operon prediction method comprises performing an intergenic distance analysis.

52. The system of Paragraph 37, wherein the program operations of application of the at least one operon prediction method comprises performing a pairwise assessment of gene conservation.

53. The system of Paragraph 52, wherein the program operations of performing the pairwise assessment of gene conservation comprises identifying gene pairs associated with the target organism that are conserved with respect to at least one comparison organism.

54. The system of Paragraph 52, wherein the program operations of performing the pairwise assessment of gene conservation further comprises identifying conserved gene pairs associated with the target organism that are adjacent and oriented in the same direction and further possess an adjacent gene pair homolog in at least one comparison organism having a similar orientation.

55. The system of Paragraph 54, the program operations of performing the pairwise assessment of gene conservation further comprises ordering the plurality of conserved gene pairs to reflect their relative coordinates with respect to the target prokaryotic organism genome.

56. The system of Paragraph 37, wherein the database further stores information describing a plurality of genes relating to at least one other comparison organism and at least one operon prediction method performed by the program comprises an a conservation analysis wherein the program further performs the operations of:
determining an intergenic distance distribution across a selected number of gene pairs stored in the database for the target organism;
determining an intergenic distance distribution for genes stored in the database relating to the at least one other comparison organism;
comparing the intergenic distance distribution across the selected number of gene pairs with that of the comparison organism; and
associating a conservation value with each gene pair on the basis of identified similarities and differences in intergenic distance distribution between the target organism and the at least one other comparison organism.

57. The system of Paragraph 37, wherein the database further stores information describing a plurality of genes relating to at least one other comparison organism and at least one operon prediction method performed by the program comprises a conserved gene cluster analysis wherein the program further performs the operations of:
(a) identifying target organism gene pairs having an intergenic distance below a selected intergenic distance threshold;
(b) comparing the sequence and location of identified target organism gene pairs with homologous gene pairs from at least one comparison organism to identify conserved gene pairs between the target organism and the comparison organism;
(c) repeating steps (a)-(b) for each remaining comparison organism; and
(d) evaluating the conserved gene pairs between the target organism and each comparison organism with respect to one another to identify conserved gene clusters.

58. The system of Paragraph 57, wherein the selected intergenic distance threshold is approximately 300 bp.

59. The system of Paragraph 57, wherein the conserved gene cluster analysis further comprises identifying orthologs representative of conserved order between genes in each gene cluster.

60. The system of Paragraph 59, wherein orthologs are identified using a BLASTP or BLASTN sequence evaluation application.

61. The system of Paragraph 57, wherein the conserved gene cluster analysis further comprises pairwise gene assessment in the target organism relative to homologous gene pairs in at least one comparison organism to identify gene pairs whose sequence and location are conserved across the genomes of the at least one comparison organisms.

62. The system of Paragraph 37, wherein the at least one operon prediction method performed by the program comprises a transcriptional terminator analysis wherein the program further performs the operations of:
extracting a portion of the sequence on both sides of a stop codon associated with each gene from the database;
evaluating the portion of the sequence on both sides of the stop codon of each gene to identify prospective transcriptional terminators; and
associating a transcriptional terminator value with each gene pair on the basis of identified prospective transcriptional terminators.

63. The system of Paragraph 37, wherein the program further performs the operations of:
associating a numerical value with the confidence score which reflects the likelihood that the selected genes reside in the some operon, wherein said numerical value is selected from the group consisting of:
(a) a first numerical value which indicates that the genes of the gene pair are unlikely to be in the same operon if any of the following three criteria are met:
   (i) the selected genes are in different orientations;
   (ii) the intergenic distance between the selected genes is greater than approximately 300 bp; or
   (iii) the intergenic distance between the selected genes is greater than 100 bp and there are no conserved gene clusters;
(b) a second numerical value which indicates that the selected genes might be in the same operon but the confidence is low if:
   (i) the selected genes have an intergenic distance greater than approximately 60 bp,
   (ii) the selected genes are conserved over approximately five or fewer comparison organisms, and
   (iii) a predicted transcriptional terminator exists between the selected genes;
(c) a third numerical value which indicates that the selected genes are probably in the same operon if any of the following criteria are met:
   (i) the selected genes are conserved in at least approximately 10 comparison organisms;
   (ii) the intergenic distance between the selected genes is approximately less than or equal to 30 bp;
   or if at least two of the following requirements are met:
   (i) the intergenic distance between the selected genes is approximately less than or equal to 50 bp;
   (ii) no predicted transcriptional terminators exist; or
   (iii) the selected genes are conserved in approximately greater or equal to 5 comparison organisms but less than approximately 10 comparison organisms;
(d) a fourth numerical value which indicates that the selected genes are likely to reside in the same operon if the genes do not meet any of the above requirements.

64. A computer readable medium having stored thereon instructions which cause a general purpose computer to perform the steps of:
identifying consecutive genes within at least a portion of a target prokaryotic organism's genome;
determining each gene's orientation in the genome relative to its flanking genes;
segregating the genes into a plurality of bins on the basis of their orientation such that consecutive genes in the same orientation are grouped into the same bin;
performing a composite operon prediction analysis comprising pairing each gene within a selected bin with its respective flanking genes and associating a confidence score with each gene pair, wherein the confidence score reflects the likelihood that a selected gene pair resides in the same operon using the criteria of orientation and the application of at least one operon prediction method;
determining operon boundaries by identifying gene pairs having confidence scores that fall below a selected threshold; and
associating genes contained between operon boundaries as putative operons.

65. The computer readable medium of Paragraph 64, wherein the steps performed by the computer operate on the target prokaryotic organism Bacillus subtilis.

66. The computer readable medium of Paragraph 64, wherein the steps performed by the computer operate on the target prokaryotic organism Escherichia coli.

67. The computer readable medium of Paragraph 64, wherein the steps performed by the computer operate on a target prokaryotic organism selected from the group consisting of Aquifex aeolicus, Borrelia burgdorferi, Bacillus halodurans, Buchnera species, Clostridium acetobutylicum, Caulobacter crescentus, Campylobacter jejun, Chlamydia pneumoniae, Chlamydia trachomatis, Haemophilus influenzae, Helicobacter pylori, Listeria monocytogenes, Listeria innocua, Lactococcus lactis, Mycoplasma genitalium, Mycobacterium leprae, Mycoplasma pneumoniae, Mycoplasma pulmonis, Mycobacterium tuberculosis, Neisseria meningitides, Pseudomonas aeruginosa, Pasteurella multocida, Rickettsia conorii, Rickettsia prowazekii, Sinorhizobium meliloti, Streptococcus pneumoniae, Streptococcus pyogenes, Salmonella typhi, Salmonella typhimurium, Synechocystis species, Thermotoga maritime, Treponema pallidum, Ureaplasma urealyticum, Vibrio cholerae, Xylella fastidiosa, Yersinia pestis and Enterococcus facaelis.

68. The computer readable medium of Paragraph 64, wherein the steps performed by the computer operate on a target prokaryotic organism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis and any species falling within the genera of any of the above species.

69. The computer readable medium of Paragraph 64, wherein the steps performed by the computer operate on a target prokaryotic organism that is a Gram positive bacterium.

70. The computer readable medium of Paragraph 64, wherein the steps performed by the computer operate on a target prokaryotic organism selected from the group consisting of Bacillus anthracis, Clostridium acetobutylicum, Clostridium botulinum, Clostridium difficile, Clostridium perfringens, Corynebacterium diptheriae, Enterococcus faecalis, Enterococcus faecium, Listeria monocytogenes, Mycobacterium avium, Mycobacterium bovis, Mycobacterium leprae, Mycobacterium tuberculosis, Nocardia asteroides, Staphylococcus aureus, Staphylococcus epidermidis, Staphylococcus haemolyticus, Streptococcus pneumoniae, Streptococcus mutans, Streptococcus pyogenes and any species falling within the genera of any of the above species.

71. The computer readable medium of Paragraph 64, wherein the steps performed by the computer operate on a target prokaryotic organism that is Staphylococcus aureus.

72. The computer readable medium of Paragraph 71, wherein said Staphylococcus aureus is strain Mu50.

73. The computer readable medium of Paragraph 64, wherein segregation of the genes into the plurality of bins further comprises identifying putative polycistronic operons comprising a gene having at least one 5′ or 3′ flanking gene in the same orientation.

74. The computer readable medium of Paragraph 64, wherein the results from the at least one operon prediction method are evaluated with respect to gene pair orientation and according to a pre-selected scoring criteria to determine the confidence score value associated with each gene pair.

75. The computer readable medium of Paragraph 64, wherein the at least one operon prediction method comprises a pairwise assessment of gene conservation.

76. The computer readable medium of Paragraph 75, wherein the pairwise assessment of gene conservation comprises identifying gene pairs associated with the target organism that are conserved with respect to at least one comparison organism.

77. The computer readable medium of Paragraph 75, wherein the pairwise assessment of gene conservation further comprises identifying conserved gene pairs associated with the target organism that are adjacent and oriented in the same direction and further possess an adjacent gene pair homolog in at least one comparison organism having a similar orientation.

78. The computer readable medium of Paragraph 64, wherein the at least one operon prediction method comprises a conserved gene cluster analysis.

79. The computer readable medium of Paragraph 78, wherein the conserved gene cluster analysis further comprises the steps of:
(a) identifying gene pairs contained in the bins for the target organism having an intergenic distance below a selected intergenic distance threshold;
(b) comparing the sequence and location of identified target organism gene pairs with homologous gene pairs from at least one comparison organism to identify conserved gene pairs between the target organism and the comparison organism;
(c) repeating steps (a)-(b) for each remaining comparison organism; and
(d) evaluating the conserved gene pairs between the target organism and each comparison organism with respect to one another to identify conserved gene clusters.

80. The method of Paragraph 79, wherein the selected intergenic distance threshold is approximately 300 bp.

81. The method of Paragraph 79, wherein the conserved gene cluster analysis further comprises pairwise gene assessment in the target organism relative to homologous gene pairs in at least one comparison organism to identify gene pairs whose sequence and location are conserved across the genomes of the at least one comparison organisms.

82. The computer readable medium of Paragraph 64, wherein the at least one operon prediction method comprises a transcriptional terminator analysis.

83. The computer readable medium of Paragraph 82, wherein the transcriptional terminator analysis further comprises:
extracting a portion of the sequence on both sides of a stop codon associated with each gene;
evaluating the portion of the sequence on both sides of the stop codon of each gene to identify prospective transcriptional terminators; and
associating a transcriptional terminator value with each gene on the basis of identified prospective transcriptional terminators.

84. The computer readable medium of Paragraph 83, wherein the portion of the sequence on both sides of the stop codon associated with each gene is evaluated to identify prospective prokaryotic factor-independent transcription terminators.

85. The computer readable medium of Paragraph 64, wherein a numerical value is associated with the confidence score and reflects the likelihood of selected genes residing in the same operon, wherein the numerical value is selected from the group consisting of:
(a) a first numerical value which indicates that the genes of the gene pair are unlikely to be in the same operon if any of the following three criteria are met:
   (i) the selected genes are in different orientations;
   (ii) the intergenic distance between the selected genes is greater than approximately 300 bp; or
   (iii) the intergenic distance between the selected genes is greater than 100 bp and there are no conserved gene clusters;
(b) a second numerical value which indicates that the selected genes might be in the same operon but the confidence is low if:
   (i) the selected genes have an intergenic distance greater than approximately 60 bp,
   (ii) the selected genes are conserved over approximately five or fewer comparison organisms, and
   (iii) a predicted transcriptional terminator exists between the selected genes;
(c) a third numerical value which indicates that the selected genes are probably in the same operon if any of the following criteria are met:
   (i) the selected genes are conserved in at least approximately 10 comparison organisms;
   (ii) the intergenic distance between the selected genes is approximately less than or equal to 30 bp;
   or if at least two of the following requirements are met:
   (i) the intergenic distance between the selected genes is approximately less than or equal to 50 bp;
   (ii) no predicted transcriptional terminators exist; or
   (iii) the selected genes are conserved in approximately greater or equal to 5 comparison organisms but less than approximately 10 comparison organisms;
(d) a fourth numerical value which indicates that the selected genes are likely to reside in the same operon if the genes do not meet any of the above requirements.

86. A computer readable medium having stored thereon instructions that cause a general purpose computer to perform operations used to predict operons for a target prokaryotic organism, the operations comprising:
(a) identifying consecutive genes within at least a portion of the genome of a target organism;
(b) determining each gene's orientation in the genome relative to its flanking genes;
(c) segregating consecutive genes in the same orientation into the same bin;
(d) identifying the intergenic distance between each consecutive gene and its flanking genes;
(e) identifying orthologous genes in at least one comparison organism that are in the same order and orientation as the corresponding genes in the target organism;
(f) predicting transcriptional terminators in the sequence flanking the stop codon of each gene;
(g) predicting the likelihood that two consecutive genes in the same bin are in the same operon by performing at least one of steps (h)-(l) below:
(h) predicting that two selected consecutive genes in the same bin are unlikely to be in the same operon if any of the following three criteria are met:
   (i) the two consecutive genes are in different orientations;
   (ii) the intergenic distance between the two consecutive genes is greater than approximately 300 bp; or
   (iii) the intergenic distance between the two consecutive genes is greater than 100 bp and there are no conserved gene clusters;
(i) predicting that two consecutive genes in a multi-gene bin have a low likelihood of being in the same operon if:
   (i) the two consecutive genes have an intergenic distance greater than approximately 60 bp,
   (ii) the two consecutive genes are conserved over approximately 5 or fewer comparison organisms, and
   (iii) a predicted transcriptional terminator exists between the two consecutive genes;
(j) predicting that two consecutive genes are probably in the same operon if any of the following criteria are met:
   (i) the two consecutive genes are conserved in at least approximately 10 comparison organisms;
   (ii) the intergenic distance between the two consecutive genes is approximately less than or equal to 30 bp;
(k) predicting that two consecutive genes are probably in the same operon if at least two of the following requirements were met:
   (i) the intergenic distance between the two consecutive genes is approximately less than or equal to 50 bp;
   (ii) no predicted transcriptional terminators between the two consecutive genes exist; or
   (iii) the two consecutive genes are conserved in approximately greater than or equal to 5 comparison organisms but less than approximately 10 comparison organisms;
(l) predicting that two consecutive genes are probably in the same operon if the consecutive genes do not meet any of the above requirements.

87. A method of inhibiting the proliferation of a microorganism, said method comprising:
identifying a gene that is required for proliferation;
using the method of Paragraph 1 to identify an operon comprising said gene; and
contacting a microorganism with an antisense nucleic acid complementary to at least a portion of said operon.

88. A method for screening a candidate compound for the ability to reduce cellular proliferation, said method comprising the steps of:
(a) identifying a gene that is required for proliferation;
(b) using the method of Paragraph 1 to identify an operon comprising said gene;
(c) sensitizing a cell by providing said cell with a sublethal level of an antisense nucleic acid complementary to at least a portion of said operon;
(d) contacting said sensitized cell with a compound; and
(e) determining the degree to which said compound inhibits proliferation of said sensitized cell relative to a nonsensitized cell or a cell which is less sensitive to said compound than said sensitized cell.

89. A vector comprising a promoter operably linked to an operon comprising a nucleotide sequence selected from the group consisting of SEQ ID NOs: 1-194.

90. The vector of Paragraph 89, wherein said promoter is active in a microorganism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis and any species falling within the genera of any of the above species.

91. The vector of Paragraph 89, wherein said promoter is active in a microorganism selected from the group consisting of Bacillus anthracis, Clostridium acetobutylicum, Clostridium botulinum, Clostridium difficile, Clostridium perfringens, Corynebacterium diptheriae, Enterococcus faecalis, Enterococcus faecium, Listeria monocytogenes, Mycobacterium avium, Mycobacterium bovis, Mycobacterium leprae, Mycobacterium tuberculosis, Nocardia asteroides, Staphylococcus aureus, Staphylococcus epidermidis, Staphylococcus haemolyticus, Streptococcus pneumoniae, Streptococcus mutans, Streptococcus pyogenes and any species falling within the genera of any of the above species.

92. The vector of Paragraph 89, wherein said promoter is active in Staphylococcus aureus.

93. The vector of Paragraph 92, wherein said Staphylococcus aureus is strain Mu50.

94. The vector of Paragraph 89, wherein said promoter is XylT5.

95. The vector of Paragraph 89, wherein said operon is operably linked to said promoter in an antisense orientation.

96. A vector comprising a promoter operably linked to an operon consisting essentially of a nucleotide sequence selected from the group consisting of SEQ ID NOs: 1-194.

97. The vector of Paragraph 96, wherein said operon is operably linked to said promoter in an antisense orientation.

98. A host cell comprising a vector which comprises a promoter operably linked to an operon comprising a nucleic acid selected from the group consisting of SEQ ID NOs: 1-194.

99. A host cell comprising a vector which comprises a promoter operably linked to an operon consisting essentially of a nucleic acid selected from the group consisting of SEQ ID NOs: 1-194.

100. A method for inhibiting the proliferation of a microorganism, said method comprising contacting a cell with an antisense nucleic acid complementary to at least a portion of an operon required for proliferation, wherein said operon comprises a nucleotide sequence selected from the group consisting of SEQ ID NOs: 1-194, and wherein said antisense nucleic acid is not complementary to one or more of SEQ ID NOs: 201-550 or a portion thereof.

101. The method of Paragraph 100, wherein said operon comprises at least one gene required for proliferation.

102. The method of Paragraph 101, wherein said antisense nucleic acid is complementary to at least a portion of said at least one gene required for proliferation.

103. The method of Paragraph 100, wherein said operon comprises at least one gene that is not required for proliferation.

104. The method of Paragraph 103, wherein said antisense nucleic acid is complementary to at least a portion of said at least one gene not required for proliferation.

105. The method of Paragraph 100, wherein said operon comprises at least one nucleic acid sequence selected from the group consisting of a 5′ untranslated sequence, an intergenic sequence, and a 3′ untranslated sequence.

106. The method of Paragraph 105, where said antisense nucleic acid is complementary to at least one nucleic acid sequence selected from the group consisting of a 5′ untranslated sequence, an intergenic sequence, and a 3′ untranslated sequence.

107. The method of Paragraph 100, wherein said cell is contacted with said antisense nucleic acid by expressing said antisense nucleic acid from an regulatable promoter.

108. The method of Paragraph 100, wherein said cell is contacted with said antisense nucleic acid by expressing said antisense nucleic acid from an inducible promoter.

109. The method of Paragraph 100, wherein said cell is contacted with said antisense nucleic acid by expressing said antisense nucleic acid ectopically.

110. The method of Paragraph 100, wherein said cell is contacted with said antisense nucleic acid by expressing said antisense nucleic acid from a chromosome of said cell.

111. The method of Paragraph 100, wherein said cell is Staphylococcus aureus.

112. The method of Paragraph 111, wherein said Staphylococcus aureus is strain Mu50.

113. A method for inhibiting the proliferation of a microorganism, said method comprising contacting a cell with an antisense nucleic acid complementary to at least a portion of an operon required for proliferation, wherein said operon comprises at least one gene which has at least 70% nucleotide sequence identity, as determined by BLASTN version 2.0 with default parameters, to a gene contained in an operon comprising a nucleotide sequence selected from the group consisting of SEQ ID NOs: 1-194, and wherein said antisense nucleic acid is not complementary to one or more of SEQ ID NOs: 201-550 or portions thereof.

114. The method of Paragraph 113, wherein said operon comprises at least one gene required for proliferation.

115. The method of Paragraph 114, wherein said antisense nucleic acid is complementary to at least a portion of said at least one gene required for proliferation.

116. The method of Paragraph 113, wherein said operon comprises at least one gene that is not required for proliferation.

117. The method of Paragraph 116, wherein said antisense nucleic acid is complementary to at least a portion of said at least one gene not required for proliferation.

118. The method of Paragraph 113, wherein said operon comprises at least one nucleic acid sequence selected from the group consisting of a 5′ untranslated sequence, an intergenic sequence, and a 3′ untranslated sequence.

119. The method of Paragraph 118, where said antisense nucleic acid is complementary to at least one nucleic acid sequence selected from the group consisting of a 5′ untranslated sequence, an intergenic sequence, and a 3′ untranslated sequence.

120. The method of Paragraph 113, wherein said cell is contacted with said antisense nucleic acid by expressing said antisense nucleic acid from an regulatable promoter.

121. The method of Paragraph 113, wherein said cell is contacted with said antisense nucleic acid by expressing said antisense nucleic acid from an inducible promoter.

122. The method of Paragraph 113, wherein said cell is contacted with said antisense nucleic acid by expressing said antisense nucleic acid ectopically.

123. The method of Paragraph 113, wherein said cell is contacted with said antisense nucleic acid by expressing said antisense nucleic acid from a chromosome of said cell.

124. The method of Paragraph 113, wherein said cell is selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis and any species falling within the genera of any of the above species.

125. The method of Paragraph 113, wherein said cell is a Gram positive bacterium.

126. The method of Paragraph 113, wherein said cell is selected from the group consisting of Bacillus anthracis, Clostridium acetobutylicum, Clostridium botulinum, Clostridium difficile, Clostridium perfringens, Corynebacterium diptheriae, Enterococcus faecalis, Enterococcus faecium, Listeria monocytogenes, Mycobacterium avium, Mycobacterium bovis, Mycobacterium leprae, Mycobacterium tuberculosis, Nocardia asteroides, Staphylococcus aureus, Staphylococcus epidermidis, Staphylococcus haemolyticus, Streptococcus pneumoniae, Streptococcus mutans, Streptococcus pyogenes and any species falling within the genera of any of the above species.

127. The method of Paragraph 113, wherein said cell is Staphylococcus aureus.

128. The method of Paragraph 127, wherein said Staphylococcus aureus is strain Mu50.

129. A method for inhibiting the activity or expression of a gene product required for proliferation, said method comprising contacting a cell with an antisense nucleic acid complementary to at least a portion of an operon comprising a gene encoding a gene product required for proliferation wherein said operon comprises a nucleotide sequence selected from the group consisting of SEQ ID NOs: 1-194, and wherein said antisense nucleic acid is not complementary to one or more of SEQ ID NOs: 201-550 or portions thereof.

130. The method of Paragraph 129, wherein said gene is selected from the group consisting of SEQ ID NOs: 201-550.

131. The method of Paragraph 129, wherein said gene product is selected from the group consisting of SEQ ID NOs: 551-824.

132. A method for inhibiting the activity or expression of a gene product required for proliferation, said method comprising contacting a cell with an antisense nucleic acid complementary to at least a portion of an operon comprising a gene which encodes a gene product required for proliferation wherein said gene has at least 70% nucleotide sequence identity, as determined using BLASTN 2.0 with default parameters, to a gene contained in an operon comprising a nucleotide sequence selected from the group consisting of SEQ ID NOs: 1-194, and wherein said antisense nucleic acid is not complementary to one or more of SEQ ID NOs: 201-550 or portions thereof.

133. The method of Paragraph 132, wherein said gene is selected from the group consisting of SEQ ID NOs: 201-550.

134. The method of Paragraph 132, wherein said gene product is selected from the group consisting of SEQ ID NOs: 551-824.

135. A method for inhibiting the activity or expression of a gene product required for proliferation, said method comprising contacting a cell with an antisense nucleic acid complementary to at least a portion of an operon comprising a gene which encodes a gene product required for proliferation wherein said gene product has at least 25% amino acid identity, as determined using FASTA version 3.0t78 with default parameters, to a gene product encoded by a gene contained in an operon selected from the group consisting of SEQ ID NOs: 1-194, and wherein said antisense nucleic acid is not complementary to one or more of SEQ ID NOs: 201-550 or portions thereof.

136. The method of Paragraph 135, wherein said gene is selected from the group consisting of SEQ ID NOs: 201-550.

137. The method of Paragraph 135, wherein said gene product is selected from the group consisting of SEQ ID NOs: 551-824.

138. A method for screening a candidate compound for the ability to reduce cellular proliferation, said method comprising the steps of:
(a) sensitizing a cell by providing said cell with a sublethal level of an antisense nucleic acid complementary to at least a portion of an operon required for proliferation, wherein said operon comprises a nucleotide sequence selected from the group consisting of SEQ ID NOs: 1-194, and wherein said antisense nucleic acid is not complementary to one or more of SEQ ID NOs: 201-550 or portions thereof;
(b) contacting said sensitized cell with a compound; and
(c) determining the degree to which said compound inhibits proliferation of said sensitized cell relative to a nonsensitized cell or a cell which is less sensitive to said compound than said sensitized cell.

139. The method of Paragraph 138, wherein said determining step comprises determining whether said compound inhibits the growth of said sensitized cell to a greater extent than said compound inhibits the growth of a nonsensitized cell or a cell which is less sensitive to said compound than said sensitized cell.

140. The method of Paragraph 138, wherein said operon comprises at least one gene required for proliferation.

141. The method of Paragraph 140, wherein said antisense nucleic acid is complementary to at least a portion of said at least one gene required for proliferation.

142. The method of Paragraph 138, wherein said operon comprises at least one gene that is not required for proliferation.

143. The method of Paragraph 142, wherein said antisense nucleic acid is complementary to at least a portion of said at least one gene not required for proliferation.

144. The method of Paragraph 138, wherein said operon comprises at least one nucleic acid sequence selected from the group consisting of a 5′ untranslated sequence, an intergenic sequence, and a 3′ untranslated sequence.

145. The method of Paragraph 144, where said antisense nucleic acid is complementary to at least one nucleic acid sequence selected from the group consisting of a 5′ untranslated sequence, an intergenic sequence, and a 3′ untranslated sequence.

146. The method of Paragraph 138, wherein said cell is contacted with said antisense nucleic acid by expressing said antisense nucleic acid from an regulatable promoter.

147. The method of Paragraph 138, wherein said cell is contacted with said antisense nucleic acid by expressing said antisense nucleic acid from an inducible promoter.

148. The method of Paragraph 138, wherein said cell is contacted with said antisense nucleic acid by expressing said antisense nucleic acid ectopically.

149. The method of Paragraph 138, wherein said cell is contacted with said antisense nucleic acid by expressing said antisense nucleic acid from a chromosome of said cell.

150. The method of Paragraph 138, wherein said cell is Staphylococcus aureus.

151. The method of Paragraph 150, wherein said Staphylococcus aureus is strain Mu50.

152. A method for screening a candidate compound for the ability to reduce cellular proliferation, said method comprising the steps of:
(a) sensitizing a cell by providing said cell with a sublethal level of an antisense nucleic acid complementary to at least a portion of an operon required for proliferation, wherein said operon comprises at least one gene which has at least 70% nucleotide sequence identity, as determined by BLASTN version 2.0 with default parameters, to a gene contained in an operon comprising a nucleotide sequence selected from the group consisting of SEQ ID NOs: 1-194, and wherein said antisense nucleic acid is not complementary to one or more of SEQ ID NOs: 201-550 or portions thereof;
(b) contacting said sensitized cell with a compound; and
(c) determining the degree to which said compound inhibits proliferation of said sensitized cell relative to a nonsensitized cell or a cell which is less sensitive to said compound than said sensitized cell.

153. The method of Paragraph 152, wherein said determining step comprises determining whether said compound inhibits the growth of said sensitized cell to a greater extent than said compound inhibits the growth of a nonsensitized cell or a cell which is less sensitive to said compound than said sensitized cell.

154. The method of Paragraph 152, wherein said operon comprises at least one gene required for proliferation.

155. The method of Paragraph 154, wherein said antisense nucleic acid is complementary to at least a portion of said at least one gene required for proliferation.

156. The method of Paragraph 152, wherein said operon comprises at least one gene that is not required for proliferation.

157. The method of Paragraph 156, wherein said antisense nucleic acid is complementary to at least a portion of said at least one gene not required for proliferation.

158. The method of Paragraph 152, wherein said operon comprises at least one nucleic acid sequence selected from the group consisting of a 5′ untranslated sequence, an intergenic sequence, and a 3′ untranslated sequence.

159. The method of Paragraph 158, where said antisense nucleic acid is complementary to at least one nucleic acid sequence selected from the group consisting of a 5′ untranslated sequence, an intergenic sequence, and a 3′ untranslated sequence.

160. The method of Paragraph 152, wherein said cell is contacted with said antisense nucleic acid by expressing said antisense nucleic acid from an regulatable promoter.

161. The method of Paragraph 152, wherein said cell is contacted with said antisense nucleic acid by expressing said antisense nucleic acid from an inducible promoter.

162. The method of Paragraph 152, wherein said cell is contacted with said antisense nucleic acid by expressing said antisense nucleic acid ectopically.

163. The method of Paragraph 152, wherein said cell is contacted with said antisense nucleic acid by expressing said antisense nucleic acid from a chromosome of said cell.

164. The method of Paragraph 152, wherein said cell is selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis and any species falling within the genera of any of the above species.

165. The method of Paragraph 152, wherein said cell is a Gram positive bacterium.

166. The method of Paragraph 152, wherein said cell is selected from the group consisting of Bacillus anthracis, Clostridium acetobutylicum, Clostridium botulinum, Clostridium difficile, Clostridium perfringens, Corynebacterium diptheriae, Enterococcus faecalis, Enterococcus faecium, Listeria monocytogenes, Mycobacterium avium, Mycobacterium bovis, Mycobacterium leprae, Mycobacterium tuberculosis, Nocardia asteroides, Staphylococcus aureus, Staphylococcus epidermidis, Staphylococcus haemolyticus, Streptococcus pneumoniae, Streptococcus mutans, Streptococcus pyogenes and any species falling within the genera of any of the above species.

167. The method of Paragraph 152, wherein said cell is Staphylococcus aureus.

168. The method of Paragraph 167, wherein said Staphylococcus aureus is strain Mu50.

169. A method for screening a candidate compound for the ability to reduce the activity or expression of a gene product required for proliferation, said method comprising the steps of:
(a) sensitizing a cell by providing said cell with a sublethal level of an antisense nucleic acid complementary to at least a portion of an operon comprising a gene encoding a gene product required for proliferation wherein said operon comprises a nucleotide sequence selected from the group consisting of SEQ ID NOs: 1-194, and wherein said antisense nucleic acid is not complementary to one or more of SEQ ID NOs: 201-550 or portions thereof;
(b) contacting said sensitized cell with a compound; and
(c) determining the degree to which said compound inhibits proliferation of said sensitized cell relative to a nonsensitized cell or a cell which is less sensitive to said compound than said sensitized cell.

170. The method of Paragraph 169, wherein said gene is selected from the group consisting of SEQ ID NOs: 201-550.

171. The method of Paragraph 169, wherein said gene product is selected from the group consisting of SEQ ID NOs: 551-824.

172. A method for screening a candidate compound for the ability to reduce the activity or expression of a gene product required for proliferation, said method comprising the steps of:
(a) sensitizing a cell by providing said cell with a sublethal level of an antisense nucleic acid complementary to at least a portion of an operon comprising a gene which encodes a gene product required for proliferation wherein said gene has at least 70% nucleotide sequence identity, as determined using BLASTN 2.0 with default parameters, to a gene contained in an operon comprising a nucleotide sequence selected from the group consisting of SEQ ID NOs: 1-194, and wherein said antisense nucleic acid is not complementary to one or more of SEQ ID NOs: 201-550 or portions thereof;
(b) contacting said sensitized cell with a compound; and
(c) determining the degree to which said compound inhibits proliferation of said sensitized cell relative to a nonsensitized cell or a cell which is less sensitive to said compound than said sensitized cell.

173. The method of Paragraph 172, wherein said gene is selected from the group consisting of SEQ ID NOs: 201-550.

174. The method of Paragraph 172, wherein said gene product is selected from the group consisting of SEQ ID NOs: 551-824.

175. A method for screening a candidate compound for the ability to reduce the activity or expression of a gene product required for proliferation, said method comprising the steps of:
(a) sensitizing a cell by providing said cell with a sublethal level of an antisense nucleic acid complementary to at least a portion of an operon comprising a gene which encodes a gene product required for proliferation wherein said gene product has at least 25% amino acid identity, as determined using FASTA version 3.0t78 with default parameters, to a gene product encoded by a gene contained in an operon comprises a nucleotide sequence selected from the group consisting of SEQ ID NOs: 1-194, and wherein said antisense nucleic acid is not complementary to one or more of SEQ ID NOs: 201-550 or portions thereof;
(b) contacting said sensitized cell with a compound; and
(c) determining the degree to which said compound inhibits proliferation of said sensitized cell relative to a nonsensitized cell or a cell which is less sensitive to said compound than said sensitized cell.

176. The method of Paragraph 175, wherein said gene is selected from the group consisting of SEQ ID NOs: 201-550.

177. The method of Paragraph 175, wherein said gene product is selected from the group consisting of SEQ ID NOs: 551-824.

Other aspects of the present invention relate to methods which include a step of providing an antisense nucleic acid complementary to at least a portion of an operon required for proliferation, wherein said operon comprises at least one gene that is homologous to a gene contained in an operon of SEQ ID NOs: 1-194, and wherein said antisense is not complementary to one or more of the coding sequences, or portions thereof, specified in U.S. patent application Ser. No. 10/282,122, entitled IDENTIFICATION OF ESSENTIAL GENES IN MICROORGANISMS, filed Oct. 25, 2002, the disclosure of which is incorporated herein by reference in its entirety. Examples of such methods are described in paragraphs 113-128, 132-134, 152-168, and 172-174 above as well as elsewhere herein.

Still other aspects of the present invention relate to methods which include a step of providing an antisense nucleic acid complementary to at least a portion of an operon required for proliferation, wherein said operon comprises at least one gene that encodes a polypeptide that is homologous to a polypeptide contained in an operon of SEQ ID NOs: 1-194, and wherein said antisense is not complementary to one or more of the genes which encode the polypeptides, or portions thereof, specified in U.S. patent application Ser. No. 10/282,122, entitled IDENTIFICATION OF ESSENTIAL GENES IN MICROORGANISMS, filed Oct. 25, 2002, the disclosure of which is incorporated herein by reference in its entirety. Examples of such methods are described in paragraphs 135-137 and 175-177 above as well as elsewhere herein.

### DEFINITIONS

By “biological pathway” is meant any discrete cell function or process that is carried out by a gene product or a subset of gene products. Biological pathways include anabolic, catabolic, enzymatic, biochemical and metabolic pathways as well as pathways involved in the production of cellular structures such as cell walls. Biological pathways that are usually required for proliferation of cells or microorganisms include, but are not limited to, cell division, DNA synthesis and replication, RNA synthesis (transcription), protein synthesis (translation), protein processing, protein transport, fatty acid biosynthesis, electron transport chains, cell wall synthesis, cell membrane production, synthesis and maintenance, and the like.

By “inhibit activity of a gene or gene product” is meant having the ability to interfere with the function of a gene or gene product in such a way as to decrease expression of the gene, in such a way as to reduce the level or activity of a product of the gene or in such a way as to inhibit the interaction of the gene or gene product with other biological molecules required for its activity. Agents which inhibit the activity of a gene include agents that inhibit transcription of the gene, agents that inhibit processing of the transcript of the gene, agents that reduce the stability of the transcript of the gene, and agents that inhibit translation of the mRNA transcribed from the gene. In microorganisms, agents which inhibit the activity of a gene can act to decrease expression of the operon in which the gene resides or alter the folding or processing of operon RNA so as to reduce the level or activity of the gene product. The gene product can be a non-translated RNA such as ribosomal RNA, a translated RNA (mRNA) or the protein product resulting from translation of the gene mRNA. Of particular utility to the present invention are antisense RNAs that have activities against the operons or genes to which they specifically hybridze.

By “activity against a gene product” is meant having the ability to inhibit the function or to reduce the level or activity of the gene product in a cell. This includes, but is not limited to, inhibiting the enzymatic activity of the gene product or the ability of the gene product to interact with other biological molecules required for its activity, including inhibiting the gene product's assembly into a multimeric structure.

By “activity against a protein” is meant having the ability to inhibit the function or to reduce the level or activity of the protein in a cell. This includes, but is not limited to, inhibiting the enzymatic activity of the protein or the ability of the protein to interact with other biological molecules required for its activity, including inhibiting the protein's assembly into a multimeric structure.

By “activity against a nucleic acid” is meant having the ability to inhibit the function or to reduce the level or activity of the nucleic acid in a cell. This includes, but is not limited to, inhibiting the ability of the nucleic acid interact with other biological molecules required for its activity, including inhibiting the nucleic acid's assembly into a multimeric structure.

By “activity against a gene” is meant having the ability to inhibit the function or expression of the gene in a cell. This includes, but is not limited to, inhibiting the ability of the gene to interact with other biological molecules required for its activity.

By “activity against an operon” is meant having the ability to inhibit the function or reduce the level of one or more products of the operon in a cell. This includes, but is not limited to, inhibiting the enzymatic activity or reducing the level of activity of one or more products of the operon or the ability of one or more products of the operon to interact with other biological molecules required for its activity.

By “antibiotic” is meant an agent which inhibits the proliferation of a cell or microorganism.

By “E. coli or Escherichia coli” is meant Escherichia coli or any organism previously categorized as a species of Shigella including Shigella boydii, Shigella flexneri, Shigella dysenteriae, Shigella sonnei, Shigella 2A.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

By “portion of an operon” is meant a fragment which comprises at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500, at least 525, at least 550, at least 575, at least 600, at least 650, at least 700, at least 750, at least 800, at least 850, at least 900, at least 950, at least 1000 or more than 1000 consecutive nucleotides of an operon but less than the total number of consecutive nucleotides present in the operon.

By “gene” is meant a nucleic acid that encodes a polypeptide or nucleic acid which is naturally produced by a cell. Genes also include nucleic acid variants of naturally occurring nucleic acids which encode polypeptides or nucleic acids which have been altered by nucleotide substitution or modification or which have been altered to include additional or fewer nucleotides than the naturally occurring nucleic acid but whose encoded polypeptides or nucleic acids still retain a function similar to those of the naturally occurring nucleic acid. In some contexts, the term gene may be used to refer to both the nucleic acid strand which encodes a polypeptide and the complementary noncoding strand. In other contexts, the term gene may be used to refer to only the nucleic acid which encodes a polypeptide. In such cases, gene is synonymous with the term “coding nucleic acid” or “coding region.”

By “operon” is meant a nucleic acid that comprises one or more genes that can be transcribed together to produce a single transcript. An operon can also comprise noncoding nucleic acid sequence that is present upstream of the 5′-most gene in the operon including, but not limited to, promoters, operators, regulatory sequences, shine dalgarno sequences and 5′-noncoding sequence. Similarly, an operon can comprise noncoding nucleic acid sequence that is present downstream of the 3′-most gene in the operon including, but not limited to, stop codons, transcriptional terminators and 3′-noncoding sequences.

By “homologous nucleic acid” is meant a nucleic acid that has at least 99%, at least 98%, at least 97%, at least 96% at least 95%, at least 94%, at least 93%, at least 92%, at least 91%, at least 90%, at least 85%, at least 80%, at least 75% or at least 70% nucleotide sequence identity to a another nucleic acid. The term “homologous nucleic acid” also includes nucleic acids which hybridize under stringent conditions to another nucleic acid. As used herein, “stringent conditions” means hybridization to filter-bound nucleic acid in 6×SSC at about 45° C. followed by one or more washes in 0.1×SSC/0.2% SDS at about 68° C. Other exemplary stringent conditions may refer, e.g., to washing in 6×SSC/0.05% sodium pyrophosphate at 37° C., 48° C., 55° C., and 60° C. as appropriate for the particular probe being used. The term “homologous nucleic acid” also includes nucleic acids which hybridize under moderate conditions to another nucleic acid. As used herein, “moderate conditions” means hybridization to filter-bound DNA in 6× sodium chloride/sodium citrate (SSC) at about 45° C. followed by one or more washes in 0.2×SSC/0.1% SDS at about 42-65° C.

By “homologous sense nucleic acid” is meant a nucleic acid that has at least 99%, at least 98%, at least 97%, at least 96% at least 95%, at least 94%, at least 93%, at least 92%, at least 91%, at least 90%, at least 85%, at least 80%, at least 75% or at least 70% nucleotide sequence identity to an operon comprising a nucleotide sequence selected from the group consisting of SEQ ID NOs: 1-194 or a portion thereof. A portion of an operon comprising a nucleotide sequence selected from the group consisting of SEQ ID NOs: 1-194 can comprise a coding region, a noncoding region, or a nucleotide sequence which includes all or portions of both coding and noncoding regions of an operon comprising a nucleotide sequence selected from the group consisting of SEQ ID NOs: 1-194. Additionally, a portion of an operon comprising a nucleotide sequence selected from the group consisting of SEQ ID NOs: 1-194 includes fragments comprising at least 15, at least 16, at least 17, at least 18, at least 19, at least 20, at least 21, at least 22, at least 23, at least 24, at least 25, at least 30, at least 35, least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500, at least 525, at least 550, at least 575, at least 600, at least 650, at least 700, at least 750, at least 800, at least 850, at least 900, at least 950, at least 1000 or more than 1000 consecutive nucleotides of a operon comprising a nucleotide sequence selected from the group consisting of SEQ ID NOs: 1-194 but less than the total number of consecutive nucleotides present in the operon. Nucleic acid identity can be measured using BLASTN version 2.0 with the default parameters or tBLASTX with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety).

The term “homologous sense nucleic acid” also includes nucleic acids which hybridize under stringent conditions to a nucleic acid that is complementary to another sense nucleic acid. In some embodiments, a homologous nucleic acid comprises a nucleic acid which hybridizes under stringent conditions to a nucleic acid that is complementary to one of the operons comprising a nucleotide sequence selected from the group consisting of SEQ ID NOs: 1-194 or a portion thereof. A portion of a nucleotide sequence complementary to an operon comprising a nucleotide sequence selected from the group consisting of SEQ ID NOs: 1-194 can comprise the complement of a coding region, a complement of a noncoding region, or a complement of a combination of coding and noncoding regions of an operon comprising a nucleotide sequence selected from the group consisting of SEQ ID NOs: 1-194. Additionally, a portion of a nucleotide sequence complementary to an operon comprising a nucleotide sequence selected from the group consisting of SEQ ID NOs: 1-194 includes fragments comprising at least 15, at least 16, at least 17, at least 18, at least 19, at least 20, at least 21, at least 22, at least 23, at least 24, at least 25, at least 30, at least 35, at least 40, least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500, at least 525, at least 550, at least 575, at least 600, at least 650, at least 700, at least 750, at least 800, at least 850, at least 900, at least 950, at least 1000 or more than 1000 consecutive nucleotides of a complement of an operon comprising a nucleotide sequence selected from the group consisting of SEQ ID NOs: 1-194 but less than the total number of consecutive nucleotides present in the complement of the operon. As used herein, “stringent conditions” means hybridization to filter-bound nucleic acid in 6×SSC at about 45° C. followed by one or more washes in 0.1×SSC/0.2% SDS at about 68° C. Other exemplary stringent conditions may refer, e.g., to washing in 6×SSC/0.05% sodium pyrophosphate at 37° C., 48° C., 55° C., and 60° C. as appropriate for the particular probe being used.

The term “homologous sense nucleic acid” also includes nucleic acids which hybridize under moderate conditions to a nucleic acid that is complementary to another nucleic acid. In some embodiments, a homologous nucleic acid comprises a nucleic acid which hybridizes under moderate conditions to a nucleic acid that is complementary to one of the operons comprising a nucleotide sequence selected from the group consisting of SEQ ID NOs: 1-194 or a portion thereof. A portion of a nucleotide sequence complementary to an operon comprising a nucleotide sequence selected from the group consisting of SEQ ID NOs: 1-194 can comprise the complement of a coding region, a complement of a noncoding region, or a complement of a combination of coding and noncoding regions of an operon comprising a nucleotide sequence selected from the group consisting of SEQ ID NOs: 1-194. Additionally, a portion of a nucleotide sequence complementary to an operon comprising a nucleotide sequence selected from the group consisting of SEQ ID NOs: 1-194 includes fragments comprising at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500, at least 525, at least 550, at least 575, at least 600, at least 650, at least 700, at least 750, at least 800, at least 850, at least 900, at least 950, at least 1000 or more than 1000 consecutive nucleotides of a complement of an operon comprising a nucleotide sequence selected from the group consisting of SEQ ID NOs: 1-194 but less than the total number of consecutive nucleotides present in the complement of the operon. As used herein, “moderate conditions” means hybridization to filter-bound DNA in 6× sodium chloride/sodium citrate (SSC) at about 45° C. followed by one or more washes in 0.2×SSC/0.1% SDS at about 42-65° C.

The term “homologous sense nucleic acid” also includes a nucleic acid comprising a nucleotide sequence which encodes a gene product whose activity is complemented by one of the gene products encoded by a gene contained in an operon comprising a nucleotide sequence selected from the group consisting of any of SEQ ID NOs: 1-194.

As used herein, a “homologous coding nucleic acid” is a nucleic acid that is homologous to a coding region contained within an operon. In some embodiments, a homologous coding nucleic acid can be identified by membership of the coding region of interest to a functional orthologue cluster. All other members of that orthologue cluster are considered homologues. Such a library of functional orthologue clusters can be found on the internet by entering the following quoted text, “www.ncbi.nlm.nih.”, in the address bar of a web browser, such as Internet Explorer or Netscape, followed immediately by “gov/COG”. A coding region, such as an open reading frame in a gene, can be classified into a cluster of orthologous groups or COG by using the COGNITOR program available at the above web site, or by direct BLASTP comparison of the gene of interest to the members of the COGs and analysis of these results as described by Tatusov, R. L., Galperin, M. Y., Natale, D. A. and Koonin, E. V. (2000) The COG database: a tool for genome-scale analysis of protein functions and evolution. Nucleic Acids Research v. 28 n. 1, pp33-36, the disclosure of which is incorporated herein by reference in its entirety.

Homologous coding nucleic acids and the homologous polypeptides which they encode may also be identified using a “reciprocal” best-hit analysis. For example, to facilitate the identification of homologous coding nucleic acids and homologous polypeptides, paralogous genes within each of 51 pathogenic bacteria are identified and clustered prior to comparison to other organisms. Briefly, the polypeptide sequence of each polypeptide encoded by each gene in a given organism is compared to the polypeptide sequence encoded by every other gene for that organism for each of the 51 pathogenic bacteria (PathoSeq Sept 2001 release) using BLASTP 2.09 algorithm without filtering. Simultaneously, the polypeptide sequence encoded by each gene of an organism is compared to the polypeptide sequences encoded by each of the genes in the remaining 51 organisms. Those polypeptides within a single organism that share a higher degree of sequence identity to one another than to polypeptide sequences obtained from any other organisms are clustered as “paralog” sequences for “reciprocal” best-hit analysis.

For each reference organism, the 50 homologous coding nucleic acids (and the 50 homologous polypeptides which they encode) can be determined by identifying the genes in each of the 50 comparison organisms which encode a polypeptide sharing the highest degree of amino acid sequence identity to the polypeptide encoded by the gene from the reference organism. The accuracy of the identification of the predicted homologous coding nucleic acids (and the homologous polypeptides which they encode) is confirmed by a “reciprocal” BLAST analysis in which the polypeptide sequence of the predicted homologous polypeptide is compared against the polypeptides encoded by each of the genes in the reference organism using BLASTP 2.09 algorithm without filtering. Only those polypeptides that share the highest degree of amino acid sequence identity in each portion of the two-way comparison are retained for further analysis.

The term “homologous coding nucleic acid” also includes nucleic acids comprising nucleotide sequences which encode polypeptides having at least 99%, at least 98%, at least 97%, at least 96%, at least 95%, at least 94%, at least 93%, at least 92%, at least 91%, at least 90%, at least 85%, at least 80%, at least 75%, at least 70%, at least 60%, at least 50%, at least 40%, at least 30% or at least 25% amino acid identity or similarity to another polypeptide. In some embodiments, a “homologous coding nucleic acid” includes nucleic acids comprising nucleotide sequences which encode polypeptides having at least 99%, at least 98%, at least 97%, at least 96%, at least 95%, at least 94%, at least 93%, at least 92%, at least 91%, at least 90%, at least 85%, at least 80%, at least 75%, at least 70%, at least 60%, at least 50%, at least 40%, at least 30% or at least 25% amino acid identity or similarity to a polypeptide encoded by a gene that is contained in any of the operons comprising a nucleotide sequence selected from the group consisting of SEQ ID NOs: 1-194. In other embodiments, a “homologous coding nucleic acid” includes nucleic acids comprising nucleotide sequences which encode polypeptides having at least 99%, at least 98%, at least 97%, at least 96%, at least 95%, at least 94%, at least 93%, at least 92%, at least 91%, at least 90%, at least 85%, at least 80%, at least 75%, at least 70%, at least 60%, at least 50%, at least 40%, at least 30% or at least 25% amino acid identity or similarity to a polypeptide encoded by a portion of a gene that is contained in any of the operons comprising a nucleotide sequence selected from the group consisting of SEQ ID NOs: 1-194. A portion of a gene that is contained in any of the operons comprising a nucleotide sequence selected from the group consisting of SEQ ID NOs: 1-194 comprises consecutive nucleotides which encode at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150 or more than 150 consecutive amino acids. In some embodiments, protein identity or similarity is determined using the FASTA version 3.0t78 algorithm with the default parameters. As an alternative, protein identity or similarity may be identified using BLASTP with the default parameters, BLASTX with the default parameters, TBLASTN with the default parameters, or tBLASTX with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety).

Homologous coding nucleic acids and the homologous polypeptides which they encode may also be identified using a “reciprocal” best-hit analysis. To facilitate the identification of homologous coding nucleic acids and homologous polypeptides, paralogous genes within each of 51 organisms are identified and clustered prior to comparison to other organisms. Briefly, the polypeptide sequence of each polypeptide encoded by each gene in a given organism is compared to the polypeptide sequence encoded by every other gene for that organism for each of the 51 pathogenic bacteria (PathoSeq September 2001 release) using BLASTP 2.09 algorithm without filtering. Simultaneously, the polypeptide sequence encoded by each gene of an organism is compared to the polypeptide sequences encoded by each of the genes in the remaining 51 organisms. Those polypeptides within a single organism that share a higher degree of sequence identity to one another than to polypeptide sequences obtained from any other organisms are clustered as “paralog” sequences for “reciprocal” best-hit analysis.

For each reference organism, the 50 homologous coding nucleic acids (and the 50 homologous polypeptides which they encode) can be determined by identifying the genes in each of the 50 comparison organisms which encode a polypeptide sharing the highest degree of amino acid sequence identity to the polypeptide encoded by the gene from the reference organism. The accuracy of the identification of the predicted homologous coding nucleic acids (and the homologous polypeptides which they encode) is confirmed by a “reciprocal” BLAST analysis in which the polypeptide sequence of the predicted homologous polypeptide is compared against the polypeptides encoded by each of the genes in the reference organism using BLASTP 2.09 algorithm without filtering. Only those polypeptides that share the highest degree of amino acid sequence identity in each portion of the two-way comparison are retained for further analysis.

It will be appreciated that other methods that are known in the art can also be used to identify homologous coding nucleic acids.

The term “homologous antisense nucleic acid” includes nucleic acids comprising a nucleotide sequence having at least 99%, at least 98%, at least 97%, at least 96% at least 95%, at least 94%, at least 93%, at least 92%, at least 91%, at least 90%, at least 85%, at least 80%, at least 75% or at least 70% nucleotide sequence identity to a nucleotide sequence complementary to an operon comprising a nucleotide sequence selected from the group consisting of SEQ ID NOs: 1-194 or a portion thereof but which is not complementary to one or more of SEQ ID NOs: 201-550 or portions thereof. A portion of a nucleotide sequence complementary to an operon comprising a nucleotide sequence selected from the group consisting of SEQ ID NOs: 1-194 can comprise the complement of a coding region, a complement of a noncoding region, or a complement of a combination of coding and noncoding regions of an operon comprising a nucleotide sequence selected from the group consisting of SEQ ID NOs: 1-194. Additionally, a portion of a nucleotide sequence complementary to an operon comprising a nucleotide sequence selected from the group consisting of SEQ ID NOs: 1-194 includes fragments comprising at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500, at least 525, at least 550, at least 575, at least 600, at least 650, at least 700, at least 750, at least 800, at least 850, at least 900, at least 950, at least 1000 or more than 1000 consecutive nucleotides of an operon comprising a nucleotide sequence selected from the group consisting of SEQ IN NOs: 1-194 but less than the total number of consecutive nucleotides present in the operon. Nucleic acid identity can be measured using BLASTN version 2.0 with the default parameters or tBLASTX with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety).

The term “homologous antisense nucleic acid” also includes nucleic acids which hybridize under stringent conditions to any one of the operons comprising a nucleotide sequence selected from the group consisting of SEQ ID NOs: 1-194 or a portion thereof but which are not complementary to one or more of SEQ ID NOs: 201-550 or portions thereof. A portion of an operon comprising a nucleotide sequence selected from the group consisting of SEQ ID NOs: 1-194 can comprise a coding region, a noncoding region, or a combination of coding and noncoding regions of an operon comprising a nucleotide sequence selected from the group consisting of SEQ ID NOs: 1-194. Additionally, a portion of an operon comprising a nucleotide sequence selected from the group consisting of SEQ ID NOs: 1-194 includes fragments comprising at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, least 95, at least 100, at least 125, at least 150, at least 175, at least 200, at least 225, at least 250, at least 275, at least 300, at least 325, at least 350, at least 375, at least 400, at least 425, at least 450, at least 475, at least 500, at least 525, at least 550, at least 575, at least 600, at least 650, at least 700, at least 750, at least 800, at least 850, at least 900, at least 950, at least 1000 or more than 1000 consecutive nucleotides of an operon comprising a nucleotide sequence selected from the group consisting of SEQ ID NOs: 1-194 but less than the total number of consecutive nucleotides present in the operon. As used herein, “stringent conditions” means hybridization to filter-bound nucleic acid in 6×SSC at about 45° C. followed by one or more washes in 0.1×SSC/0.2% SDS at about 68° C. Other exemplary stringent conditions may refer, e.g., to washing in 6×SSC/0.05% sodium pyrophosphate at 37° C., 48° C., 55° C. and 60° C. as appropriate for the particular probe being used.

The term “homologous antisense nucleic acid” also includes nucleic acids which hybridize under moderate conditions to any one of the operons comprising a nucleotide sequence selected from the group consisting of SEQ ID NOs: 1-194 or a portion thereof but which are not complementary one or more of SEQ ID NOs: 201-550 or portions thereof. A portion of an operon comprising a nucleotide sequence selected from the group consisting of SEQ ID NOs: 1-194 can comprise the a coding region, a noncoding region, or a combination of coding and noncoding regions of an operon comprising a nucleotide sequence selected from the group consisting of SEQ ID NOs: 1-194. Additionally, a portion of an operon comprising a nucleotide sequence selected from the group consisting of SEQ ID NOs: 1-194 includes fragments comprising at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500, at least 525, at least 550, at least 575, at least 600, at least 650, at least 700, at least 750, at least 800, at least 850, at least 900, at least 950, at least 1000 or more than 1000 consecutive nucleotides of an operon comprising a nucleotide sequence selected from the group consisting of SEQ ID NOs: 1-194 but less than the total number of consecutive nucleotides present in the operon. As used herein, “moderate conditions” means hybridization to filter-bound DNA in 6× sodium chloride/sodium citrate (SSC) at about 45° C. followed by one or more washes in 0.2×SSC/0.1% SDS at about 42-65° C.

The term “homologous polypeptide” includes polypeptides having at least 99%, at least 98%, at least 97%, at least 96%, at least 95%, at least 94%, at least 93%, at least 92%, at least 91%, at least 90%, at least 85%, at least 80%, at least 75%, at least 70%, at least 60%, at least 50%, at least 40%, at least 30% or at least 25% amino acid identity or similarity to another polypeptide. In some embodiments, a homologous polpypeptide has at least 99%, at least 98%, at least 97%, at least 96%, at least 95%, at least 94%, at least 93%, at least 92%, at least 91%, at least 90%, at least 85%, at least 80%, at least 75%, at least 70%, at least 60%, at least 50%, at least 40%, at least 30% or at least 25% amino acid identity to a polypeptide that is encoded by a gene contained in an operon comprising a nucleotide sequence selected from the group consisting of SEQ ID NOs: 1-194 or a portion thereof. A portion of a polypeptide encoded by a gene contained in an operon comprising a nucleotide sequence selected from the group consisting of SEQ ID NOs: 1-194 can comprise at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, at least 170, at least 180, at least 190, at least 200, at least 225, at least 250 or more than 250 consecutive amino acids of a polypeptide encoded by a gene contained in an operon comprising a nucleotide sequence selected from the group consisting of SEQ ID NOs: 1-194 but less than the total number of amino acids present in the polypeptide. Identity or similarity may be determined using the FASTA version 3.0t78 algorithm with the default parameters. Alternatively, protein identity or similarity may be identified using BLASTP with the default parameters, BLASTX with the default parameters, or TBLASTN with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety).

In some embodiments of the present invention, nucleic acids that hybridize to any one of the nucleic acids comprising a nucleotide sequence selected from the group consisting of SEQ ID NOs.: 1-194 or complements thereof are utilized. Such hybridization may be under stringent or moderate conditions as defined above or under other conditions which permit hybridization. The nucleic acid molecules utilized in the present invention that hybridize to these DNA sequences include oligodeoxynucleotides (“oligos”) which hybridize to the target gene under highly stringent or stringent conditions. In general, for oligos between 14 and 70 nucleotides in length the melting temperature (Tm) is calculated using the formula:
Tm(° C.)=81.5+16.6(log[monovalent cations(molar)]+0.41(% G+C)−(500/N)
where N is the length of the probe. If the hybridization is carried out in a solution containing formamide, the melting temperature may be calculated using the equation:
Tm(° C.)=81.5 +16.6(log[monovalent cations(molar)]+0.41(% G+C)−(0.61) (% formamide)−(500/N)
where N is the length of the probe. In general, hybridization is carried out at about 20-25 degrees below Tm (for DNA-DNA hybrids) or about 10-15 degrees below Tm (for RNA-DNA hybrids).

Other hybridization conditions are apparent to those of skill in the art (see, for example, Ausubel, F. M. et al., eds., 1989, Current Protocols in Molecular Biology, Vol. I, Green Publishing Associates, Inc. and John Wiley & Sons, Inc., New York, at pp. 6.3.1-6.3.6 and 2.10.3, the disclosure of which is incorporated herein by reference in its entirety).

The term, Salmonella, is the generic name for a large group of gram negative enteric bacteria that are closely related to Escherichia coli. The diseases caused by Salmonella are often due to contamination of foodstuffs or the water supply and affect millions of people each year. Traditional methods of Salmonella taxonomy were based on assigning a separate species name to each serologically distinguishable strain (Kauffmann, F 1966 The bacteriology of the Enterobacteriaceae. Munksgaard, Copenhagen). Serology of Salmonella is based on surface antigens (O [somatic] and H [flagellar]). Over 2,400 serotypes or serovars of Salmonella are known (Popoff, et al. 2000 Res. Microbiol. 151:63-65). Therefore, each serotype was considered to be a separate species and often given names, accordingly (e.g. S. paratyphi, S. typhimurium, S. typhi, S. enteriditis, etc.).

However, by the 1970s and 1980s it was recognized that this system was not only cumbersome, but also inaccurate. Then, many Salmonella species were lumped into a single species (all serotypes and subgenera I, II, and IV and all serotypes of Arizona) with a second subspecies, S. bongorii also recognized (Crosa, et al., 1973, J. Bacteriol. 115:307-315). Though species designations are based on the highly variable surface antigens, the Salmonella are very similar otherwise with a major exception being pathogenicity determinants.

There has been some debate on the correct name for the Salmonella species. Currently (Brenner, et al. 2000 J. Clin. Microbiol. 38:2465-2467), the accepted name is Salmonella enterica. S. enterica is divided into six subspecies (I, S. enterica subsp. enterica; II, S. enterica, subsp. salamae; IIIa, S. enterica subsp. arizonàe; IIIb, S. enterica subsp. diarizonae; IV, S. enterica subsp. houtenae; and VI, S. enterica subsp. indica). Within subspecies I, serotypes are used to distinguish each of the serotypes or serovars (e.g. S. enterica serotype Enteriditis, S. enterica serotype Typhimurium, S. enterica serotype Typhi, and S. enterica serotype Choleraesuis, etc.). Current convention is to spell this out on first usage (Salmonella enterica ser. Typhimurium) and then use an abbreviated form (Salmonella Typhimurium or S. Typhimurium). Note, the genus and species names (Salmonella enterica) are italicized but not the serotype/serovar name (Typhimurium). Because the taxonomic committees have yet to officially approve of the actual species name, this latter system is what is employed by the CDC (Brenner, et al. 2000 J. Clin. Microbiol. 38:2465-2467). Due to the concerns of both taxonomic priority and medical importance, some of these serotypes might ultimately receive full species designations (S. typhi would be the most notable).

Therefore, as used herein “Salmonella enterica or S. enterica” includes serovars Typhi, Typhimurium, Paratyphi, Choleraesuis, etc.” However, appeals of the “official” name are in process and the taxonomic designations may change (S. choleraesuis is the species name that could replace S. enterica based solely on priority).

By “identifying a compound” is meant to screen one or more compounds in a collection of compounds such as a combinatorial chemical library or other library of chemical compounds or to characterize a single compound by testing the compound in a given assay and determining whether it exhibits the desired activity.

By “inducer” is meant an agent or solution which, when placed in contact with a cell or microorganism, increases transcription, or inhibitor and/or promoter clearance/fidelity, from a desired promoter.

As used herein, “nucleic acid” means DNA, RNA, or modified nucleic acids. Thus, the terminology “the nucleic acid of SEQ ID NO: X” or “the nucleic acid comprising the nucleotide sequence” includes both the DNA sequence of SEQ ID NO: X and an RNA sequence in which the thymidines in the DNA sequence have been substituted with uridines in the RNA sequence and in which the deoxyribose backbone of the DNA sequence has been substituted with a ribose backbone in the RNA sequence. Modified nucleic acids are nucleic acids having nucleotides or structures which do not occur in nature, such as nucleic acids in which the internucleotide phosphate residues have been replaced with methylphosphonates, phosphorothioates, phosphoramidates, and phosphate esters. Nonphosphate internucleotide analogs such as siloxane bridges, carbonate brides, thioester bridges, as well as many others known in the art may also be used in modified nucleic acids. Modified nucleic acids may also comprise, α-anomeric nucleotide units and modified nucleotides such as 1,2-dideoxy-d-ribofuranose, 1,2-dideoxy-1-phenylribofuranose, and N⁴,N⁴-ethano-5-methyl-cytosine are contemplated for use in the present invention. Modified nucleic acids may also be peptide nucleic acids in which the entire deoxyribose-phosphate backbone has been exchanged with a chemically completely different, but structurally homologous, polyamide (peptide) backbone containing 2-aminoethyl glycine units.

As used herein, “sensitized cell” means a cell which has been exposed to a product or process which produces a sub-lethal effect.

As used herein, “sub-lethal” means a concentration of an agent below the concentration required to inhibit all cell growth.

The complete genome sequence for numerous strains of Staphylococcus aureus is publicly available. For example, the sequence of the complete genome of Staphylcoccus aureus strain Mu50 is deposited in Genbank under accession number BA000017; the sequence of the complete genome of Staphylcoccus aureus strain N315 is deposited in Genbank under accession number BA000018 and the sequence of the complete genome of Staphylcoccus aureus strain MW2 is deposited in Genbank under accession number BA000033. Each of the above-identified genomic sequences is incorporated herein by reference in their entireties.

### BRIEF DESCRIPTION OF THE DRAWINGS

FIG. 1 illustrates an exemplary consensus model for operon identification.

FIG. 2 illustrates a computation pipeline 200 for operon prediction.

FIG. 3A illustrates a method for distance threshold selection.

FIG. 3B illustrates the method of distance threshold selection of FIG. 3A for an exemplary target organism, Staphylococcus aureus compared to a plurality of other reference organisms.

FIG. 4 illustrates a method for conserved gene cluster analysis that may be used in operon identification.

FIG. 5A illustrates a method for boundary analysis comprising a transcriptional terminator identification approach.

FIG. 5B illustrates a relative distribution of transcription terminators predicted according to the transcriptional terminator identification approach of FIG. 5A.

FIG. 6 illustrates a method for gene pair scoring wherein a numerical score is used to represent the degree of confidence that the genes of a gene pair reside in the same operon.

FIG. 7 is an IPTG dose response curve in E. coli transformed with an IPTG-inducible plasmid containing either an antisense clone to the E. coli ribosomal protein rplW (AS-rplW) which is required for protein synthesis and essential for cell proliferation, or an antisense clone to the elaD (AS-elaD) gene which is not known to be involved in protein synthesis and which is also essential for proliferation.

FIG. 8A is a tetracycline dose response curve in E. coli transformed with an IPTG-inducible plasmid containing antisense to rplW (AS-rplW) in the absence (0) or presence of IPTG at concentrations that result in 20% and 50% growth inhibition.

FIG. 8B is a tetracycline dose response curve in E. coli transformed with an IPTG-inducible plasmid containing antisense to elaD (AS-elaD)in the absence (0) or presence of IPTG at concentrations that result in 20% and 50% growth inhibition.

FIG. 9 is a graph showing the fold increase in tetracycline sensitivity of E. coli transfected with antisense clones to essential ribosomal proteins L23 (AS-rplW) and L7/L12 and L10 (AS-rplLrplJ). Antisense clones to genes known to not be directly involved in protein synthesis, atpB/E (AS-atpB/E), visC (AS-visC), elaD (AS-elaD), yohH (AS-yohH), are much less sensitive to tetracycline.

FIG. 10 illustrates the results of an assay in which Staphylococcus aureus cells transcribing an antisense nucleic acid complementary to the gyrB gene encoding the β subunit of gyrase were contacted with several antibiotics whose targets were known.

### DETAILED DESCRIPTION OF EMBODIMENTS OF THE INVENTION

In various embodiments, the present teachings can be used to map some or all of the operons within a selected organism's genome while reducing the complexity of deconvolution resulting from polar effects created by conventional analytical methods. Unlike conventional methods which typically lack accuracy, specificity, and/or sensitivity, the present consensus-based model desirably improves the reliability of operon identification and capitalizes on a variety of different predictive methods. Using a novel approach to results analysis, improved usability is attained as compared to conventional operon identification methods when used independently of one another. Consequently, the task of operon prediction is significantly improved, especially when evaluating organisms for which limited experimental information exists.

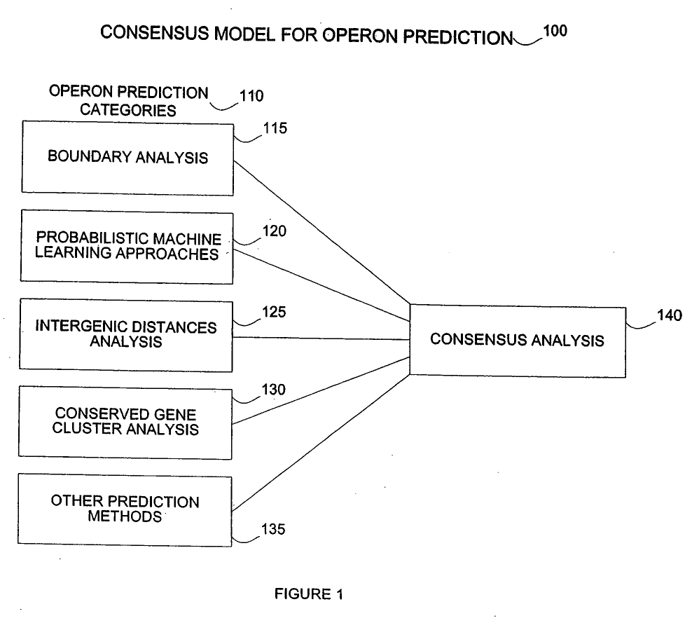

FIG. 1 illustrates an exemplary consensus model for operon identification 100 incorporating a plurality of analytical categories 110 used in operon prediction. As described above, the present teachings offer improved predictive quality over singular approaches to operon prediction and may be used to selectively weight the contribution each singular approach has on the overall result obtained through consensus analysis. Exemplary operon prediction categories 110 from which the consensus model 100 may be derived include: promoter and boundary analysis 115; probabilistic machine learning approaches 120; intergenic distances analysis 125; and conserved gene cluster analysis 130. Additionally, other prediction methods 135 may be readily incorporated into the consensus model 100 to thereby maintain flexibility in operon identification that may include analytical methods and approaches other than those disclosed. As such, the disclosed operon prediction methods 110 used in conjunction with the consensus model 100 are conceived to represent but some of the various embodiments of operon prediction according to the present teachings and additional models using other operon prediction methods may be readily incorporated or substituted for those illustrated in FIG. 1.

In one aspect, boundary analysis 115 may be utilized to aid in operon identification. For example, applications and software algorithms are available for the prediction of transcriptional terminator motifs including rho-independent transcription terminators. Additionally, prokaryotic promoter-searching applications and algorithms may available. While conventional approaches may construct hidden Markov models (HMM) based on known promoters and terminators to aid in the prediction of operon structures, these methods are of limited utility when used alone and may yield marginal operon prediction accuracy. Furthermore, use of solely boundary analysis approaches 115 for operon prediction may be difficult to apply in prokaryotic organisms for which promoters and terminators are not well characterized. Additional details of this operon prediction category can be found in the following publications: (1) Brendel,V. and Trifonov, E. N. (1984) A computer algorithm for testing potential prokaryotic terminators. Nucleic Acids Res., 12, 4411-4427. (2) Brendel,V. and Trifonov,E. N. (1984) Computer-aided mapping of DNA-protein interaction sites. CODATA Conference Proc. (3) Ermolaeva,M. D., Khalak,H. G., White,O., Smith,H. O. and Salzberg,S. L. (2000) Prediction of transcription terminators in bacterial genomes. J. Mol. Biol, 301, 27-33. (4) Unniraman,S., Prakash,R. and Nagaraja,V. (2002) Conserved economics of transcription termination in eubacteria. Nucleic Acids Res., 30, 675-684. (5) Ozoline,O. N., Deev,A. A. and Arkhipova,M. V. (1997) Non-canonical sequence elements in the promoter structure. Cluster analysis of promoters recognized by Escherichia coli RNA polymerase. Nucleic Acids Res., 23, 4703X709. (6) Yada,T., Nakao,M., Totoki,Y. and Nakai,K. (1999) Modeling and predicting transcriptional units of Escherichia coli genes using hidden Markov models. Bioinformatics, 15, 987-993, the disclosures of which are incorporated herein by reference in their entireties.

Probabilistic machine learning approaches 120 may also be used to deduce operon composition and structure using a variety of data types including sequence data, gene expression data and functional annotation data. In one aspect, these methods may be used to estimate the probability that a consecutive sequence of genes residing on the same strand comprise an operon. As with boundary analysis 115, this method when used alone identifies operons with only marginal accuracy.

In one aspect, probabilistic machine learning approaches 120 may directed to operon identification through analysis of gene expression data obtained from microarrays and other multi-gene expression analysis methodologies. For example, operon structure may be deduced by co-expression pattern identification provided sufficient expression data is available. One potential difficulty encountered by conventional methods using solely this approach is that it is generally applicable only in organisms for which significant amounts of experimental data are available. Additional details of this operon prediction category can be found in the following publications: (1) Craven,M., Page,D., Shavlik,J., Bockhorst,J. and Glasner,J. (2000) A probabilistic learning approach to whole-genome operon prediction. ISMB, 8, 116-127. (2) Sabatti,C., Rohlin,L., Oh,M. and Liao,J. C. (2002) Co-expression pattern from DNA microarray experiments as a tool for operon prediction. Nucleic Acids Res., 30, 2886-2893, the disclosures of which are incorporated herein by reference in their entireties.

Intergenic distance analysis 125 represents another singular means for operon prediction 110. Using this approach, the intergenic distance between adjacent genes may be determined and operons predicted on the basis of the observation that genes within operons typically have substantially shorter intergenic distances as compared to genes at the borders of transcription units. This approach has been reported to provide a somewhat more efficient means to identify adjacent gene pairs contained within operons, however, the overall accuracy of this approach is still lacking and its use alone is not necessarily appropriate in all prokaryotic organisms. Additional details of this operon prediction category can be found in the following publication: Salgado,H., Moreno-Hagelsieb,G., Smith,T. F. and Collado-Vides,J. (2000) Operons in Escherichia coli: genomic analyses and predictions. Proc. Natl Acad. Sci. USA, 97, 6652-6657, the disclosures of which are incorporated herein by reference in their entireties.

Conserved gene cluster analysis 130 using a comparative genomics approach has been described as having a relatively high specificity in identifying co-transcribed gene pairs. Like the other approaches for operon prediction 110 described above, this approach 130 generally does not provide sufficient results by itself and may fail to predict all genes contained within identified operons. Additionally, the sensitivity of operon prediction when using solely this approach may be attenuated to a certain degree. Additional details of this operon prediction category can be found in the following publications: (1) Ermolaeva,M., White,O. and Salzberg,S. L. (2001) Prediction of operons in microbial genomes. Nucleic Acids Res., 29, 1216-1221. (2) Wolf,Y. I., Rogozin,I. B., Kondrashov,A. S. and Koonin,E. V. (2001) Genome alignment, evolution of prokaryotic genome organization, and prediction of gene function using genomic context. Genome Res. 11, 356-372, the disclosures of which are incorporated herein by reference in their entireties.

In various embodiments, the present teachings describe methods to obtain improved results in operon prediction over utilization of the foregoing approaches 110 in singular or independent manners. By developing a directed strategy to perform integrated analysis using various operon prediction methods in concert, for example, combined approaches using gene orientation analysis, intergenic distance analysis, conserved gene cluster analysis and terminator detection, significant improvements in accuracy and sensitivity can be obtained over utilization of any given technique alone. This integrated strategy or consensus analysis 140 serves as a basis for operon identification in which confidence scoring is performed using the results of one or more of the aforementioned operon prediction categories.

Various embodiments of the operon prediction methods described herein can be used to predict operons in any prokaryotic organism. These methods are especially useful for predicting operons in pathogenic microorganisms including, but not limited to, Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis and any species falling within the genera of any of the above species.

In some embodiments, the methods described herein have been used to predict operons in Staphylococcus aureus. For example, the methods of operon prediction described herein were used to predict over 1397 operons from the protein-encoding genes in the genome of Staphylococcus aureus strain Mu50. Of the identified operons, approximately 62% were predicted to be monocistronic and approximately 38% were predicted to be polycistronic. Comparison with experimentally determined values for Staphylococcus aureus operons from literature sources indicated that the disclosed methods successfully predicted operon boundaries and genes contained within the each operon interior with a high degree of accuracy. The Staphylococcus aureus operons that are predicted by the methods described herein are provided in the Examples below in Table V.

Some embodiments of the present invention describe operons that are required for cellular proliferation. In some embodiments, the proliferation-required operon comprises one or more genes that are required for proliferation. In other embodiments, the operon may contain no genes that are singly required for proliferation but rather the operon contains a plurality of genes that, in combination, are required for proliferation. For example, an operon may consist of gene A and gene B, neither of which alone is required for proliferation. Thus, inhibition of the expression of either gene A or gene B alone would not be sufficient to inhibit the growth of the cell in which it is present. However, inhibiting the expression of both gene A and gene B together, for example, by inhibiting the expression of the polycistronic transcript by modulating the activity of the operon, would inhibit cell growth. For example, in some embodiments, an antisense nucleic acid complementary to all or a portion of one operon may be used to reduce the activity or level of the gene products encoded by gene A and gene B, or by reducing the activity or level of the polypeptides encoded by gene A and gene B. Certain embodiments of the present invention describe operons in Staphylococcus aureus that are required for cellular proliferation. Each of these operons is listed in the Examples below in Table V (SEQ ID NOs: 1-194). In some embodiments, a proliferation-required operon comprises a plurality of genes wherein one or more of the genes contained in the operon are required for the proliferation of Staphylococcus aureus. In other cases, the proliferation-required operon is monocistronic, and thus, comprises only a single gene, wherein this single gene is required for proliferation.

A proliferation-required gene is one where, in the absence or substantial reduction of a gene transcript and/or gene product, growth or viability of the cell or microorganism is reduced or eliminated. Thus, as used herein, the terminology “proliferation-required” or “required for proliferation” encompasses instances where the absence or substantial reduction of a gene transcript and/or gene product completely eliminates cell growth as well as instances where the absence of a gene transcript and/or gene product merely reduces cell growth. Typically, a proliferation required operon is one that contains one or more genes required for proliferation. However, in some embodiments, a proliferation-required operon includes those operons which contain no single gene that is required for proliferation, but rather, contains a plurality of genes that, in combination, are required for proliferation.

In accordance with another aspect of the present invention, vectors are disclosed which comprise a promoter operably linked to a operon that has been identified by an operon prediction method as described herein or operably linked to all or a portion of the noncoding strand of an operon that has been identified by an operon prediction method as described herein. In some embodiments, the operon can be an operon that is required for proliferation, for example, an operon which comprises at least one gene that is required for proliferation. In some embodiments of the present invention, the vectors include an operon which has been identified from a pathogenic microorganism including, but not limited to, Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species. In some embodiments, the vectors include an operon comprising a nucleotide sequence selected from the group consisting of SEQ ID NOs: 1-194 or all or a portion of the noncoding strand of a nucleotide sequence selected from the group consisting of SEQ ID NOs: 1-194.

Some aspects of the present invention contemplate that the operon-containing vectors described herein are transformed into a microorganism. The one or more genes contained in the operon may or may not be expressed in the host microbe.

Additionally, described herein are methods for identifying genes that are required for proliferation in an organism are described. These methods, which can be applied in a wide range of organisms, have been used as a preliminary step in the identification of proliferation-required operons. For example, once a gene has been identified as required for proliferation, its chromosomal location can be identified with respect to other adjacent genes. The operon prediction methods described herein are then used to determine whether one or more of the adjacent genes are contained in the same operon as the proliferation-required gene.

Some embodiments of the present invention contemplate methods of inhibiting cellular proliferation by inhibiting the activity or reducing the amount of one or more gene products that are produced by an operon that has been identified by an operon prediction method described herein. Although numerous methods of inhibiting proliferation are contemplated, such as, down-regulating the expression of the genes in an operon by controlling promoter activity or creating null mutants in a gene required for proliferation, modulation of operon activity using an antisense nucleic acid is of particular relevance. For example, an antisense nucleic acid which is complementary to at least a portion of a proliferation-required gene that is contained in an operon can be used to inhibit the activity or reduce the amount of the corresponding proliferation-required gene product thereby inhibiting proliferation of the cell in which the gene is present. However, since the proliferation-required gene is contained in an operon, an antisense nucleic acid which is complementary to a portion of the operon that does not include the identified proliferation-required gene can also be used to inhibit the activity or reduce the amount of product of the proliferation-required gene. In some embodiments, the antisense is complementary to at least a portion of another gene in the operon that is not required for proliferation. In other embodiments, the antisense is complementary to a nontranslated region of the operon. In many cases, the inhibition of the activity or reduction in the amount of the product of the proliferation-required gene will be due to a polar effect.

Further embodiments of the present invention relate to the inhibition of cellular proliferation by inhibiting the activity or reducing the amount of an operon that is required for proliferation wherein the operon does not contain a single gene that required for proliferation but rather the operon contains a plurality of genes that are, in combination, required for proliferation. In such embodiments, the inhibition or activity or reduction in the amount of multiple gene products which are not required for proliferation results in the inhibition of proliferation of a cell that is exposed to an antisense nucleic acid that is complementary to at least a portion of an the operon. Thus, the ability of a single antisense to simultaneously inhibit the activity or reduce the amount of a plurality of gene products can result in the inhibition of cellular proliferation even though none of the gene products are individually required for cellular proliferation.

Additional aspects of the present invention relate to methods for identifying compounds that possess the ability to inhibit cellular proliferation. As described further herein, cell-based methods can be used to identify compounds that inhibit proliferation. Compounds that are screened can be of any type but of particular relevance are products of natural product, semi-synthetic or fully synthetic chemical libraries. In some embodiments, compounds that inhibit cellular proliferation are identified by determining the identity of compounds which have the ability to reduce the proliferation of sensitized cells to a greater extent than cells which have not been sensitized. Cells are sensitized by providing a sublethal level an antisense nucleic acid that is complementary to at least a portion of an operon that is required for proliferation. In some embodiments of the present invention, the operon comprises a nucleotide sequence selected from the group consisting of SEQ ID NOs: 1-194. In other embodiments, the antisense is complementary to least a portion of an operon comprising a nucleotide sequence selected from the group consisting of SEQ ID NOs: 1-194, wherein the portion does not include a nucleotide sequence selected from the group consisting of SEQ ID NOs: 201-550.

In addition to the foregoing methods, antisense nucleic acids which are complementary to at least a portion of a proliferation-required operon in one species can be used in methods of inhibiting proliferation and methods of screening for compounds which inhibit proliferation in a second species which possesses one or more homologous sense nucleic acids, one or more homologous coding nucleic acids or one or more homologous polypeptides. For example, in one embodiment of the present invention, an antisense nucleic acid that is complementary to at least a portion of a proliferation-required operon comprising a nucleotide sequence selected from the group consisting of SEQ ID NOs: 1-194, wherein the portion does not include a nucleotide sequence selected from the group consisting of SEQ ID NOs: 201-550, can be used to inhibit the proliferation of a prokaryote which possesses a proliferation-required gene that is homologous to at least one nucleotide sequence selected from the group consisting of SEQ ID NOs: 201-550. In other embodiments, an antisense nucleic acid that is complementary to at least a portion of a proliferation-required operon comprising a nucleotide sequence selected from the group consisting of SEQ ID NOs: 1-194, wherein the portion does not include a nucleotide sequence selected from the group consisting of SEQ ID NOs: 201-550, can be used to inhibit the proliferation of a prokaryote which possesses a gene that encodes a gene product that is homologous to at least one amino acid sequence selected from the group consisting of SEQ ID NOs: 551-824.

It will be appreciated that the methods described herein for the inhibition of cellular proliferation and the identification of compounds that inhibit cellular proliferation can be performed in any prokaryotic microorganism that possesses one or more proliferation-required operons. For example, the methods described herein for the inhibition of cellular proliferation and the identification of compounds that inhibit cellular proliferation can be performed in Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species. In some embodiments, the methods described herein for the inhibition of cellular proliferation and the identification of compounds that inhibit cellular proliferation are performed in Staphylococcus aureus using an antisense nucleic acid that is complementary to at least a portion of an operon comprising a nucleotide sequence selected from the group consisting of SEQ ID NOs: 1-194, wherein the portion does not include a nucleotide sequence selected from the group consisting of SEQ ID NOs: 201-550.

Methods of Identifying Essential Genes

In some embodiments of the present invention, genes required for proliferation are identified. Thereafter, the operon in which the proliferation-required gene lies is identified using the methods described above. Antisense nucleic acids complementary to all or a portion of the identified operons are used in cell-based screening methods to identify compounds which inhibit proliferation. Methods of identifying genes required for proliferation have been previously described in U.S. patent application Ser. Nos. 09/492,709, entitled GENES IDENTIFIED AS REQUIRED FOR PROLIFERATION IN ESCHERICHIA COLI, filed Jan. 27, 2000; 09/711,164, entitled GENES ESSENTIAL FOR MICROBIAL PROLIFERATION AND ANTISENSE THERETO, filed Nov. 9, 2000; 09/741,669, entitled GENES IDENTIFIED AS REQUIRED FOR PROLIFERATION OF E. COLI, filed Dec. 19, 2000; 09/815,242, entitled IDENTIFICATION OF ESSENTIAL GENES IN PROKARYOTES, filed Mar. 21, 2001; 09/948,993, entitled RAPID METHOD FOR REGULATING GENE EXPRESSION, filed Sep. 6, 2001 and 10/282,122, entitled IDENTIFICATION OF ESSENTIAL GENES IN MICROORGANISMS, filed Oct. 25, 2002, the disclosures of which are incorporated herein by reference in their entireties. Exemplary methods are briefly described below.

Generally, a library of nucleic acid sequences from a given source are subcloned or otherwise inserted immediately downstream of an inducible promoter on an appropriate vector, such as a Staphylococcus aureus/E. coli or Pseudomonas aeruginosa/E. coli shuttle vector, or a vector which will replicate in both Salmonella typhimurium and Klebsiella pneumoniae, or other vector or shuttle vector capable of functioning in the intended organism, thus forming an expression library. It is generally preferred that expression is directed by a regulatable promoter sequence such that expression level can be adjusted by addition of variable concentrations of an inducer molecule or of an inhibitor molecule to the medium. For example, a number of regulatable promoters useful for regulating the expression of nucleic acid sequences over a wide range of expression levels are described in U.S. patent application Ser. No. 10/032,393, entitled BACTERIAL PROMOTERS AND METHODS OF USE, filed Dec. 21, 2001, the disclosure of which is incorporated herein by reference in its entirety. Temperature activated promoters, such as promoters regulated by temperature sensitive repressors, such as the lambda C₁₈₅₇ repressor, can also be used. Although the insert nucleic acids may be derived from the chromosome of the cell or microorganism into which the expression vector is to be introduced, because the insert is not in its natural chromosomal location, the insert nucleic acid is an exogenous nucleic acid. As used throughout this specification, the term “expression” is defined as the production of a sense or antisense RNA molecule from a gene, gene fragment, genomic fragment, chromosome, operon or portion thereof. Expression can also be used to refer to the process of peptide or polypeptide synthesis. An expression vector is defined as a vehicle by which a ribonucleic acid (RNA) sequence is transcribed from a nucleic acid sequence carried within the expression vehicle. The expression vector can also contain features that permit translation of a protein product from the transcribed RNA message expressed from the exogenous nucleic acid sequence carried by the expression vector. Accordingly, an expression vector can produce an RNA molecule as its sole product or the expression vector can produce a RNA molecule that is ultimately translated into a protein product.

Once generated, the expression library containing the exogenous nucleic acid sequences is introduced into a population of cells (such as the organism from which the exogenous nucleic acid sequences were obtained) to search for genes that are required for bacterial proliferation. Because the library molecules are foreign, in context, to the population of cells, the expression vectors and the nucleic acid segments contained therein are considered exogenous nucleic acid.

Expression of the exogenous nucleic acid fragments in the test population of cells containing the expression library is then activated. Activation of the expression vectors consists of subjecting the cells containing the vectors to conditions that result in the expression of the exogenous nucleic acid sequences carried by the expression library. The test population of cells is then assayed to determine the effect of expressing the exogenous nucleic acid fragments on the test population of cells. Those expression vectors that negatively impact the growth of the cells upon induction of expression of the random sequences contained therein are identified, isolated, and purified for further study.

Although general model for the identification of proliferation-required genes has been described above, it will be appreciated that modifications to this general method can be envisioned. A few exemplary modifications, which have been described elsewhere, are provided below.

In one example, the expression library of exogenous nucleic acids is inserted into a vector which comprises a regulatable fusion promoter selected from a suite of fusion promoters, wherein the promoter suite is useful for modulating both the basal and maximal levels of transcription of a nucleic acid over a wide dynamic range thus allowing the desired level of production of a transcript. Such promoters are described in U.S. patent application Ser. No. 10/032,393, entitled BACTERIAL PROMOTERS AND METHODS OF USE, filed Dec. 21, 2001, the disclosure of which is incorporated herein by reference in its entirety.

In another example, the expression library of exogenous nucleic acids is inserted into a vector useful for the production of stabilized mRNA having an increased lifetime (including antisense RNA) in Gram negative organisms. Such vectors are described in U.S. patent application Ser. No. 10/327592, entitled STABILIZED NUCLEIC ACIDS IN GENE AND DRUG DISCOVERY AND METHODS OF USE, filed Dec. 20, 2002, the disclosure of which is incorporated herein by reference in its entirety. Briefly, the antisense RNA which is produced from such expression vectors comprises at least one stem loop flanking each end of the antisense nucleic acid. In some versions of the vector, the at least one stem-loop structure formed at the 5′ end of the stabilized antisense nucleic acid comprises a flush, double stranded 5′ end. In other versions, one or more of the stem loops comprises a rho independent terminator. Further versions of the vectors permits transcription of a stabilized antisense RNA which lacks a ribosome binding site, which lacks sites that are cleaved by one or more RNAses, such as RNAse E or RNAse III, or which lacks both a ribosomal binding site and RNAse cleavage sites.

In other examples, expressed antisense RNAs are stabilized by transcribing the these nucleic acids in a cell in which the activity of at least one enzyme involved in RNA degradation has been reduced. For example, the activity of an enzyme such as RNase E, RNase II, RNase III, polynucleotide phosphorylase, and poly(A) polymerase, RNA helicase, enolase or an enzyme having similar functions may be reduced in the cell.

As an alternative to the above-described approach for the identification of proliferation-required genes, such genes may be identified by replacing the natural promoter for the proliferation required gene with a regulatable promoter. The growth of such strains under conditions in which the promoter is active or non-repressed is compared to the growth under conditions in which the promoter is inactive or repressed. If the strains fail to grow or grow at a substantially reduced rate under conditions in which the promoter is inactive or repressed but grow normally under conditions in which the promoter is active or non-repressed, then the gene which is operably linked to the regulatable promoter encodes a gene product required for proliferation. For example, proliferation-required genes and gene products identified using promoter replacement are described in U.S. patent application Ser. No. 09/948,993, entitled RAPID METHOD FOR REGULATING GENE EXPRESSION, filed Sep. 6, 2001, the disclosure of which is incorporated herein by reference in its entirety. An exemplary promoter replacement method is described below.

In promoter replacement methods for identifying genes required for proliferation, the natural promoter can be replaced using techniques which employ homologous recombination to exchange a promoter present on the chromosome of the cell with the desired promoter which is introduced into the cell on a promoter replacement cassette. The promoter replacement cassette comprises a 5′ region homologous to the sequence which is 5′ of the natural promoter in the chromosome, the promoter which is to replace the chromosomal promoter and a 3′ region which is homologous to sequences 3′ of the natural promoter in the chromosome. In some versions of promoter replacement, the promoter replacement cassette can also include a nucleic acid encoding an identifiable or selectable marker disposed between the 5′ region which is homologous to the sequence 5′ of the natural promoter and the promoter which is to replace the chromosomal promoter. If desired, the promoter replacement cassette can also contain a transcriptional terminator 3′ of the gene encoding an identifiable or selectable marker. After uptake of the promoter replacement cassette by the cell, homologous recombination is allowed to occur between the cassette and the chromosomal region containing the natural promoter. Cells in which the promoter replacement cassette has integrated into the chromosome are identified or selected. To confirm that homologous recombination has occurred, the chromosomal structure of the cells can be verified by Southern analysis or PCR.

In some versions of promoter replacement, the promoter replacement cassette can be introduced into the cell as a linear nucleic acid, such a PCR product or a restriction fragment. Alternatively, the promoter replacement can be introduced into the cell on a plasmid.

In other versions of promoter replacement, the cell into which the promoter replacement cassette is introduced can carry mutations which enhance its ability to be transformed with linear DNA or which enhance the frequency of homologous recombination. For example, if the cell is an Escherichia coli cell it can have a mutation in the gene encoding Exonuclease V of the RecBCD recombination complex. If the cell is an Escherichia coli cell it can have a mutation that activates the RecET recombinase of the Rac prophage and/or a mutation that enhances recombination through the RecF pathway. For example, the Escherichia coli cells can be RecB or RecC mutants carrying an sbcA or sbcB mutation. Alternatively, the Escherichia coli cells can be recD mutants. In other embodiments the Escherichia coli cells can express the λ Red recombination genes. For example, Escherichia coli cells suitable for use in techniques employing homologous recombination have been described in Datsenko, K. A. and Wanner, B. L., PNAS 97:6640-6645 (2000); Murphy, K. C., J. Bact 180: 2053-2071 (1998); Zhang, Y., et al., Nature Genetics 20: 123-128 (1998); and Muyrers, J. P. P. et al., Genes & Development 14: 1971-1982 (2000), the disclosures of which are incorporated herein by reference in their entireties. Additionally, a skilled artisan will appreciate that cells carrying mutations in similar genes can be constructed in organisms other than Escherichia coli.

In other versions of promoter replacement, a regulatable fusion promoter selected from a suite of fusion promoters, as described above and in U.S. patent application Ser. No. 10/032,393, BACTERIAL PROMOTERS AND METHODS OF USE, filed Dec. 21, 2001, can be used in the construction of the replacement cassette.

It will be appreciated that promoter replacement cassettes can be integrated anywhere in the bacterial genome. Accordingly, the cassette need not necessarily replace at native promoter, but rather, can be integrated upstream of one or more genes which lack a nearby promoter.

In addition to promoter replacement methods, regulatory element insertion/replacement methods can also be used in methods to identify genes required for proliferation. For example, a nonregulatable, native promoter which directs the transcription of a proliferation-required gene can be rendered regulatable by inserting a regulatory element into the chromosome of the cell via homologous recombination in a location where the regulatory element is capable of regulating the level of transcription from the promoter. A variety of regulatory elements can be used to regulate the expression of proliferation-required gene products. In some versions of this method, the regulatory element can be an operator which is recognized by a repressor (e.g. lac, tet, araBAD repressors), a transcriptional terminator or a nucleotide sequence which introduces a bend in the DNA.

To bring the proliferation-required gene under regulatory control, a linear regulatory element insertion cassette comprising a regulatory element, which is flanked by nucleotide sequences having homology to the natural promoter or other relevant locations upstream, downstream, or within the gene of interest, is introduced into the cell. In some embodiments, the cassette also comprises a nucleotide sequence encoding a selectable marker or a marker whose expression is readily identified. The cassette can be a double stranded nucleic acid or a single stranded nucleic acid as described in U.S. patent application Ser. No. 09/948,993, entitled RAPID METHOD FOR REGULATING GENE EXPRESSION, filed Sep. 6, 2001, the disclosure of which is incorporated herein by reference in its entirety. Upon homologous recombination, the regulatory element is inserted into the chromosome, leaving the gene required for proliferation under the control of the regulatory element. Strains in which the gene required for proliferation is under control of the regulatory element are grown under conditions in which the level of the proliferation-required gene product produced is less than the level in a wild type cell.

In some embodiments, the cell into which the promoter replacement cassette or regulatory element insertion cassette is introduced has an enhanced frequency of recombination. For example, the cells can lack or have a reduced level or activity of one or more exonucleases which would ordinarily degrade the DNA to be inserted into the chromosome. In further embodiments, the cells can both lack or have reduced levels of exonucleases and express or overexpress proteins involved in mediating homologous recombination. For example, if the methods are performed in Escherichia coli or other enteric prokaryotes, cells in which the activity of exonuclease V of the RecBCD recombination pathway, which degrades linear nucleic acids, has been reduced or eliminated, such as recB, recC, or recD mutants can be used. In some embodiments, the cells have mutations in more than one of the recB, recC, and recD genes which enhance the frequency of homologous recombination. For example the cells can have mutations in both the recB and recC genes.

The promoter replacement or regulatory element insertion methods can also be performed in Escherichia coli cells in which the activity of the RecET recombinase system of the Rac prophage has been activated, such as cells which carry an sbcA mutation. The RecE gene of the rac prophage encodes ExoVIII a 5′-3′ exonuclease, while the RecT gene of the Rac prophage encodes a single stranded DNA binding protein which facilitates renaturation and D-loop formation. Thus, the gene products of the RecE and RecT genes or proteins with analogous functions facilitate homologous recombination. The RecE and RecT genes lie in the same operon but are normally not expressed. However, sbcA mutants activate the expression the RecE and RecT genes. In some embodiments, the methods can be performed in cells which carry mutations in the recB and recC genes as well as the sbcA mutation. The RecE and RecT gene can be constitutively or conditionally expressed. For example, the methods can be performed in E. coli strain JC8679, which carries the sbcA23, recB21 and recC22 mutations.

In some embodiments, the methods can be performed in Escherichia coli cells in which recombination via the RecF pathway has been enhanced, such as cells which carry an sbcB mutation.

It will be appreciated that the RecE and RecT gene products, or proteins with analogous functions can be conditionally or constitutively expressed in prokaryotic organisms other than E. coli. In some embodiments, these proteins can be conditionally or constitutively expressed in Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species. For example, plasmids encoding these gene products can be introduced into the organism. If desired, the coding sequences encoding these gene products can be optimized to reflect the codon preferences of the organism in which they are to be expressed. Similarly, in some embodiments, the organism can contain mutations analogous to the recB, recC, recD, sbcA or sbcB mutations which enhance the frequency of homologous recombination.

In further embodiments, the promoter replacement or regulatory element insertion methods can be conducted in cells which utilize the Red system of bacteriophage lambda (λ) or analogous systems from other phages to enhance the frequency of homologous recombination. The Red system contains three genes, (γ,β and exo whose products are the Gam, Bet and Exo proteins (see Ellis et al. PNAS 98:6742-6746, 2001, the disclosure of which is incorporated herein by reference in its entirety). The Gam protein inhibits the RecBCD exonuclease V, thus permitting Beta and Exo to gain. access to the ends of the DNA to be integrated and facilitating homologous recombination. The Beta protein is a single stranded DNA binding protein that promotes the annealing of a single stranded nucleic acid to a complementary single stranded nucleic acid and mediates strand exchange. The Exo protein is a double-stranded DNA dependent 5′-3′ exonuclease that leaves 3′ overhangs that can act as substrates for recombination. Thus, constitutive or conditional expression of the λ Red proteins or proteins having analogous functions facilitates homologous recombination. It will be appreciated that the λ Beta, Gam and Exo proteins, or proteins with analogous functions can be expressed constitutively or conditionally in prokaryotic organisms other than E. coli. In some embodiments, these proteins can be conditionally or constitutively expressed in Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallet, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnet, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species. For example, plasmids encoding these gene products can be introduced into the organism. If desired, the coding sequences encoding these gene products can be optimized to reflect the codon preferences of the organism in which they are to be expressed.

In some embodiments, the cells can have an increased frequency of homologous recombination as a result of more than one of the aforementioned characteristics. In some embodiments, the enhanced frequency of recombination can be a conditional characteristic of the cells which depends on the culture conditions in which the cells are grown. For example, in some embodiments, expression of the λ Red Gam, Exo, and Beta proteins or recE and recT proteins can be regulated. Thus, the cells can have an increased frequency of homologous recombination as a result of any combination of the aforementioned characteristics. For example, in some embodiments, the cell can carry the sbcA and recBC mutations.

In some embodiments, a linear double stranded DNA to be inserted into the chromosome of the organism is introduced into an organism constitutively or conditionally expressing the recE and recT or the λ Beta, Gam and Exo proteins or proteins with analogous functions as described above. In some embodiments, the organism can be Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species. In some embodiments, the double stranded DNA can be introduced into an organism having the recBC and sbcA mutations or analogous mutations.

In other embodiments, a single stranded DNA to be inserted into the chromosome of the organism is introduced into an organism expressing the λ Beta protein or a protein with an analogous function. In some embodiments the single stranded DNA is introduced into an organism expressing both the λ Beta and Gam proteins or proteins with analogous functions. In further embodiments, the single stranded DNA is introduced into an organism expressing the λ Beta, Gam and Exo proteins or proteins with analogous functions. The λ proteins or analogous proteins can be expressed constitutively or conditionally. In some embodiments, the organism can be Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

Regardless of which of the above-described methods are selected, a variety of assays can be used to identify nucleic acid sequences that negatively impact growth upon expression. For example, with respect to the approach based on expression of a library of exogenous nucleic acids, growth in cultures expressing exogenous nucleic acid sequences is compared to growth in cultures not expressing these sequences. Growth measurements are assayed by examining the extent of growth by measuring optical densities. Alternatively, enzymatic assays can be used to measure bacterial growth rates to identify exogenous nucleic acid sequences of interest. Colony size, colony morphology, and cell morphology are additional factors used to evaluate growth of the host cells. Those cultures that fail to grow or grow at a reduced rate under expression conditions are identified as containing an expression vector encoding a nucleic acid fragment that negatively affects a proliferation-required gene.

Once a nucleic acids of interest is identified, it can be analyzed. The first step of the analysis is to acquire the nucleotide sequence of the nucleic acid fragment of interest. In cases where expression libraries were used, the exogenous nucleic acid insert contained in an expression vector that is identified as containing a nucleotide sequence that negatively affects proliferation is sequenced using standard techniques well known in the art. The next step of the process is to determine the source of the nucleotide sequence. As used herein “source” means the genomic region containing the cloned fragment.

Determination of the gene(s) corresponding to the nucleotide sequence is achieved by comparing the obtained sequence data with databases containing known protein and nucleotide sequences from various microorganisms. Thus, initial gene identification is made on the basis of significant sequence similarity or identity to either characterized or predicted Escherichia coli, Staphylococcus aureus, Enterococcus faecalis, Klebsiella pneumoniae, Pseudomonas aeruginosa, and Salmonella typhimurium genes or their encoded proteins and/or homologues in other species.

The number of nucleotide and protein sequences available in database systems has been growing exponentially for years. For example, the complete nucleotide sequences of several bacterial genomes, including E. coli, Aeropyrum pernix, Aquifex aeolicus, Archaeoglobus fulgidus, Bacillus subtilis, Borrelia burgdorferi, Chlamydia pneumoniae, Chlamydia trachomatis, Clostridium tetani, Corynebacterium diptheria, Deinococcus radiodurans, Haemophilus influenzae, Helicobacter pylori 26695, Helicobacter pylori J99, Methanobacterium thermoautotrophicum, Methanococcus jannaschii, Mycobacterium tuberculosis, Mycoplasma genitalium, Mycoplasma pneumoniae, Pseudomonas aeruginosa, Pyrococcus abyssi, Pyrococcus horikoshii, Rickettsia prowazekii, Synechocystis PCC6803, Thermotoga maritima, Treponema pallidum, Bordetella pertussis, Campylobacter jejuni, Clostridium acetobutylicum, Mycobacterium tuberculosis CSU#93, Neisseria gonorrhoeae, Neisseria meningitidis, Pseudomonas aeruginosa, Pyrobaculum aerophilum, Pyrococcus furiosus, Rhodobacter capsulatus, Salmonella typhimurium, Streptococcus mutans, Streptococcus pyogenes, Ureaplasma urealyticum and Vibrio cholera are available. This nucleotide sequence information is stored in a number of databanks, such as GenBank, the National Center for Biotechnology Information (NCBI), the Genome Sequencing Center and the Sanger Centre, which are publicly available for searching. For example, genomic sequences and related information from the Genome Sequencing Center is available on the internet and can be accessed by entering the following quoted text, “genome.wustl”, in the address bar of a web browser, such as Internet Explorer or Netscape, followed immediately by “.edu/gsc/salmonella/shtml”. Likewise sequence data from the Sanger Center can be accessed by entering the following quoted text, “www.sanger.ac”, in the address bar of a web browser, such as Internet Explorer or Netscape, followed immediately by “.uk/projects/S_{—}typhi”.

A variety of computer programs are available to assist in the analysis of the sequences stored within these databases. FASTA, (W. R. Pearson (1990) “Rapid and Sensitive Sequence Comparison with FASTP and FASTA” Methods in Enzymology 183:63-98), Sequence Retrieval System (SRS), (Etzold & Argos, SRS an indexing and retrieval tool for flat file data libraries. Comput. Appl. Biosci. 9:49-57, 1993) are two examples of computer programs that can be used to analyze sequences of interest. In one embodiment of the present invention, the BLAST family of computer programs, which includes BLASTN version 2.0 with the default parameters, or BLASTX version 2.0 with the default parameters, is used to analyze nucleotide sequences.

BLAST, an acronym for “Basic Local Alignment Search Tool,” is a family of programs for database similarity searching. The BLAST family of programs includes: BLASTN, a nucleotide sequence database searching program, BLASTX, a protein database searching program where the input is a nucleic acid sequence; and BLASTP, a protein database searching program. BLAST programs embody a fast algorithm for sequence matching, rigorous statistical methods for judging the significance of matches, and various options for tailoring the program for special situations. tBLASTX can be used to translate a nucleotide sequence in all three potential reading frames into an amino acid sequence.

The above described methods have previously been used to identify numerous proliferation-required genes in Staphylococcus aureus as well as numerous other organisms. For example, these methods have been used to identify a group of genes, each having a nucleotide sequence selected from the group consisting of SEQ ID NOs: 201-550, as required for cellular proliferation. Each of these genes encodes a proliferation-required gene product which has an amino acid sequence selected from the group consisting of SEQ ID NOs: 551-824. Proliferation required genes have also been identified from numerous other bacteria. It will be appreciated that the above-described methods can be used in any microorganisms including, but not limited to, pathogenic bacteria such as, Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis and any species falling within the genera of any of the above species.

Although the above methods are useful for determining the identity of genes required for proliferation, there are times when the nucleic acid that is identified corresponds to a nucleotide sequence other than a proliferation-required gene or a portion thereof. For example, when using the exogenous library method, the provided nucleic acid can inhibit the activity or reduce the amount of a product of a proliferation-required gene that is contained in an operon by inhibiting the expression of the entire polycistronic transcript that is produced by the operon or by inhibiting the expression of a portion of the polycistronic transcript which comprises the proliferation-required gene. Such an inhibition of the production of multiple gene products from a polycistronic transcript is known as a polar effect. Thus, in the context of an operon, it is possible that the exogenous nucleic acid, which has been identified as inhibiting the activity or reducing the amount of a proliferation-required gene product, corresponds to a gene that is required for proliferation or portion thereof, a 5′ noncoding region or a 3′ noncoding region located upstream or downstream from the actual gene that is required for proliferation or a portion thereof, an intergenic sequence (i.e. a sequence between genes) or a portion thereof, a gene that is not required for proliferation or a portion thereof, or a nucleotide sequence spanning at least a portion of two or more genes.

Given the nature of the screening protocol, it would be useful to identify operon boundaries and then determine the which gene(s) contained in the operon are individually required for proliferation. The methods described below can be used to identify operons in prokaryotic organisms. These techniques are especially useful in organisms for which substantial genomic sequence is available but for which little or no data regarding operon structure exists.

Methods of Operon Prediction

One aspect of the present invention relates to methods for predicting operons in the genome of an organism, such as a prokaryotic organism. As will be described in greater detail hereinbelow, sequence data can be used to demonstrate how certain methods and approaches are implemented by the present teachings to perform operon prediction and identification. The methods described herein may be utilized with any organism. In a preferred embodiment, the organism is a prokaryotic organism. In one aspect, the complete genomic sequence of Staphylococcus aureus Mu50 strain serves as an exemplary prokaryotic genome used in consensus analysis and operon prediction. Additional details of this genome may be found in the publication: Kuroda, M., et al., “Whole genome sequencing of methicillin-resistant Staphylococcus aureus, the major hospital pathogen.” Lancet 357, 1225-1240, (2001). The genomic information from this bacterial strain may be obtained from NCBI by entering the following quoted text, “www.ncbi.nlm.nih.”, in the address bar of a web browser, such as Internet Explorer or Netscape, followed immediately by “gov:80/PMGifs/Genomes/org.html”. The genomic information can then be imported into a local database. In one embodiment, the microbial sequence database, PathoSeq™ (Elitra, San Diego, Calif.) was used as the local database for storage of information used in operon prediction according to the present teachings.

Sequences for other bacterial genomes used in the below-described exemplary comparative genome analysis were also obtained from The National Center for Biotechnology Information (NCBI) or The Institute for Genomic Research (TIGR) and imported to the local database. The genome sequences obtained from NCBI include: Aquifex aeolicus strain VF5, Borrelia burgdorferi strain B31, Bacillus halodurans C-125, Bacillus subtilis strain 168, Buchnera sp. APS, Clostridium acetobutylicum ATCC824, Caulobacter crescentus CB 15, Campylobacter jejuni strain NCTC 11168, Chlamydiapneumoniae strain AR-39, Chlamydia trachomatis strain MoPn, Escherichia coli strain K-12, Haemophilus influenzae strain KW20, Helicobacter pylori strain 26695, Listeria monocytogenes, Listeria innocua, Lactococcus lactis strain IL1403, Mycoplasma genitalium isolate G37, Mycobacterium leprae strain TN, Mycoplasma pneumoniae strain M129, Mycoplasma pulmonis UAB CTIP, Mycobacterium tuberculosis CDC1551, Neisseria meningitides strain MC58, Pseudomonas aeruginosa strain PAO1, Pasteurella multocida Pm70, Rickettsia conorii Malish 7, Rickettsia prowazekii strain Madrid E, Sinorhizobium meliloti 1021, Streptococcus pneumoniae strain R6 hex, Streptococcus pyogenes strain M1 GAS, Salmonella typhi strain CT18, Salmonella typhimurium strain SGSC1412, Synechocystis sp. strain PCC 6803, Thermotoga maritime strain MSB8, Treponema pallidum strain Nichols, Ureaplasma urealyticum, Vibrio cholerae strain N16961, Xylella fastidiosa 9a5c and Yersinia pestis strain CO-92 Biovar Orientalis. The genome sequence of Enterococcus facaelis strain V583 was downloaded from TIGR.

With reference to FIG. 1, one aspect of the present invention provides a consensus model for operon prediction 100 which defines a computational pipeline to annotate operons in a prokaryotic organism's genome. As will be described in greater detail hereinbelow, the consensus model 100 utilizes a technique for pairwise analysis of genes using the various operon prediction methods 110 to assess the likelihood that adjacent gene pairs reside within the same operon. Operon prediction in this manner provides a relatively rapid and reliable means for identifying co-transcribed genes with greater accuracy and sensitivity. As will be appreciated by one of skill in the art, the validity of this model can be readily assessed by evaluation of known operons for selected organisms and comparison of the results obtained with literature values. Additionally, operon prediction may proceed without known information regarding operon structure and therefore may desirably be used in connection with prokaryotic organisms for which little or no operon information currently exists.

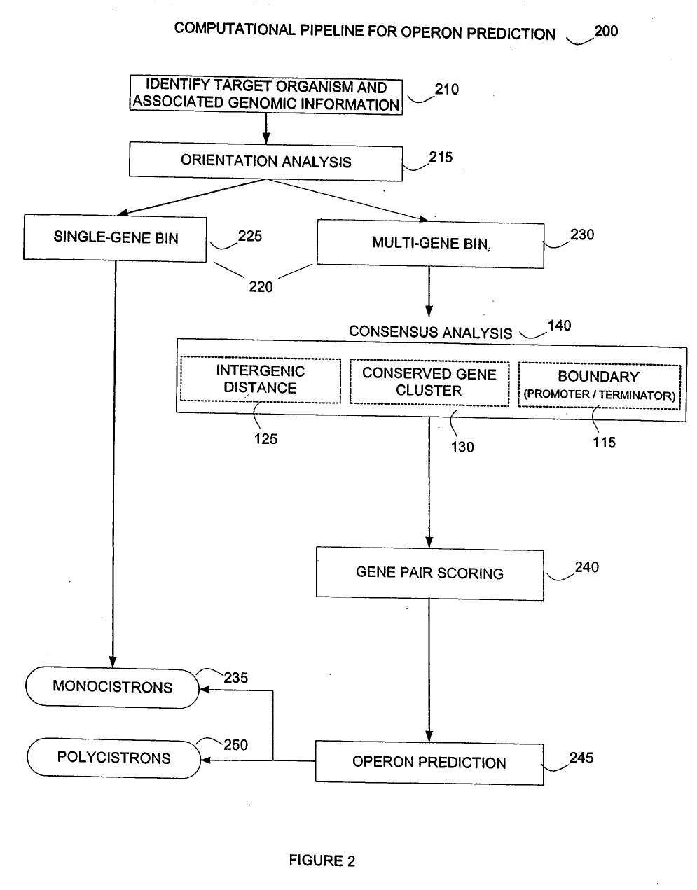

FIG. 2 illustrates a computation pipeline 200 for operon prediction in which the aforementioned consensus model 100 is utilized. In one aspect, this method 200 is capable of performing rapid and reliable analysis for one or more target organisms whose genomic composition is to be evaluated. It will be appreciated that the below-described methods may be readily transformed or adapted to a corresponding software application or system to be used in computerized analysis. Furthermore, these methods may be integrated into other software and hardware components to provide increased functionality in operon identification. It is therefore conceived that software and hardware implementations which integrate the use of the consensus analysis model 100 or computational pipeline 200 are but other embodiments of the present teachings.

The method 200 commences in a state 210 wherein at least one target prokaryotic organism and associated genomic information is identified. The genomic information may further comprise either a portion or the entirety of the nucleotide sequence representative of the organism's genomic makeup. In one aspect, it is desirable to utilize the complete nucleotide sequence for the organism's genome, if available, to aid in the identification of substantially all operons. The disclosed methods however, will also operate with a selected portion of the genomic sequence.

Following selection of the target prokaryotic organism genomic information 210, the method 200 proceeds to state 215 wherein an orientation analysis is performed. In one aspect, the orientation analysis comprises identifying pairs of genes and assessing their orientation relative to one another. For example, with respect to a gene having a 5′ - - - 3′ orientation, a paired gene may either be oriented in the same direction (i.e. 5′ - - - 3′) or alternatively the paired gene may be oriented in the opposing direction (i.e. 3′ - - - 5′). Thus, a complete orientation analysis may be performed for each gene in the identified genomic information 210 by identifying flanking or consecutive gene pairs and determining the orientation of the constituent genes with respect to one another. The results of such an orientation analysis 215 are representative of the plurality of consecutive gene pairings and their respective orientations (being either similarly or oppositely aligned).

Using the information provided by the orientation analysis, each gene obtained from the genomic information of the target organism may then be segregated into one of two categories comprising bins or groupings 220. The first grouping category comprises a single-gene bin 225 containing genes having an opposite alignment relative to their 5′ and 3′ flanking genes. Genes which fall into this category are considered to form monocistronic operons which may be indicative of a transcription unit containing a single gene. The second grouping category comprises a multi-gene bin 230 containing genes having at least one flanking or consecutive gene aligned in the same orientation as the selected gene. Genes which fall into this category 230 are further evaluated to determine whether they lie in polycistronic operons having a transcription unit containing more than one gene. Thus, for a selected target organism, each gene identified in the genomic information is desirably segregated into one of two principal bins being either the single-gene bin 225 or the multi-gene bin 230. In the instance of genes which are segregated into the multi-gene bin 230 these genes are further segregated based on their composition and similarity in orientation as will be described in greater detail hereinbelow.

In one aspect, the multi-gene bin 230 comprises a plurality of discrete groupings with each grouping containing two or more consecutive genes that are determined to be aligned in the same orientation with respect to one another. Segregation of consecutive genes possessing similar orientations into the discrete groupings of the multi-gene bin 230 provides a means for associating genes which may be part of the same putative polycistronic operon. Additionally, by separating each of the genes contained in the supplied genomic information into the single-gene bin 225 or the multi-gene bin 230 desirably reduces the actual number of genes which will undergo more rigorous consensus analysis whereby genes associated with the single-gene bin 225 may be identified as monocistrons in state 235 and excluded from further analysis. In one aspect, the above-described manner of initial segregation desirably improves the speed and performance of co-transcriptional or operon analysis by focusing rigorous computation efforts on those genes which have a significant possibility of being part of a polycistronic operon. In one aspect, the analysis of the genes contained in the single gene bin 225 is desirably completed early in the computational pipeline 200 resulting in less downstream data and information to be processed.

Applying the aforementioned approach to the genes contained in the genome for the exemplary organism, Staphylococcus aureus, a total of approximately 2790 genes (including tRNA and rRNA genes) were resolved to a total of approximately 670 discrete bins 220. Of these bins 220, approximately 273 contain a single gene with no flanking gene in the same orientation and the remaining 297 bins were identified as comprising multiple consecutive genes having similar orientations. For the identified multi-gene bins 230, the number of consecutive genes contained in the bin may range from 2 to approximately 63 genes or more with an average of approximately 6.34 genes per multi-gene bin 230. The preceding example demonstrates the pipeline's ability to rapidly identify a significant number of monocistrons from the genomic information and furthermore shows how genes which may be part of a polycistronic operon may be segregated into discrete groupings in preparation for further analysis.

Having identified those gene pairs associated with the multi-gene bin 230 the pipeline 200 proceeds to the consensus analysis state 140. As previously described, consensus analysis may comprise subjecting each gene or gene pair to various analytical approaches used in operon prediction. As shown in FIG. 2, three exemplary approaches which may be used alone or in conjunction with one or more of the other approaches during consensus analysis 140 include: intergenic distance analysis 125, conserved gene cluster analysis 130, and boundary analysis 115.

In various embodiments, intergenic distance analysis 125 may be performed using some or all of the genes and associated gene pairings contained in the multi-gene bin 230 wherein the genes are identified by orientation relative to proximal 5′ and 3′ flanking genes. The intergenic distances between adjacent genes in the same orientation may then be calculated from the corresponding genomic coordinates in the target organism or strain. For example, the intergenic distance between a first gene_{A} and a second gene_{B} may be calculated according to the following formula: distance_{AB}=gene_{B}_{_{—}}start_position-gene_{A}_{_{—}}end_position. The orientation and distance information for of each gene pair may be subsequently saved (for example in a database or other data structure) and used for purposes of operon identification in the manner described below.

In one aspect, the intergenic distance analysis 125 takes into account the observation that the intergenic distance between selected genes contained within an operon may be shorter than distances between genes not contained within the same operon. In applying this approach, an intergenic distance analysis 125 may be conducted according to a prediction scheme in which gene pairs in the same discrete grouping in the multi-gene bin 230 are considered to be in the same putative operon if their intergenic distance falls below a selected distance threshold.

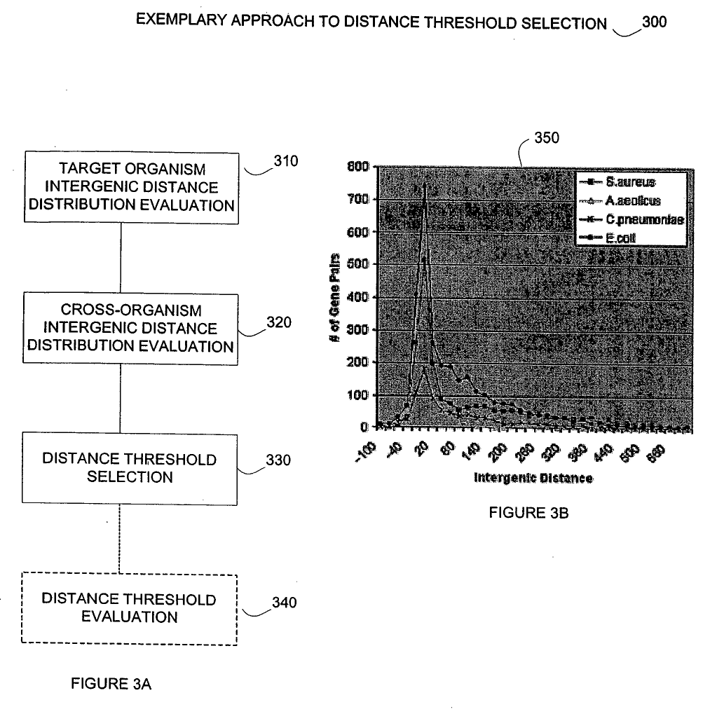

In one aspect, an ideal distance threshold may not be readily apparent due to insufficient experimental or literature data for a selected organism. Consequently, if desired, as shown in FIGS. 3A and 3B the distance threshold may be determined by comparison of the genomic information for the selected or target organism with one or more reference organisms.

In FIG. 3A, a method 300 for distance threshold selection is illustrated. The method 300 commences in a state 310 with a determination of the intergenic distance distribution across adjacent gene pairs contained in the multi-gene bin 230 for the target or selected organism. In state 320, this information may then be compared with an assessment of overall intergenic distance distributions for one or more reference organisms for which the genome organizations are known in part or whole. By comparing the target organism's intergenic distance distribution with that of the one or more reference organisms whose genomic organizations bear relative similarities in terms of gene spacing, an approximate distance threshold may be identified in state 330.

FIG. 3B, further illustrates the method 300 of distance threshold selection wherein the overall intergenic distribution for an exemplary target organism, Staphylococcus aureus, is compared to the overall intergenic distributions for a plurality of other reference organisms including for example: Aquifex aeolicus Chlamydia pneumoniae and Escherichia coli. As shown in this figure, the intergenic distances between adjacent gene pairs for the comparison organism Escherichia coli were similar in many instances to adjacent gene pairs in the same orientation for the target organism Staphylococcus aureus. Using this information, as well as that for the other reference organisms, an approximation of the intergenic distribution for the target organism may be determined to reside between approximately −20 bp and 60 bp around stop codons for the first genes with a prominent peak occurring at approximately 20 bp. Using this information, it may be reasonable to infer that the distribution of intergenic distances for the target organism may bear similarities to that of the other comparison organisms and that it may be feasible to use “generic” or cross-organism distance thresholds for operon prediction in a broad number of bacteria species.

To further refine the selected distance threshold obtained in state 330, the effects of selecting different distance thresholds for operon prediction in the target organism may be evaluated and their results compared. Table I illustrates by way of example, the effects of selecting different distance thresholds for the target organism Staphylococcus aureus. As shown in the table, comparison of putative operons at different thresholds including 30 bp, 50 bp, 75 bp, 100 bp, 150 bp and 200 bp using intergenic distance analysis 125 alone may be used to aid in the identification of an appropriate stringency and corresponding distance threshold to be selected in the consensus analysis 140. From the data, both the 30 bp and 50 bp thresholds appear to be highly stringent whereas the 150 bp and 200 bp thresholds appear to be significantly less stringent with the 75 bp and 100 bp thresholds falling somewhere in between in terms of overall stringency.

TABLE I
Comparison of putative operons at different intergenic distance
thresholds using the intergenic distance analysis method alone

| | | Single-Gene Bins | Muti-gene Bins | | Largest |
|---|---|---|---|---|---|
| Distance | Identified | Identified | Identified | Identified | Identified |
| Threshold (bp) | Operons | Monocistrons | Monocistrons | Polycistrons | Operon Size |
| 30 | 1820 | 273 | 757 | 517 | 14 |
| 50 | 1718 | 273 | 632 | 540 | 25 |
| 75 | 1610 | 273 | 502 | 562 | 25 |
| 100 | 1541 | 273 | 428 | 567 | 25 |
| 150 | 1385 | 273 | 265 | 574 | 29 |
| 200 | 1237 | 273 | 113 | 578 | 46 |

Using this information along with literature and/or experimental information a distance threshold may be selected which balances the expected number of false positive and false negatives. For example, a distance threshold of 75 bp or 100 bp may be selected; however, other values may be also selected in order to achieve other desired stringencies.

Referring again to FIG. 2, another consensus analysis model 140 comprises the conserved gene cluster approach 130. This method 130 is based upon the observation that evaluation of conserved gene order across various bacterial genomes may reveal gene clusters which may have an increased probability of residing within the same operon. In one aspect, this method 130 uses available sequence information from one or more prokaryotic genomes that is compared to the target organism's genomic information. As will be described in greater detail hereinbelow, using the criteria of sequence composition and location, operon prediction in the target organism can be performed by this method and compliment or supplement other methods used in consensus analysis 140.

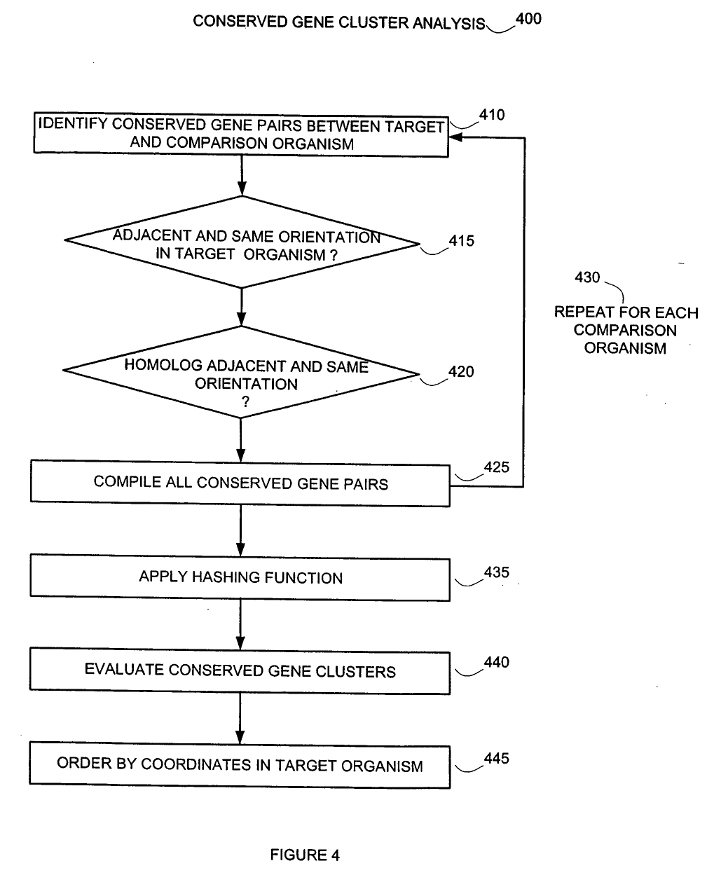

FIG. 4 illustrates a method 400 for conserved gene cluster analysis 130 that may be used in operon identification. In one aspect, this method utilizes genomic information from at least one comparison organism which is analyzed against the genes contained in the target organism. Gene conservation between the organisms can be performed using sequence analysis applications including for example, BLASTP or BLASTN, wherein orthologs for a gene in the target organism are desirably identified as the reciprocal best BLAST hits between compared genomes.

As shown in the figure, the method 400 commences in state 410 wherein one or more comparison genomes and associated information are selected and conserved gene pairs between the comparison organism(s) and the target organism genomic information identified. Conservation is identified, in state 415 wherein gene pairs in the target organism that are both adjacent and in the same orientation are ascertained. Subsequently, in state 420 the identified gene pairs from state 415 are assessed against homologous gene pairs in the comparison organism based on similarities in adjacency and orientation. Conserved gene pairs that are adjacent to one another and in the same orientation in both the target organism and the comparison organism determined according to the aforementioned criteria are then compiled in a table (e.g. hash table) or other data structure in state 425 to reflect conserved gene pairs between the target and comparison organism. These steps are repeated 430 for each comparison organism available to generate additional compilations of conserved genes.

In state 435, a hashing function is applied to the conserved gene pair data to cluster the data into one or more conserved gene arrays wherein a hash value is associated with gene pairs contained in the table to provide a means for grouping the genes. Subsequently, the conserved gene clusters identified by the hashing analysis 435 may be evaluated in state 440 and ordered by coordinates in the target organism in state 445 to serve as a means for operon prediction.

As an example of how these operations may proceed, the hashing function and subsequent evaluations and orderings may be performed according to the following steps for each row from the gene pair table/hash table for each selected comparison between the target and comparison organisms:

For each row in the hash table, the genes for a selected gene pair are retrieved (e.g. gene_{A} and gene_{B}) and the following criteria for generating the associated hash value are applied:
a) Each entry contained within the table for both genes of the gene pair is initialized with a principal hash value (for example, 0).
b) If the hash value entries contained within the table for both genes of the gene pair are equivalent to the principal hash value (e.g. the value 0), then these two genes are considered to belong to a new cluster together and these genes may be assigned a discrete cluster number. In one aspect, the hash table entry may be representative of an offset into a cluster array or other data structure that contains the designated cluster value.
c) If only a single gene of the gene pair possesses a hash value corresponding to the principal hash value, then the hash value corresponding to the selected gene is assigned a value equivalent to that of the other gene pair member.
d) If both genes of the gene pair possess an identical non-principal hash value, this may be deemed to signify that the same gene pairs have been identified by a prior comparison with another organism and as a consequence this row may be skipped without hash value modification.
e) If both genes of the gene pair possess a different non-principal hash value, then each occurrence of the greater cluster value may be replaced with the lesser cluster value in the cluster array.
f) Genes that possess the same hash value in the hash table may be considered to belong to the same conserved gene clusters and these genes may be ordered by their relative coordinates in the target organism genome.

In one aspect, the rate or frequency of false positives may be moderated by restricting the analysis of gene pairs to those which fall within a desirable range of intergenic distances. For example, an intergenic distance range between approximately 0 and 300 bp may be selected for evaluation of gene pairs contained within the same multi-gene bins.

Table II illustrates the results of applying the conserved gene cluster analysis method 400 to evaluate an exemplary target organism comprising Staphylococcus aureus relative to other available organisms and their corresponding genomic information. In this table, the values of conserved gene clusters between Staphylococcus aureus and other exemplary genomes are shown wherein 39 total bacterial genomes were used in the analysis which comprised: 14 Gram-positive (G+), 23 Gram-negative (G−), and 2 others organism types. As shown by this data, use of multiple genomes in comparison to the target organism desirably increases the predicted number of conserved gene clusters.

TABLE II
Conserved gene clusters analysis in Staphylococcus aureus when
compared to different bacterial genomes

| | | No. of | | Percent of | Average | Largest |
|---|---|---|---|---|---|---|
| | | Conserved | No. of | Staphylococcus | Cluster | Cluster |
| Organism Compared | Type | Gene Clusters | Genes | aureus Genes | Size | Size |
| Aquifex aeolicus | G− | 27 | 76 | 2.8% | 2.8 | 10 |
| Bacillus halodurans | G+ | 245 | 714 | 26.3% | 2.9 | 23 |
| Bacillus subtilis | G+ | 244 | 722 | 26.6% | 3.0 | 23 |
| Borrelia burgdorferi | Spirochete | 39 | 114 | 4.2% | 2.9 | 22 |
| Buchnera sp. | G− | 40 | 119 | 4.4% | 3.0 | 14 |
| Campylobacter jejuni | G− | 42 | 121 | 4.5% | 2.9 | 19 |
| Caulobacter crescentus | G− | 75 | 190 | 7.0% | 2.5 | 14 |
| Chlamydia pneumoniae | G− | 40 | 101 | 3.7% | 2.5 | 8 |
| Chlamydia trachomatis | G− | 42 | 105 | 3.9% | 2.5 | 8 |
| Clostridium acetobutylicum | G+ | 138 | 384 | 14.1% | 2.8 | 23 |
| Enterococcus faecalis | G+ | 200 | 545 | 20.1% | 2.7 | 23 |
| Escherichia coli | G− | 98 | 249 | 9.2% | 2.5 | 14 |
| Haemophilus influenzae | G− | 74 | 191 | 7.0% | 2.6 | 11 |
| Helicobacter pylori | G− | 29 | 85 | 3.1% | 2.9 | 15 |
| Lactococcus lactis | G+ | 151 | 382 | 14.1% | 2.5 | 15 |
| Listeria innocua | G+ | 238 | 718 | 26.5% | 3.0 | 23 |
| Listeria monocytogenes | G+ | 243 | 732 | 27.0% | 3.0 | 23 |
| Mycobacterium leprae | G+ | 72 | 181 | 6.7% | 2.5 | 11 |
| Mycobacterium tuberculosis | G+ | 84 | 209 | 7.7% | 2.5 | 10 |
| Mycoplasma genitalium | G+ (Acid Fast) | 49 | 139 | 5.1% | 2.8 | 19 |
| Mycoplasma pneumoniae | G+ (Acid Fast) | 51 | 143 | 5.3% | 2.8 | 19 |
| Mycoplasma pulmonis | G+ (Acid Fast) | 51 | 133 | 4.9% | 2.6 | 12 |
| Neisseria meningitidis | G− | 46 | 126 | 4.6% | 2.7 | 13 |
| Pasteurella multocida | G− | 73 | 185 | 6.8% | 2.5 | 14 |
| Pseudomonas aeruginosa | G− | 86 | 222 | 8.2% | 2.6 | 14 |
| Rickettsia conorii | G− | 35 | 97 | 3.6% | 2.8 | 14 |
| Rickettsia prowazekii | G− | 36 | 99 | 3.6% | 2.8 | 14 |
| Salmonella typhi | G− | 91 | 233 | 8.6% | 2.6 | 14 |
| Salmonella typhimurium | G− | 86 | 220 | 8.1% | 2.6 | 14 |
| Sinorhizobium meliloti | G− | 69 | 179 | 6.6% | 2.6 | 14 |
| Streptococcus pneumoniae | G+ | 155 | 369 | 13.6% | 2.6 | 14 |
| Streptococcus pyogenes | G+ | 137 | 396 | 14.6% | 2.7 | 28 |
| Synechocystis sp. | G− | 28 | 84 | 3.1% | 3.0 | 13 |
| Thermotoga maritima | G− | 70 | 192 | 7.1% | 2.7 | 23 |
| Treponema pallidum | Spirochete | 32 | 94 | 3.5% | 2.9 | 19 |
| Ureaplasma urealyticum | G− | 48 | 143 | 5.3% | 3.0 | 19 |
| Vibrio cholerae | G− | 80 | 203 | 7.5% | 2.6 | 14 |
| Xylella fastidiosa | G− | 60 | 163 | 6.0% | 2.7 | 14 |
| Yersinia pestis | G− | 89 | 230 | 8.5% | 2.6 | 14 |

Referring again to FIG. 2, another consensus analysis model 140 comprises the boundary analysis 115. This method 115 is based upon the detection of signals at transcription unit boundaries which may include identification of promoter and terminator sequences and structures. As will be described in greater detail hereinbelow, software applications familiar to those skilled in the art, including the GCG Wisconsin Package of programs, may be used to aid in boundary analysis or alternatively custom developed searching algorithms or other software applications may be used to perform similar operations to aid in the identification of boundary sequence information. As with other operon predication schemes 110, this method 115 may be used to aid in operon prediction in the target organism and furthermore compliment or supplement other methods used in consensus analysis 140.

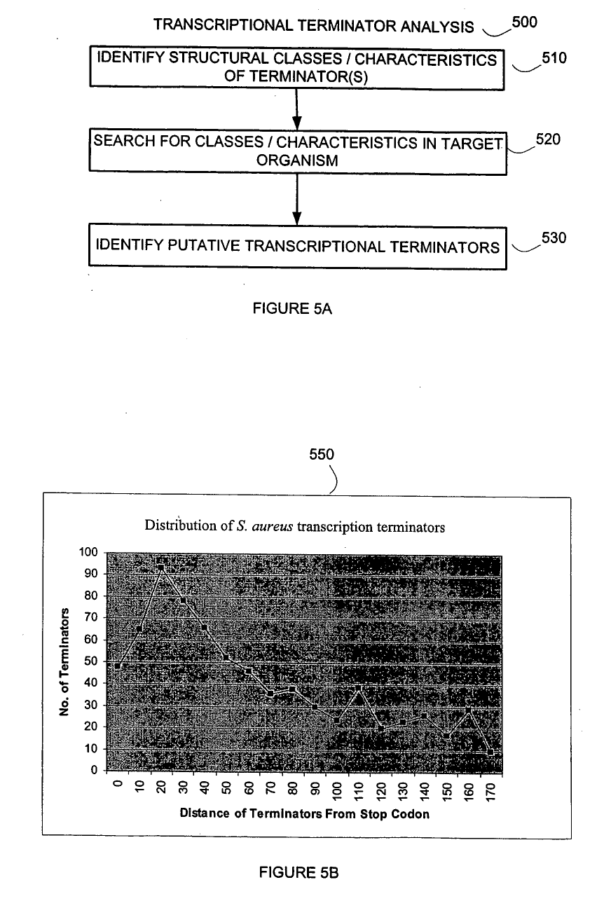

FIG. 5A illustrates an exemplary method 500 for boundary analysis 115 comprising a transcriptional terminator identification approach. Generally, transcription terminators such as Rho-independent transcription terminators possess distinct secondary structures in nascent RNA. Putative terminators from a number of available prokaryotic genomes may be grouped by structure into classes including: L-shaped, I-shaped, V-shaped, U-shaped, and X-shaped. In one aspect, the majority of transcription terminators detected in the target organism may comprise one or more selected classes.

For example in the case of Staphylococcus aureus, a significant portion of the rho-independent transcription terminators detected comprise L-shaped structures having a stem-loop followed by a poly U-tail. Accordingly, the method 500 commences in state 510 with the identification of one or more classes of transcriptional terminators. The structural characteristics for the selected classes of transcriptional terminators may then be used as a search criterion within the target organism's genomic information in state 520. In various embodiments, searching may be performed using software applications familiar to those skilled in the art, including GCG Terminator, part of the GCG Wisconsin Package of programs.

In the exemplary target organism, Staphylococcus aureus, searching of the genomic information indicative of putative terminator structure may comprise searching for GC-rich dyad symmetry near a U-rich region. In performing this search, a selected portion of sequence information downstream of each gene's stop codon may be used in the analysis of putative transcriptional terminators. For example, the sequence ranging between approximately −20 bp and 200 bp downstream of each gene's stop codon may be used in the terminator search in state 520.

Subsequently, in state 530 putative transcriptional terminators may be determined on the basis of identification of expected terminator structure(s). In one aspect, predicted terminators are called and extracted on the basis of exceeding a selected S-value of approximately greater than 0. While a significant number of putative terminators may be located in proximity to the stop codons, other terminators may be located further downstream.

FIG. 5B illustrates the relative distribution of transcription terminators 550 predicted using the aforementioned methods. As shown in this figure, the predicted transcription terminators have a higher distribution in the sequence range of between approximately 0 bp and 70 bp downstream of the stop codon. However, significant numbers of transcriptional terminators may be predicted further downstream. Consequently, the sequence range searched by the method 500 may be adjusted to modify the stringency of analysis to capture more or less putative transcriptional terminators. In some embodiments, the sequence range may be adjusted to reflect the distribution of stop codons in the target organism.

While the foregoing approach describes a method 500 by which transcription terminators may be identified, it will be appreciated that other boundary analysis approaches 115 may include analysis of the genomic information for the target organism to include promoter regions as well as terminal region identification. For example, in one exemplary promoter analysis approach, a portion of the sequence upstream of a start codon for one or more operons may be identified as a putative promoter and its orientation evaluated with respect to the orientation of the start codon. Conserved motifs may be further identified using a sequence analysis application such as MEME (Multiple EM for Motif Elicitation) used in conjunction with MotifSearch, both part of the GCG Wisconsin Package. Sequence analysis in this manner may be used to identify conserved motifs in the promoter regions of the genes associated with selected start codons to help identify canonical and promoter elements. Promoter evaluation in this manner may further be used to elucidate operon boundaries and structure in a manner similar to that described above for terminator analysis.

Returning again to FIG. 2, consensus analysis 140 continues in state 240 wherein the results obtained from one or more of the operon prediction methods are combined and a consensus score is assigned to each gene-pair. As will be appreciated by one of skill in the art, each operon prediction method has its own inherent strengths and by combining the results an investigator may obtain better overall coverage and accuracy in genome-wide operon prediction. For example, the method of intergenic distance analysis 125 provides greater specificity with a relatively short distance cut-off length. Conserved gene cluster analysis 130 provides high specificity with somewhat lower sensitivity when fewer comparison organisms are utilized. Boundary analysis 115 generally provides useful information when available as the identified promoters and terminators are natural signals of operon boundaries.

As previously indicated it is generally desirable to combine the results obtained from each operon prediction method 110 in a rational manner to improve the predictive quality of the analysis. In one aspect, applying a gene-pair scoring approach 240 provides a means to weight the contribution of the selected operon prediction categories 110 and generate a value representative of the relative degree of confidence that adjacent gene pairs may reside in the same operon. As will be described in greater detail hereinbelow, this manner of operon prediction occurs in state 245 following gene pair scoring 240 to distinguish genes predicted to be associated with polycistronic operons 250 containing more than one gene from genes predicted to be associated with monocistronic operons 235 containing a single gene.

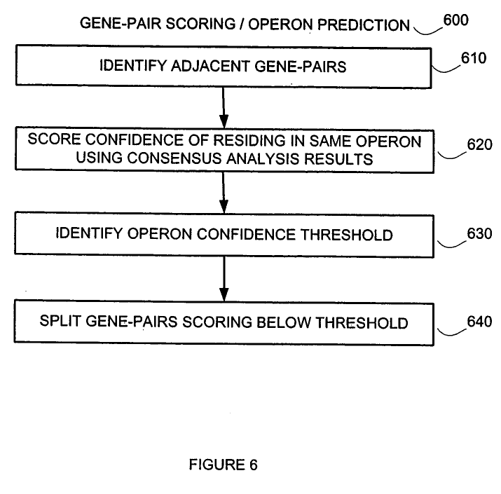

FIG. 6 illustrates a method 600 for gene pair scoring wherein a numerical score is used to represent the degree of confidence that the genes of a gene pair reside in the same operon. The method 600 commences in state 610 wherein adjacent gene-pairs are identified and subsequently in state 620 a confidence score is associated with each gene pair. In one aspect, the confidence score desirably aids in operon prediction and identification by ranking the likelihood that a selected gene-pair comprises two genes that reside in the same operon. Additional details of one method of scoring selected gene pairs will be described in greater detail hereinbelow.

Following gene-pair scoring 620, a confidence threshold is selected in state 630 which may be representative of an empirically derived or user-defined value which designates a threshold confidence level used to evaluate each gene pair in state 640 and distinguish paired genes which have a selected degree of probability of residing in the same operon from those genes which have a selected degree of probability of not residing in the same operon. In one aspect, gene-pair scores are evaluated against the selected threshold confidence level and those gene-pairs which do not possess at least a confidence value greater than the threshold confidence level are split. In other words, splitting of the gene-pairs in this manner dissociates gene-pairs whose confidence value suggests the constituent genes contained therein are not part of the same operon.

As an exemplary application of the confidence scoring method 600 described above, a numerical score of “0” may be indicative a selected gene-pair having genes that are very likely to reside in separate operons. A gene pair with a score of “1” may indicate that the gene of the gene pair may be in the same operon, but the degree of confidence is relatively low. In one aspect, the intersections of such gene pairs may also be construed as potential operon boundaries. A gene pair with a score of “2” may be indicative of the gene having a somewhat likely probably of residing in the same operon. A score of “3” may be indicative of the genes of the gene pair having a relatively high likelihood of residing in the same operon.

In one aspect, the aforementioned scoring scheme may be roughly translated as reflecting the following levels of confidence in pairwise or adjacent gene multicistron operon identification: “relatively no confidence”, “relatively little confidence”, “some degree of confidence”, and “relatively high confidence”. Similarly, a score of “0” may also be considered to reflect a “relatively high confidence” that two adjacent genes reside in discrete operons. In another aspect, the scoring scheme reflects the following principles: stringent on a score “0” (operon boundaries) and score 3 (operon interior), less stringent on score 1 (potential operon boundaries) and score 2 (probable operon).

In various embodiments, using the above-indicated scoring range, empirical criteria may be used to establish the boundaries for scoring of individual gene-pairs. An exemplary set of criteria for adjacent gene or pairwise scoring comprises the following:
a) A score of “0” is assigned to a gene pair if any of the following three criteria are met:
   I. The two adjacent genes possess different orientations; or the intergenic distance is approximately greater than 300 bp; or the intergenic distance is approximately greater than 100 bp and the number of conserved organisms is approximately equivalent to 0.
b) A score of “1” is assigned to a gene pair if the following criteria are met:
   I. The gene pair comprises an intergenic distance approximately greater than 60 bp which is conserved in approximately less than 5 organisms and a predicted terminator exists between the genes of the gene pair.
c) A score of “3” is assigned to a gene pair if any of the following criteria are met:
   I. The gene pair is conserved in approximately 10 organisms or more; or the intergenic distance is approximately less than or equal to 30 bp; or if at least two of the following requirements are met:
      i. The intergenic distance within the gene pair is approximately less than or equivalent to 50 bp; no predicted terminators are identified which correspond to the gene pair; and the relative number of organisms conserved is approximately greater than or equal to 5 but approximately less than 10.
d) Finally, a score of “2” may be assigned to a gene pair that does not meet any of the above requirements.

As will be appreciated by one of skill in the art, the foregoing scoring criteria is representative of but one of many possible methods by which scores may be assigned to each gene pair. It is conceived that other scoring systems as well as other criteria or modifications to the criteria presented herein may be applied in a similar manner to achieve a means by which each gene pair may associated with respect to residing within the same operon. As such, alternative scoring systems, modifications to the existing scoring criteria, or the establishment of alternative scoring criteria are considered but other embodiments of the present teachings.

In one aspect, operon prediction 600 by gene pair scoring 620 and gene-pair splitting 640 may be used to define putative operon boundaries. In various embodiments, operon boundaries may be established by evaluating the scores associated with each gene pair and the resulting splits for gene pairs whose confidence values fall below the confidence threshold. As previously noted, gene pair division on the basis of the confidence threshold may be observed as splitting the genes into distinct operons.

As an exemplary application of the gene pair splitting approach, division of gene pairs may be performed when the confidence score associated with a selected gene-pair is approximately equal to or below a confidence threshold having a value 0 or 1. According to the exemplified confidence scoring scale, using a confidence threshold of 0 would tend to be more inclusive of gene-pairs to be positively identified in operon prediction whereas a confidence threshold of 1 would tend to be more stringent in operon prediction. In various embodiments, these empirically derived values yield reasonable results; however, it will be appreciated that other confidence threshold cutoffs may be readily used in a manner similar to that described above.

Table III further illustrates the results of operon prediction for the exemplary organism, Staphylococcus aureus using a confidence threshold selection criteria of 0 or less than or equal to 1 as the maximum threshold. As shown in the table, when using a threshold selection score of 0 as a criterion to break or split the gene pairs, a total of approximately 864 monocistrons and 533 polycistrons may be identified. Of these different operon types, approximately 80% of gene pairs in the approximately 533 polycistrons possessed a confidence score of 3. Furthermore, approximately 60% of the polycistrons have an average score of 3, suggesting that the majority of the gene pairs in these polycistrons may be identified as having a high confidence of proper operon identification. Additionally, only a relatively small number of polycistrons possess one or more gene pairs with a confidence score of 1.

In one aspect, a confidence threshold of 0 is desirably selected to generate a relatively large percentage of operon predictions with gene pairs possessing scores of 1 to 3 being flagged or identified to indicate their confidence values. Confidence value flagging in this manner may aid in experimental design and also provides a means to not only be able to predict the largest transcription unit within a specified region, but also to predict other potential smaller transcription units in approximately the same region.

TABLE III
Operon prediction based on threshold
selection using gene-pair scoring

| | threshold = 0 | threshold <= 1 |
|---|---|---|
| # of total predicted operons | 1397 | 1459 |
| # of moncistronic operons | 864 | 921 |
| # of polycistronic operons | 533 | 538 |
| # of polycistronic operons | 53 | 0 |
| with minimum gene pair score = 1 | | |
| # of polycistronic operons | 160 | 162 |
| with minimum gene pair score = 2 | | |
| # of polycistronic operons | 320 | 376 |
| with every gene pair score = 3 | | |

Examples of Improved Operon Prediction in Staphylococcus aureus

One significant feature of the present teachings is that through consensus analysis wherein various operon prediction methods are integrated, prediction of operons is improved and operons can be identified in instances where they might be otherwise missed when using conventional singular approaches.

For example, as will be described in greater detail hereinbelow in connection with Example 7, using the methods described herein, it can be readily predicted that dnaA and dnaN reside in the same operon in Staphylococcus aureus with high confidence despite the relatively large intergenic distance. In one aspect, such a prediction is achieved by the observation that this gene pair is relatively highly conserved in the genomes to which Staphylococcus aureus may be compared.

As another example, using the operon prediction methods described herein, the genes SAV1419, murG and SAV1417 may be predicted to reside in the same operon with a relatively high degree of confidence due in part to their relatively short intergenic distance (approximately. 12 bp and 17 bp, respectively) and lack of identified terminators. In conventional operon prediction methods employing solely comparative genomics or grouping by functions, this operon might not be predicted.

It will be appreciated that operon organization is an important aspect of prokaryotic genome annotation and that by using an integrated approach which may include a combined analysis by gene orientation, distance, conserved gene clusters, and transcription terminator identification; operon prediction power can be improved over conventional identification techniques. Use of the gene-pair scoring scheme further improves the flexibility of operon identification by allowing the stringency of analysis to be modified to be more “inclusive” or “exclusive”. One desirable feature of the present teachings is that these methods do not substantially rely on experimental data and thus provide a means to predict operons in genomes where training data are not easily accessible or available.

Assignment of Essential Genes within an Operon

In one embodiment of the present invention, the methods described herein are used to predict the boundaries of an operon containing at least one proliferation-required gene and then the operon is dissected to determine which gene or genes in the operon are required for proliferation. A number of techniques that are, well known in the art can be used to dissect the operon. For example, analysis of RNA transcripts by Northern blot or primer extension techniques are commonly used to analyze operon transcripts. In one aspect of this embodiment, gene disruption by homologous recombination is used to individually inactivate the genes of an operon that is thought to contain a gene required for proliferation.

Several gene disruption techniques have been described for the replacement of a functional gene with a mutated, non-functional (null) allele. These techniques generally involve the use of homologous recombination. One technique using homologous recombination in Staphylococcus aureus is described in Xia et al. 1999, Plasmid 42: 144-149, the disclosure of which is incorporated herein by reference in its entirety. This technique uses crossover PCR to create a null allele with an in-frame deletion of the coding region of a target gene. The null allele is constructed in such a way that nucleotide sequences adjacent to the wild type gene are retained. These homologous sequences surrounding the deletion null allele provide targets for homologous recombination so that the wild type gene on the Staphylococcus aureus chromosome can be replaced by the constructed null allele. This method can be used with other bacteria as well, including Escherichia coli, Salmonella and Klebsiella species. Similar gene disruption methods that employ the counter selectable marker sacB (Schweizer, H. P., Klassen, T. and Hoang, T. (1996) Mol. Biol. of Pseudomonas. ASM press, 229-237, the disclosure of which is incorporated herein by reference in its entirety) are available for Pseudomonas, Salmonella and Klebsiella species. Enterococcus faecalis genes can be disrupted by recombining in a non-replicating plasmid that contains an internal fragment to that gene (Leboeuf, C., L. Leblanc, Y. Auffray and A. Hartke. 2000. J. Bacteriol. 182:5799-5806, the disclosure of which is incorporated herein by reference in its entirety).

The crossover PCR amplification product is subcloned into a suitable vector having a selectable marker, such as a drug resistance marker. In some embodiments the vector may have an origin of replication which is functional in Escherichia coli or another organism distinct from the organism in which homologous recombination is to occur, allowing the plasmid to be grown in Escherichia coli or the organism other than that in which homologous recombination is to occur, but may lack an origin of replication functional in Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species such that selection of the selectable marker requires integration of the vector into the homologous region of the Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species chromosome. Usually a single crossover event is responsible for this integration event such that the Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species chromosome now contains a tandem duplication of the target gene consisting of one wild type allele and one deletion null allele separated by vector sequence. Subsequent resolution of the duplication results in both removal of the vector sequence and either restoration of the wild type gene or replacement by the in-frame deletion. The latter outcome will not occur if the gene should prove essential. A more detailed description of this method is provided in the Examples below. It will be appreciated that this method may be practiced with any of the nucleic acids or organisms described herein.

Vectors, Promoters and Host Cells

Other aspects of the present invention relate to vectors that comprise a promoter which is operably linked to an operon that is identified using an operon prediction method described herein or to a nucleotide sequence complementary to all or a portion of an operon identified using an operon prediction method described herein. In some embodiments, the operon comprises a nucleic acid having a nucleotide sequence selected from a group consisting of SEQ ID NOs: 1-194. Each of the nucleotide sequences of SEQ ID NOs: 1-194 are Staphylococcus aureus operons that were predicted using the operon prediction method described herein. Furthermore, each of these operons comprise at least one gene that has been identified to be required for proliferation.

It will be appreciated that a vector which comprises an operon as described herein can be constructed from any vector into which exogenous nucleic acid can be inserted. With respect to a vector “exogenous nucleic acid” refers to any nucleic acid that is not part of the original vector. In some embodiments, the operons are inserted into cloning vectors, sequencing vectors, shuttle vectors, transfer vectors, suicide vectors or expression vectors. The vector may be a vector which replicates extrachromosomally or a vector which integrates into the chromosome. For example, the vector may be a pBR322 based vector or a bacteriophage based vector such as P1 or lambda. In other embodiments, the vectors can replicate in one or more of the microbes selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis and any species falling within the genera of any of the above species.

In some embodiments of the present invention, the vector comprises a promoter that is operably linked to an operon comprising a nucleic acid having a nucleotide sequence selected from the group consisting of SEQ ID NOs: 1-194 or to a nucleotide sequence complementary to all or a portion of the operon. A number of promoters are known in the art and have been previously described. The promoters used to construct the operon containing vectors described herein can be relatively strong promoters, promoters which possess a moderate level of activity, or relatively weak promoters. Additionally, the promoters can be either constitutive or regulatable promoters.

The promoter to which the operon is linked should be active in the bacterium in which the gene products are to be expressed. For example, the operon can be operably linked to the SP6 promoter, T3 promoter, trc promoter, lac promoter, temperature regulated lambda promoters, the Bacillus aprE and nprE promoters (U.S. Pat. No. 5,387,521), the bacteriophage lambda P_{L} and P_{R} promoters (Renaut, et al., (1981) Gene 15: 81) the trp promoter (Russell, et al., (1982) Gene 20: 23), the tac promoter (de Boer et al., (1983) Proc. Natl. Acad. Sci. USA 80: 21), B. subtilis alkaline protease promoter (Stahl et al, (1984) J. Bacteriol. 158, 411-418) alpha amylase promoter of B. subtilis (Yang et al., (1983) Nucleic Acids Res. 11, 237-249) or B. amyloliquefaciens (Tarkinen, et al, (1983) J. Biol. Chem. 258, 1007-1013), the neutral protease promoter from B. subtilis (Yang et al, (1984) J. Bacteriol. 160, 15-21), T7 RNA polymerase promoter (Studier and Moffatt (1986) J Mol Biol. 189(1):113-30), B. subtilis xyl promoter or mutant tetR promoter active in bacilli (Geissendorfer & Hillen (1990) Appl. Microbiol. Biotechnol. 33:657-663), Staphylococcal enterotoxin D promoter (Zhang and Stewart (2000) J. Bacteriol. 182(8):2321-5), cap8 operon promoter from Staphylococcus aureus (Ouyang et al., (1999) J. Bacteriol. 181(8):2492-500), the lactococcal nisA promoter (Eichenbaum (1998) Appl Environ Microbiol. 64(8):2763-9), promoters from in Acholeplasma laidlawii (Jarhede et al., (1995) Microbiology 141 (Pt 9):2071-9), porA promoter of Neisseria meningitidis (Sawaya et al., (1999) Gene 233:49-57), the fbpA promoter of Neisseria gonorrhoeae (Fomg et al., (1997) J. Bacteriol. 179:3047-3052), Corynebacterium diphtheriae toxin gene promoter (Schmitt and Holmes (1994) J. Bacteriol. 176(4):1141-9), the hasA operon promoter from Group A Streptococci (Alberti et al., (1998) Mol Microbiol 28(2):343-53), the rpoS promoter of Pseudomonas putida (Kojic and Venturi (2001) J. Bacteriol. 183:3712-3720), the Acinetobacter baumannii phosphate regulated ppk gene promoter (Gavigan et al., Microbiology 145:2931-7 (1999)); the Acinetobacter baumannii adhC1 promoter which is induced under iron limitation and repressed when the cells are cultured in the presence of free inorganic iron (Echenique et al., Microbiology 147:2805-15 (2001)); the flaB promoter of pGK12 active in Borrelia burgdorferi(Sartakova et al., Proc Natl Acad Sci U S A. 97(9):4850-5 (2000)); the use of Ptrc promoter results in strong inducer-dependent expression in Burkholderia spp (Santos et al., FEMS Microbiol Lett 195(1):91-6 (2001)); the iron regulated sodA promoter of Bordetella pertussis (Graeff-Wohlleben et al., J Bacteriol 179(7):2194-201 (1997)); UV-inducible bcn and uviAB promoters in Clostrdia spp (Garnier and Cole Mol Microbiol 2(5):607-14 (1988)); the heat-inducible clpB promoter of Campylobacter jejuni (Thies et al., Gene 230(1):61-7 (1999)); promoters carrying bacteriophage C1 operator sites in Klebsiella pneumoniae (Schoefield et al, J Bacteriol 183(23):6947-50 (2001)); the Proteus mirabilis ureR promoter (Poore et al., J Bacteriol 183(15):4526-35 (2001)); and the heat-inducible groESL promoter in Listeria monocytogenes, and the IPTG inducible promoter in pLEX5BA (Krause et al., J. Mol. Biol. 274: 365 (1997). In another embodiment, which is useful in Staphylococcus aureus, the promoter is a novel inducible promoter system, XylT5, comprising a modified T5 promoter fused to the xylO operator from the xylA promoter of Staphylococcus aureus. This promoter is described in U.S. patent application Ser. No. 10/032,393, entitled BACTERIAL PROMOTERS AND METHODS OF USE, filed Dec. 21, 2001, the disclosure of which is incorporated herein by reference in its entirety. In another embodiment the promoter may be a two-component inducible promoter system in which the T7 RNA polymerase gene is integrated on the chromosome and is regulated by lacUV5/lacO (Brunschwig, E. and Darzins, A. 1992. Gene 111:35-41, the disclosure of which is incorporated herein by reference in its entirety) and a T7 gene 10 promoter, which is transcribed by T7 RNA polymerase, is fused with a lacO operator. In another embodiment the promoter may be the promoters from the plasmids pEPEF3 or pEPEF14, which harbor xylose inducible promoters functional in E. faecalis, described in U.S. patent application Ser. No. 10/032,393, entitled BACTERIAL PROMOTERS AND METHODS OF USE, filed Dec. 21, 2001, the disclosure of which is incorporated herein by reference in its entirety. Other promoters which may be used are familiar to those skilled in the art. It will appreciated that other combinations of organisms and promoters may also be used in the present invention.

A skilled artisan will appreciate that an vector which comprises an operon as described herein or a nucleotide sequence complementary to all or a portion of the operon can be created for use in any cell by providing a vector which includes features necessary for maintenance in the selected cell type. Such features can include, but are not limited to, origins of replication, resistance genes, repressor genes, ribosomal binding sequences, termination sequences, as well other features. To construct an appropriate vector to provide for replication and/or expression of the operon sequence, one of ordinary skill in the art would know to use standard molecular biology techniques to isolate vectors containing the sequences of interest from cultured bacterial cells, isolate and purify those sequences, and subclone those sequences into a vector adapted for use in the selected species of bacteria.

Methods of Inhibiting Proliferation

Embodiments of the present invention also relate to method of inhibiting cellular proliferation by inhibiting the activity or reducing the amount of a proliferation-required gene product contained in a proliferation-required operon. In one embodiment of the present invention, an antisense nucleic acid that is complementary to at least a portion of a proliferation-required operon identified using the operon prediction methods described herein is provided to a cell thereby inhibiting proliferation of the cell. In another embodiment of the present invention, an antisense nucleic acid that is complementary to at least a portion of an operon comprising a nucleotide sequence selected from the group consisting of SEQ ID NOs: 1-194, wherein the portion does not include a nucleotide sequence selected from the group consisting of SEQ ID NOs: 201-550 is provided to a cell thereby inhibiting proliferation of the cell. Methods of inhibiting cellular proliferation can be performed in one or more of the microbes selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis and any species falling within the genera of any of the above species.

In one exemplary method of inhibiting proliferation, the antisense nucleic acid is provided directly to the cell. Methods for providing antisense nucleic acids directly to a cell are well known in the art. For example, methods such as transformation and electroporation can be used to introduce the antisense nucleic acids into the cell. The proliferation-inhibiting antisense nucleic acids that are provided directly to the cell can be comprised of RNA, DNA, modified polynucleotides or analogs thereof. For example, an antisense nucleic acid can be modified to increase its stability using methods commonly known in the art. Modified nucleic acids can include modifications to one or more nucleotide bases, to one or more sugar molecules or to the phosphate backbone of the nucleic acid. Alternatively, stabilized antisense nucleic acid analogs can be synthesized from peptide nucleic acid (PNA). Methods of producing modified antisense nucleic acids that are stabilized and methods of producing PNA analogs are described further in the Examples below.

In another exemplary method of inhibiting proliferation, a antisense nucleic acid that is complementary to at least a portion of a proliferation-required operon can be introduced into a vector under the control of a promoter. The vector is then transformed into the cell wherein an antisense transcript is produced. Because the antisense transcript is complementary to at least a portion of an operon that is required for proliferation, expression of the antisense transcript results in an inhibition of cellular proliferation. In another embodiment of the present invention, an antisense nucleic acid that is complementary to at least a portion of a proliferation-required operon comprising a nucleotide sequence selected from the group consisting of SEQ ID NOs: 1-194, wherein the portion does not include a nucleotide sequence selected from the group consisting of SEQ ID NOs: 201-550 can be expressed from a vector so as to inhibit cellular proliferation. In particular, the antisense nucleic acid is introduced into a vector under the control of a promoter. The vector is then transformed into the cell wherein an antisense transcript is produced. In some embodiments, the promoter which controls expression of the antisense nucleic acid is constitutive. In other embodiments, the promoter which controls expression of the antisense nucleic acid is regulatable. When a cell containing a vector comprising an antisense nucleic acid under the control of a regulatable promoter is grown under appropriate conditions, such as media containing an inducer of transcription or an agent which alleviates repression of transcription, the antisense transcript is expressed in the cell, thereby reducing the level or activity of the gene product within the cell. In some embodiments, the concentration of the inducer of transcription or the agent which alleviates repression of transcription may be varied to provide optimal results.

Several promoters that can be used in the construction of vectors that are used to implement the above-described methods for inhibiting proliferation. For example, the antisense nucleic acid can be operably linked to the SP6 promoter, T3 promoter, trc promoter, lac promoter, temperature regulated lambda promoters, the Bacillus aprE and nprE promoters (U.S. Pat. No. 5,387,521), the bacteriophage lambda P_{L} and P_{R} promoters (Renaut, et al., (1981) Gene 15: 81) the trp promoter (Russell, et al., (1982) Gene 20: 23), the tac promoter (de Boer et al., (1983) Proc. Natl. Acad. Sci. USA 80: 21), B. subtilis alkaline protease promoter (Stahl et al, (1984) J. Bacteriol. 158, 411-418) alpha amylase promoter of B. subtilis (Yang et al., (1983) Nucleic Acids Res. 11, 237-249) or B. amyloliquefaciens (Tarkinen, et al, (1983) J. Biol. Chem. 258, 1007-1013), the neutral protease promoter from B. subtilis (Yang et al, (1984) J. Bacteriol. 160, 15-21), T7 RNA polymerase promoter (Studier and Moffatt (1986) J Mol Biol. 189(1):113-30), B. subtilis xyl promoter or mutant tetR promoter active in bacilli (Geissendorfer & Hillen (1990) Appl. Microbiol. Biotechnol. 33:657-663), Staphylococcal enterotoxin D promoter (Zhang and Stewart (2000) J. Bacteriol. 182(8):2321-5), cap8 operon promoter from Staphylococcus aureus (Ouyang et al., (1999) J. Bacteriol. 181(8):2492-500), the lactococcal nisA promoter (Eichenbaum (1998) Appl Environ Microbiol. 64(8):2763-9), promoters from in Acholeplasma laidlawii (Jarhede et al., (1995) Microbiology 141 ( Pt 9):2071-9), porA promoter of Neisseria meningitidis (Sawaya et al., (1999) Gene 233:49-57), the fbpA promoter of Neisseria gonorrhoeae (Forng et al., (1997) J. Bacteriol. 179:3047-3052), Corynebacterium diphtheriae toxin gene promoter (Schmitt and Holmes (1994) J. Bacteriol. 176(4):1141-9), the hasA operon promoter from Group A Streptococci (Alberti et al., (1998) Mol Microbiol 28(2):343-53), the rpoS promoter of Pseudomonas putida (Kojic and Venturi (2001) J. Bacteriol. 183:3712-3720), the Acinetobacter baumannii phosphate regulated ppk gene promoter (Gavigan et al., Microbiology 145:2931-7 (1999)); the Acinetobacter baumannii adhC1 promoter which is induced under iron limitation and repressed when the cells are cultured in the presence of free inorganic iron (Echenique et al., Microbiology 147:2805-15 (2001)); the flaB promoter of pGK12 active in Borrelia burgdorferi (Sartakova et al., Proc Natl Acad Sci U S A. 97(9):4850-5 (2000)); the use of Ptrc promoter results in strong inducer-dependent expression in Burkholderia spp (Santos et al., FEMS Microbiol Lett 195(1):91-6 (2001)); the iron regulated sodA promoter of Bordetella pertussis (Graeff-Wohlleben et al., J Bacteriol 179(7):2194-201 (1997)); UV-inducible bcn and uviAB promoters in Clostrdia spp (Garnier and Cole Mol Microbiol 2(5):607-14 (1988)); the heat-inducible clpB promoter of Campylobacter jejuni (Thies et al., Gene 230(1):61-7 (1999)); promoters carrying bacteriophage C1 operator sites in Klebsiella pneumoniae (Schoefield et al, J Bacteriol 183(23):6947-50 (2001)); the Proteus mirabilis ureR promoter (Poore et al., J Bacteriol 183(15):4526-35 (2001)); and the heat-inducible groESL promoter in Listeria monocytogenes, and the IPTG inducible promoter in pLEX5BA (Krause et al., J. Mol. Biol. 274: 365 (1997). In another embodiment, which may be useful in Staphylococcus aureus, the promoter is a novel inducible promoter system, XylT5, comprising a modified T5 promoter fused to the xylO operator from the xylA promoter of Staphylococcus aureus. This promoter is described in U.S. patent application Ser. No. 10/032,393, entitled BACTERIAL PROMOTERS AND METHODS OF USE, filed Dec. 21, 2001, the disclosure of which is incorporated herein by reference in its entirety. In another embodiment the promoter may be a two-component inducible promoter system in which the T7 RNA polymerase gene is integrated on the chromosome and is regulated by lacUV5/lacO (Brunschwig, E. and Darzins, A. 1992. Gene 111:35-41, the disclosure of which is incorporated herein by reference in its entirety) and a T7 gene 10 promoter, which is transcribed by T7 RNA polymerase, is fused with a lacO operator. In another embodiment the promoter may be the promoters from the plasmids pEPEF3 or pEPEF14, which harbor xylose inducible promoters functional in E. faecalis, described in U.S. patent application Ser. No. 10/032,393, entitled BACTERIAL PROMOTERS AND METHODS OF USE, filed Dec. 21, 2001, the disclosure of which is incorporated herein by reference in its entirety. Other promoters which may be used are familiar to those skilled in the art. It will appreciated that other combinations of organisms and promoters may also be used in the present invention.

Expression vectors for a variety of species are known in the art. For example, numerous vectors which function in Escherichia coli are known. Also, Pla et al. have reported an expression vector that is functional in a number of relevant hosts including: Salmonella typhimurium, Pseudomonas putida, and Pseudomonas aeruginosa. J. Bacteriol. 172(8):4448-55 (1990), the disclosure of which is incorporated herein by reference in its entirety. Brunschwig and Darzins (Gene (1992) 111:35-4, the disclosure of which is incorporated herein by reference in its entirety) described a shuttle expression vector for Pseudomonas aeruginosa. Vectors useful for the production of stabilized mRNA having an increased lifetime (including antisense RNA) in Gram negative organisms are described in U.S. patent application No. 10/327592, entitled STABILIZED NUCLEIC ACIDS IN GENE AND DRUG DISCOVERY AND METHODS OF USE, filed Dec. 20, 2002, the disclosure of which is incorporated herein by reference in its entirety. Similarly many examples exist of expression vectors that are freely transferable among various Gram positive microorganisms. Expression vectors for Enterococcus faecalis may be engineered by incorporating suitable promoters into a pAK80 backbone (Israelsen, H., S. M. Madsen, A. Vrang, E. B. Hansen and E. Johansen. 1995. Appl. Environ. Microbiol. 61:2540-2547, the disclosure of which is incorporated herein by reference in its entirety). A number of vectors useful for nucleic acid expression (including antisense nucleic acid expression) in Enterococcus faecalis, Staphylococcus areus as well as other Gram positive organisms are described in U.S. patent application Ser. No. 10/032,393, entitled BACTERIAL PROMOTERS AND METHODS OF USE, filed Dec. 21, 2001, the disclosure of which is incorporated herein by reference in its entirety.

In some embodiments of the present invention, an antisense nucleic acid that is complementary to at least a portion a proliferation-required operon is inserted into a vector that is used for the production of stabilized mRNA having an increased lifetime in Gram negative organisms. Such vectors are described in U.S. patent application Ser. No. 10/327592, entitled STABILIZED NUCLEIC ACIDS IN GENE AND DRUG DISCOVERY AND METHODS OF USE, filed Dec. 20, 2002, the disclosure of which is incorporated herein by reference in its entirety. Briefly, the antisense RNA which is produced from such expression vectors comprises at least one stem loop flanking each end of the antisense nucleic acid. In some versions of the vector, the at least one stem-loop structure formed at the 5′ end of the stabilized antisense nucleic acid comprises a flush, double stranded 5′ end. In other versions, one or more of the stem loops comprises a rho independent terminator. Further versions of the vectors permits transcription of a stabilized antisense RNA which lacks a ribosome binding site, which lacks sites that are cleaved by one or more RNAses, such as RNAse E or RNAse III, or which lacks both a ribosomal binding site and RNAse cleavage sites.

It will be appreciated by one of ordinary skill in the art, vectors may contain certain elements that are species specific. These elements can include promoter sequences, operator sequences, repressor genes, origins of replication, ribosomal binding sequences, termination sequences, and others. To generate a vector for the expression of any antisense nucleic acid which has been described above in any desired bacterial species, one of ordinary skill in the art would know to use standard molecular biology techniques to isolate vectors containing the sequences of interest from cultured bacterial cells, isolate and purify those sequences, and subclone those sequences into a vector adapted for use in the species of bacteria wherein proliferation is to be inhibited.

In addition to the above-described methods of inhibiting proliferation by providing an antisense nucleic acid that is complementary to at least a portion of a proliferation-required operon, some embodiments of the present invention contemplate inhibiting cellular proliferation by replacing the native promoter of an essential operon with a regulatable promoter. Alternatively, a regulatory element can be inserted in or near the native promoter of an essential operon thereby making the expression of genes encoded by the proliferation-required operon regulatable. Both promoter replacement and regulatory element insertion methods are described above in connection with the identification of proliferation-required genes.

Methods of Identifying Compounds that Inhibit Proliferation

The methods described above for inhibiting cellular proliferation by inhibiting the activity or reducing the amount of one or more proliferation-required gene products encoded by one or more genes contained in a proliferation-required operon identified using the methods described herein can be used to screen libraries of compounds, such as combinatorial chemical libraries and natural product libraries, to identify compounds that inhibit cellular proliferation. Methods of constructing combinatorial chemical libraries and natural product libraries is well known in the art.

In one embodiment of the present invention, a cell that has been sensitized by providing an antisense nucleic acid that is complementary to at least a portion of a proliferation-required operon which has been identified using the operon prediction methods described herein is contacted with one or more candidate compounds from a small molecule library. In another embodiment of the present invention, a cell that has been sensitized by providing an antisense nucleic acid that is complementary to at least a portion of a proliferation-required operon comprising a nucleotide sequence selected from the group consisting of SEQ ID NOs.: 1-194 is contacted with one or more candidate compounds from a small molecule library. In other embodiments, the antisense nucleic acid that is used to produce a sensitized cell is complementary to at least a portion of a proliferation-required operon comprising a nucleotide sequence selected from the group consisting of SEQ ID NOs.: 1-194, wherein the portion does not include a nucleotide sequence selected from the group consisting of SEQ ID NOs.: 201-550. Candidate compounds which further inhibit the proliferation of the sensitized cell may be identified as possessing inhibitory activity against one or more gene products encoded by the proliferation-required operon. Methods which can be used to sensitize cells by providing an antisense nucleic acid that is complementary to at least a portion of a proliferation-required operon have been described above in connection with methods of inhibiting cellular proliferation.

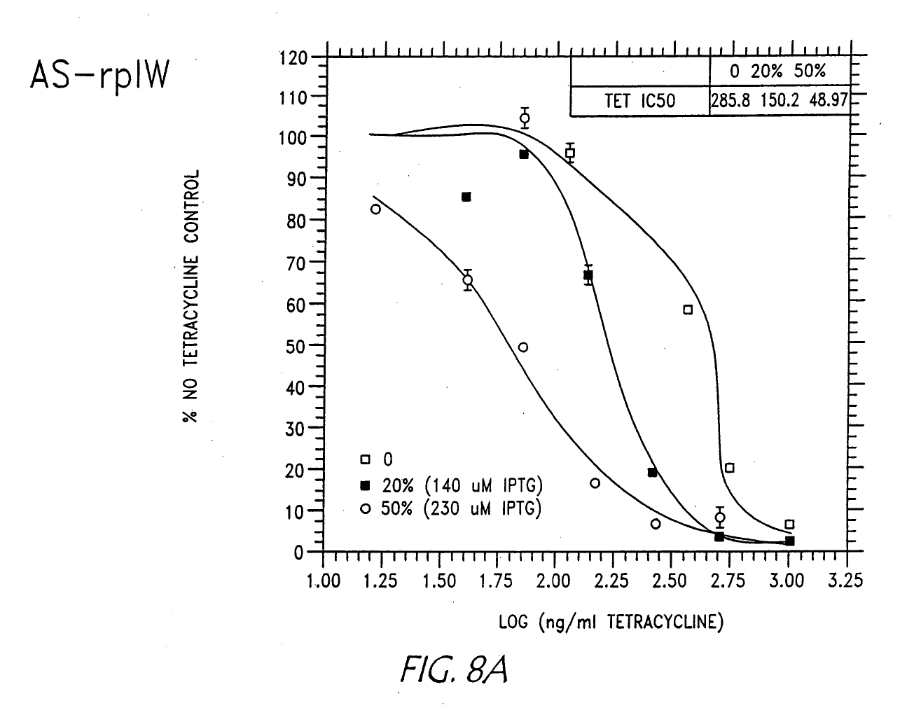

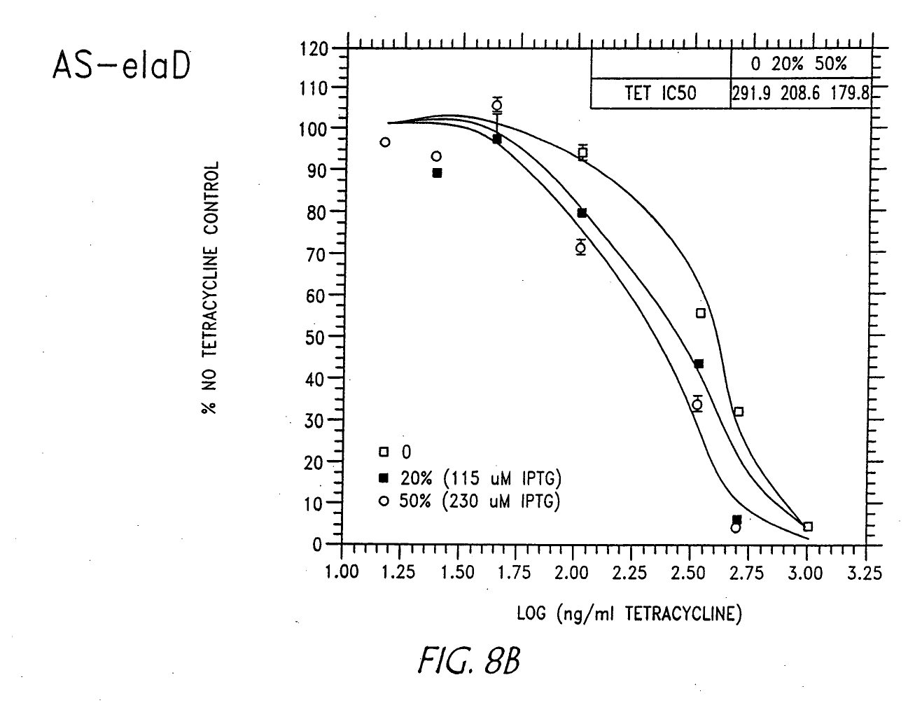

In some embodiments of the present invention, a first cell is provided with an antisense nucleic acid that is complementary to at least a portion of a proliferation-required operon. Production of a sublethal level of the antisense nucleic acid within the cell causes the cell to be sensitized to the effects of compounds that may have little or no proliferation inhibiting activity against an unsensitized cell. Both the cell that has been sensitized with the antisense nucleic acid and an unsensitized cell, preferably of the same species as the sensitized cells, are then contacted with a compound and the effect of the compound on the proliferation of each cell is measured. A greater reduction in the extent of proliferation of the sensitized after contact with the compound compared to the reduction in the extent of proliferation of the unsensitized cell after contact with the compound indicates that the compound possesses the ability to reduce cellular proliferation. It will be appreciated that the above methods can also be performed by comparing the effect of a compound on the extent of proliferation between a cells that have been sensitized at different levels (see FIGS. 8A and 8B). The level of sensitization of a cell can be controlled by providing more or less of an antisense nucleic acid that is complementary to a proliferation-required operon.

In some embodiments of the present invention, promoter replacement or regulatory element insertion methods can be used to produce sensitized cells by inhibiting the activity or reducing the amount of one or more proliferation-required genes that are contained within a proliferation-required operon. Such sensitized cells can be used to identify compounds that possess the ability to inhibit cellular proliferation. Promoter replacement and operon insertion methods are described above in connection with methods of identifying proliferation-required genes and methods of inhibiting cellular proliferation.

It will be appreciated that the methods of identifying compounds which inhibit microbial proliferation as described herein can be performed with any microbe including, but not limited to, the pathogenic microbes selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis and any species falling within the genera of any of the above species.

Methods of Using Homologous Sequences

In one embodiment of the present invention, the above-described methods of inhibiting proliferation and methods of identifying compounds that inhibit proliferation can be implemented by providing an antisense nucleic acid that is complementary to at least a portion of an operon which comprises a coding nucleic acid sequence that is homologous to a proliferation-required nucleic acid having a nucleotide sequence selected from the group consisting of SEQ ID NOs: 201-550. Additionally, in another embodiment the antisense nucleic acid is complementary to at least a portion of an operon which comprises a gene that encodes a polypeptide that is homologous to a polypeptide having an amino acid sequence selected from the group consisting of SEQ ID NOs: 551-824. In either of these embodiments, the antisense nucleic acid can be complementary to at least a portion of an operon that is identified by an operon prediction method described herein and which is required for the proliferation of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

The present invention also contemplates the use of antisense nucleic acids that are complementary to at least a portion of an operon comprising a nucleotide sequence selected from the group consisting of SEQ ID NOs: 1-194, wherein the portion does not include a nucleotide sequence selected from a group consisting of SEQ IN NOs: 201-550 in organisms other than Staphylococcus aureus. For example, one or more of such antisense nucleic acids, can be provided to a pathogenic microbe including, but not limited to, Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

The antisense nucleic acids described above can be provided to the cell using a variety of methods well known in the art. For example, the above-described antisense nucleic acids can be directly provided to the cell using methods such as transformation or electroporation. Alternatively, such antisense nucleic acid may be provided on an expression vector. In one embodiment of the present invention, the antisense nucleic acids are transferred to vectors capable of function within a species other than the species from which the sequences were obtained. For example, the vector may be functional in Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species. In some embodiments of the present invention, the vector may be functional in an organism other than Staphylococcus aureus. Vectors that can be used for the expression of antisense nucleic acids in numerous organisms have been described above in connection with methods for inhibiting cellular proliferation and methods for identifying compounds which possess antimicrobial activity.

It will be appreciated by one of ordinary skill in the art, vectors may contain certain elements that are species specific. These elements can include promoter sequences, operator sequences, repressor genes, origins of replication, ribosomal binding sequences, termination sequences, and others. To generate a vector for the expression of any antisense nucleic acid which has been described above in any desired bacterial species, one of ordinary skill in the art would know to use standard molecular biology techniques to isolate vectors containing the sequences of interest from cultured bacterial cells, isolate and purify those sequences, and subclone those sequences into a vector adapted for use in the species of bacteria to be screened.

The antisense nucleic acids which are provided to a cell, either directly or via expression vector, can be used to inhibit cellular proliferation or to produce sensitized cells for use in identifying compounds having antimicrobial activity. Each of these methods have been described in detail above.

Therapeutics

In still another embodiment of the present invention, an antisense nucleic acid that is complementary to at least a portion of a proliferation-required operon identified using the operon prediction methods described herein is provided to a cell as an antisense therapeutic. In some embodiments, an antisense nucleic acid that is complementary to at least a portion of an operon comprising a nucleotide sequence selected from the group consisting of SEQ ID NOs: 1-194, wherein the portion does not include a nucleotide sequence selected from the group consisting of SEQ ID NOs: 201-550 is provided to a cell as an antisense therapeutic. Antisense therapeutics can be complementary to operons in which proliferation-required genes are contained or to operons in which the combination of a plurality genes is required for proliferation. Accordingly, the antisense therapeutic can mediate its effect by inhibiting the activity or reducing the amount of one or more gene products contained within the proliferation-required operon. Thus, the antisense therapeutic can be complementary to at least a portion of an operon that corresponds to a gene that is required for proliferation or portion thereof, a 5′ noncoding region or a 3′ noncoding region located upstream or downstream from the actual gene that is required for proliferation or a portion thereof, an intergenic sequence (i.e. a sequence between genes) or a portion thereof, a gene that is not required for proliferation or a portion thereof, or a nucleotide sequence spanning at least a portion of two or more genes.

It will be appreciated that by performing the methods described herein, antisense therapeutics can be developed against pathogenic microbes which include, but are not limited to, Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis and any species falling within the genera of any of the above species.

The following examples teach methods of identifying the operons which comprise at least one gene that is required for proliferation as well as the use of antisense nucleic acids complementary to at least a portion of proliferation-required operon to inhibit cellular proliferation and to identify compounds that inhibit cellular proliferation. These examples are illustrative only and are not intended to limit the scope of the present invention.

### EXAMPLES

The following examples are directed to the identification of genes required for proliferation as well as identification of the proliferation-required operons in which these genes are contained. A variety of methods to utilize the identified proliferation-required operons are also discussed. It will be appreciated that the operons comprising a nucleotide sequence selected from the group consisting of SEQ ID NOs: 1-194 can be used in methods below which utilize proliferation-required operons. Likewise, operons containing one or more genes that are homologous to a proliferation-required gene contained within any of these operons can also be used in the methods below which utilize proliferation-required operons.

Genes Identified as Required for Proliferation of Escherichia coli, Staphylococcus Aureus, Enterococcus Faecalis, Klebsiella Pneumoniae, Pseudomonas Aeruginosa and Salmonella Typhimurium.

Genomic fragments were operably linked to an inducible promoter in a vector and assayed for growth inhibition activity. Example 1 describes the examination of a library of genomic fragments cloned into vectors comprising inducible promoters. Upon induction with xylose or IPTG, the vectors produced an RNA molecule corresponding to the subcloned genomic fragments. In those instances where the genomic fragments were in an antisense orientation with respect to the promoter, the transcript produced was complementary to at least a portion of an mRNA (messenger RNA) encoding a Escherichia coli, Staphylococcus aureus, Enterococcus faecalis, Klebsiella pneumoniae, Pseudomonas aeruginosa or Salmonella typhimurium gene product such that they interacted with sense mRNA produced from various Escherichia coli, Staphylococcus aureus, Enterococcus faecalis, Klebsiella pneumoniae, Pseudomonas aeruginosa or Salmonella typhimurium genes and thereby decreased the translation efficiency or the level of the sense messenger RNA thus decreasing production of the protein encoded by these sense mRNA molecules. In cases where the sense mRNA encoded a protein required for proliferation, bacterial cells containing a vector from which transcription from the promoter had been induced failed to grow or grew at a substantially reduced rate. Additionally, in cases where the transcript produced was complementary to at least a portion of a non-translated RNA and where that non-translated RNA was required for proliferation, bacterial cells containing a vector from which transcription from the promoter had been induced also failed to grow or grew at a substantially reduced rate. In contrast, cells grown under non-inducing conditions grow at a normal rate.

The above method was used to identify genes required for cellular proliferation in Escherichia coli, Staphylococcus aureus, Enterococcus faecalis, Klebsiella pneumoniae, Pseudomonas aeruginosa and Salmonella typhimurium. Additionally, a number of genes required for cellular proliferation in Escherichia coli, Staphylococcus aureus, Enterococcus faecalis, Klebsiella pneumoniae, Pseudomonas aeruginosa and Salmonella typhimurium, which have been described in the following U.S. Patent Applications, the disclosures of which are incorporated herein by reference in their entireties: U.S. patent application Ser. No. 09/492,709, entitled GENES IDENTIFIED AS REQUIRED FOR PROLIFERATION IN ESCHERICHIA COLI, filed Jan. 27, 2000; Ser. No. 09/711,164, entitled GENES ESSENTIAL FOR MICROBIAL PROLIFERATION AND ANTISENSE THERETO, filed Nov. 9, 2000; Ser. No. 09/741,669, entitled GENES IDENTIFIED AS REQUIRED FOR PROLIFERATION OF E. COLI, filed Dec. 19, 2000; Ser. No. 09/815,242, entitled IDENTIFICATION OF ESSENTIAL GENES IN PROKARYOTES, filed Mar. 21, 2001 and Ser. No. 10/282,122, entitled IDENTIFICATION OF ESSENTIAL GENES IN MICROORGANISMS, filed Oct. 25, 2002, have been previously identified using the above method.

### Example 1

### Inhibition of Bacterial Proliferation after Induction of Antisense Expression

To identify genes required for proliferation of E. coli, random genomic fragments were cloned into the IPTG-inducible expression vector pLEX5BA (Krause et al., J. Mol. Biol. 274: 365 (1997), the disclosure of which is incorporated herein by reference in its entirety) or a modified version of pLEX5BA, pLEX5BA-3′ in which a synthetic linker containing a T7 terminator was ligated between the PstI and HindIII sites of pLEX5BA. In particular, to construct pLEX5BA-3′, the following oligonucleotides were annealed and inserted into the PstI and HindIII sites of pLEX5BA:

| | |
|---|---|
| 5′-GTCTAGCATAACCCCTTGGGGCCTCTAAACG | (SEQ ID NO: 825) |
| GGTCCTTGAGGGGTTTTTTGA-3′ | |
| 5′-AGCTTCAAAAAACCCCTCAAGGACCCGTTTA | (SEQ ID NO: 826) |
| GAGGCCCCAAGGGGTTATGCTAGACTGCA-3′ | |

Random fragments of E. coli genomic DNA were generated by DNAseI digestion or sonication, filled in with T4 polymerase, and cloned into the SmaI site of pLEX5BA or pLEX5BA-3′. Upon activation or induction, the promoter transcribed the random genomic fragments.

A number of vectors which allow the production of transcripts which have an extended lifetime in E. coli as well as other Gram negative bacteria can also be utilized in conjunction with these antisense inhibition experiments. Such vectors are described in U.S. patent application Ser. No. 10/327592, entitled STABILIZED NUCLEIC ACIDS IN GENE AND DRUG DISCOVERY AND METHODS OF USE, filed Dec. 20, 2002, the disclosure of which is incorporated herein by reference in its entirety. Briefly, the stabilized antisense RNA may comprise an antisense RNA which was identified as inhibiting proliferation as described above which has been engineered to contain at least one stem loop flanking each end of the antisense nucleic acid. In some embodiments, the at least one stem-loop structure formed at the 5′ end of the stabilized antisense nucleic acid comprises a flush, double stranded 5′ end. In some embodiments, one or more of the stem loops comprises a rho independent terminator. In additional embodiments, the stabilized antisense RNA lacks a ribosome binding site. In further embodiments, the stabilized RNA lacks sites which are cleaved by one or more RNAses, such as RNAse E or RNAse III. In some embodiments, the stabilized antisense RNA may be transcribed in a cell which the activity of at least one enzyme involved in RNA degradation has been reduced. For example, the activity of an enzyme such as RNase E, RNase II, RNase III, polynucleotide phosphorylase, and poly(A) polymerase, RNA helicase, enolase or an enzyme having similar functions may be reduced in the cell.

To study the effects of transcriptional induction in liquid medium, growth curves were carried out by back diluting cultures 1:200 into fresh media with or without 1 mM IPTG and measuring the OD₄₅₀ every 30 minutes (min). To study the effects of transcriptional induction on solid medium, 10², 10³, 10⁴, 10⁵, 10⁶, 10⁷ and 10⁸ fold dilutions of overnight cultures were prepared. Aliquots of from 0.5 to 3 μl of these dilutions were spotted on selective agar plates with or without 1 mM IPTG. After overnight incubation, the plates were compared to assess the sensitivity of the clones to IPTG.

Of the numerous clones tested, some clones were identified as containing a sequence that inhibited E. coli growth after IPTG induction. Accordingly, the gene to which the inserted nucleic acid sequence corresponds, or a gene within the operon containing the inserted nucleic acid, is required for proliferation in E. coli.

Nucleic acids involved in proliferation of Staphylococcus aureus, Enterococcus faecalis, Klebsiella pneumoniae, Pseudomonas aeruginosa and Salmonella typhimurium were identified as follows. Randomly generated fragments of Staphylococcus aureus, Enterococcus faecalis, Klebsiella pneumoniae, Pseudomonas aeruginosa or Salmonella typhimurium genomic DNA were transcribed from inducible promoters.

In the case of Staphylococcus aureus, a novel inducible promoter system, XylT5, comprising a modified T5 promoter fused to the xylO operator from the xylA promoter of Staphylococcus aureus was used. The promoter is described in U.S. patent application Ser. No. 10/032,393, entitled BACTERIAL PROMOTERS AND METHODS OF USE, filed Dec. 21, 2001, the disclosure of which is incorporated herein by reference in its entirety. Transcription from this hybrid promoter is inducible by xylose.

Randomly generated fragments of Salmonella typhimurium genomic DNA were transcribed from an IPTG inducible promoter in pLEX5BA (Krause et al., J. Mol. Biol. 274: 365 (1997) or a derivative thereof. Randomly generated fragments of Klebsiella pneumoniae genomic DNA were expressed from an IPTG inducible promoter in pLEX5BA-Kan. To construct pLEX5BA-kan, pLEX5BA was digested to completion with ClaI in order to remove the bla gene. Then the plasmid was treated with a partial NotI digestion and blunted with T4 DNA polymerase. A 3.2 kbp fragment was then gel purified and ligated to a blunted 1.3 kbp kan gene from pKanπ. Kan resistant transformants were selected on Kan plates. Orientation of the kan gene was checked by SmaI digestion. A clone, which had the kan gene in the same orientation as the bla gene, was used to identify genes required for proliferation of Klebsiella pneumoniae.

Randomly generated fragments of Pseudomonas aeruginosa genomic DNA were transcribed from a two-component inducible promoter system. Integrated on the chromosome was the T7 RNA polymerase gene regulated by lacUV5/lacO (Brunschwig, E. and Darzins, A. 1992. Gene 111:35-41, the disclosure of which is incorporated herein by reference in its entirety). On a separate plasmid, a T7 gene 10 promoter, which is transcribed by T7 RNA polymerase, was fused with a lacO operator followed by a multiple cloning site.

Should the genomic DNA downstream of the promoter contain, in an antisense orientation, at least a portion of an mRNA or a non-translated RNA encoding a gene product involved in proliferation, then induction of transcription from the promoter will result in detectable inhibition of proliferation.

In the case of Staphylococcus aureus, a shotgun library of Staphylococcus aureus genomic fragments was cloned into the vector pXyIT5-P15a, which harbors the XylT5 inducible promoter. The vector was linearized at a unique BamHI site immediately downstream of the XyIT5 promoter/operator. The linearized vector was treated with shrimp alkaline phosphatase to prevent reclosure of the linearized ends. Genomic DNA isolated from Staphylococcus aureus strain RN450 was fully digested with the restriction enzyme Sau3A , or , alternatively, partially digested with DNaseI and “blunt-ended” by incubating with T4 DNA polymerase. Random genomic fragments between 200 and 800 base pairs in length were selected by gel purification. The size-selected genomic fragments were added to the linearized and dephosphorylated vector at a molar ratio of 0.1 to 1, and ligated to form a shotgun library.

The ligated products were transformed into electrocompetent E. coli strain XL1-Blue MRF′ (Stratagene) and plated on LB medium with supplemented with carbenicillin at 100 μg/ml. Resulting colonies numbering 5×10⁵ or greater were scraped and combined, and were then subjected to plasmid purification.

The purified library was then transformed into electrocompetent Staphylococcus aureus RN4220. Resulting transformants were plated on agar containing LB+0.2% glucose (LBG medium)+chloramphenicol at 15 μg/ml (LBG+CM15 medium) in order to generate 100 to 150 platings at 500 colonies per plating. The colonies were subjected to robotic picking and arrayed into wells of 384 well culture dishes. Each well contained 100 μl of LBG+CM15 liquid medium. Inoculated 384 well dishes were incubated 16 hours at 37° C., and each well was robotically gridded onto solid LBG+CM15 medium with or without 2% xylose. Gridded plates were incubated 16 hours at 37° C., and then manually scored for arrayed colonies that were growth-compromised in the presence of xylose.

Arrayed colonies that were growth-sensitive on medium containing 2% xylose, yet were able to grow on similar medium lacking xylose, were subjected to further growth sensitivity analysis as follows: Colonies from the plate lacking xylose were manually picked and inoculated into individual wells of a 96 well culture dish containing LBG+CM15, and were incubated for 16 hours at 37° C. These cultures were robotically diluted 1/100 into fresh medium and allowed to incubate for 4 hours at 37° C., after which they were subjected to serial dilutions in a 384 well array and then gridded onto media containing 2% xylose or media lacking xylose. After growth for 16 hours at 37° C., the arrays that resulted on the two media were compared to each other. Clones that grew similarly at all dilutions on both media were scored as a negative and were no longer considered. Clones that grew on xylose medium but failed to grow at the same serial dilution on the non-xylose plate were given a score based on the differential, i.e. should the clone grow at a serial dilution of 10⁴ or less on the xylose plate and grow at a serial dilution of 10⁸ or less on the non-xylose plate, then the corresponding clone received a score of “4” representing the log difference in growth observed.

For Salmonella typhimurium and Klebsiella pneumoniae growth curves were carried out by back diluting cultures 1:200 into fresh media containing 1 mM IPTG or media lacking IPTG and measuring the OD₄₅₀ every 30 minutes (min). To study the effects of transcriptional induction on solid medium, 10², 10³, 10⁴, 10⁵, 10⁶, 10⁷ and 10⁸ fold dilutions of overnight cultures were prepared. Aliquots of from 0.5 to 3 μl of these dilutions were spotted on selective agar plates with or without 1 mM IPTG. After overnight incubation, the plates were compared to assess the sensitivity of the clones to IPTG.

Nucleic acids involved in proliferation of Pseudomonas aeruginosa were identified as follows. Randomly generated fragments of Pseudomonas aeruginosa genomic DNA were transcribed from a two-component inducible promoter system. Integrated on the chromosome was the T7 RNA polymerase gene regulated by lacUV5/lacO (Brunschwig, E. and Darzins, A. 1992. Gene 111:35-41). On an expression plasmid there was a T7 gene 10 promoter, which is transcribed by T7 RNA polymerase, fused with a lacO operator followed by a multiple cloning site. Transcription from this hybrid promoter is inducible by IPTG. Should the genomic DNA downstream of the promoter contain, in an antisense orientation, at least a portion of an mRNA encoding a gene product involved in proliferation, then induction of expression from the promoter will result in detectable inhibition of proliferation.

A shotgun library of Pseudomonas aeruginosa genomic fragments was cloned into the vectors pEP5, pEP5S, or other similarly constructed vectors which harbor the T7lacO inducible promoter. The vector was linearized at a unique SmaI site immediately downstream of the T7lacO promoter/operator. The linearized vector was treated with shrimp alkaline phosphatase to prevent reclosure of the linearized ends. Genomic DNA isolated from Pseudomonas aeruginosa strain PAO1 was partially digested with DNaseI and “blunt-ended” by incubating with T4 DNA polymerase. Random genomic fragments between 200 and 800 base pairs in length were selected by gel purification. The size-selected genomic fragments were added to the linearized and dephosphorylated vector at a molar ratio of 2 to 1, and ligated to form a shotgun library.

The ligated products were transformed into electrocompetent E. coli strain XL1-Blue MRF′ (Stratagene) and plated on LB medium with carbenicillin at 100 μg/ml or Streptomycin 100 μg/ml. Resulting colonies numbering 5×10⁵ or greater were scraped and combined, and were then subjected to plasmid purification.

The purified library was then transformed into electrocompetent Pseudomonas aeruginosa strain PAO1. Resulting transformants were plated on LB agar with carbenicillin at 100 μg/ml or Streptomycin 40 μg/ml in order to generate 100 to 150 platings at 500 colonies per plating. The colonies were subjected to robotic picking and arrayed into wells of 384 well culture dishes. Each well contained 100 μl of LB+CB100 or Streptomycin 40 liquid medium. Inoculated 384 well dishes were incubated 16 hours at room temperature, and each well was robotically gridded onto solid LB+CB100 or Streptomycin 40 medium with or without 1 mM IPTG. Gridded plates were incubated 16 hours at 37° C., and then manually scored for arrayed colonies that were growth-compromised in the presence of IPTG.

Arrayed colonies that were growth-sensitive on medium containing 1 mM IPTG, yet were able to grow on similar medium lacking IPTG, were subjected to further growth sensitivity analysis as follows: Colonies from the plate lacking IPTG were manually picked and inoculated into individual wells of a 96 well culture dish containing LB+CB100 or Streptomycin 40, and were incubated for 16 hours at 30° C. These cultures were robotically diluted 1/100 into fresh medium and allowed to incubate for 4 hours at 37° C., after which they were subjected to serial dilutions in a 384 well array and then gridded onto media with and without 1 mM IPTG. After growth for 16 hours at 37° C., the arrays of serially diluted spots that resulted were compared between the two media. Clones that grew similarly at all dilutions on both media were scored as a negative and were no longer considered. Clones that grew on IPTG medium but failed to grow at the same serial dilution on the non-IPTG plate were given a score based on the differential, i.e. should the clone grow at a serial dilution of 10⁴ or less on the IPTG plate and grow at a serial dilution of 10⁸ or less on the IPTG plate, then the corresponding clone received a score of “4” representing the log difference in growth observed.

Following the identification of those vectors that, upon induction, negatively impacted Pseudomonas aeruginosa growth or proliferation, the inserts or nucleic acid fragments contained in those vectors were isolated for subsequent characterization. Vectors of interest were subjected to nucleic acid sequence determination.

Nucleic acids involved in proliferation of E. faecalis were identified as follows. Randomly generated fragments of genomic DNA were expressed from the vectors pEPEF3 or pEPEF14, which contain the CP25 or P59 promoter, respectively, regulated by the xyl operator/repressor. These plasmids as well as other vectors useful for the expression of nucleic acids in Enterococcus faecalis and other Gram positive organisms are described in U.S. patent application Ser. No. 10/032,393, entitled BACTERIAL PROMOTERS AND METHODS OF USE, filed Dec. 21, 2001, the disclosure of which is incorporated herein by reference in its entirety. Should the genomic DNA downstream of the promoter contain, in an antisense orientation, at least a portion of a mRNA encoding a gene product involved in proliferation, then induction of expression from the promoter will result in detectable inhibition of proliferation.

A shotgun library of E. faecalis genomic fragments was cloned into the vector pEPEF3 or pEPEF14, which harbor xylose inducible promoters. The vector was linearized at a unique SmaI site immediately downstream of the promoter/operator. The linearized vector was treated with alkaline phosphatase to prevent reclosure of the linearized ends. Genomic DNA isolated from E. faecalis strain OG1RF was partially digested with DNaseI and “blunt-ended” by incubating with T4 DNA polymerase. Random genomic fragments between 200 and 800 base pairs in length were selected by gel purification. The size-selected genomic fragments were added to the linearized and dephosphorylated vector at a molar ratio of 2 to 1, and ligated to form a shotgun library.

The ligated products were transformed into electrocompetent E. coli strain TOP10 cells (Invitrogen) and plated on LB medium with erythromycin (Erm) at 150 μg/ml. Resulting colonies numbering 5×10⁵ or greater were scraped and combined, and were then subjected to plasmid purification.

The purified library was then transformed into electrocompetent E. faecalis strain OG1RF. Resulting transformants were plated on Todd-Hewitt (TH) agar with erythromycin at 10 μg/ml in order to generate 100 to 150 platings at 500 colonies per plating. The colonies were subjected to robotic picking and arrayed into wells of 384 well culture dishes. Each well contained 100 μl of THB+Erm 10 μg/ml. Inoculated 384 well dishes were incubated 16 hours at room temperature, and each well was robotically gridded onto solid TH agar+Erm with or without 5% xylose. Gridded plates were incubated 16 hours at 37° C., and then manually scored for arrayed colonies that were growth-compromised in the presence of xylose.

Arrayed colonies that were growth-sensitive on medium containing 5% xylose, yet were able to grow on similar medium lacking xylose, were subjected to further growth sensitivity analysis. Colonies from the plate lacking xylose were manually picked and inoculated into individual wells of a 96 well culture dish containing THB+Erm 10, and were incubated for 16 hours at 30° C. These cultures were robotically diluted 1/100 into fresh medium and allowed to incubate for 4 hours at 37° C., after which they were subjected to serial dilution on plates containing 5% xylose or plates lacking xylose. After growth for 16 hours at 37° C., the arrays of serially diluted spots that resulted were compared between the two media. Colonies that grew similarly on both media were scored as a negative and corresponding colonies were no longer considered. Colonies on xylose medium that failed to grow to the same serial dilution compared to those on the non-xylose plate were given a score based on the differential. For example, colonies on xylose medium that only grow to a serial dilution of −4 while they were able to grow to −8 on the non-xylose plate, then the corresponding transformant colony received a score of “4” representing the log difference in growth observed.

Following the identification of those vectors that, upon induction, negatively impacted E. faecalis growth or proliferation, the inserts or nucleic acid fragments contained in those expression vectors were isolated for subsequent characterization. The inserts in the vectors of interest were subjected to nucleotide sequence determination.

It will be appreciated that other restriction enzymes and other endonucleases or methodologies may be used to generate random genomic fragments. In addition, random genomic fragments may be generated by mechanical shearing. Sonication and nebulization are two such techniques commonly used for mechanical shearing of DNA.

### Example 2

### Nucleotide Sequence Determination of Identified Clones Transcribing Nucleic Acid Fragments with Detrimental Effects on Escherichia coli, Staphylococcus aureus, Enterococcus faecalis, Klebsiella pneumoniae, Pseudomonas aeruginosa or Salmonella typhimurium Proliferation

Plasmids from clones that received a dilution plating score of “2” or greater were isolated to obtain the genomic DNA insert responsible for growth inhibition as follows.

The nucleotide sequences of the nucleic acid sequences which inhibited the growth of Escherichia coli were determined using plasmid DNA isolated using QIAPREP (Qiagen, Valencia, Calif.) and methods supplied by the manufacturer. The primers used for sequencing the inserts were 5′-TGTTTATCAGACCGCTT-3′ (SEQ ID NO: 827) and 5′-ACAATTTCACACAGCCTC-3′ (SEQ ID NO: 828). These sequences flank the polylinker in pLEX5BA.

The nucleotide sequences of the nucleic acid sequences which inhibited the growth of Staphylococcus aureus were determined as follows. Staphylococcus aureus were grown in standard laboratory media (LB or TB with 15 μg/ml Chloramphenicol to select for the plasmid). Growth was carried out at 37° C. overnight in culture tubes or 2 ml deep well microtiter plates.

Lysis of Staphylococcus aureus was performed as follows. Cultures (2-5 ml) were centrifuged and the cell pellets resuspended in 1.5 mg/ml solution of lysostaphin (20 μl/ml of original culture) followed by addition of 250 μl of resuspension buffer (Qiagen). Alternatively, cell pellets were resuspended directly in 250 μl of resuspension buffer (Qiagen) to which 5-20 μl of a 1 mg/ml lysostaphin solution were added.

DNA was isolated using Qiagen miniprep kits or Wizard (Qiagen) miniprep kits according to the instructions provided by the manufacturer.

The genomic DNA inserts were amplified from the purified plasmids by PCR as follows.

1 μl of Qiagen purified plasmid was put into a total reaction volume of 25 μl Qiagen Hot Start PCR mix. For Staphylococcus aureus, the following primers were used in the PCR reaction:

| | | |
|---|---|---|
| pXylT5F: | CAGCAGTCTGAGTTATAAAATAG | (SEQ ID NO: 829) |
| LexL | TGTTTTATCAGACCGCTT | (SEQ ID NO: 830) |

Similar methods were conducted for Salmonella typhimurium and Klebsiella pneumoniae. For Salmonella typhimurium and Klebsiella pneumoniae the following primers were used:

| | |
|---|---|
| 5′-TGTTTTATCAGACCGCTT-3′ | (SEQ ID NO: 830) |
| and | |
| 5′-ACAATTTCACACAGCCTC-3′ | (SEQ ID NO: 828) |

PCR was carried out in a PE GenAmp with the following cycle times:
Step 1. 95° C. 15 min
Step 2. 94° C. 45 sec
Step 3. 54° C. 45 sec
Step 4. 72° C. 1 minute
Step 5. Return to step 2, 29 times
Step 6. 72° C. 10 minutes
Step 7. 4° C. hold
   The PCR products were cleaned using Qiagen Qiaquick PCR plates according to the manufacturer's instructions.

For Pseudomonas aeruginosa, plasmids from transformant colonies that received a dilution plating score of “2” or greater were isolated to obtain the genomic DNA insert responsible for growth inhibition as follows. Pseudomonas aeruginosa were grown in standard laboratory media (LB with carbenicillin at 100 μg/ml or Streptomycin 40 μg/ml to select for the plasmid). Growth was carried out at 30° C. overnight in 100 μl culture wells in microtiter plates. To amplify insert DNA 2 μl of culture were placed into 25 μl Qiagen Hot Start PCR mix. PCR reactions were in 96 well microtiter plates. For plasmid pEP5S the following primers were used in the PCR reaction:

| | | |
|---|---|---|
| T7L1+: | GTCGGCGATATAGGCGCCAGCAACCG | (SEQ ID NO: 831) |
| pStrA3: | ATAATCGAGCATGAGTATCATACG | (SEQ ID NO: 832) |

PCR was carried out in a PE GenAmp with the following cycle times:
Step 1. 95° C. 15 min
Step 2. 94° C. 45 sec
Step 3. 54° C. 45 sec
Step 4. 72° C. 1 minute
Step 5. Return to step 2, 29 times
Step 6. 72° C. 10 minutes
Step 7. 4° C. hold
   The PCR products were cleaned using Qiagen Qiaquick PCR plates according to the manufacturer's instructions.

The purified PCR products were then directly cycle sequenced with Qiagen Hot Start PCR mix. The following primers were used in the sequencing reaction:

| | | |
|---|---|---|
| T7/L2: | ATGCGTCCGGCGTAGAGGAT | (SEQ ID NO: 833) |

PCR was carried out in a PE GenAmp with the following cycle times:
Step 1. 94° C. 15 min
Step 2. 96° C. 10 sec
Step 3. 50° C. 5 sec
Step 4. 60° C. 4 min
Step 5. Return to step 2, 24 times
Step 6. 40° C. hold
   The PCR products were cleaned using Qiagen Qiaquick PCR plates according to the manufacturer's instructions.

For E. faecalis, plasmids from transformant colonies that received a dilution plating score of “2” or greater were isolated to obtain the genomic DNA insert responsible for growth inhibition as follows. E. faecalis were grown in THB 10 μg/ml Erm at 30° C. overnight in 100 μl culture wells in microtiter plates. To amplify insert DNA 2 μl of culture were placed into 25 μl Qiagen Hot Start PCR mix. PCR reactions were in 96 well microtiter plates. The following primers were used in the PCR reaction:
pXylT5: CAGCAGTCTGAGTTATAAAATAG (SEQ ID NO: 829) and the pEP/pAK1 primer.
   PCR was carried out in a PE GenAmp with the following cycle times:
Step 1. 95° C. 15 min
Step 2. 94° C. 45 sec
Step 3. 54° C. 45 sec
Step 4. 72° C. 1 minute
Step 5. Return to step 2, 29 times
Step 6. 72° C. 10 minutes
Step 7. 4° C. hold
   The PCR products were cleaned using Qiagen Qiaquick PCR plates according to the manufacturer's instructions.

The purified PCR products were then directly cycle sequenced with Qiagen Hot Start PCR mix. The following primers were used in the PCR reaction:

| | | |
|---|---|---|
| pXylT5: | CAGCAGTCTGAGTTATAAAATAG | (SEQ ID NO: 829) |

PCR was carried out in a PE GenAmp with the following cycle times:
Step 1. 94° C. 15 min
Step 2. 96° C. 10 sec
Step 3. 50° C. 5 sec
Step 4. 60° C. 4 min
Step 5. Return to step 2, 24 times
Step 6. 4° C. hold

The PCR products were cleaned using Qiagen Qiaquick PCR plates according to the manufacturer's instructions. The amplified genomic DNA inserts from each of the above procedures were subjected to automated sequencing.

### Example 3

### Comparison Of Isolated Nucleic Acids to Known Sequences

The nucleotide sequences of the subcloned fragments from Escherichia coli, Staphylococcus aureus, Enterococcus faecalis, Klebsiella pneumoniae, Pseudomonas aeruginosa and Salmonella typhimurium obtained from the expression vectors discussed above were compared to known sequences from Escherichia coli, Staphylococcus aureus, Enterococcus faecalis, Klebsiella pneumoniae, Pseudomonas aeruginosa, Salmonella typhimurium and other microorganisms as follows. First, to confirm that each clone originated from one location on the chromosome and was not chimeric, the nucleotide sequences of the selected clones were compared against the Escherichia coli, Staphylococcus aureus, Enterococcus faecalis, Klebsiella pneumoniae, Pseudomonas aeruginosa or Salmonella typhimurium genomic sequences to align the clone to the correct position on the chromosome. The NCBI BLASTN v 2.0.9 program was used for this comparison, and the incomplete Staphylococcus aureus genomic sequences licensed from TIGR, as well as the NCBI nonredundant GenBank database were used as the source of genomic data. Salmonella typhimurium sequences were compared to sequences available from the Genome Sequencing Center, and the Sanger Centre. Pseudomonas aeruginosa sequences were compared to a proprietary database and the NCBI GenBank database. The E. faecalis sequences were compared to a proprietary database.

The BLASTN analysis was performed using the default parameters except that the filtering was turned off. No further analysis was performed on inserts which resulted from the ligation of multiple fragments.

In general, antisense molecules and their complementary genes are identified as follows. First, all possible full length open reading frames (ORFs) are extracted from available genomic databases. Such databases include the GenBank nonredundant (nr) database, the unfinished genome database available from TIGR and the PathoSeq database developed by Incyte Genomics. The latter database comprises over 40 annotated bacterial genomes including complete ORF analysis. If databases are incomplete with regard to the bacterial genome of interest, it is not necessary to extract all ORFs in the genome but only to extract the ORFs within the portions of the available genomic sequences which are complementary to the clones of interest. Computer algorithms for identifying ORFs, such as GeneMark, are available and well known to those in the art. Comparison of the clone DNA to the complementary ORF(s) allows determination of whether the clone is a sense or antisense clone. Furthermore, each ORF extracted from the database can be compared to sequences in well annotated databases including the GenBank (nr) protein database, SWISSPROT and the like. A description of the gene or of a closely related gene in a closely related microorganism is often available in these databases. Similar methods are used to identify antisense clones corresponding to genes encoding non-translated RNAs.

Each of the cloned nucleic acid sequences discussed above was used to identify the corresponding Escherichia coli, Staphylococcus aureus, Enterococcus faecalis, Klebsiella pneumoniae, Pseudomonas aeruginosa or Salmonella typhimurium ORFs in the PathoSeq v.4.1 (March 2000 release) database of microbial genomic sequences. For this purpose, the NCBI BLASTN 2.0.9 computer algorithm was used. The default parameters were used except that filtering was turned off. The default parameters for the BLASTN and BLASTX analyses were:
Expectation value (e)=10
Alignment view options: pairwise
Filter query sequence (DUST with BLASTN, SEG with others)=T
Cost to open a gap (zero invokes behavior)=0
Cost to extend a gap (zero invokes behavior)=0
X dropoff value for gapped alignment (in bits) (zero invokes behavior)=0
Show GI's in deflines=F
Penalty for a nucleotide mismatch (BLASTN only)=!3
Reward for a nucleotide match (BLASTN only)=1
Number of one-line descriptions (V)=500
Number of alignments to show (B)=250
Threshold for extending hits=default
Perform gapped alignment (not available with BLASTX)=T
Query Genetic code to use=1
DB Genetic code (for TBLAST[nx]only=1
Number of processors to use=1
SeqAlign file
Believe the query defline=F
Matrix=BLOSUM62
Word Size=default
Effective length of the database (use zero for the real size)=0
Number of best hits from a region to keep=100
Length of region used to judge hits=20
Effective length of the search space (use zero for the real size)=0
Query strands to search against database (for BLAST[nx] and TBLASTX), 3 is both, 1 is top, 2 is bottom=3
Produce HTML output=F

Alternatively, ORFs were identified and refined by conducting a survey of the public and private data sources. Full-length gene protein and nucleotide sequences for these organisms were assembled from various sources. For Pseudomonas aeruginosa, gene sequences were adopted from the Pseudomonas genome sequencing project. The Pseudomonas genome sequencing project can be accessed on the internet by entering the following quoted text, “www.pseudomonas”, in the address bar of a web browser, such as Internet Explorer or Netscape, followed immediately by “.com”. For Klebsiella pneumoniae, Staphylococcus aureus, Streptococcus pneumoniae and Salmonella typhi, genomic sequences from PathoSeq v 4.1 (March 2000 release) was reanalyzed for ORFs using the gene finding software GeneMark v 2.4a, which was purchased from GenePro Inc. 451 Bishop St., N.W., Suite B, Atlanta, Ga., 30318, USA. It will be appreciated that ORFs may also be identified using databases other than PathoSeq. For example, the ORFs may be identified using the methods described in U.S. Provisional Patent Application Ser. No. 60/191,078, filed Mar. 21, 2000, the disclosure of which is incorporated herein by reference in its entirety.

Antisense clones were identified as those clones for which transcription from the inducible promoter would result in the expression of an RNA antisense to a complementary ORF, intergenic or intragenic sequence. Using the above methods, antisense numerous genes required for cellular proliferation were identified. Genes that were identified as required for proliferation in Staphylococcus aureus are provided herein as SEQ ID NOs: 201-550 (see also Table VI).

The next Example shows that once a proliferation-required gene has been identified using the above methods, homologous proliferation-required genes can be identified from heterologous organisms.

### Example 4

### Identification of Nucleic Acids Homologous to Nucleic Acids Required for the Proliferation of Staphylococcus aureus in other Bacterial Species

Nucleic acids homologous to proliferation-required nucleic acids from Staphylococcus aureus were identified as follows. For example, thirty-nine antisense nucleic acids which inhibited the growth of Staphylococcus aureus were identified using methods such as those described herein and were inserted into an expression vector such that their expression was under the control of a xylose-inducible Xyl-T5 promoter. A vector with a reporter gene under control of the Xyl-T5 promoter was used to show that expression from the Xyl-T5 promoter in Staphylococcus epidermidis was comparable to that in Staphylococcus aureus.

The vectors were introduced into Staphylococcus epidermidis by electroporation as follows: Staphylococcus epidermidis was grown in liquid culture to mid-log phase and then harvested by centrifugation. The cell pellet was resuspended in ⅓ culture volume of ice-cold EP buffer (0.625 M sucrose, 1 mM MgCl₂, pH=4.0), and then harvested again by centrifugation. The cell pellet was then resuspended with 1/40 volume EP buffer and allowed to incubate on ice for 1 hour. The cells were then frozen for storage at −80° C. For electroporation, 50 μl of thawed electrocompetent cells were combined with 0.5 μg plasmid DNA and then subjected to an electrical pulse of 10 kV/cm, 25 uFarads, 200 ohm using a Biorad gene pulser electroporation device. The cells were immediately resuspended with 200 μl outgrowth medium and incubated for 2 hours prior to plating on solid growth medium with drug selection to maintain the plasmid vector. Colonies resulting from overnight growth of these platings were selected, cultured in liquid medium with drug selection, and then subjected to dilution plating analysis as described for Staphylococcus aureus in Example 1 above to test growth sensitivity in the presence of the inducer xylose.

The results are shown in Table IV below. The first column indicates the Molecule Number of the Staphylococcus aureus antisense nucleic acid which was introduced into Staphylococcus epidermidis. The second column indicates whether the antisense nucleic acid inhibited the growth of Staphylococcus epidermidis, with a indicating that growth was inhibited. Of the 39 Staphylococcus aureus antisense nucleic acids evaluated, 20 inhibited the growth of Staphylococcus epidermidis.

TABLE IV
Sensitivity of Other Microorganisms to Antisense Nucleic
Acids That Inhibit Proliferation of Staphylococcus aureus

| Mol. No. | S. epidermidis |
|---|---|
| SaXA005 | + |
| SaXA007 | + |
| SaXA008 | + |
| SaXA009 | + |
| SaXA010 | + |
| SaXA011 | − |
| SaXA012 | − |
| SaXA013 | − |
| SaXA015 | + |
| SaXA017 | − |
| SaXA022 | + |
| SaXA023 | − |
| SaXA024 | − |
| SaXA025 | + |
| SaXA026 | + |
| SaXA027 | − |
| SaXA027b | − |
| SaXA02c | − |
| SaXA028 | − |
| SaXA029 | + |
| SaXA030 | + |
| SaXA032 | + |
| SaXA033 | + |
| SaXA034 | − |
| SaXA035 | + |
| SaXA037 | + |
| SaXA039 | − |
| SaXA042 | − |
| SaXA043 | − |
| SaXA044 | − |
| SaXA045 | + |
| SaXA051 | + |
| SaXA053 | − |
| SaXA056b | − |
| SaXA059a | + |
| SaXA060 | − |
| SaXA061 | + |
| SaXA062 | + |
| SaXA063 | − |
| SaXA065 | − |

Although the results shown above were obtained using a subset of proliferation-required nucleic acids from Staphylococcus aureus, it will be appreciated that similar analyses may be performed using other antisense nucleic acids obtained using the methods described herein to determine whether they inhibit the proliferation of cells or microorganisms including Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis and any species falling within the genera of any of the above species.

The next Example demonstrates methods for identifying homologs to proliferation-required genes by functional complementation.

### Example 5

### Identification of Homologous Nucleic Acids by Functional Complementation

Homologous coding nucleic acids, homologous antisense nucleic acids or nucleic acids encoding homologous polypeptides can be identified as follows. Gene products whose activities can be complemented by a proliferation-required gene product from Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species are identified using merodiploids, created by introducing a plasmid or Bacterial Artificial Chromosome into an organism having a mutation in the essential gene which reduces or eliminates the activity of the gene product. In some embodiments, the mutation may be a conditional mutation, such as a temperature sensitive mutation, such that the organism proliferates under permissive conditions but is unable to proliferate under non-permissive conditions in the absence of complementation by the gene on the plasmid or Bacterial Artificial Chromosome. Alternatively, duplications may be constructed as described in Roth et al. (1987) Biosynthesis of Aromatic Amino Acids in Escherichia coli and Salmonella typhimurium, F. C. Neidhardt, ed., American Society for Microbiology, publisher, pp. 2269-2270, the disclosure of which is incorporated herein by reference in its entirety. Such methods are familiar to those skilled in the art. Alternatively, homologous coding nucleic acids, homologous antisense nucleic acids or nucleic acids encoding homologous polypeptides may be identified by placing a gene required for proliferation or a nucleic acid complementary to at least a portion of a gene required for proliferation under the control of a regulatable promoter as described above, introducing a plasmid or Bacterial Artificial Chromosome into the cell, and identifying cells which are able to proliferate under conditions which would prevent or reduce proliferation in the absence of the plasmid or Bacterial Artificial Chromosome.

Homologous coding nucleic acids, homologous antisense nucleic acids or nucleic acids encoding homologous polypeptides can also be identified using databases as described in the next Example.

### Example 6

### Identification of Homologous Nucleic Acids by Database Analysis

As a demonstration of the methodology required to find homologues to an essential gene, fifty-one prokaryotic organisms were analyzed and compared in detail. First, the most reliable source of gene sequences for each organism was assessed by conducting a survey of the public and private data sources. The fifty-one organisms studied were Escherichia coli, Staphylococcus aureus, Enterococcus faecalis, Klebsiella pneumoniae, Pseudomonas aeruginosa, Salmonella typhimurium, Acinetobacter baumannii, Bacillus anthracis, Bacteroides fragilis, Bordetella pertussis, Borrelia burgdorferi, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Corynebacterium diptheriae, Enterobacter cloacae, Enterococcus faecium, Haemophilus influenzae, Helicobacter pylori, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Pasteurella multocida, Proteus mirabilis, Pseudomonas putida, Pseudomonas syringae, Salmonella paratyphi, Salmonella typhi, Staphylococcus epidermidis, Staphylococcus haemolyticus, Streptococcus mutans, Streptococcus pneumoniae, Streptococcus pyogenes, Treponema pallidum, Ureaplasma urealyticum, Vibrio cholerae and Yersinia pestis. Full-length gene, protein and nucleotide sequences for these organisms were assembled from various sources. For Escherichia coli, Haemophilus influenzae and Helicobacter pylori, gene sequences were adopted from the public sequencing projects, and derived from the GenPept 115 database (available from NCBI). For Pseudomonas aeruginosa, gene sequences were adopted from the Pseudomonas genome sequencing project. For Klebsiella pneumoniae, Staphylococcus aureus, Streptococcus pneumoniae and Salmonella typhi, genomic sequences from PathoSeq v 4.1 (March 2000 release) were reanalyzed for ORFs using the gene finding software GeneMark v 2.4a, which was purchased from GenePro Inc. 451 Bishop St., N.W., Suite B, Atlanta, Ga., 30318, USA. Similar analyses were conducted for the other organisms using publicly available and proprietary databases.

Homologous coding nucleic acids and the homologous polypeptides which they encode can be identified using a “reciprocal” best-hit analysis. To facilitate the identification of homologous coding nucleic acids and homologous polypeptides, paralogous genes within each of 51 organisms were identified and clustered prior to comparison to other organisms. Briefly, the polypeptide sequence of each polypeptide encoded by each open reading frame (ORF) in a given organism was compared to the polypeptide sequence encoded by every other ORF for that organism for each of the 51 pathogenic organisms (PathoSeq September 2001 release) using BLASTP 2.09 algorithm without filtering. Simultaneously, the polypeptide sequence encoded by each ORF of an organism was compared to the polypeptide sequences encoded by each of the ORFs in the remaining 51 organisms. Those polypeptides within a single organism that shared a higher degree of sequence identity to one another than to polypeptide sequences obtained from any other organisms were clustered as “paralog”. sequences for “reciprocal” best-hit analysis.

For each reference organism, the 50 homologous coding nucleic acids (and the 50 homologous polypeptides which they encode) were determined by identifying the ORFs in each of the 50 comparison organisms which encode a polypeptide sharing the highest degree of amino acid sequence identity to the polypeptide encoded by the ORF from the reference organism. The accuracy of the identification of the predicted homologous coding nucleic acids (and the homologous polypeptides which they encode) was confirmed by a “reciprocal” BLAST analysis in which the polypeptide sequence of the predicted homologous polypeptide was compared against the polypeptides encoded by each of the ORFs in the reference organism using BLASTP 2.09 algorithm without filtering. Only those polypeptides that shared the highest degree of amino acid sequence identity in each portion of the two-way comparison were retained for further analysis.

Amino acid identity or similarity can also be determined using the FASTA version 3.0t78 algorithm with the default parameters. Alternatively, protein identity or similarity may be identified using BLASTP with the default parameters, BLASTX with the default parameters, or TBLASTN with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety).

Nucleotide identity can be measured using BLASTN version 2.0 with the default parameters or tBLASTX with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety). Alternatively a gene can be classified into a cluster of orthologous groups (COG) by using the COGNITOR program available at the above web site, or by direct BLASTP comparison of the gene of interest to the members of the COGs and analysis of these results as described by Tatusov, R. L., Galperin, M. Y., Natale, D. A. and Koonin, E. V. (2000) The COG database: a tool for genome-scale analysis of protein functions and evolution. Nucleic Acids Research v. 28 n. 1, pp33-36, the disclosure of which is incorporated herein by reference in its entirety. Several genes homologous to genes contained the operons of SEQ ID NOs: 1-194 and several polypeptides homologous to polypeptides encoded by gene contained in the operons of SEQ ID NOs: 1-194 are described in U.S. patent application Ser. No. 10/282,122, entitled IDENTIFICATION OF ESSENTIAL GENES IN MICROORGANISMS, filed Oct. 25, 2002, the disclosure of which is incorporated herein by reference in its entirety.

The following Examples demonstrate methods used to identify proliferation-required operons that contain one or more of the identified proliferation-required genes.

### Example 7

### Prediction of Proliferation-Required Operons in Staphylococcus aureus

Operons containing one or more of the of the genes identified as being required for proliferation in Staphylococcus aureus Mu50 were identified using the operon prediction methods described herein.

The methods of operon prediction described herein were used to predict over 1397 operons from the protein-encoding genes in the genome of Staphylococcus aureus strain Mu50. Of the identified operons, approximately 62% were predicted to be monocistronic and approximately 38% were predicted to be polycistronic. Of the 1397 operons predicted, 194 of these operons were found to comprise at least one gene proliferation-required gene. Each of these proliferation-required operons predicted from Staphylococcus aureus is listed in Table V. In a few instances, the above prediction method resulted in very similar high scores for two or more distinct operon constructions. This result is likely due to naturally occurring differential termination within a single operon which results in the prediction of the full-length operon as well as one or more shorter operon sequences.

Starting from the left of Table V, the first column lists the SEQ ID NO for each nucleotide sequence for each predicted operon. The second column indicates the chromosomal strand which corresponds to the coding strand of the operon. The start and stop locations, with reference to the position on the standardized Staphylococcus aureus Mu 50 chromosomal map, are provided in the third and fourth columns. The final column provides the generic name for each gene contained within the operon.

TABLE V
Proliferation-Required Operons from Staphylococcus aureus

| | | Start- | Stop- | |
|---|---|---|---|---|
| SeqId | Strand | Location | Location | OperonMember |
| 1 | + | 3670 | 9674 | SAV0003, recF, gyrB, gyrA |
| 2 | + | 521792 | 525676 | treP, SAV0475, SAV0476 |
| 3 | + | 526319 | 529619 | SAV0477, dnaX, SAV0479, recR |
| 4 | + | 536410 | 543112 | SAV0481, tmk, SAV0483, holB, SAV0485, SAV0486, SAV0487, SAV0488, SAV0489 |
| 5 | + | 543397 | 548143 | metS, SAV0491, SAV0492, ksgA, veg |
| 6 | + | 543397 | 546174 | metS, SAV0491 |
| 7 | + | 543397 | 547772 | metS, SAV0491, SAV0492, ksgA |
| 8 | + | 554895 | 569447 | pth, mfd, SAV0504, SAV0505, SAV0506, SAV0507, SAV0508, SAV0509, SAV0510, ftsH, SAV0512, |
| | | | | cysK |
| 9 | + | 554895 | 562858 | pth, mfd, SAV0504, SAV0505, SAV0506, SAV0507, SAV0508 |
| 10 | + | 563038 | 568336 | SAV0509, SAV0510, ftsH, SAV0512 |
| 11 | + | 563038 | 564877 | SAV0509, SAV0510 |
| 12 | + | 569663 | 571282 | folP, folB, folK |
| 13 | + | 573866 | 579838 | rRNA-5S, tRNA-Val, tRNA-Thr, tRNA-Lys, tRNA-Gly, tRNA-Leu, tRNA-Arg, tRNA-Pro, tRNA-Ala, |
| | | | | rRNA-16S, tRNA-Ile, rRNA-23S, rRNA-5S |
| 14 | + | 594778 | 599127 | cysE, cysS, SAV0531, SAV0532, SAV0533 |
| 15 | + | 594778 | 598477 | cysE, cysS, SAV0531, SAV0532 |
| 16 | + | 600322 | 616222 | SAV0536, rplK, rplA, rplJ, rplL, SAV0541, rpoB, rpoC, SAV0544, rpsL, rpsG, fus, tufA |
| 17 | + | 600322 | 602870 | SAV0536, rplK, rplA, rplJ, rplL |
| 18 | + | 603868 | 611179 | rpoB, rpoC |
| 19 | + | 611668 | 616222 | rpsL, rpsG, fus, tufA |
| 20 | + | 662589 | 664414 | pta, SAV0589 |
| 21 | + | 665082 | 667998 | mvaK1, mvaD, mvaK2 |
| 22 | + | 681080 | 683166 | SAV0606, argS |
| 23 | − | 704990 | 707489 | SAV0633, SAV0632, SAV0631 |
| 24 | + | 770582 | 774224 | SAV0698, fruB, fruA |
| 25 | + | 807305 | 810860 | nrdI, nrdE, nrdF |
| 26 | − | 815371 | 816626 | SAV0738, SAV0737 |
| 27 | + | 842639 | 847358 | hprK, lgt, SAV0762, SAV0763, trxB |
| 28 | + | 855814 | 863055 | gapR, gap, pgk, tpi, pgm, eno |
| 29 | + | 962954 | 965075 | SAV0919, SAV0920 |
| 30 | + | 1006830 | 1016883 | SAV0963, spsA, spsB, SAV0966, SAV0967, SAV0968 |
| 31 | + | 1006830 | 1008525 | SAV0963, spsA, spsB |
| 32 | + | 1031458 | 1033655 | fabH, SAV0984 |
| 33 | + | 1034324 | 1040023 | oppB, SAV0987, oppD, oppF, SAV0990 |
| 34 | + | 1054132 | 1061116 | SAV1005, SAV1006, SAV1007, SAV1008, SAV1009, SAV1010, fabI |
| 35 | + | 1068803 | 1070746 | murE, SAV1019, SAV1020 |
| 36 | − | 1078988 | 1079153 | tRNA-Asn, tRNA-Ser |
| 37 | + | 1135082 | 1136850 | SAV1081, SAV1082 |
| 38 | + | 1137004 | 1138991 | ptsH, ptsI |
| 39 | − | 1143077 | 1144946 | SAV1090, SAV1089 |
| 40 | + | 1177683 | 1178709 | SAV1124, SAV1125 |
| 41 | + | 1190656 | 1194116 | pheS, pheT |
| 42 | + | 1207185 | 1209080 | murI, SAV1152, SAV1153 |
| 43 | + | 1222207 | 1225891 | argF, SAV1170, SAV1171 |
| 44 | + | 1230909 | 1243344 | SAV1177, SAV1178, SAV1179, ftsL, pbpA, mraY, murD, div1b, ftsA, ftsZ |
| 45 | + | 1232666 | 1243344 | SAV1178, SAV1179, ftsL, pbpA, mraY, murD, div1b, ftsA, ftsZ |
| 46 | + | 1232666 | 1236678 | SAV1178, SAV1179, ftsL, pbpA |
| 47 | + | 1236970 | 1243344 | mraY, murD, div1b, ftsA, ftsZ |
| 48 | + | 1236970 | 1239286 | mraY, murD |
| 49 | + | 1239302 | 1243344 | div1b, ftsA, ftsZ |
| 50 | + | 1267422 | 1272086 | gmk, SAV1210, SAV1211, priA |
| 51 | + | 1274210 | 1283164 | SAV1215, SAV1216, SAV1217, SAV1218, SAV1219, SAV1220, SAV1221, cfxE, SAV1223 |
| 52 | + | 1284176 | 1291873 | SAV1225, SAV1226, recG, SAV1228, plsX, fabD, fabG |
| 53 | + | 1286401 | 1291873 | recG, SAV1228, plsX, fabD, fabG |
| 54 | + | 1288679 | 1291873 | SAV1228, plsX, fabD, fabG |
| 55 | + | 1292308 | 1300063 | hmrB, rnc, smc, SAV1235, SAV1236, ffh |
| 56 | + | 1292308 | 1292541 | hmrB |
| 57 | + | 1292657 | 1300063 | rnc, smc, SAV1235, SAV1236, ffh |
| 58 | + | 1300498 | 1302655 | rpsP, rimM, trmD, rplS |
| 59 | + | 1305906 | 1307541 | SAV1243, rnhB |
| 60 | + | 1317539 | 1321244 | xerC, clpQ, clpY, codY |
| 61 | + | 1321591 | 1324853 | rpsB, SAV1257, smbA, frr |
| 62 | + | 1325226 | 1339274 | uppS, cdsA, SAV1262, proS, polC, SAV1265, nusA, SAV1267, SAV1268, infB |
| 63 | + | 1325226 | 1334580 | uppS, cdsA, SAV1262, proS, polC |
| 64 | + | 1334870 | 1339274 | SAV1265, nusA, SAV1267, SAV1268, infB |
| 65 | + | 1339660 | 1342466 | rbfA, truB, ribC, rpsO |
| 66 | + | 1347097 | 1353467 | spoIIIE, SAV1277, SAV1278, SAV1279, SAV1280 |
| 67 | + | 1380432 | 1384897 | SAV1307, SAV1308, glnR, glnA |
| 68 | + | 1380432 | 1382927 | SAV1307, SAV1308 |
| 69 | + | 1430727 | 1435126 | parE, parC |
| 70 | − | 1466891 | 1471456 | SAV1389, SAV1388, SAV1387, pstB, SAV1385 |
| 71 | + | 17319 | 23732 | SAV0013, SAV0014, rplI, dnaC, purA |
| 72 | + | 17319 | 22171 | SAV0013, SAV0014, rplI, dnaC |
| 73 | − | 1501456 | 1503678 | SAV1419, murG, SAV1417 |
| 74 | − | 1505760 | 1510022 | thyA, dfrA, SAV1425, SAV1424, SAV1423, SAV1422, SAV1421 |
| 75 | + | 1562515 | 1565321 | recU, pbp2 |
| 76 | − | 1569510 | 1575534 | SAV1458, SAV1457, SAV1456, dinG |
| 77 | − | 1583551 | 1586762 | SAV1472, gerCB, gerCC, ndk, SAV1468, SAV1467 |
| 78 | − | 1587636 | 1591358 | SAV1476, SAV1475, gpsA |
| 79 | − | 1587636 | 1589961 | SAV1475, gpsA |
| 80 | − | 1627042 | 1629649 | SAV1512, gnd |
| 81 | − | 1632000 | 1636699 | SAV1518, bfmBAA, bfmBAB, bmfBB |
| 82 | − | 1639429 | 1644510 | accB, accC, SAV1525, SAV1524, SAV1523, SAV1522, ispA |
| 83 | − | 1667591 | 1676522 | SAV1564, SAV1563, dnaG, sigA, SAV1560, SAV1559, SAV1558, SAV1557 |
| 84 | − | 1671862 | 1676522 | SAV1564, SAV1563, dnaG, sigA |
| 85 | − | 1667591 | 1671731 | SAV1560, SAV1559, SAV1558, SAV1557 |
| 86 | − | 1678398 | 1682249 | phoH, SAV1570, SAV1569, cdd, bex, SAV1566 |
| 87 | − | 1685671 | 1694755 | hemN, hrcA, grpE, dnaK, dnaJ, SAV1578, SAV1577, SAV1576 |
| 88 | − | 1697786 | 1707969 | pfs, SAV1598, SAV1597, aroE, SAV1595, SAV1594, SAV1593, SAV1592, SAV1591, SAV1590, |
| | | | | comEB, SAV1588, SAV1587 |
| 89 | − | 1717660 | 1721631 | SAV1614, SAV1613, SAV1612, udk, greA |
| 90 | − | 1721916 | 1725624 | alaS, SAV1617, SAV1616, SAV1615 |
| 91 | − | 1722994 | 1725624 | alaS |
| 92 | − | 1721916 | 1722931 | SAV1617, SAV1616, SAV1615 |
| 93 | − | 1729809 | 1732070 | SAV1622, SAV1621 |
| 94 | − | 1737277 | 1740321 | hisS, aspS |
| 95 | − | 1744736 | 1756186 | obg, SAV1643, ruvA, ruvB, queA, tgt, SAV1638, secF, SAV1636, apt |
| 96 | − | 1756540 | 1757470 | rplU, SAV1646, rpmA |
| 97 | − | 1767593 | 1773035 | valS, folC, SAV1661, truncated-radC |
| 98 | − | 1774221 | 1787036 | SAV1677, SAV1676, tig, clpX, SAV1673, hemA, hemX, hemC, hemD, hemB, hemL, SAV1666, |
| | | | | SAV1665 |
| 99 | − | 1775490 | 1787036 | SAV1677, SAV1676, tig, clpX, SAV1673, hemA, hemX, hemC, hemD, hemB, hemL |
| 100 | − | 1781859 | 1787036 | SAV1677, SAV1676, tig, clpX, SAV1673 |
| 101 | − | 1787178 | 1788328 | infC, rpmI, rplT |
| 102 | − | 1792834 | 1795625 | SAV1686, dnaB, dnaI |
| 103 | − | 1795836 | 1801188 | polA, SAV1689, SAV1688, gapB |
| 104 | − | 1810718 | 1813420 | pfk, pykA |
| 105 | − | 1815254 | 1817055 | SAV1701, accA |
| 106 | + | 167374 | 183942 | capA, capB, capC, capD, capE, capF, capG, capH, capI, capJ, capK, capL, capM, capN, capO, capP |
| 107 | − | 1818929 | 1823088 | SAV1704, dnaE |
| 108 | − | 1865490 | 1875703 | SAV1745, SAV1744, SAV1743, SAV1742, SAV1741, murC, SAV1739, SAV1738 |
| 109 | − | 1867487 | 1875703 | SAV1745, SAV1744, SAV1743, SAV1742, SAV1741, murC |
| 110 | − | 1882358 | 1885150 | SAV1754, SAV1753, SAV1752 |
| 111 | + | 1885515 | 1886706 | SAV1755, SAV1756 |
| 112 | − | 1893708 | 1896455 | leuS, SAV1759 |
| 113 | − | 1959708 | 1960455 | tRNA-Met, tRNA, tRNA-Phe, tRNA-His, tRNA-Gly, tRNA-Asn, tRNA-Glu, tRNA-Ser |
| 114 | + | 24161 | 24315 | tRNA-Glu, tRNA-Asp |
| 115 | − | 1994154 | 1994350 | tRNA-Gly, tRNA-Leu |
| 116 | − | 1994457 | 2001747 | rRNA-16S, tRNA-Ile, tRNA-Ala, 23S-rRNA, rRNA-5S, tRNA-Val, tRNA-Thr, tRNA-Lys, tRNA-Leu, |
| | | | | tRNA-Gly, tRNA-Leu, tRNA-Arg, tRNA-Pro, tRNA-Ala, tRNA-Met, tRNA-Met, tRNA-Ser, tRNA-Asp, |
| | | | | tRNA-Ser, tRNA-Met, tRNA-Asp, tRNA-Phe, tRNA-Thr, tRNA-Tyr, tRNA-Trp, tRNA-His, tRNA-Gln, |
| | | | | tRNA-Cys, tRNA-Gly |
| 117 | − | 2030928 | 2032974 | SAV1892, SAV1891 |
| 118 | − | 2036795 | 2039184 | SAV1898, SAV1897 |
| 119 | − | 2039952 | 2043153 | SAV1901, SAV1900, SAV1899 |
| 120 | − | 2045147 | 2051247 | pcrB, pcrA, dnlJ, SAV1903 |
| 121 | − | 2056827 | 2059110 | SAV1913, nadE |
| 122 | + | 2064607 | 2066149 | SAV1918, SAV1919 |
| 123 | − | 2148841 | 2150817 | groES, groEL |
| 124 | − | 2190799 | 2190973 | tRNA-Leu, tRNA-Gly |
| 125 | − | 2197673 | 2202397 | SAV2074, SAV2073, SAV2072, dpj, air, SAV2069, SAV2068 |
| 126 | − | 2214440 | 2216883 | ddlA, murF |
| 127 | − | 2222584 | 2226472 | SAV2094, thiD, thiM, thiE, SAV2090 |
| 128 | − | 2222584 | 2223456 | SAV2090 |
| 129 | − | 2229031 | 2231115 | SAV2101, murA, murA, SAV2098 |
| 130 | − | 2231677 | 2238577 | SAV2110, atpB, atpE, atpF, atpH, atpA, atpG, atpD, atpC |
| 131 | − | 2238744 | 2242341 | SAV2114, glyA, upp, mnaA |
| 132 | − | 2242448 | 2246493 | tdk, prfA, SAV2117, SAV2116, SAV2115 |
| 133 | − | 2250440 | 2252123 | murZ, SAV2123 |
| 134 | − | 2250864 | 2252123 | murZ |
| 135 | − | 2295658 | 2298783 | SAV2163, SAV2162, glmM(femD) |
| 136 | − | 2300018 | 2305665 | rRNA-16S, rRNA-23S, rRNA-5S, tRNA-Asn, tRNA-Glu, tRNA-Val, tRNA-Tyr, tRNA-Gln, tRNA-Lys |
| 137 | − | 2359399 | 2363758 | SAV2222, SAV2221, SAV2220, truA, rplM, rpsI |
| 138 | − | 2364296 | 2378211 | rpsJ, rplC, rplD, rplW, rplB, rpsS, rplV, rpsC, rplP, rpmC, rpsQ, rplN, rplX, rplE, rpsN, rpsH, rplF, rplR, |
| | | | | rpsE, rpmD, rplO, secY, adk, infA, rpmJ, rpsM, rpsK, rpoA, rplQ |
| 139 | + | 24929 | 29591 | vicR, vicK, SAV0020, SAV0021 |
| 140 | − | 2470474 | 2472501 | SAV2347, fni |
| 141 | − | 2764088 | 2766471 | nrdD, SAV2616 |
| 142 | + | 285731 | 292354 | SAV0244, SAV0245, SAV0246, gatC, SAV0248, SAV0249, SAV0250 |
| 143 | + | 517 | 3289 | dnaA, dnaN |
| 144 | + | 292882 | 296332 | SAV0251, SAV0252, SAV0253 |
| 145 | + | 551317 | 554584 | gcaD, prs, rplY |
| 146 | + | 1312537 | 1317121 | SAV1249, SAV1250, gid |
| 147 | − | 1662306 | 1665101 | sodA, pbp3 |
| 148 | − | 1662306 | 1664381 | pbp3 |
| 149 | + | 1838194 | 1839504 | SAV1718, rpsD |
| 150 | + | 298205 | 303403 | SAV0255, SAV0256, SAV0257, SAV0258 |
| 151 | − | 2877215 | 2877826 | rpmH, rnpA |
| 152 | + | 411837 | 412951 | rpsF, ssb, rpsR |
| 153 | − | 2687157 | 2688434 | mvaA |
| 154 | + | 2855908 | 2856864 | SAV2689 |
| 155 | + | 2570287 | 2572365 | SAV2439 |
| 156 | + | 2217201 | 2218403 | SAV2084 |
| 157 | − | 2252589 | 2253449 | fbaA |
| 158 | − | 2256243 | 2256773 | rpoE |
| 159 | − | 2279873 | 2281678 | glmS |
| 160 | − | 2380594 | 2382729 | topB |
| 161 | − | 2390725 | 2391990 | fmhB |
| 162 | − | 2185545 | 2190678 | rRNA-16S, rRNA-23S, rRNA-5S |
| 163 | − | 2027584 | 2028342 | map |
| 164 | − | 1850510 | 1851772 | tyrS |
| 165 | − | 1921077 | 1922273 | metK |
| 166 | − | 1836363 | 1838057 | SAV1717 |
| 167 | − | 1848353 | 1848970 | SAV1727 |
| 168 | − | 1617898 | 1618818 | SAV1504 |
| 169 | + | 1619131 | 1620615 | SAV1505 |
| 170 | + | 1676857 | 1678248 | glyS |
| 171 | − | 1685202 | 1685378 | rpsU |
| 172 | − | 1790484 | 1792421 | thrS |
| 173 | + | 1410706 | 1410975 | rpsN |
| 174 | − | 1567896 | 1569188 | asnS |
| 175 | − | 1587193 | 1587465 | hu |
| 176 | + | 1345167 | 1346840 | SAV1275 |
| 177 | − | 1145474 | 1146025 | pdf1 |
| 178 | + | 1165912 | 1167033 | SAV1113 |
| 179 | − | 1228583 | 1228656 | tRNA-Arg |
| 180 | + | 1005174 | 1006505 | pgi |
| 181 | − | 1045860 | 1046849 | trpS |
| 182 | − | 755146 | 756021 | bacA |
| 183 | + | 778476 | 779459 | SAV0704 |
| 184 | + | 819167 | 820291 | SAV0742 |
| 185 | + | 830606 | 833137 | secA |
| 186 | + | 833570 | 834562 | prfB |
| 187 | − | 851567 | 851638 | tRNA-Arg |
| 188 | + | 521231 | 521320 | tRNA-Ser |
| 189 | + | 530479 | 535507 | rRNA-16S, rRNA-23S, rRNA-5S |
| 190 | + | 592895 | 594349 | gltX |
| 191 | + | 12793 | 14079 | serS |
| 192 | + | 1243604 | 1250453 | SAV1187, SAV1188, SAV1189, SAV1190, SAV1191, SAV1192, ileS |
| 193 | + | 1243604 | 1247479 | SAV1187, SAV1188, SAV1189, SAV1190, SAV1191, SAV1192 |
| 194 | + | 1247700 | 1250453 | ileS |

Comparison of the predicted operons listed in Table V with experimentally determined values for Staphylococcus aureus operons from literature sources indicated that the disclosed methods successfully predicted operon boundaries and genes contained within the each operon interior with a high degree of accuracy.

For each of the Staphylococcus aureus operons that are listed in Table V (SEQ ID NOs: 1-194), Table VI indicates which gene or genes are required for proliferation. The proliferation-required genes displayed in Table VI are listed by both their generic gene name (EEG_GeneName) and their SEQ ID NO (EEG_DNASeqID). In those instances where the proliferation-required gene encodes a protein product, the SEQ ID NO of the encoded polypeptide is also included (EEG_PrtSeqID). For example, Table VI indicates that the proliferation-required gene gyrB, which has the nucleotide sequence of SEQ ID NO: 201, is contained in the operon having the nucleotide sequence of SEQ ID NO: 1 and encodes the polypeptide having the amino acid sequence of SEQ ID NO: 551.

TABLE VI
Proliferation-Required Genes Contained in Proliferation-Required
Operons and Proliferation-Required Polypeptides Encoded
by the Proliferation-Required Genes

| OperonSeqID | EEG_PathoSeqID | EEG_GeneName | EEG_DNASeqID | EEG_PrtSeqID |
|---|---|---|---|---|
| 1 | SAU800005 | gyrB | 201 | 551 |
| 1 | SAU800006 | gyrA | 202 | 552 |
| 2 | SAU800475 | SAV0475 | 203 | 553 |
| 3 | SAU800478 | dnaX | 204 | 554 |
| 3 | SAU800479 | SAV0479 | 205 | 555 |
| 4 | SAU800482 | tmk | 206 | 556 |
| 4 | SAU800484 | holB | 207 | 557 |
| 4 | SAU800489 | SAV0489 | 208 | 558 |
| 5 | SAU800490 | metS | 209 | 559 |
| 6 | SAU800490 | metS | 209 | 559 |
| 7 | SAU800490 | metS | 209 | 559 |
| 8 | SAU800502 | pth | 210 | 560 |
| 8 | SAU800506 | SAV0506 | 211 | 561 |
| 8 | SAU800507 | SAV0507 | 212 | 562 |
| 8 | SAU800509 | SAV0509 | 213 | 563 |
| 8 | SAU800510 | SAV0510 | 214 | 564 |
| 9 | SAU800502 | pth | 210 | 560 |
| 9 | SAU800506 | SAV0506 | 211 | 561 |
| 9 | SAU800507 | SAV0507 | 212 | 562 |
| 10 | SAU800509 | SAV0509 | 213 | 563 |
| 10 | SAU800510 | SAV0510 | 214 | 564 |
| 11 | SAU800509 | SAV0509 | 213 | 563 |
| 11 | SAU800510 | SAV0510 | 214 | 564 |
| 12 | SAU800514 | folP | 215 | 565 |
| 12 | SAU800515 | folB | 216 | 566 |
| 12 | SAU800516 | folK | 217 | 567 |
| 13 | SAU802716 | rRNA-5S | 218 | |
| 13 | SAU802749 | tRNA-Val | 219 | |
| 13 | SAU802767 | tRNA-Thr | 220 | |
| 13 | SAU802784 | tRNA-Lys | 221 | |
| 13 | SAU802725 | tRNA-Gly | 222 | |
| 13 | SAU802742 | tRNA-Leu | 223 | |
| 13 | SAU802760 | tRNA-Arg | 224 | |
| 13 | SAU802773 | tRNA-Pro | 225 | |
| 13 | SAU802717 | tRNA-Ala | 226 | |
| 13 | SAU802734 | rRNA-16S | 227 | |
| 13 | SAU802754 | tRNA-Ile | 228 | |
| 13 | SAU802788 | rRNA-23S | 229 | |
| 13 | SAU802745 | rRNA-5S | 230 | |
| 14 | SAU800530 | cysS | 231 | 568 |
| 15 | SAU800530 | cysS | 231 | 568 |
| 16 | SAU800537 | rplK | 232 | 569 |
| 16 | SAU800539 | rplJ | 233 | 570 |
| 16 | SAU800540 | rplL | 234 | 571 |
| 16 | SAU800542 | rpoB | 235 | 572 |
| 16 | SAU800543 | rpoC | 236 | 573 |
| 16 | SAU800545 | rpsL | 237 | 574 |
| 16 | SAU800546 | rpsG | 238 | 575 |
| 16 | SAU800547 | fus | 239 | 576 |
| 16 | SAU800548 | tufA | 240 | 577 |
| 17 | SAU800537 | rplK | 232 | 569 |
| 17 | SAU800539 | rplJ | 233 | 570 |
| 17 | SAU800540 | rplL | 234 | 571 |
| 18 | SAU800542 | rpoB | 235 | 572 |
| 18 | SAU800543 | rpoC | 236 | 573 |
| 19 | SAU800545 | rpsL | 237 | 574 |
| 19 | SAU800546 | rpsG | 238 | 575 |
| 19 | SAU800547 | fus | 239 | 576 |
| 19 | SAU800548 | tufA | 240 | 577 |
| 20 | SAU800588 | pta | 241 | 578 |
| 21 | SAU800590 | mvaK1 | 242 | 579 |
| 21 | SAU800591 | mvaD | 243 | 580 |
| 21 | SAU800592 | mvaK2 | 244 | 581 |
| 22 | SAU800607 | argS | 245 | 582 |
| 23 | SAU800633 | SAV0633 | 246 | 583 |
| 24 | SAU800698 | SAV0698 | 247 | 584 |
| 25 | SAU800731 | nrdE | 248 | 585 |
| 25 | SAU800732 | nrdF | 249 | 586 |
| 26 | SAU800738 | SAV0738 | 250 | 587 |
| 27 | SAU800760 | hprK | 251 | 588 |
| 27 | SAU800764 | trxB | 252 | 589 |
| 28 | SAU800772 | gap | 253 | 590 |
| 28 | SAU800773 | pgk | 254 | 591 |
| 28 | SAU800774 | tpi | 255 | 592 |
| 28 | SAU800776 | eno | 256 | 593 |
| 29 | SAU800920 | SAV0920 | 257 | 594 |
| 30 | SAU800965 | spsB | 258 | 595 |
| 31 | SAU800965 | spsB | 258 | 595 |
| 32 | SAU800983 | fabH | 259 | 596 |
| 32 | SAU800984 | SAV0984 | 260 | 597 |
| 33 | SAU800989 | oppF | 261 | 598 |
| 34 | SAU801007 | SAV1007 | 262 | 599 |
| 34 | SAU801011 | fabI | 263 | 600 |
| 35 | SAU801018 | murE | 264 | 601 |
| 36 | SAU802752 | tRNA-Asn | 265 | |
| 36 | SAU802738 | tRNA-Ser | 266 | |
| 37 | SAU801081 | SAV1081 | 267 | 602 |
| 38 | SAU801083 | ptsH | 268 | 603 |
| 38 | SAU801084 | ptsI | 269 | 604 |
| 39 | SAU801089 | SAV1089 | 270 | 605 |
| 40 | SAU801124 | SAV1124 | 271 | 606 |
| 40 | SAU801125 | SAV1125 | 272 | 607 |
| 41 | SAU801138 | pheS | 273 | 608 |
| 41 | SAU801139 | pheT | 274 | 609 |
| 42 | SAU801151 | murI | 275 | 610 |
| 42 | SAU801152 | SAV1152 | 276 | 611 |
| 43 | SAU801170 | SAV1170 | 277 | 612 |
| 44 | SAU801179 | SAV1179 | 278 | 613 |
| 44 | SAU801180 | ftsL | 279 | 614 |
| 44 | SAU801181 | pbpA | 280 | 615 |
| 44 | SAU801182 | mraY | 281 | 616 |
| 44 | SAU801183 | murD | 282 | 617 |
| 44 | SAU801185 | ftsA | 283 | 618 |
| 44 | SAU801186 | ftsZ | 284 | 619 |
| 45 | SAU801179 | SAV1179 | 278 | 613 |
| 45 | SAU801180 | ftsL | 279 | 614 |
| 45 | SAU801181 | pbpA | 280 | 615 |
| 45 | SAU801182 | mraY | 281 | 616 |
| 45 | SAU801183 | murD | 282 | 617 |
| 45 | SAU801185 | ftsA | 283 | 618 |
| 45 | SAU801186 | ftsZ | 284 | 619 |
| 46 | SAU801179 | SAV1179 | 278 | 613 |
| 46 | SAU801180 | ftsL | 279 | 614 |
| 46 | SAU801181 | pbpA | 280 | 615 |
| 47 | SAU801182 | mraY | 281 | 616 |
| 47 | SAU801183 | murD | 282 | 617 |
| 47 | SAU801185 | ftsA | 283 | 618 |
| 47 | SAU801186 | ftsZ | 284 | 619 |
| 48 | SAU801182 | mraY | 281 | 616 |
| 48 | SAU801183 | murD | 282 | 617 |
| 49 | SAU801185 | ftsA | 283 | 618 |
| 49 | SAU801186 | ftsZ | 284 | 619 |
| 50 | SAU801209 | gmk | 285 | 620 |
| 51 | SAU801216 | SAV1216 | 286 | 621 |
| 51 | SAU801219 | SAV1219 | 287 | 622 |
| 51 | SAU801223 | SAV1223 | 288 | 623 |
| 52 | SAU801230 | fabD | 289 | 624 |
| 52 | SAU801231 | fabG | 290 | 625 |
| 53 | SAU801230 | fabD | 289 | 624 |
| 53 | SAU801231 | fabG | 290 | 625 |
| 54 | SAU801230 | fabD | 289 | 624 |
| 54 | SAU801231 | fabG | 290 | 625 |
| 55 | SAU801232 | hmrB | 291 | 626 |
| 55 | SAU801233 | rnc | 292 | 627 |
| 55 | SAU801234 | smc | 293 | 628 |
| 55 | SAU801235 | SAV1235 | 294 | 629 |
| 56 | SAU801232 | hmrB | 291 | 626 |
| 57 | SAU801233 | rnc | 292 | 627 |
| 57 | SAU801234 | smc | 293 | 628 |
| 57 | SAU801235 | SAV1235 | 294 | 629 |
| 58 | SAU801238 | rpsP | 295 | 630 |
| 58 | SAU801240 | trmD | 296 | 631 |
| 58 | SAU801241 | rplS | 297 | 632 |
| 59 | SAU801243 | SAV1243 | 298 | 633 |
| 60 | SAU801252 | xerC | 299 | 634 |
| 61 | SAU801256 | rpsB | 300 | 635 |
| 61 | SAU801257 | SAV1257 | 301 | 636 |
| 61 | SAU801258 | smbA | 302 | 637 |
| 61 | SAU801259 | frr | 303 | 638 |
| 62 | SAU801260 | uppS | 304 | 639 |
| 62 | SAU801263 | proS | 305 | 640 |
| 62 | SAU801264 | polC | 306 | 641 |
| 62 | SAU801266 | nusA | 307 | 642 |
| 62 | SAU801267 | SAV1267 | 308 | 643 |
| 62 | SAU801269 | infB | 309 | 644 |
| 63 | SAU801260 | uppS | 304 | 639 |
| 63 | SAU801263 | proS | 305 | 640 |
| 63 | SAU801264 | polC | 306 | 641 |
| 64 | SAU801266 | nusA | 307 | 642 |
| 64 | SAU801267 | SAV1267 | 308 | 643 |
| 64 | SAU801269 | infB | 309 | 644 |
| 65 | SAU801272 | ribC | 310 | 645 |
| 66 | SAU801280 | SAV1280 | 311 | 646 |
| 67 | SAU801307 | SAV1307 | 312 | 647 |
| 68 | SAU801307 | SAV1307 | 312 | 647 |
| 69 | SAU801354 | parE | 313 | 648 |
| 69 | SAU801355 | parC | 314 | 649 |
| 70 | SAU801389 | SAV1389 | 315 | 650 |
| 70 | SAU801388 | SAV1388 | 316 | 651 |
| 71 | SAU800016 | dnaC | 317 | 652 |
| 72 | SAU800016 | dnaC | 317 | 652 |
| 73 | SAU801418 | murG | 318 | 653 |
| 74 | SAU801427 | thyA | 319 | 654 |
| 74 | SAU801426 | dfrA | 320 | 655 |
| 75 | SAU801449 | recU | 321 | 656 |
| 76 | SAU801458 | SAV1458 | 322 | 657 |
| 76 | SAU801457 | SAV1457 | 323 | 658 |
| 77 | SAU801470 | gerCC | 324 | 659 |
| 78 | SAU801475 | SAV1475 | 325 | 660 |
| 79 | SAU801475 | SAV1475 | 325 | 660 |
| 80 | SAU801511 | gnd | 326 | 661 |
| 81 | SAU801518 | SAV1518 | 327 | 662 |
| 81 | SAU801516 | bfmBAB | 328 | 663 |
| 82 | SAU801527 | accB | 329 | 664 |
| 82 | SAU801526 | accC | 330 | 665 |
| 82 | SAU801524 | SAV1524 | 331 | 666 |
| 83 | SAU801562 | dnaG | 332 | 667 |
| 83 | SAU801561 | sigA | 333 | 668 |
| 83 | SAU801560 | SAV1560 | 334 | 669 |
| 83 | SAU801558 | SAV1558 | 335 | 670 |
| 84 | SAU801562 | dnaG | 332 | 667 |
| 84 | SAU801561 | sigA | 333 | 668 |
| 85 | SAU801560 | SAV1560 | 334 | 669 |
| 85 | SAU801558 | SAV1558 | 335 | 670 |
| 86 | SAU801567 | bex | 336 | 671 |
| 87 | SAU801581 | grpE | 337 | 672 |
| 87 | SAU801580 | dnaK | 338 | 673 |
| 88 | SAU801598 | SAV1598 | 339 | 674 |
| 88 | SAU801597 | SAV1597 | 340 | 675 |
| 88 | SAU801596 | aroE | 341 | 676 |
| 88 | SAU801594 | SAV1594 | 342 | 677 |
| 88 | SAU801587 | SAV1587 | 343 | 678 |
| 89 | SAU801610 | greA | 344 | 679 |
| 90 | SAU801618 | alaS | 345 | 680 |
| 90 | SAU801616 | SAV1616 | 346 | 681 |
| 91 | SAU801618 | alaS | 345 | 680 |
| 92 | SAU801616 | SAV1616 | 346 | 681 |
| 93 | SAU801622 | SAV1622 | 347 | 682 |
| 93 | SAU801621 | SAV1621 | 348 | 683 |
| 94 | SAU801631 | hisS | 349 | 684 |
| 95 | SAU801644 | obg | 350 | 685 |
| 95 | SAU801642 | ruvA | 351 | 686 |
| 95 | SAU801641 | ruvB | 352 | 687 |
| 96 | SAU801647 | rplU | 353 | 688 |
| 96 | SAU801645 | rpmA | 354 | 689 |
| 97 | SAU801663 | valS | 355 | 690 |
| 98 | SAU801674 | clpX | 356 | 691 |
| 98 | SAU801673 | SAV1673 | 357 | 692 |
| 99 | SAU801674 | clpX | 356 | 691 |
| 99 | SAU801673 | SAV1673 | 357 | 692 |
| 100 | SAU801674 | clpX | 356 | 691 |
| 100 | SAU801673 | SAV1673 | 357 | 692 |
| 101 | SAU801680 | infC | 358 | 693 |
| 101 | SAU801678 | rplT | 359 | 694 |
| 102 | SAU801685 | dnaB | 360 | 695 |
| 102 | SAU801684 | dnaI | 361 | 696 |
| 103 | SAU801690 | polA | 362 | 697 |
| 103 | SAU801688 | SAV1688 | 363 | 698 |
| 104 | SAU801698 | pfk | 364 | 699 |
| 104 | SAU801697 | pykA | 365 | 700 |
| 105 | SAU801701 | SAV1701 | 366 | 701 |
| 105 | SAU801700 | accA | 367 | 702 |
| 106 | SAU800152 | capD | 368 | 703 |
| 107 | SAU801703 | dnaE | 369 | 704 |
| 108 | SAU801740 | murC | 370 | 705 |
| 109 | SAU801740 | murC | 370 | 705 |
| 110 | SAU801754 | SAV1754 | 371 | 706 |
| 110 | SAU801753 | SAV1753 | 372 | 707 |
| 111 | SAU801755 | SAV1755 | 373 | 708 |
| 112 | SAU801760 | leuS | 374 | 709 |
| 113 | SAU802757 | tRNA-Met | 375 | |
| 113 | SAU802741 | tRNA | 376 | |
| 113 | SAU802724 | tRNA-Phe | 377 | |
| 113 | SAU802781 | tRNA-His | 378 | |
| 113 | SAU802765 | tRNA-Gly | 379 | |
| 113 | SAU802747 | tRNA-Asn | 380 | |
| 113 | SAU802732 | tRNA-Glu | 381 | |
| 113 | SAU802772 | tRNA-Ser | 382 | |
| 114 | SAU802761 | tRNA-Glu | 383 | |
| 114 | SAU802774 | tRNA-Asp | 384 | |
| 115 | SAU802743 | tRNA-Gly | 385 | |
| 115 | SAU802726 | tRNA-Leu | 386 | |
| 116 | SAU802715 | rRNA-16S | 387 | |
| 116 | SAU802756 | tRNA-Ile | 388 | |
| 116 | SAU802721 | tRNA-Ala | 389 | |
| 116 | SAU802723 | rRNA-23S | 390 | |
| 116 | SAU802766 | rRNA-5S | 391 | |
| 116 | SAU802748 | tRNA-Val | 392 | |
| 116 | SAU802731 | tRNA-Thr | 393 | |
| 116 | SAU802790 | tRNA-Lys | 394 | |
| 116 | SAU802771 | tRNA-Leu | 395 | |
| 116 | SAU802755 | tRNA-Gly | 396 | |
| 116 | SAU802740 | tRNA-Leu | 397 | |
| 116 | SAU802722 | tRNA-Arg | 398 | |
| 116 | SAU802778 | tRNA-Pro | 399 | |
| 116 | SAU802764 | tRNA-Ala | 400 | |
| 116 | SAU802729 | tRNA-Met | 401 | |
| 116 | SAU802787 | tRNA-Met | 402 | |
| 116 | SAU802770 | tRNA-Ser | 403 | |
| 116 | SAU802753 | tRNA-Asp | 404 | |
| 116 | SAU802739 | tRNA-Ser | 405 | |
| 116 | SAU802720 | tRNA-Met | 406 | |
| 116 | SAU802777 | tRNA-Asp | 407 | |
| 116 | SAU802763 | tRNA-Phe | 408 | |
| 116 | SAU802746 | tRNA-Thr | 409 | |
| 116 | SAU802728 | tRNA-Tyr | 410 | |
| 116 | SAU802769 | tRNA-Trp | 411 | |
| 116 | SAU802750 | tRNA-His | 412 | |
| 116 | SAU802735 | tRNA-Gln | 413 | |
| 116 | SAU802718 | tRNA-Cys | 414 | |
| 116 | SAU802775 | tRNA-Gly | 415 | |
| 117 | SAU801892 | SAV1892 | 416 | 710 |
| 117 | SAU801891 | SAV1891 | 417 | 711 |
| 118 | SAU801898 | SAV1898 | 418 | 712 |
| 119 | SAU801901 | SAV1901 | 419 | 713 |
| 119 | SAU801900 | SAV1900 | 420 | 714 |
| 119 | SAU801899 | SAV1899 | 421 | 715 |
| 120 | SAU801904 | dnlJ | 422 | 716 |
| 121 | SAU801913 | SAV1913 | 423 | 717 |
| 121 | SAU801912 | nadE | 424 | 718 |
| 122 | SAU801919 | SAV1919 | 425 | 719 |
| 123 | SAU802030 | groES | 426 | 720 |
| 123 | SAU802029 | groEL | 427 | 721 |
| 124 | SAU802785 | tRNA-Leu | 428 | |
| 124 | SAU802768 | tRNA-Gly | 429 | |
| 125 | SAU802071 | dpj | 430 | 722 |
| 125 | SAU802070 | alr | 431 | 723 |
| 126 | SAU802083 | ddlA | 432 | 724 |
| 126 | SAU802082 | murF | 433 | 725 |
| 127 | SAU802090 | SAV2090 | 434 | 726 |
| 128 | SAU802090 | SAV2090 | 434 | 726 |
| 129 | SAU802100 | murA | 435 | 727 |
| 129 | SAU802098 | SAV2098 | 436 | 728 |
| 130 | SAU802109 | atpB | 437 | 729 |
| 130 | SAU802107 | atpF | 438 | 730 |
| 130 | SAU802106 | atpH | 439 | 731 |
| 130 | SAU802105 | atpA | 440 | 732 |
| 130 | SAU802104 | atpG | 441 | 733 |
| 130 | SAU802103 | atpD | 442 | 734 |
| 130 | SAU802102 | atpC | 443 | 735 |
| 131 | SAU802112 | upp | 444 | 736 |
| 132 | SAU802118 | prfA | 445 | 737 |
| 133 | SAU802124 | murZ | 446 | 738 |
| 134 | SAU802124 | murZ | 446 | 738 |
| 135 | SAU802163 | SAV2163 | 447 | 739 |
| 135 | SAU802161 | glmM(femD) | 448 | 740 |
| 136 | SAU802733 | rRNA-16S | 449 | |
| 136 | SAU802786 | rRNA-23S | 450 | |
| 136 | SAU802751 | rRNA-5S | 451 | |
| 136 | SAU802737 | tRNA-Asn | 452 | |
| 136 | SAU802719 | tRNA-Glu | 453 | |
| 136 | SAU802776 | tRNA-Val | 454 | |
| 136 | SAU802762 | tRNA-Tyr | 455 | |
| 136 | SAU802744 | tRNA-Gln | 456 | |
| 136 | SAU802727 | tRNA-Lys | 457 | |
| 137 | SAU802222 | SAV2222 | 458 | 741 |
| 137 | SAU802221 | SAV2221 | 459 | 742 |
| 137 | SAU802218 | rplM | 460 | 743 |
| 138 | SAU802251 | rpsJ | 461 | 744 |
| 138 | SAU802250 | rplC | 462 | 745 |
| 138 | SAU802249 | rplD | 463 | 746 |
| 138 | SAU802248 | rplW | 464 | 747 |
| 138 | SAU802247 | rplB | 465 | 748 |
| 138 | SAU802246 | rpsS | 466 | 749 |
| 138 | SAU802245 | rplV | 467 | 750 |
| 138 | SAU802244 | rpsC | 468 | 751 |
| 138 | SAU802243 | rplP | 469 | 752 |
| 138 | SAU802241 | rpsQ | 470 | 753 |
| 138 | SAU802240 | rplN | 471 | 754 |
| 138 | SAU802239 | rplX | 472 | 755 |
| 138 | SAU802238 | rplE | 473 | 756 |
| 138 | SAU802236 | rpsH | 474 | 757 |
| 138 | SAU802235 | rplF | 475 | 758 |
| 138 | SAU802234 | rplR | 476 | 759 |
| 138 | SAU802233 | rpsE | 477 | 760 |
| 138 | SAU802230 | secY | 478 | 761 |
| 138 | SAU802229 | adk | 479 | 762 |
| 138 | SAU802228 | infA | 480 | 763 |
| 138 | SAU802226 | rpsM | 481 | 764 |
| 138 | SAU802225 | rpsK | 482 | 765 |
| 138 | SAU802224 | rpoA | 483 | 766 |
| 138 | SAU802223 | rplQ | 484 | 767 |
| 139 | SAU800018 | vicR | 485 | 768 |
| 139 | SAU800019 | vicK | 486 | 769 |
| 140 | SAU802346 | fni | 487 | 770 |
| 141 | SAU802617 | nrdD | 488 | 771 |
| 141 | SAU802616 | SAV2616 | 489 | 772 |
| 142 | SAU800248 | SAV0248 | 490 | 773 |
| 143 | SAU800001 | dnaA | 491 | 774 |
| 143 | SAU800002 | dnaN | 492 | 775 |
| 144 | SAU800251 | SAV0251 | 493 | 776 |
| 144 | SAU800252 | SAV0252 | 494 | 777 |
| 145 | SAU800499 | gcaD | 495 | 778 |
| 145 | SAU800500 | prs | 496 | 779 |
| 146 | SAU801250 | SAV1250 | 497 | 780 |
| 147 | SAU801552 | pbp3 | 498 | 781 |
| 148 | SAU801552 | pbp3 | 498 | 781 |
| 149 | SAU801719 | rpsD | 499 | 782 |
| 150 | SAU800255 | SAV0255 | 500 | 783 |
| 150 | SAU800256 | SAV0256 | 501 | 784 |
| 150 | SAU800258 | SAV0258 | 502 | 785 |
| 151 | SAU802713 | rnpA | 503 | 786 |
| 152 | SAU800366 | ssb | 504 | 787 |
| 152 | SAU800367 | rpsR | 505 | 788 |
| 153 | SAU802545 | mvaA | 506 | 789 |
| 154 | SAU802689 | SAV2689 | 507 | 790 |
| 155 | SAU802439 | SAV2439 | 508 | 791 |
| 156 | SAU802084 | SAV2084 | 509 | 792 |
| 157 | SAU802125 | fbaA | 510 | 793 |
| 158 | SAU802128 | rpoE | 511 | 794 |
| 159 | SAU802154 | glmS | 512 | 795 |
| 160 | SAU802254 | topB | 513 | 796 |
| 161 | SAU802262 | fmhB | 514 | 797 |
| 162 | SAU802780 | rRNA-16S | 515 | |
| 162 | SAU802759 | rRNA-23S | 516 | |
| 162 | SAU802782 | rRNA-5S | 517 | |
| 163 | SAU801888 | map | 518 | 798 |
| 164 | SAU801729 | tyrS | 519 | 799 |
| 165 | SAU801790 | metK | 520 | 800 |
| 166 | SAU801717 | SAV1717 | 521 | 801 |
| 167 | SAU801727 | SAV1727 | 522 | 802 |
| 168 | SAU801504 | SAV1504 | 523 | 803 |
| 169 | SAU801505 | SAV1505 | 524 | 804 |
| 170 | SAU801565 | glyS | 525 | 805 |
| 171 | SAU801575 | rpsU | 526 | 806 |
| 172 | SAU801683 | thrS | 527 | 807 |
| 173 | SAU801336 | rpsN | 528 | 808 |
| 174 | SAU801454 | asnS | 529 | 809 |
| 175 | SAU801473 | hu | 530 | 810 |
| 176 | SAU801275 | SAV1275 | 531 | 811 |
| 177 | SAU801091 | pdf1 | 532 | 812 |
| 178 | SAU801113 | SAV1113 | 533 | 813 |
| 179 | SAU802736 | tRNA-Arg | 534 | |
| 180 | SAU800962 | pgi | 535 | 814 |
| 181 | SAU800996 | trpS | 536 | 815 |
| 182 | SAU800683 | bacA | 537 | 816 |
| 183 | SAU800704 | SAV0704 | 538 | 817 |
| 184 | SAU800742 | SAV0742 | 539 | 818 |
| 185 | SAU800753 | secA | 540 | 819 |
| 186 | SAU800754 | prfB | 541 | 820 |
| 187 | SAU802783 | tRNA-Arg | 542 | |
| 188 | SAU802730 | tRNA-Ser | 543 | |
| 189 | SAU802779 | rRNA-16S | 544 | |
| 189 | SAU802758 | rRNA-23S | 545 | |
| 189 | SAU802789 | rRNA-5S | 546 | |
| 190 | SAU800528 | gltX | 547 | 821 |
| 191 | SAU800009 | serS | 548 | 822 |
| 192 | SAU801192 | SAV1192 | 549 | 823 |
| 192 | SAU801193 | ileS | 550 | 824 |
| 193 | SAU801192 | SAV1192 | 549 | 823 |
| 194 | SAU801193 | ileS | 550 | 824 |

Each of the proliferation-required operons identified in Tables V and VI can be further studied using the methods described in Example 9 to identify any additional proliferation-required genes contained in each operon.

Analysis of an Exemplary Operon Comprising dnaA and dnaN in Staphylococcus Aureus

As previously indicated, one aspect of the present teachings is that a high confidence prediction of operons can be achieved even in instances where the overall operon structure and/or genes contained within the operon possess characteristics which increase the likelihood of misidentification when using conventional singular operon prediction methods. In an exemplary application of the aforementioned methods, in the target organism, Staphylococcus aureus, the genes dnaA and dnaN were predicted to reside in the same operon as indicated in Tables V and VI above (excerpts from each table shown below):

dnaA/dnaN operon entry from Table V

| SeqId | Strand | StartLocation | StopLocation | OperonMember |
|---|---|---|---|---|
| 143 | + | 517 | 3289 | dnaA, dnaN |

dnaA/dnaN operon entries from Table VI

| OperonSeqID | EEG_PathoSeqID | EEG_GeneName | EEG_DNASeqID | EEG_PrtSeqID |
|---|---|---|---|---|
| 143 | SAU800001 | dnaA | 491 | 774 |
| 143 | SAU800002 | dnaN | 492 | 775 |

Further information obtained during operon analysis indicated that dnaA has a sequence length of 1362 bp (mapped between a contig start of 517 and a contig end of 1878) and dnaN has a sequence length of 1134 bp (mapped between a contig start of 2156 and a contig end of 3289). The resulting intergenic distance between these two genes was found to be approximately 278 bp.

The distance between the dnaA/dnaN pair, is substantially greater than the distance between the majority of gene pairs for many known operons. For example, in Escherichia coli, the dnaA and dnaN genes reside only 5 bp apart and have previously been identified as being contained in the same operon. When genes are separated by large intergenic distances such as that described above, conventional approaches may fail to properly associate the genes with the same operon or may do so only with a low degree of confidence.

Using the methods described herein, it was predicted with high confidence that dnaA and dnaN genes reside in the same operon in Staphylococcus aureus despite the relatively large intergenic distance. Such a prediction was achieved in part by the observation that this gene pair is relatively highly conserved in a number of genomes to which Staphylococcus aureus may be compared. As an example, when comparing the dnaA/dnaN gene pair results in Staphylococcus aureus to those obtained for 39 distinct prokaryotic organism genomes comprising: Aquifex aeolicus, Bacillus halodurans, Bacillus subtilis, Borrelia burgdorferi, Buchnera sp., Campylobacter jejuni, Caulobacter crescentus, Chlamydia pneumoniae, Chlamydia trachomatis, Clostridium acetobutylicum, Enterococcus faecalis, Escherichia coli, Haemophilus influenzae, Helicobacter pylori, Lactococcus lactis, Listeria innocua, Listeria monocytogenes, Mycobacterium leprae, Mycobacterium tuberculosis, Mycoplasma genitalium, Mycoplasma pneumoniae, Mycoplasma pulmonis, Neisseria meningitides, Pasteurella multocida, Pseudomonas aeruginosa, Rickettsia conorii, Rickettsia prowazekii, Salmonella typhi, Salmonella typhimurium, Sinorhizobium meliloti, Streptococcus pneumoniae, Streptococcus pyogenes, Synechocystis sp., Thermotoga maritime, Treponema pallidum, Ureaplasma urealyticum, Vibrio cholerae, Xylella fastidiosa, and Yersinia pestis; it was observed that in approximately 24 of the comparison organisms the dnaA/dnaN gene pair was conserved. Furthermore, in certain organisms, for example Bacillus subtillis, dnaA and dnaN were found to be in the same operon despite an intergenic distance of approximately 189 bp.

Using conservation information such as that described above, it will be appreciated that high confidence operon prediction can be obtained even for gene pairs that might possess characteristics that would suggest that the genes of the gene pair reside in distinct operons when using conventional singular analysis approaches such as solely intergenic distance analysis.

The next Example illustrates how the operon prediction methods described herein can be used to identify proliferation-required operons from other bacterial species.

### Example 8

### Identification of Proliferation-Required Operons in Other Organisms

In this Example, the operon prediction methods described herein are used to predict the boundaries of proliferation-required operons from Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

Once the full length ORFs and the operons containing them have been identified using the methods described above, they can be obtained from a genomic library by performing a PCR amplification using primers at each end of the desired sequence. Those skilled in the art will appreciate that, if desired, the ORFs or operaons can be compared to homologous sequences in other cells or microorganisms to confirm the start and stop codons at the ends of the ORFs.

In some embodiments, the primers may contain restriction sites which facilitate the insertion of the gene or operon into a desired vector. For example, the gene may be inserted into an expression vector and used to produce the proliferation-required protein as described below. Other methods for obtaining the full length ORFs and/or operons are familiar to those skilled in the art. For example, natural restriction sites may be employed to insert the full length ORFs and/or operons into a desired vector.

The proliferation-required operons that are predicted are then further examined to identify any additional proliferation-required genes contained within the predicted operons by using the methods described in the following Example.

### Example 9

### Identification of Individual Genes within an Operon Required for Proliferation

This example illustrates a method for determining if a targeted gene within an operon is required for cellular proliferation by replacing the targeted allele in the chromosome with an in-frame deletion of the coding region of the targeted gene.

Deletion inactivation of a chromosomal copy of a gene in Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species can be accomplished by integrative gene replacement. The principles of this method are described in Xia, M., et al. 1999 Plasmid 42:144-149 and Hamilton, C. M., et al 1989. J. Bacteriol. 171: 4617-4622, the disclosures of which are incorporated herein by reference in their entireties. A similar gene disruption method is available for Pseudomonas aeruginosa, except the counter selectable marker is sacB (Schweizer, H. P., Klassen, T. and Hoang, T. (1996) Mol. Biol. of Pseudomonas. ASM press, 229-237, the disclosure of which is incorporated herein by reference in its entirety). In this approach, a mutant allele of the targeted gene is constructed by way of an in-frame deletion and introduced into the chromosome using a suicide vector. This results in a tandem duplication comprising a deleted (null) allele and a wild type allele of the target gene. Cells in which the vector sequences have been deleted are isolated using a counter-selection technique. Removal of the vector sequence from the chromosomal insertion results in either restoration of the wild-type target sequence or replacement of the wild type sequence with the deletion (null) allele. E. faecalis genes can be disrupted using a suicide vector that contains an internal fragment to a gene of interest. With the appropriate selection this plasmid will homologously recombine into the chromosome (Nallapareddy, S. R., X. Qin, G. M. Weinstock, M. Hook, B. E. Murray. 2000. Infect. Immun. 68:5218-5224, the disclosure of which is incorporated herein by reference).

The resultant population of colonies of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnet, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species can then be evaluated to determine whether the target sequence is required for proliferation by PCR amplification of the affected target sequence. If the targeted gene is not required for proliferation, then PCR analysis will show that roughly equal numbers of colonies have retained either the wild-type or the mutant allele. If the targeted gene is required for proliferation, then only wild-type alleles will be recovered in the PCR analysis.

The method of cross-over PCR is used to generate the mutant allele by amplification of nucleotide sequences flanking but not including the coding region of the gene of interest, using specifically designed primers such that overlap between the resulting two PCR amplification products allows them to hybridize. Further PCR amplification of this hybridization product using primers representing the extreme 5′ and 3′ ends can produce an amplification product containing an in-frame deletion of the coding region but retaining substantial flanking sequences.

For Staphylococcus aureus, this amplification product is subcloned into the suicide vector pSA3182 (Xia, M., et al. 1999 Plasmid 42:144-149, the disclosure of which is incorporated herein by reference in its entirety) which is host-dependent for autonomous replication. This vector includes a tetC tetracycline-resistance marker and the origin of replication of the well-known Staphylococcus aureus plasmid pT181 (Mojumdar, M and Kahn, S. A., Characterization of the Tetracycline Resistance Gene of Plasmid pT181, J. Bacteriol. 170: 5522 (1988), the disclosure of which is incorporated herein by reference in its entirety). The vector lacks the repC gene which is required for autonomous replication of the vector at the pT181 origin. This vector can be propagated in a Staphylococcus aureus host strain such as SA3528, which expresses repC in trans. Once the amplified truncated target gene sequence is cloned and propagated in the pSA3182 vector, it can then be introduced into a repC minus strain such as RN4220 (Kreiswirth, B. N. et al., The Toxic Shock Syndrome Exotoxin Structural Gene is Not Detectably Transmitted by a Prophage, Nature 305:709-712 (1983), the disclosure of which is incorporated herein by reference in its entirety) by electroporation with selection for tetracycline resistance. In this strain, the vector must integrate by homologous recombination at the targeted gene in the chromosome to impart drug resistance. This results in a inserted truncated copy of the allele, followed by pSA3182 vector sequence, and finally an intact and functional allele of the targeted gene.

Once a tetracycline resistant Staphylococcus aureus strain is isolated using the above technique and shown to include truncated and wild-type alleles of the targeted gene as described above, a second plasmid, pSA7592 (Xia, M., et al. 1999 Plasmid 42:144-149, the disclosure of which is incorporated herein by reference in its entirety) is introduced into the strain by electroporation. This gene includes an erythromycin resistance gene and a repC gene that is expressed at high levels. Expression of repC in these transformants is toxic due to interference of normal chromosomal replication at the integrated pT181 origin of replication. This selects for strains that have removed the vector sequence by homologous recombination, resulting in either of two outcomes: The selected cells either possess a wild-type allele of the targeted gene or a gene in which the wild-type allele has been replaced by the engineered in-frame deletion of the truncated allele.

PCR amplification can be used to determine the genetic outcome of the above process in the resulting erythromycin resistant, tet sensitive transformant colonies. If the targeted gene is not required for cellular replication, then PCR evidence for both wild-type and mutant alleles will be found among the population of resultant transformants. However, if the targeted gene is required for cellular proliferation, then only the wild-type form of the gene will be evident among the resulting transformants.

Similarly, for Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallet, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnet, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species the PCR products containing the mutant allele of the target sequence may be introduced into an appropriate knockout vector and cells in which the wild type target has been disrupted are selected using the appropriate methodology.

The above methods have the advantage that insertion of an in-frame deletion mutation is far less likely to cause downstream polar effects on genes in the same operon as the targeted gene. However, it will be appreciated that other methods for disrupting Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species genes which are familiar to those skilled in the art may also be used.

Each gene in the operon can be disrupted using the methodology above to determine whether it is required for proliferation.

The following example describes methods for inhibiting cellular proliferation of Staphylococcus aureus by expressing an antisense nucleic acid that is complementary to a portion of an operon that contains a proliferation-required gene.

### Example 10

### Inhibition of Proliferation by Providing an Antisense Nucleic Acid Complementary to a Portion of a Proliferation-Require Operon in Staphylococcus aureus

Several proliferation-inhibiting antisense nucleic acids which are complementary to portions of the Staphylococcus aureus cap operon (SEQ ID NO: 106), but which are not complementary to any of the currently known proliferation-required genes that are contained within this operon, are identified using the methods described in Example 1. An antisense nucleic acid that is complementary to a 30 nucleotide portion of the cap operon (SEQ ID NO: 106) located just upstream of the capA gene is selected. Additionally, an antisense nucleic acid that is complementary to a 30 nucleotide portion of the 5′-end of the capA gene is selected. Each of the two selected 30 nucleotide antisense nucleic acids are separately subcloned into the vector pXyIT5-P15a, which harbors the xylose inducible XylT5 promoter, in an orientation such that the strand that is complementary to the sense strand of the cap operon is transcribed. The ligated products are then separately transformed into electrocompetent Staphylococcus aureus. Resulting transformants are plated on agar containing LB+0.2% glucose (LBG medium)+chloramphenicol at 15 μg/ml (LBG+CM15 medium). After an overnight incubation, colonies are picked and inoculated into flasks containing fresh LBG+CM15 liquid medium. Each of the inoculated cultures containing an antisense construct are grown for 16 hours at 37° C. then 100 μl is transferred to fresh 5 ml LBG+CM15 liquid cultures with or without 2% xylose. The optical density of each newly inoculated liquid culture is measured and the cultures are then incubated for 16 hours at 37° C. At the end of the incubation, the optical density of each culture is again measured to determine the effect of inducing the expression of each of the antisense nucleic acids on cellular proliferation.

There is no change in the optical density of the cultures of Staphylococcus aureus grown in the presence of 2% xylose at the end of the 16 hour incubation whereas the optical density of the cultures grown in the absence of xylose changes by more than two absorbance units. These results are observed for both cultures transformed with the pXylT5-P15a vector harboring the 30 nucleotide antisense fragment complementary to the 5′-noncoding region upstream of the capA gene and cultures transformed with the pXylT5-P15a vector harboring the 30 nucleotide antisense fragment complementary to the 5′-coding region of the capA gene. Accordingly, these results indicate that induction of expression of an antisense transcript complementary to a noncoding portion of the cap operon or an antisense transcript complementary to a coding region of a gene that is contained in the cap operon, but which is not required for proliferation, causes an inhibition of the activity or reduction in the amount of one or more proliferation-required gene products encoded by one or more proliferation-required genes that are contained within the cap operon.

It will be appreciated that antisense nucleic acids of lengths other than 30 nucleotides and antisense nucleic acids which are complementary to other portions of the cap operon can also be used to inhibit proliferation. Furthermore, it will be appreciated that antisense nucleic acids complementary to portions of other proliferation-required operons from Staphylococcus aureus, including portions of the operons of SEQ ID NOs: 1-194, can be used to inhibit cellular proliferation.

### Example 11

### Inhibition of Proliferation in Other Bacterial Species

The ability of an antisense molecule identified in a first organism to inhibit the proliferation of a second organism was validated using antisense nucleic acids which inhibit the growth of E. coli which were identified using methods similar to those described above. Expression vectors which inhibited growth of E. coli upon induction of antisense RNA expression with IPTG were transformed directly into Enterobacter cloacae, Klebsiella pneumonia or Salmonella typhimurium. The transformed cells were then assayed for growth inhibition according to the method of Example 1. After growth in liquid culture, cells were plated at various serial dilutions and a score determined by calculating the log difference in growth for INDUCED vs. UNINDUCED antisense RNA expression as determined by the maximum 10 fold dilution at which a colony was observed. The results of these experiments are listed below in Table VII. If there was no effect of antisense RNA expression in a microorganism, the clone is minus in Table VII. In contrast, a positive in Table VII means that at least 10-fold more cells were required to observe a colony on the induced plate than on the non-induced plate under the conditions used and in that microorganism.

TABLE VII
Sensitivity of Other Microorganisms to Antisense Nucleic
Acids That Inhibit Proliferation in E. coli

| Mol. No. | S. typhimurium | E. cloacae | K. pneumoniae |
|---|---|---|---|
| EcXA001 | + | + | − |
| EcXA004 | + | − | − |
| EcXA005 | + | + | + |
| EcXA006 | − | − | − |
| EcXA007 | − | + | − |
| EcXA008 | + | − | + |
| EcXA009 | − | − | − |
| EcXA010 | + | + | + |
| EcXA011 | − | + | − |
| EcXA012 | − | + | − |
| EcXA013 | + | + | + |
| EcXA014 | + | + | − |
| EcXA015 | + | + | + |
| EcXA016 | + | + | + |
| EcXA017 | + | + | + |
| EcXA018 | + | + | + |
| EcXA019 | + | + | + |
| EcXA020 | + | + | + |
| EcXA021 | + | + | + |
| EcXA023 | + | + | + |
| EcXA024 | + | − | + |
| EcXA025 | − | − | − |
| EcXA026 | + | + | − |
| EcXA027 | + | + | − |
| EcXA028 | + | − | − |
| EcXA029 | − | − | − |
| EcXA030 | + | + | + |
| EcXA031 | + | − | − |
| EcXA032 | + | + | − |
| EcXA033 | + | + | + |
| EcXA034 | + | + | + |
| EcXA035 | − | − | − |
| EcXA036 | + | − | + |
| EcXA037 | + | + | − |
| EcXA038 | + | + | + |
| EcXA039 | + | − | − |
| EcXA041 | + | + | + |
| EcXA042 | − | + | + |
| EcXA043 | − | − | − |
| EcXA044 | − | − | − |
| EcXA045 | + | + | + |
| EcXA046 | − | − | − |
| EcXA047 | + | + | − |
| EcXA048 | − | − | − |
| EcXA049 | + | − | − |
| EcXA050 | − | − | − |
| EcXA051 | + | − | − |
| EcXA052 | + | − | − |
| EcXA053 | + | + | + |
| EcXA054 | − | − | + |
| EcXA055 | + | − | − |
| EcXA056 | + | − | + |
| EcXA057 | + | + | − |
| EcXA058 | − | − | − |
| EcXA059 | + | + | + |
| EcXA060 | − | − | − |
| EcXA061 | − | − | − |
| EcXA062 | − | − | − |
| EcXA063 | + | + | − |
| EcXA064 | − | − | − |
| EcXA065 | + | + | − |
| EcXA066 | − | − | − |
| EcXA067 | − | + | − |
| EcXA068 | − | − | − |
| EcXA069 | − | + | − |
| EcXA070 | − | − | − |
| EcXA071 | + | − | − |
| EcXA072 | + | − | + |
| EcXA073 | + | + | + |
| EcXA074 | + | + | + |
| EcXA075 | + | − | − |
| EcXA076 | − | + | − |
| EcXA077 | + | + | − |
| EcXA079 | + | + | + |
| EcXA080 | + | − | − |
| EcXA082 | − | + | − |
| EcXA083 | − | − | − |
| EcXA084 | − | + | − |
| EcXA086 | − | − | − |
| EcXA087 | − | − | − |
| EcXA088 | − | − | − |
| EcXA089 | − | − | − |
| EcXA090 | − | − | − |
| EcXA091 | − | − | − |
| EcXA092 | − | − | − |
| EcXA093 | − | − | − |
| EcXA094 | + | + | + |
| EcXA095 | + | + | − |
| EcXA096 | − | − | − |
| EcXA097 | + | − | − |
| EcXA098 | + | − | − |
| EcXA099 | − | − | − |
| EcXA100 | − | − | − |
| EcXA101 | − | − | − |
| EcXA102 | − | − | − |
| EcXA103 | − | + | − |
| EcXA104 | + | + | + |
| EcXA106 | + | + | − |
| EcXA107 | − | − | − |
| EcXA108 | − | − | − |
| EcXA109 | − | − | − |
| EcXA110 | + | + | − |
| EcXA111 | − | − | − |
| EcXA112 | − | + | − |
| EcXA113 | + | + | + |
| EcXA114 | − | + | − |
| EcXA115 | − | + | − |
| EcXA116 | + | + | − |
| EcXA117 | + | − | − |
| EcXA118 | − | − | − |
| EcXA119 | + | + | − |
| EcXA120 | − | − | − |
| EcXA121 | − | − | − |
| EcXA122 | + | − | + |
| EcXA123 | + | − | − |
| EcXA124 | − | − | − |
| EcXA125 | − | − | − |
| EcXA126 | − | − | − |
| EcXA127 | + | + | − |
| EcXA128 | − | − | − |
| EcXA129 | − | + | − |
| EcXA130 | + | + | − |
| EcXA132 | − | − | − |
| EcXA133 | − | − | − |
| EcXA136 | − | − | − |
| EcXA137 | − | − | − |
| EcXA138 | + | − | − |
| EcXA139 | − | − | − |
| EcXA140 | + | − | − |
| EcXA141 | + | − | − |
| EcXA142 | − | − | − |
| EcXA143 | − | + | − |
| EcXA144 | + | + | − |
| EcXA145 | − | − | − |
| EcXA146 | − | − | − |
| EcXA147 | − | − | − |
| EcXA148 | − | − | − |
| EcXA149 | + | + | + |
| EcXA150 | − | − | − |
| EcXA151 | + | − | − |
| EcXA152 | − | − | − |
| EcXA153 | + | + | − |
| EcXA154 | − | − | − |
| EcXA155 | − | − | ND |
| EcXA156 | − | + | − |
| EcXA157 | − | − | − |
| EcXA158 | − | − | − |
| EcXA159 | + | − | − |
| EcXA160 | + | − | − |
| EcXA162 | − | − | − |
| EcXA163 | − | − | − |
| EcXA164 | − | − | − |
| EcXA165 | − | − | − |
| EcXA166 | − | − | − |
| EcXA167 | − | − | − |
| EcXA168 | − | − | − |
| EcXA169 | − | + | − |
| EcXA171 | − | − | − |
| EcXA172 | − | − | − |
| EcXA173 | − | − | − |
| EcXA174 | − | − | − |
| EcXA175 | − | − | − |
| EcXA176 | − | − | − |
| EcXA178 | − | − | − |
| EcXA179 | − | − | − |
| EcXA180 | + | − | − |
| EcXA181 | − | − | − |
| EcXA182 | − | − | − |
| EcXA183 | − | − | − |
| EcXA184 | − | − | − |
| EcXA185 | − | − | − |
| EcXA186 | − | − | − |
| EcXA187 | + | + | + |
| EcXA189 | + | − | − |
| EcXA190 | + | + | + |
| EcXA191 | + | + | − |
| EcXA192 | − | + | − |

Given the above results, a skilled artisan will appreciate that the ability of an antisense nucleic acid which inhibits the proliferation of Staphylococcus aureus, including antisense nucleic acids complementary to at least a portion of an operon comprising a nucleotide sequence selected from the group consisting of SEQ ID NOs: 1-194, can be evaluated for the ability to inhibit the proliferation of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

It will also be appreciated that the above methods for evaluating the ability of an antisense nucleic acid to inhibit the proliferation of a heterologous organism can be performed using antisense nucleic acids complementary to at least a portion of any of the proliferation-required operons identified using the operon prediction methods described herein. For example, an antisense nucleic acid complementary to at least a portion of a proliferation-required operon from Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species can be evaluated for its ability to inhibit the proliferation of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

Those skilled in the art will appreciate that an antisense molecule which works in the microorganism from which it was obtained will not always work in a heterologous cell or microorganism. A skilled artisan will also appreciate that a negative result in a heterologous cell or microorganism does not necessarily mean that that cell or microorganism is missing a homologous proliferation-required operon nor does it necessarily mean that the homologous operon is not required for proliferation. However, a positive result means that the heterologous cell or microorganism contains a proliferation-required operon which is homologous, at least in part, to the proliferation-required operon against which the antisense nucleic acid was originally identified.

A skilled artisan will also recognize that heterologous cells in which proliferation is inhibited by antisense nucleic acids may be used in cell-based assays as described herein for the identification and characterization of compounds in order to develop antibiotics effective in such cells or microorganisms.

The following Example demonstrates methods for the inhibition of cellular proliferation in a first organism by using an antisense nucleic acid that is complementary to at least a portion of an operon that contains a gene that is homologous to a gene in the operon of a second organism.

### Example 12

### Inhibition of the Proliferation in Organisms Possessing a Proliferation-Required Gene that is Homologous to a Proliferation-Required Gene Contained in a Staphylococcus aureus Proliferation-Required Operon

In this example, an organism identified as possessing a proliferation-required gene that is homologous to a gene required for proliferation in Staphylococcus aureus (SEQ ID NOs: 201-550) or possessing a proliferation-required polypeptide that is homologous to a polypeptide required for proliferation in Staphylococcus aureus (SEQ ID NOs: 551-824) is provided with an antisense nucleic acid that is complementary to at least a portion of a proliferation-required operon which contains the homologous proliferation-required gene.

A nucleic acid that is homologous to a Staphylococcus aureus proliferation-required gene of SEQ ID NO: 491 is identified in Enterococcus faecalis using the method described in Example 6. Alternatively, a polypeptide that is homologous to a Staphylococcus aureus proliferation-required polypeptide encoded by SEQ ID NO: 491 (the polypeptide of SEQ ID NO: 774) is identified in Enterococcus faecalis using the methods decribed herein. Using genomic sequence information and the method described in Example 7, the operon boundaries for the operon containing the gene homologous to the gene SEQ ID NO: 491 are predicted.

Using the above identified operon sequence as a template, a pool of antisense nucleic acids, which is complementary to the 5′-end of the operon and ranges in size from 20 to 100 nucleotides, is generated using DNA synthesis and PCR technologies that are well known in the art. The antisense nucleic acid pool is then cloned into the expression vector pEPEF3 in an orientation such that transcripts complementary to the 5′-portion of the proliferation-required operon identified from Enterococcus faecalis is produced.

After transformation of the antisense expression vector into Enterococcus faecalis, the cells are grown and induction of expression of the antisense transcript is induced by adding xylose as described in Example 10.

Expression of the antisense transcripts results in inhibition of the proliferation of Enterococcus faecalis.

### Example 13

### Screening Chemical Libraries

Having determined the identity of proliferation-required operons, the present invention further contemplates the use of these operons in assays to screen libraries of compounds for potential drug candidates. The generation of chemical libraries is well known in the art. For example, combinatorial chemistry can be used to generate a library of compounds to be screened in the assays described herein. A combinatorial chemical library is a collection of diverse chemical compounds generated by either chemical synthesis or biological synthesis by combining a number of chemical “building block” reagents. For example, a linear combinatorial chemical library such as a polypeptide library is formed by combining amino acids in every possible combination to yield peptides of a given length. Millions of chemical compounds theoretically can be synthesized through such combinatorial mixings of chemical building blocks. For example, one commentator observed that the systematic, combinatorial mixing of 100 interchangeable chemical building blocks results in the theoretical synthesis of 100 million tetrameric compounds or 10 billion pentameric compounds. (Gallop et al., “Applications of Combinatorial Technologies to Drug Discovery, Background and Peptide Combinatorial Libraries,” Journal of Medicinal Chemistry, Vol. 37, No. 9, 1233-1250 (1994). Other chemical libraries known to those in the art may also be used, including natural product libraries. Once generated, combinatorial libraries can be screened for compounds that possess the ability to inhibit cellular proliferation.

Current cell-based assays used to identify or to characterize compounds for drug discovery and development frequently depend on detecting the ability of a test compound to modulate the activity of a target molecule located within a cell or located on the surface of a cell. An advantage of cell-based assays is that they allow the effect of a compound on a target molecule's activity to be detected within the physiologically relevant environment of the cell as opposed to an in vitro environment. Most often such target molecules are proteins such as enzymes, receptors and the like. However, target molecules may also include other molecules such as DNAs, lipids, carbohydrates and RNAs including messenger RNAs, ribosomal RNAs, tRNAs, regulatory RNAs and the like. A number of highly sensitive cell-based assay methods are available to those of skill in the art to detect binding and interaction of test compounds with specific target molecules. However, these methods are generally not highly effective when the test compound binds to or otherwise interacts with its target molecule with moderate or low affinity. In addition, the target molecule may not be readily accessible to a test compound in solution, such as when the target molecule is located inside the cell or within a cellular compartment. Thus, current cell-based assay methods are limited in that they are not effective in identifying or characterizing compounds that interact with their targets with moderate to low affinity or compounds that interact with targets that are not readily accessible.

The cell-based assay methods of the present invention have substantial advantages over current cell-based assays. These advantages derive from the use of sensitized cells in which the level or activity of at least one proliferation-required product encoded by a gene contained within a proliferation-required operon has been specifically reduced to the point where the presence or absence of its function becomes a rate-determining step for cellular proliferation. The cells in which the level or activity of at least one proliferation-required gene product has been reduced become much more sensitive to compounds that are active against the affected target molecule (the proliferation-required gene product). Thus, cell-based assays of the present invention are capable of detecting compounds exhibiting low or moderate potency against the target molecule of interest because such compounds are substantially more potent on sensitized cells than on non-sensitized cells. The effect may be such that a test compound may be two to several times more potent, at least 10 times more potent, at least 20 times more potent, at least 50 times more potent, at least 100 times more potent, at least 1000 times more potent, or even more than 1000 times more potent when tested on the sensitized cells as compared to the non-sensitized cells. Antisense nucleic acids complementary to at least a portion of a proliferation-required operon comprising a nucleotide sequence selected from the group consisting of SEQ ID NOs: 1-194 can be used to inhibit the proliferation of Staphylococcus aureus thereby sensitizing the cells for use in any of the cell-based assays described herein. Additionally, antisense nucleic acids complementary to proliferation-required operons, or portions thereof, from Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species can be used to inhibit the proliferation Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species thereby sensitizing the cells for use in any of the cell-based assays described herein.

Due in part to the increased appearance of antibiotic resistance in pathogenic microorganisms and to the significant side-effects associated with some currently used antibiotics, novel antibiotics acting at new targets are highly sought after in the art. Yet, another limitation in the current art related to cell-based assays is the problem of repeatedly identifying hits against the same kinds of target molecules in the same limited set of biological pathways. This may occur when compounds acting at such new targets are discarded, ignored or fail to be detected because compounds acting at the “old” targets are encountered more frequently and are more potent than compounds acting at the new targets. As a result, the majority of antibiotics in use currently interact with a relatively small number of target molecules within an even more limited set of biological pathways.

The use of sensitized cells of the current invention provides a solution to the above problem in two ways. First, desired compounds acting at a target of interest, whether a new target or a previously known but poorly exploited target, can now be detected above the “noise” of compounds acting at the “old” targets due to the specific and substantial increase in potency of such desired compounds when tested on the sensitized cells of the current invention. Second, the methods used to sensitize cells to compounds acting at a target of interest may also sensitize these cells to compounds acting at other target molecules within the same biological pathway. For example, expression of an antisense molecule to a an operon encoding one or more ribosomal proteins is expected to sensitize the cell to compounds acting on those ribosomal proteins and may also sensitize the cells to compounds acting at any of the ribosomal components (proteins or rRNA) or even to compounds acting at any target which is part of the protein synthesis pathway. Thus an important advantage of the present invention is the ability to reveal new targets and pathways that were previously not readily accessible to drug discovery methods.

Sensitized cells of the present invention are prepared by reducing the activity or level of a proliferation-required target that is encoded by a gene that is contained within a proliferation-required operon. The target molecule may be a gene product, such as an RNA or polypeptide produced from a proliferation-required gene, including the genes of SEQ ID NOs: 201-550, or a gene in a proliferation-required operon predicted by the methods described herein from Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species. An antisense complementary to at least a portion of a proliferation-required operon which encodes the target molecule can be used to inhibit the proliferation thereby sensitizing Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species. Biological pathways which can be sensitized by antisense-based operon inhibition include, but are not limited to, enzymatic, biochemical and metabolic pathways as well as pathways involved in the production of cellular structures such as the cell wall.

Current methods employed in the arts of medicinal and combinatorial chemistries are able to make use of structure-activity relationship information derived from testing compounds in various biological assays including direct binding assays and cell-based assays. Occasionally compounds are directly identified in such assays that are sufficiently potent to be developed as drugs. More often, initial hit compounds exhibit moderate or low potency. Once a hit compound is identified with low or moderate potency, directed libraries of compounds are synthesized and tested in order to identify more potent leads. Generally these directed libraries are combinatorial chemical libraries consisting of compounds with structures related to the hit compound but containing systematic variations including additions, subtractions and substitutions of various structural features. When tested for activity against the target molecule, structural features are identified that either alone or in combination with other features enhance or reduce activity. This information is used to design subsequent directed libraries containing compounds with enhanced activity against the target molecule. After one or several iterations of this process, compounds with substantially increased activity against the target molecule are identified and may be further developed as drugs. This process is facilitated by use of the sensitized cells of the present invention since compounds acting at the selected targets exhibit increased potency in such cell-based assays, thus; more compounds can now be characterized providing more useful information than would be obtained otherwise.

Thus, it is now possible using cell-based assays of the present invention to identify or characterize compounds that previously would not have been readily identified or characterized including compounds that act at targets that previously were not readily exploited using cell-based assays. The process of evolving potent drug leads from initial hit compounds is also substantially improved by the cell-based assays of the present invention because, for the same number of test compounds, more structure-function relationship information is likely to be revealed.

The method of sensitizing a cell entails selecting a suitable operon. A suitable operon is one whose transcription and/or expression is required for the proliferation of the cell that is to be sensitized. The next step is to introduce into the cells to be sensitized, an antisense RNA capable of hybridizing to at least a portion of the suitable operon or to the RNA encoded by the suitable operon. Introduction of the antisense RNA can be in the form of a vector in which antisense RNA is produced under the control of an inducible promoter. The amount of antisense RNA produced is modulated by varying an inducer concentration to which the cell is exposed and thereby varying the activity of the promoter driving transcription of the antisense RNA. Thus, cells are sensitized by exposing them to an inducer concentration that results in a sub-lethal level of antisense RNA expression. The requisite amount of inducer may be derived empirically by one of skill in the art.

In one embodiment of the cell-based assays, an antisense nucleic acid that is complementary to at least a portion of a proliferation-required operon comprising a nucleotide sequence selected from the group consisting of SEQ ID NOs: 1-194 can be used to sensitize Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species for use in cell-based assays.

In another embodiment of the cell-based assays, an antisense nucleic acid complementary to at least a portion of an operon that contains a proliferation-required gene that is homologous to a gene having a nucleotide sequence selected from the group consisting of SEQ ID NOs: 201-550 or complementary to at least a portion of an operon that contains a gene encoding a proliferation-required gene product that is homologous to a polypeptide having an amino acid sequence selected from the group consisting of SEQ ID NOs: 551-824 can be used can be used to sensitize Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species for use in cell-based assays.

In another embodiment of the cell-based assays, antisense nucleic acids complementary to identified proliferation-required operons, or portions thereof, in Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species can be used to sensitize Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species for use in cell-based assays.

Vectors producing antisense RNA complementary to identified operons required for proliferation, or portions thereof, are used to limit the concentration of a proliferation-required protein without severely inhibiting growth. The proliferation-required protein may be one of the proteins of SEQ ID NOs.: 551-824 or a homologous polypeptide. To achieve that goal, a growth inhibition dose curve of inducer is calculated by plotting various doses of inducer against the corresponding growth inhibition caused by the antisense expression. From this S curve, the concentration of inducer needed to achieve various percentages of antisense induced growth inhibition, from 1 to 100% can be determined.

A variety of different regulatable promoters may be used to produce the proliferation-inhibiting antisense nucleic acid. Transcription from the regulatable promoters may be modulated by controlling the activity of a transcription factor repressor which acts at the regulatable promoter. For example, if transcription is modulated by affecting the activity of a repressor, the choice of inducer to be used depends on the repressor/operator responsible for regulating transcription of the antisense nucleic acid. If the regulatable promoter comprises a T5 promoter fused to a xylO (xylose operator; e.g. derived from Staphylococcus xylosis (Schnappinger, D. et al., FEMS Microbiol. Let. 129: 126214-423978 (1995), the disclosure of which is incorporated herein by reference in its entirety) then transcription of the antisense nucleic acid may be regulated by a xylose repressor. The xylose repressor may be provided by ectoptic expression within an S. aureus cell of an exogenous xylose repressor gene, e.g. derived from S. xylosis DNA. In such cases transcription of antisense RNA from the promoter is inducible by adding xylose to the medium and the promoter is thus “xylose inducible.” Similarly, IPTG inducible promoters may be used. For example, the highest concentration of the inducer that does not reduce the growth rate significantly can be estimated from the curve. Cellular proliferation can be monitored by growth medium turbidity via OD measurements. In another example, the concentration of inducer that reduces growth by 25% can be predicted from the curve. In still another example, a concentration of inducer that reduces growth by 50% can be calculated. Additional parameters such as colony forming units (cfu) can be used to measure cellular viability. Some embodiments of the present invention contemplate the use of a vector that comprises a regulatable fusion promoter selected from a suite of fusion promoters wherein the promoter suite is useful for modulating both the basal and maximal levels of transcription of a nucleic acid over a wide dynamic range thus allowing the desired level of production of a transcript which corresponds to a nucleic acid described herein. Such promoters are described in U.S. patent application Ser. No. 10/032,393, entitled BACTERIAL PROMOTERS AND METHODS OF USE, filed Dec. 21, 2001, the disclosure of which is incorporated herein by reference in its entirety.

In some embodiments, the methods for the production of stabilized RNA in Gram-negative organisms, as described in U.S. patent application Ser. No. 10/327592, entitled STABILIZED NUCLEIC ACIDS IN GENE AND DRUG DISCOVERY AND METHODS OF USE, filed Dec. 20, 2002, the disclosure of which is incorporated herein by reference in its entirety, are used for the production of proliferation-inhibiting transcripts corresponding to the proliferation-required operon. Briefly, the stabilized antisense RNA may comprise an antisense RNA which was identified as inhibiting proliferation as described above which has been engineered to contain at least one stem loop flanking each end of the antisense nucleic acid. In some embodiments, the at least one stem-loop structure formed at the 5′ end of the stabilized antisense nucleic acid comprises a flush, double stranded 5′ end. In some embodiments, one or more of the stem loops comprises a rho independent terminator. In additional embodiments, the stabilized antisense RNA lacks a ribosome binding site. In further embodiments, the stabilized RNA lacks sites which are cleaved by one or more RNAses, such as RNAse E or RNAse III. In some embodiments, the stabilized antisense RNA may be transcribed in a cell which the activity of at least one enzyme involved in RNA degradation has been reduced. For example, the activity of an enzyme such as RNase E, RNase II, RNase III, polynucleotide phosphorylase, and poly(A) polymerase, RNA helicase, enolase or an enzyme having similar functions may be reduced in the cell.

In some embodiments of the present invention, promoter replacement methods, such as those describe above and in U.S. patent application Ser. No. 09/948,993, entitled RAPID METHOD FOR REGULATING GENE EXPRESSION, filed Sep. 6, 2001, the disclosure of which is incorporated herein by reference in its entirety, are used to regulate expression of the proliferation-required operon or of proliferation-required genes contained within the operon.

Cells to be assayed are exposed to the above-determined concentrations of inducer. The presence of the inducer at this sub-lethal concentration reduces the amount of the proliferation required gene product to a sub-optimal amount in the cell that will still support growth. Cells grown in the presence of this concentration of inducer are therefore specifically more sensitive to inhibitors of the proliferation-required protein or RNA of interest or to inhibitors of proteins or RNAs in the same biological pathway as the proliferation-required protein or RNA of interest but not to inhibitors of unrelated proteins or RNAs.

Cells pretreated with sub-inhibitory concentrations of inducer and thus containing a reduced amount of proliferation-required target gene product are then used to screen for compounds that reduce cell growth. The sub-lethal concentration of inducer may be any concentration consistent with the intended use of the assay to identify candidate compounds to which the cells are more sensitive. For example, the sub-lethal concentration of the inducer may be such that growth inhibition is at least about 5%, at least about 8%, at least about 10%, at least about 20%, at least about 30%, at least about 40%, at least about 50%, at least about 60% at least about 75%, or more. Cells which are pre-sensitized using the preceding method are more sensitive to inhibitors of the target protein because these cells contain less target protein to inhibit than do wild-type cells.

When screening for antimicrobial agents against a gene product required for proliferation, growth inhibition of cells containing a limiting amount of that proliferation-required gene product can be assayed. Growth inhibition can be measured by directly comparing the amount of growth, measured by the optical density of the growth medium, between an experimental sample and a control sample. Alternative methods for assaying cell proliferation include measuring the signal from a reporter construct, various enzymatic activity assays, and other methods well known in the art.

It will be appreciated that the above method may be performed in solid phase, liquid phase or a combination of the two. For example, cells grown on nutrient agar containing the inducer of the antisense construct may be exposed to compounds spotted onto the agar surface. If desired, the cells may be grown on agar containing varying concentrations of the inducer. A compound's effect may be judged from the diameter of the resulting killing zone, the area around the compound application point in which cells do not grow. Multiple compounds may be transferred to agar plates and simultaneously tested using automated and semi-automated equipment including but not restricted to multi-channel pipettes (for example the Beckman Multimek) and multi-channel spotters (for example the Genomic Solutions Flexys). In this way multiple plates and thousands to millions of compounds may be tested per day.

The compounds may also be tested entirely in liquid phase using microtiter plates as described below. Liquid phase screening may be performed in microtiter plates containing 96, 384, 1536 or more wells per microtiter plate to screen multiple plates and thousands to millions of compounds per day. Automated and semi-automated equipment may be used for addition of reagents (for example cells and compounds) and determination of cell density.

### Example 14

### Cell-Based Assay Using Antisense Complementary to Genes Encoding Ribosomal Proteins

The effectiveness of the above cell-based assay was validated using constructs transcribing antisense RNA to the proliferation required Escherichia coli genes rplL, rplJ, and rplW encoding ribosomal proteins L7/L12, L10 and L23 respectively. These proteins are essential components of the protein synthesis apparatus of the cell and as such are required for proliferation. These constructs were used to test the effect of antisense transcription on cell sensitivity to antibiotics known to bind to the ribosome and thereby inhibit protein synthesis. Constructs transcribing antisense RNA to several other genes (elaD, visC, yohH, and atpE/B), the products of which are not involved in protein synthesis were used for comparison.

First, pLex5BA (Krause et al., J. Mol. Biol. 274: 365 (1997), the disclosure of which is incorporated herein by reference in its entirety) vectors containing antisense constructs to either rplW or to elaD were introduced into separate E. coli cell populations. Vector introduction is a technique well known to those of ordinary skill in the art. The vectors of this example contain IPTG inducible promoters that drive the transcription of the antisense RNA in the presence of the inducer. However, those skilled in the art will appreciate that other inducible promoters may also be used. Suitable vectors are also well known in the art. For example, a number of promoters useful for nucleic acid transcription (including the nucleic acids described herein) in Enterococcus faecalis, Staphylococcus aureus as well as other Gram positive organisms are described in U.S. patent application Ser. No. 10/032,393, filed Dec. 21, 2001, the disclosure of which is incorporated herein by reference in its entirety. Antisense clones to genes encoding different ribosomal proteins or to genes encoding proteins that are not involved in protein synthesis were utilized to test the effect of antisense transcription on cell sensitivity to the antibiotics known to bind to ribosomal proteins and inhibit protein synthesis. Antisense nucleic acids comprising a nucleotide sequence complementary to the elaD, atpB&atpE, visC and yohH genes are referred to as AS-elaD, AS-atpB/E, AS-visC, AS-yohH respectively. These genes are not known to be involved in protein synthesis. Antisense nucleic acids to the rplL, rplL&rplJ and rplW genes are referred to as AS-rplL, AS-rpIL/J, and AS-rplW respectively. These genes encode ribosomal proteins L7/L12 (rplL) L10(rplJ) and L23 (rplW). Vectors containing these antisense nucleic acids were introduced into separate E. coli cell populations.

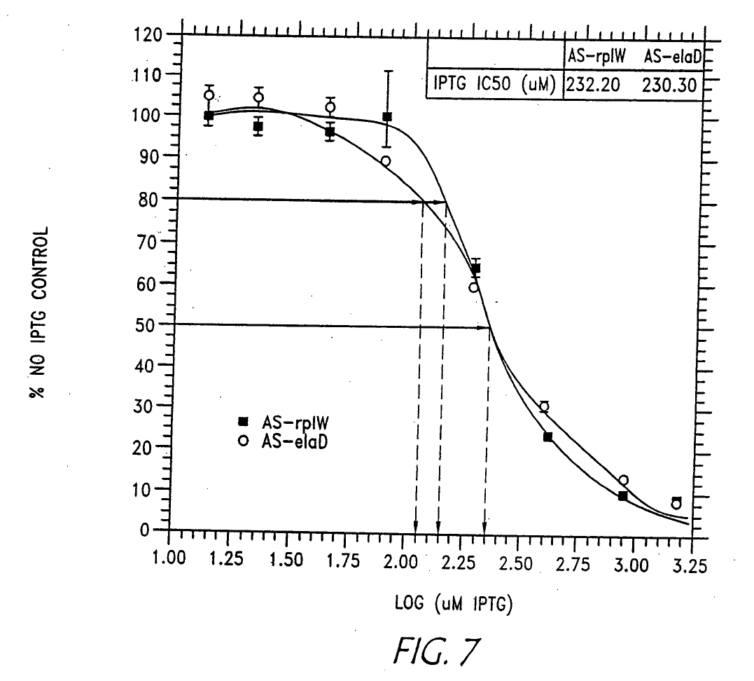

The cell populations containing vectors producing AS-elaD or AS-rplW were exposed to a range of IPTG concentrations in liquid medium to obtain the growth inhibitory dose curve for each clone (FIG. 7). First, seed cultures were grown to a particular turbidity measured by the optical density (OD) of the growth solution. The OD of the solution is directly related to the number of bacterial cells contained therein. Subsequently, sixteen 200 μl liquid medium cultures were grown in a 96 well microtiter plate at 37° C. with a range of IPTG concentrations in duplicate two-fold serial dilutions from 1600 μM to 12.5 μM (final concentration). Additionally, control cells were grown in duplicate without IPTG. These cultures were started from an inoculum of equal amounts of cells derived from the same initial seed culture of a clone of interest. The cells were grown for up to 15 hours and the extent of growth was determined by measuring the optical density of the cultures at 600 nm. When the control culture reached mid-log phase the percent growth (relative to the control culture) for each of the IPTG containing cultures was plotted against the log concentrations of IPTG to produce a growth inhibitory dose response curve for the IPTG. The concentration of IPTG that inhibits cell growth to 50% (IC₅₀) as compared to the 0 mM IPTG control (0% growth inhibition) was then calculated from the curve. Under these conditions, an amount of antisense RNA was produced that reduced the expression levels of rplW or elaD to a degree such that growth of cells containing their respective antisense vectors was inhibited by 50%.

Alternative methods of measuring growth are also contemplated. Examples of these methods include measurements of proteins, the expression of which is engineered into the cells being tested and can readily be measured. Examples of such proteins include luciferase and various enzymes.

Cells were pretreated with the selected concentration of IPTG and then used to test the sensitivity of cell populations to tetracycline, erythromycin and other known protein synthesis inhibitors. FIG. 7 is an IPTG dose response curve in E. coli transformed with an IPTG-inducible plasmid containing either an antisense clone to the E. coli rplW gene (AS-rplW) which encodes ribosomal protein L23 which is required for protein synthesis and essential for cell proliferation, or an antisense clone to the elaD (AS-elaD) gene which is not known to be involved in protein synthesis.

An example of a tetracycline dose response curve is shown in FIGS. 8A and 13B for the rplW and elaD genes, respectively. Cells were grown to log phase and then diluted into medium alone or medium containing IPTG at concentrations which give 20% and 50% growth inhibition as determined by IPTG dose response curves. After 2.5 hours, the cells were diluted to a final OD₆₀₀ of 0.002 into 96 well plates containing (1) +/− IPTG at the same concentrations used for the 2.5 hour pre-incubation; and (2) serial two-fold dilutions of tetracycline such that the final concentrations of tetracycline range from 1 μg/ml to 15.6 ng/ml and 0 μg/ml. The 96 well plates were incubated at 37° C. and the OD₆₀₀ was read by a plate reader every 5 minutes for up to 15 hours. For each IPTG concentration and the no IPTG control, tetracycline dose response curves were determined when the control (absence of tetracycline) reached 0.1 OD₆₀₀.

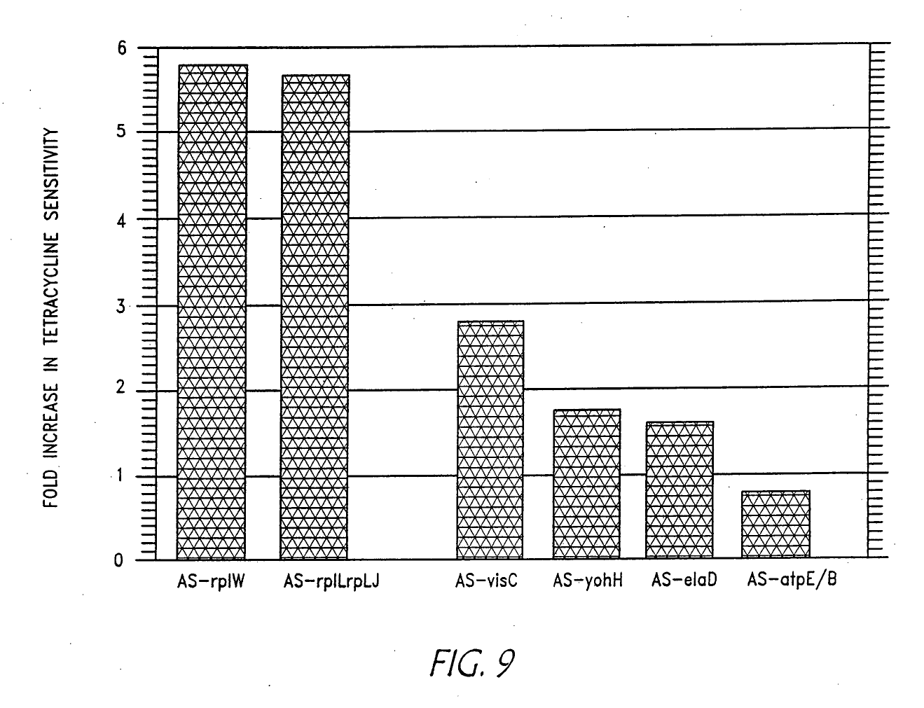

To compare tetracycline sensitivity with and without IPTG, tetracycline IC₅₀ₛ were determined from the dose response curves (FIGS. 8A-B). Cells transcribing antisense nucleic acids AS-rplL or AS-rplW to genes encoding ribosomal proteins L7/L12 and L23 respectively showed increased sensitivity to tetracycline (FIG. 8A) as compared to cells with reduced levels of the elaD gene product (AS-elaD) (FIG. 8B). FIG. 9 shows a summary bar chart in which the ratios of tetracycline IC₅₀ₛ, determined in the presence of IPTG which gives 50% growth inhibition versus tetracycline IC₅₀ₛ determined without IPTG (fold increase in tetracycline sensitivity) were plotted. Cells with reduced levels of either L7/L12 (encoded by genes rplL, rplJ) or L23 (encoded by the rplW gene) showed increased sensitivity to tetracycline (FIG. 9). Cells expressing antisense to genes not known to be involved in protein synthesis (AS-atpB/E, AS-visC, AS-elaD, AS-yohH) did not show the same increased sensitivity to tetracycline, validating the specificity of this assay (FIG. 9).

In addition to the above, it has been observed in initial experiments that clones transcribing antisense RNA to genes involved in protein synthesis (including genes encoding ribosomal proteins L7/L12 & L10, L7/L12 alone, L22, and L18, as well as genes encoding rRNA and Elongation Factor G) have increased sensitivity to the macrolide, erythromycin, whereas clones transcribing antisense to the non-protein synthesis genes elaD, atpB/E and visC do not. Furthermore, the clone transcribing antisense to rplL and rplJ (AS-rplL/J) does not show increased sensitivity to nalidixic acid and ofloxacin, antibiotics which do not inhibit protein synthesis.

The results with the ribosomal protein genes rplL, rplJ, and rplW as well as the initial results using various other antisense clones and antibiotics show that limiting the concentration of an antibiotic target makes cells more sensitive to the antimicrobial agents that specifically interact with that protein. The results also show that these cells are sensitized to antimicrobial agents that inhibit the overall function in which the protein target is involved but are not sensitized to antimicrobial agents that inhibit other functions.

It will be appreciated that the cell-based assays described above may be implemented by providing an antisense nucleic acid complementary to at least a portion of an operon comprising a nucleotide sequence selected from the group consisting of SEQ ID NOs: 1-194, wherein the portion does not include a nucleotide sequence selected from the group consisting of SEQ ID NOs: 201-550 to Staphylococcus aureus or an organism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

It will also be appreciated that the cell-based assays described above may be implemented by providing an antisense nucleic acid complementary to at least a portion of a proliferation-required operon comprising a gene that is homologous to a proliferation-required gene having a nucleotide sequence selected from the group consisting of SEQ ID NOs: 201-550 or by providing an antisense nucleic acid complementary to at least a portion of a proliferation-required operon comprising a gene encoding a polypeptide that is homologous to a proliferation-required polypeptide having an amino acid sequence selected from the group consisting of SEQ ID NOs: 551-824 to an organism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

It will also be appreciated that the cell-based assays described above may be implemented by providing an antisense nucleic acid complementary to at least a portion of a proliferation-required operon, which has been identified by the methods described herein, to an organism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

In some embodiments of the present invention, the methods for the production of stabilized RNA, as described in U.S. patent application Ser. No. 10/327592, entitled STABILIZED NUCLEIC ACIDS IN GENE AND DRUG DISCOVERY AND METHODS OF USE, filed Dec. 20, 2002, the disclosure of which is incorporated herein by reference in its entirety, can be used in the above cell-based assays in Gram-negative organisms to extend the lifetime of transcripts corresponding to the nucleic acids described herein.

The cell-based assay described above may also be used to identify the biological pathway in which a proliferation-required nucleic acid or its gene product lies. In such methods, cells transcribing a sub-lethal level of antisense complementary to at least a portion of a proliferation-required operon which contains a target proliferation-required nucleic acid, and control cells in which transcription of the antisense has not been induced are contacted with a panel of antibiotics known to act in various pathways. If the antibiotic acts in the pathway in which the target proliferation-required nucleic acid or its gene product lies, cells in which transcription of the antisense has been induced will be more sensitive to the antibiotic than cells in which expression of the antisense has not been induced.

As a control, the results of the assay may be confirmed by contacting a panel of cells transcribing antisense nucleic acids to many different operons containing proliferation-required genes other than the target proliferation-required gene. If the antibiotic is acting specifically, heightened sensitivity to the antibiotic will be observed only in the cells transcribing antisense to an operon containing the target proliferation-required gene but will not be observed generally in all cells expressing antisense to proliferation-required genes.

Similarly, the above method may be used to determine the pathway on which a test compound, such as a test antibiotic acts. A panel of cells, each of which transcribes an antisense to at least a portion of a proliferation-required operon, which contains a proliferation-required nucleic acid in a known pathway, is contacted with a compound for which it is desired to determine the pathway on which it acts. The sensitivity of the panel of cells to the test compound is determined in cells in which transcription of the antisense has been induced and in control cells in which expression of the antisense has not been induced. If the test compound acts on the pathway on which an antisense nucleic acid acts, cells in which expression of the antisense has been induced will be more sensitive to the compound than cells in which expression of the antisense has not been induced. In addition, control cells in which expression of antisense to proliferation-required genes in other pathways has been induced will not exhibit heightened sensitivity to the compound. In this way, the pathway on which the test compound acts may be determined.

It will be appreciated that any of the cell-based assays described above may be implemented by providing an antisense nucleic acid complementary to at least a portion of an operon comprising a nucleotide sequence selected from the group consisting of SEQ ID NOs: 1-194, wherein the portion does not include a nucleotide sequence selected from the group consisting of SEQ ID NOs: 201-550 to Staphylococcus aureus or an organism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

It will also be appreciated that any of the cell-based assays described above may be implemented by providing an antisense nucleic acid complementary to at least a portion of a proliferation-required operon comprising a gene that is homologous to a proliferation-required gene having a nucleotide sequence selected from the group consisting of SEQ ID NOs: 201-550 or by providing an antisense nucleic acid complementary to at least a portion of a proliferation-required operon comprising a gene encoding a polypeptide that is homologous to a proliferation-required polypeptide having an amino acid sequence selected from the group consisting of SEQ ID NOs: 551-824 to an organism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

It will also be appreciated that any of the cell-based assays described above may be implemented by providing an antisense nucleic acid complementary to at least a portion of a proliferation-required operon, which has been identified by the methods described herein, to an organism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

In some embodiments of the present invention, the methods for the production of stabilized RNA, as described in U.S. patent application Ser. No. 10/327592, entitled STABILIZED NUCLEIC ACIDS IN GENE AND DRUG DISCOVERY AND METHODS OF USE, filed Dec. 20, 2002, the disclosure of which is incorporated herein by reference in its entirety, can be used in the above cell-based assays in Gram-negative organisms to extend the lifetime of transcripts corresponding to the nucleic acids described herein.

The Example below provides one method for performing assays to identify the pathway in which a proliferation-required gene lie or the pathway on which an antibiotic acts.

### Example 15

### Identification of the Pathway in which a Proliferation-Required Gene Lies or the Pathway on which an Antibiotic Acts

A. Preparation of Bacterial Stocks for Assay

To provide a consistent source of cells to screen, frozen stocks of host bacteria containing the desired antisense construct are prepared using standard microbiological techniques. For example, a single clone of the microorganism can be isolated by streaking out a sample of the original stock onto an agar plate containing nutrients for cell growth and an antibiotic for which the antisense construct contains a selectable marker which confers resistance. After overnight growth an isolated colony is picked from the plate with a sterile needle and transferred to an appropriate liquid growth medium containing the antibiotic required for maintenance of the plasmid. The cells are incubated at 30° C. to 37° C. with vigorous shaking for 4 to 6 hours to yield a culture in exponential growth. Sterile glycerol is added to 15% (volume to volume) and 100 μL to 500 μL aliquots are distributed into sterile cryotubes, snap frozen in liquid nitrogen, and stored at −80° C. for future assays.

B. Growth of Bacteria for Use in the Assay

A day prior to an assay, a stock vial is removed from the freezer, rapidly thawed (37° C. water bath) and a loop of culture is streaked out on an agar plate containing nutrients for cell growth and an antibiotic to which the selectable marker of the antisense construct confers resistance. After overnight growth at 37° C., ten randomly chosen, isolated colonies are transferred from the plate (sterile inoculum loop) to a sterile tube containing 5 mL of LB medium containing the antibiotic to which the antisense vector confers resistance. After vigorous mixing to form a homogeneous cell suspension, the optical density of the suspension is measured at 600 nm (OD₆₀₀) and if necessary an aliquot of the suspension is diluted into a second tube of 5 mL, sterile, LB medium plus antibiotic to achieve an OD₆₀₀≦0.02 absorbance units. The culture is then incubated at 37° C. for 1-2 hrs with shaking until the OD₆₀₀ reaches OD 0.2 -0.3. At this point the cells are ready to be used in the assay.

C. Selection of Media to be Used in Assay

Two-fold dilution series of the inducer are generated in culture media containing the appropriate antibiotic for maintenance of the antisense construct. Several media are tested side by side and three to four wells are used to evaluate the effects of the inducer at each concentration in each media. For example, LB broth, TBD broth and Muller-Hinton media may be tested with the inducer xylose at the following concentrations, 5 mM, 10 mM, 20 mM, 40 mM, 80 mM, 120 mM and 160 mM. Equal volumes of test media-inducer and cells are added to the wells of a 384 well microtiter plate and mixed. The cells are prepared as described above and diluted 1:100 in the appropriate media containing the test antibiotic immediately prior to addition to the microtiter plate wells. For a control, cells are also added to several wells of each media that do not contain inducer, for example 0 mM xylose. Cell growth is monitored continuously by incubation at 37° C. in a microtiter plate reader monitoring the OD₆₀₀ of the wells over an 18-hour period. The percent inhibition of growth produced by each concentration of inducer is calculated by comparing the rates of logarithmic growth against that exhibited by cells growing in medium without inducer. The medium yielding greatest sensitivity to inducer is selected for use in the assays described below.

D. Measurement of Test Antibiotic Sensitivity in the Absence of Antisense Construct Induction

Two-fold dilution series of antibiotics of known mechanism of action are generated in the culture medium selected for further assay development that has been supplemented with the antibiotic used to maintain the construct. A panel of test antibiotics known to act on different pathways is tested side by side with three to four wells being used to evaluate the effect of a test antibiotic on cell growth at each concentration. Equal volumes of test antibiotic and cells are added to the wells of a 384 well microtiter plate and mixed. Cells are prepared as described above using the medium selected for assay development supplemented with the antibiotic required to maintain the antisense construct and are diluted 1:100 in identical medium immediately prior to addition to the microtiter plate wells. For a control, cells are also added to several wells that lack antibiotic, but contain the solvent used to dissolve the antibiotics. Cell growth is monitored continuously by incubation at 37° C. in a microtiter plate reader monitoring the OD₆₀₀ of the wells over an 18-hour period. The percent inhibition of growth produced by each concentration of antibiotic is calculated by comparing the rates of logarithmic growth against that exhibited by cells growing in medium without antibiotic. A plot of percent inhibition against log[antibiotic concentration] allows extrapolation of an IC₅₀ value for each antibiotic.

E. Measurement of Test Antibiotic Sensitivity in the Presence of Antisense Construct Inducer

The culture medium selected for use in the assay is supplemented with inducer at concentrations shown to inhibit cell growth by 50% and 80% as described above, as well as the antibiotic used to maintain the construct. Two-fold dilution series of the panel of test antibiotics used above are generated in each of these media. Several antibiotics are tested side by side in each medium with three to four wells being used to evaluate the effects of an antibiotic on cell growth at each concentration. Equal volumes of test antibiotic and cells are added to the wells of a 384 well microtiter plate and mixed. Cells are prepared as described above using the medium selected for use in the assay supplemented with the antibiotic required to maintain the antisense construct. The cells are diluted 1:100 into two 50 mL aliquots of identical medium containing concentrations of inducer that have been shown to inhibit cell growth by 50% and 80% respectively and incubated at 37° C. with shaking for 2.5 hours. Immediately prior to addition to the microtiter plate wells, the cultures are adjusted to an appropriate OD₆₀₀ (typically 0.002) by dilution into warm (37° C.) sterile medium supplemented with identical concentrations of the inducer and antibiotic used to maintain the antisense construct. For a control, cells are also added to several wells that contain solvent used to dissolve test antibiotics but which contain no antibiotic. Cell growth is monitored continuously by incubation at 37° C. in a microtiter plate reader monitoring the OD₆₀₀ of the wells over an 18-hour period. The percent inhibition of growth produced by each concentration of antibiotic is calculated by comparing the rates of logarithmic growth against that exhibited by cells growing in medium without antibiotic. A plot of percent inhibition against log[antibiotic concentration] allows extrapolation of an IC₅₀ value for each antibiotic.

F. Determining the Specificity of the Test Antibiotics

A comparison of the IC₅₀s generated by antibiotics of known mechanism of action under antisense induced and non-induced conditions allows the pathway in which a proliferation-required nucleic acid lies to be identified. If cells, which express an antisense nucleic acid comprising a nucleotide sequence complementary to at least a portion of an operon comprising the proliferation-required gene, are selectively sensitive to an antibiotic acting via a particular pathway, then the gene contained on the operon against which the antisense acts is involved in the pathway on which the antibiotic acts.

G. Identification of Pathway in which a Test Antibiotic Acts

As discussed above, the cell-based assay may also be used to determine the pathway against which a test antibiotic acts. In such an analysis, the pathways against which each member of a panel of antisense nucleic acids acts are identified as described above. A panel of cells, each containing an inducible vector which transcribes an antisense nucleic acid comprising a nucleotide sequence complementary to at least a portion of an operon comprising a proliferation-required gene in a known proliferation-required pathway, is contacted with a test antibiotic for which it is desired to determine the pathway on which it acts under inducing and non-inducing conditions. If heightened sensitivity is observed in induced cells transcribing antisense complementary to at least a portion of an operon comprising a proliferation-required gene in a particular pathway but not in induced cells transcribing antisense nucleic acids comprising nucleotide sequences complementary to at least a portion of an operon comprising a gene involved in other pathways, then the test antibiotic acts against the pathway for which heightened sensitivity was observed.

One skilled in the art will appreciate that further optimization of the assay conditions, such as the concentration of inducer used to induce antisense transcription and/or the growth conditions used for the assay (for example incubation temperature and medium components) may further increase the selectivity and/or magnitude of the antibiotic sensitization exhibited.

It will be appreciated that the cell-based assays described above may be implemented by providing an antisense nucleic acid complementary to at least a portion of an operon comprising a nucleotide sequence selected from the group consisting of SEQ ID NOs: 1-194, wherein the portion does not include a nucleotide sequence selected from the group consisting of SEQ ID NOs: 201-550 to Staphylococcus aureus or an organism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

It will also be appreciated that the cell-based assays described above may be implemented by providing an antisense nucleic acid complementary to at least a portion of a proliferation-required operon comprising a gene that is homologous to a proliferation-required gene having a nucleotide sequence selected from the group consisting of SEQ ID NOs: 201-550 or by providing an antisense nucleic acid complementary to at least a portion of a proliferation-required operon comprising a gene encoding a polypeptide that is homologous to a proliferation-required polypeptide having an amino acid sequence selected from the group consisting of SEQ ID NOs: 551-824 to an organism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigellaflexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

It will also be appreciated that the cell-based assays described above may be implemented by providing an antisense nucleic acid complementary to at least a portion of a proliferation-required operon, which has been identified by the methods described herein, to an organism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigellaflexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

In some embodiments of the present invention, the methods for the production of stabilized RNA, as described in U.S. patent application Ser. No. 10/327592, entitled STABILIZED NUCLEIC ACIDS IN GENE AND DRUG DISCOVERY AND METHODS OF USE, filed Dec. 20, 2002, the disclosure of which is incorporated herein by reference in its entirety, can be used in the above cell-based assays in Gram-negative organisms to extend the lifetime of transcripts corresponding to the nucleic acids described herein.

The following example confirms the effectiveness of the methods described above.

### Example 16

### Identification of the Biological Pathway in which a Proliferation-Required Gene Lies

The effectiveness of the above assays was validated using proliferation-required genes from E. coli which were identified using procedures similar to those described above. Antibiotics of various chemical classes and modes of action were purchased from Sigma Chemicals (St. Louis, Mo.). Stock solutions were prepared by dissolving each antibiotic in an appropriate aqueous solution based on information provided by the manufacturer. The final working solution of each antibiotic contained no more than 0.2% (w/v) of any organic solvent. To determine their potency against a bacterial strain engineered for transcription of an antisense comprising a nucleotide sequence complementary to a proliferation-required 50S ribosomal protein, each antibiotic was serially diluted two- or three- fold in growth medium supplemented with the appropriate antibiotic for maintenance of the antisense construct. At least ten dilutions were prepared for each antibiotic. 25 μL aliquots of each dilution were transferred to discrete wells of a 384-well microplate (the assay plate) using a multi-channel pipette. Quadruplicate wells were used for each dilution of an antibiotic under each treatment condition (plus and minus inducer). Each assay plate contained twenty wells for cell growth controls (growth medium replacing antibiotic), ten wells for each treatment (plus and minus inducer, in this example IPTG). Assay plates were usually divided into the two treatments: half the plate containing induced cells and an appropriate concentrations of inducer (in this example IPTG) to maintain the state of induction, the other half containing non-induced cells in the absence of IPTG.

Cells for the assay were prepared as follows. Bacterial cells containing a construct, from which transcription of antisense nucleic acid comprising a nucleotide sequence complementary to rplL and rplJ (AS-rplL/J), which encode proliferation-required 50S ribosomal subunit proteins, is inducible in the presence of IPTG, were grown into exponential growth (OD₆₀₀ 0.2 to 0.3) and then diluted 1:100 into fresh medium containing either 400 μM or 0 μM inducer (IPTG). These cultures were incubated at 37° C. for 2.5 hr. After a 2.5 hr incubation, induced and non-induced cells were respectively diluted into an assay medium at a final OD₆₀₀ value of 0.0004. The medium contained an appropriate concentration of the antibiotic for the maintenance of the antisense construct. In addition, the medium used to dilute induced cells was supplemented with 800 μM IPTG so that addition to the assay plate would result in a final IPTG concentration of 400 μM. Induced and non-induced cell suspensions were dispensed (25 μl/well) into the appropriate wells of the assay plate as discussed previously. The plate was then loaded into a plate reader, incubated at constant temperature, and cell growth was monitored in each well by the measurement of light scattering at 595 nm. Growth was monitored every 5 minutes until the cell culture attained a stationary growth phase. For each concentration of antibiotic, a percentage inhibition of growth was calculated at the time point corresponding to mid-exponential growth for the associated control wells (no antibiotic, plus or minus IPTG). For each antibiotic and condition (plus or minus IPTG), a plot of percent inhibition versus log of antibiotic concentration was generated and the IC₅₀ determined. A comparison of the IC₅₀ for each antibiotic in the presence and absence of IPTG revealed whether induction of the antisense construct sensitized the cell to the mechanism of action exhibited by the antibiotic. Cells which exhibited a statistically significant decrease in the IC₅₀ value in the presence of inducer were considered to have an increased sensitivity to the test antibiotic.

The results are provided in Table VIII below, which lists the classes and names of the antibiotics used in the analysis, the targets of the antibiotics, the IC₅₀ in the absence of IPTG, the IC₅₀ in the presence of IPTG, the concentration units for the IC₅₀ₛ, the fold increase in IC₅₀ in the presence of IPTG, and whether increased sensitivity was observed in the presence of IPTG.

TABLE VIII
Effect of Expression of Antisense RNA to rplL and rplJ on Antibiotic Sensitivity

| | | | | | | |
|---|---|---|---|---|---|---|
| | | | | | Fold | |
| | | IC₅₀ | IC₅₀ | Conc. | Increase in | Sensitivity |
| ANTIBIOTIC CLASS/Names | TARGET | (−IPTG) | (+IPTG) | Unit | Sensitivity | Increased? |

PROTEIN SYNTHESIS INHIBITOR
AMINOGLYCOSIDES

| | | | | | | |
|---|---|---|---|---|---|---|
| Gentamicin | 30S ribosome function | 2715 | 19.19 | ng/ml | 141 | Yes |
| Streptomycin | 30S ribosome function | 11280 | 161 | ng/ml | 70 | Yes |
| Spectinomycin | 30S ribosome function | 18050 | <156 | ng/ml | | Yes |
| Tobramycin | 30S ribosome function | 3594 | 70.58 | ng/ml | 51 | Yes |

MACROLIDES

| | | | | | | |
|---|---|---|---|---|---|---|
| Erythromycin | 50S ribosome function | 7467 | 187 | ng/ml | 40 | Yes |

AROMATIC POYKETIDES

| | | | | | | |
|---|---|---|---|---|---|---|
| Tetracycline | 30S ribosome function | 199.7 | 1.83 | ng/ml | 109 | Yes |
| Minocycline | 30S ribosome function | 668.4 | 3.897 | ng/ml | 172 | Yes |
| Doxycycline | 30S ribosome function | 413.1 | 27.81 | ng/ml | 15 | Yes |

OTHER PROTEIN SYNTHESIS INHIBITORS

| | | | | | | |
|---|---|---|---|---|---|---|
| Fusidic acid | Elongation Factor G function | 59990 | 641 | ng/ml | 94 | Yes |
| Chloramphenicol | 30S ribosome function | 465.4 | 1.516 | ng/ml | 307 | Yes |
| Lincomycin | 50S ribosome function | 47150 | 324.2 | ng/ml | 145 | Yes |

OTHER ANTIBIOTIC MECHANISMS
B-LACTAMS

| | | | | | | |
|---|---|---|---|---|---|---|
| Cefoxitin | Cell wall biosynthesis | 2782 | 2484 | ng/ml | 1 | No |
| Cefotaxime | Cell wall biosynthesis | 24.3 | 24.16 | ng/ml | 1 | No |

DNA SYNTHESIS INHIBITORS

| | | | | | | |
|---|---|---|---|---|---|---|
| Nalidixic acid | DNA Gyrase activity | 6973 | 6025 | ng/ml | 1 | No |
| Ofloxacin | DNA Gyrase activity | 49.61 | 45.89 | ng/ml | 1 | No |

OTHER

| | | | | | | |
|---|---|---|---|---|---|---|
| Bacitracin | Cell membrane function | 4077 | 4677 | mg/ml | 1 | No |
| Trimethoprim | Dihydrofolate Reductase | 128.9 | 181.97 | ng/ml | 1 | No |
| | activity | | | | | |
| Vancomycin | Cell wall biosynthesis | 145400 | 72550 | ng/ml | 2 | No |

The above results demonstrate that induction of an antisense RNA complementary to genes encoding 50S ribosomal subunit proteins results in a selective and highly significant sensitization of cells to antibiotics that inhibit ribosomal function and protein synthesis. The above results further demonstrate that induction of an antisense to an essential gene sensitizes a cell or microorganism to compounds that interfere with that gene product's biological role. This sensitization is restricted to compounds that interfere with pathways associated with the targeted gene and its product.

It will be appreciated that the cell-based assays described above may be implemented by providing an antisense nucleic acid complementary to at least a portion of an operon comprising a nucleotide sequence selected from the group consisting of SEQ ID NOs: 1-194, wherein the portion does not include a nucleotide sequence selected from the group consisting of SEQ ID NOs: 201-550 to Staphylococcus aureus or an organism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsi, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigellaflexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

It will also be appreciated that the cell-based assays described above may be implemented by providing an antisense nucleic acid complementary to at least a portion of a proliferation-required operon comprising a gene that is homologous to a proliferation-required gene having a nucleotide sequence selected from the group consisting of SEQ ID NOs: 201-550 or by providing an antisense nucleic acid complementary to at least a portion of a proliferation-required operon comprising a gene encoding a polypeptide that is homologous to a proliferation-required polypeptide having an amino acid sequence selected from the group consisting of SEQ ID NOs: 551-824 to an organism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettshi, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigellaflexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

It will also be appreciated that the cell-based assays described above may be implemented by providing an antisense nucleic acid complementary to at least a portion of a proliferation-required operon, which has been identified by the methods described herein, to an organism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigellaflexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

In some embodiments of the present invention, the methods for the production of stabilized RNA, as described in U.S. patent application Ser. No. 10/327592, entitled STABILIZED NUCLEIC ACIDS IN GENE AND DRUG DISCOVERY AND METHODS OF USE, filed Dec. 20, 2002, the disclosure of which is incorporated herein by reference in its entirety, can be used in the above cell-based assays in Gram-negative organisms to extend the lifetime of transcripts corresponding to the nucleic acids described herein.

The example below describes an analysis performed in Staphylococcus aureus.

### Example 17

### Identification of the Biological Pathway in which a Gene Required for Proliferation of Staphylococcus aureus Lies

Antibiotics of various chemical classes and modes of action were purchased from chemical suppliers, for example Sigma Chemicals (St. Louis, Mo). Stock solutions were prepared by dissolving each antibiotic in an appropriate aqueous solution based on information provided by the manufacturer. The final working solution of each antibiotic contained no more than 0.2% (w/v) of any organic solvent.

To determine its potency against a bacterial strain containing an antisense nucleic acid comprising a nucleotide sequence complementary to the nucleotide sequence encoding the Beta subunit of DNA gyrase (which is required for proliferation) under the control of a xylose inducible promoter, each antibiotic was serially diluted two- or three- fold in growth medium supplemented with the appropriate antibiotic for maintenance of the antisense construct. At least ten dilutions were prepared for each antibiotic.

Aliquots (25 μL) of each dilution were transferred to discrete wells of a 384-well microplate (the assay plate) using a multi-channel pipette. Quadruplicate wells were used for each dilution of an antibiotic under each treatment condition (plus and minus inducer). Each assay plate contained twenty wells for cell growth controls (growth medium, no antibiotic), ten wells for each treatment (plus and minus inducer, xylose, in this example). Half the assay plate contained induced cells (in this example Staphylococcus aureus cells) and appropriate concentrations of inducer (xylose, in this example) to maintain the state of induction while the other half of the assay plate contained non-induced cells maintained in the absence of inducer.

Preparation of Bacterial Cells

Cells of a bacterial clone containing a construct in which transcription of antisense comprising a nucleotide sequence complementary to the sequence encoding the Beta subunit of DNA gyrase under the control of the xylose inducible promoter (S1M10000001F08) were grown into exponential growth (OD₆₀₀ 0.2 to 0.3) and then diluted 1:100 into fresh medium containing either 12 mM or 0 mM inducer (xylose). These cultures were incubated at 37° C. for 2.5 hr. The presence of inducer (xylose) in the medium initiates and maintains production of antisense RNA from the antisense construct. After a 2.5 hr incubation, induced and non-induced cells were respectively diluted into an assay medium containing an appropriate concentration of the antibiotic for the maintenance of the antisense construct. In addition, medium used to dilute induced cells was supplemented with 24 mM xylose so that addition to the assay plate would result in a final xylose concentration of 12 mM. The cells were diluted to a final OD₆₀₀ value of 0.0004.

Induced and non-induced cell suspensions were dispensed (25 μl/well) into the appropriate wells of the assay plate as discussed previously. The plate was then loaded into a plate reader and incubated at constant temperature while cell growth was monitored in each well by the measurement of light scattering at 595 nm. Growth was monitored every 5 minutes until the cell culture attained a stationary growth phase. For each concentration of antibiotic, a percentage inhibition of growth was calculated at the time point corresponding to mid-exponential growth for the associated control wells (no antibiotic, plus or minus xylose). For each antibiotic and condition (plus or minus xylose), plots of percent inhibition versus Log of antibiotic concentration were generated and IC₅₀ₛ, determined.

A comparison of each antibiotic's IC₅₀ in the presence and absence of inducer (xylose, in this example) reveals whether induction of the antisense construct sensitized the cell to the antibiotic's mechanism of action. If the antibiotic acts against the β subunit of DNA gyrase, the IC₅₀ of induced cells will be significantly lower than the IC₅₀ of uninduced cells.

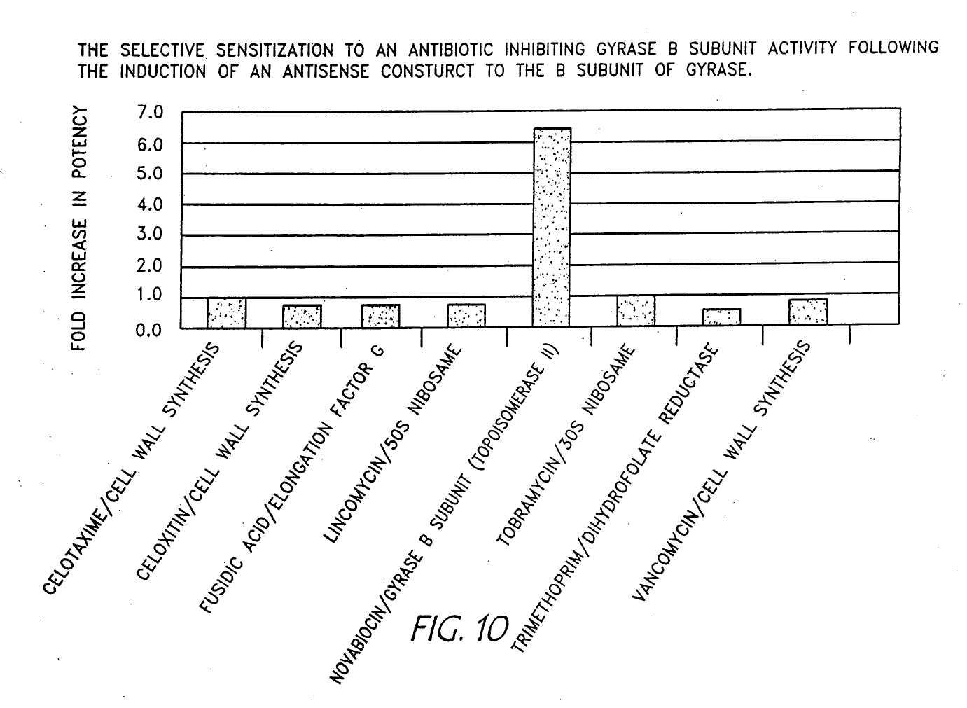

FIG. 10 lists the antibiotics tested, their targets, and their fold increase in potency between induced cells and uninduced cells. As illustrated in FIG. 10, the potency of cefotaxime, cefoxitin, fusidic acid, lincomycin, tobramycin, trimethoprim and vancomycin, each of which act on targets other than the β subunit of gyrase, was not significantly different in induced cells as compared to uninduced cells. However, the potency of novobiocin, which is known to act against the Beta subunit of DNA gyrase, was significantly different between induced cells and uninduced cells.

Thus, induction of an antisense nucleic acid comprising a nucleotide sequence complementary to the sequence encoding the, subunit of gyrase results in a selective and significant sensitization of Staphylococcus aureus cells to an antibiotic which inhibits the activity of this protein. Furthermore, the results demonstrate that induction of an antisense construct to an essential gene sensitizes a cell or microorganism to compounds that interfere with that gene product's biological role. This sensitization is apparently restricted to compounds that interfere with the targeted gene and its product.

It will be appreciated that the cell-based assays described above may be implemented by providing an antisense nucleic acid complementary to at least a portion of an operon comprising a nucleotide sequence selected from the group consisting of SEQ ID NOs: 1-194, wherein the portion does not include a nucleotide sequence selected from the group consisting of SEQ ID NOs: 201-550 to Staphylococcus aureus or an organism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigellaflexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

It will also be appreciated that the cell-based assays described above may be implemented by providing an antisense nucleic acid complementary to at least a portion of a proliferation-required operon comprising a gene that is homologous to a proliferation-required gene having a nucleotide sequence selected from the group consisting of SEQ ID NOs: 201-550 or by providing an antisense nucleic acid complementary to at least a portion of a proliferation-required operon comprising a gene encoding a polypeptide that is homologous to a proliferation-required polypeptide having an amino acid sequence selected from the group consisting of SEQ ID NOs: 551-824 to an organism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigellaflexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

It will also be appreciated that the cell-based assays described above may be implemented by providing an antisense nucleic acid complementary to at least a portion of a proliferation-required operon, which has been identified by the methods described herein, to an organism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

In some embodiments of the present invention, the methods for the production of stabilized RNA, as described in U.S. patent application Ser. No. 10/327592, entitled STABILIZED NUCLEIC ACIDS IN GENE AND DRUG DISCOVERY AND METHODS OF USE, filed Dec. 20, 2002, the disclosure of which is incorporated herein by reference in its entirety, can be used in the above cell-based assays in Gram-negative organisms to extend the lifetime of transcripts corresponding to the nucleic acids described herein.

Assays utilizing antisense constructs to operons comprising essential genes or portions thereof can be used to identify compounds that interfere with the activity of those gene products. Such assays could be used to identify drug leads, for example antibiotics.

Panels of cells transcribing different antisense nucleic acids can be used to characterize the point of intervention of a compound affecting an essential biochemical pathway including antibiotics with no known mechanism of action.

Assays utilizing antisense constructs to operons comprising essential genes can be used to identify compounds that specifically interfere with the activity of multiple targets in a pathway. Such constructs can be used to simultaneously screen a sample against multiple targets in one pathway in one reaction (Combinatorial HTS).

Furthermore, as discussed above, panels of antisense construct-containing cells may be used to characterize the point of intervention of any compound affecting an essential biological pathway including antibiotics with no known mechanism of action.

It will be appreciated that the cell-based assays described above may be implemented by providing an antisense nucleic acid complementary to at least a portion of an operon comprising a nucleotide sequence selected from the group consisting of SEQ ID NOs: 1-194, wherein the portion does not include a nucleotide sequence selected from the group consisting of SEQ ID NOs: 201-550 to Staphylococcus aureus or an organism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigellaflexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

It will also be appreciated that the cell-based assays described above may be implemented by providing an antisense nucleic acid complementary to at least a portion of a proliferation-required operon comprising a gene that is homologous to a proliferation-required gene having a nucleotide sequence selected from the group consisting of SEQ ID NOs: 201-550 or by providing an antisense nucleic acid complementary to at least a portion of a proliferation-required operon comprising a gene encoding a polypeptide that is homologous to a proliferation-required polypeptide having an amino acid sequence selected from the group consisting of SEQ ID NOs: 551-824 to an organism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigellaflexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

It will also be appreciated that the cell-based assays described above may be implemented by providing an antisense nucleic acid complementary to at least a portion of a proliferation-required operon, which has been identified by the methods described herein, to an organism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigellaflexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

In some embodiments of the present invention, the methods for the production of stabilized RNA, as described in U.S. patent application Ser. No. 10/327592, entitled STABILIZED NUCLEIC ACIDS IN GENE AND DRUG DISCOVERY AND METHODS OF USE, filed Dec. 20, 2002, the disclosure of which is incorporated herein by reference in its entirety, can be used in the above cell-based assays in Gram-negative organisms to extend the lifetime of transcripts corresponding to the nucleic acids described herein.

### Example 18

### Identification of Genes Required for Escherichia coli Proliferation, Operon Prediction, Inhibition of Proliferation and Cell-Based Assays

Genes required for proliferation in Escherichia coli are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 19

### Identification of Genes Required for Staphylococcus aureus Proliferation, Operon Prediction, Inhibition of Proliferation and Cell-Based Assays

Genes required for proliferation in Staphylococcus aureus are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 20

### Identification of Genes Required for Enterococcus faecalis Proliferation, Operon Prediction, Inhibition of Proliferation and Cell-Based Assays

Genes required for proliferation in Enterococcus faecalis are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 21

### Identification of Genes Required for Klebsiella pneumoniae Proliferation, Operon Prediction, Inhibition of Proliferation and Cell-Based Assays

Genes required for proliferation in Klebsiella pneumoniae are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 22

### Identification of Genes Required for Pseudomonas aeruginosa Proliferation, Operon Prediction, Inhibition of Proliferation and Cell-Based Assays

Genes required for proliferation in Pseudomonas aeruginosa are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 23

### Identification of Genes Required for Salmonella typhimurium Proliferation, Operon Prediction, Inhibition of Proliferation and Cell-Based Assays

Genes required for proliferation in Salmonella typhimurium are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 24

### Identification of Genes Required for Acinetobacter baumannii Proliferation, Operon Prediction, Inhibition of Proliferation and Cell-Based Assays

Genes required for proliferation in Acinetobacter baumannii are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 25

### Identification of Genes Required for Bacillus anthracis Proliferation, Operon Prediction, Inhibition of Proliferation and Cell-Based Assays

Genes required for proliferation in Bacillus anthracis are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 26

### Identification of Genes Required for Bordetella pertussis Proliferation, Operon Prediction, Inhibition of Proliferation and Cell-Based Assays

Genes required for proliferation in Bordetella pertussis are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 27

### Identification of Genes Required for Borrelia burgdorferi Proliferation, Operon Prediction, Inhibition of Proliferation and Cell-Based Assays

Genes required for proliferation in Borrelia burgdorferi are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 28

### Identification of Genes Required for Burkholderia cepacia Proliferation, Operon Prediction, Inhibition of Proliferation and Cell-Based Assays

Genes required for proliferation in Burkholderia cepacia are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 29

### Identification of Genes Required for Burkholderia fungorum Proliferation, Operon Prediction, Inhibition of Proliferation and Cell-Based Assays

Genes required for proliferation in Burkholderia fungorum are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 30

### Identification of Genes Required for Burkholderia mallei Proliferation, Operon Prediction, Inhibition of Proliferation and Cell-Based Assays

Genes required for proliferation in Burkholderia mallei are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 31

### Identification of Genes Required for Campylobacter jejuni Proliferation, Operon Prediction, Inhibition of Proliferation and Cell-Based Assays

Genes required for proliferation in Campylobacter jejuni are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 32

### Identification of Genes Required for Chlamydia pneumoniae Proliferation, Operon Prediction, Inhibition of Proliferation and Cell-Based Assays

Genes required for proliferation in Chlamydia pneumoniae are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 33

### Identification of Genes Required for Chlamydia trachomatis Proliferation, Operon Prediction, Inhibition of Proliferation and Cell-Based Assays

Genes required for proliferation in Chlamydia trachomatis are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 34

### Identification of Genes Required for Clostridium acetobutylicum Proliferation, Operon Prediction, Inhibition of Proliferation and Cell-Based Assays

Genes required for proliferation in Clostridium acetobutylicum are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 35

### Identification of Genes Required for Clostridium botulinum Proliferation, Operon Prediction, Inhibition of Proliferation and Cell-Based Assays

Genes required for proliferation in Clostridium botulinum are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 36

### Identification of Genes Required for Clostridium difficile Proliferation, Operon Prediction, Inhibition of Proliferation and Cell-Based Assays

Genes required for proliferation in Clostridium difficile are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 37

### Identification of Genes Required for Corynebacterium diptheriae Proliferation, Operon Prediction, Inhibition of Proliferation and Cell-Based Assays

Genes required for proliferation in Corynebacterium diptheriae are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 38

### Identification of Genes Required for Enterobacter cloacae Proliferation, Operon Prediction, Inhibition of Proliferation and Cell-Based Assays

Genes required for proliferation in Enterobacter cloacae are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 39

### Identification of Genes Required for Enterococcus faecium Proliferation, Operon Prediction, Inhibition of Proliferation and Cell-Based Assays

Genes required for proliferation in Enterococcus faecium are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 40

### Identification of Genes Required for Haemophilus influenzae Proliferation, Operon Prediction, Inhibition of Proliferation and Cell-Based Assays

Genes required for proliferation in Haemophilus influenzae are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 41

### Identification of Genes Required for Helicobacter pylori Proliferation, Operon Prediction, Inhibition of Proliferation and Cell-Based Assays

Genes required for proliferation in Helicobacter pylori are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 42

### Identification of Genes Required for Legionella pneumophila Proliferation, Operon Prediction, Inhibition of Proliferation and Cell-Based Assays

Genes required for proliferation in Legionella pneumophila are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 43

### Identification of Genes Required for Listeria monocytogenes Proliferation, Operon Prediction, Inhibition of Proliferation and Cell-Based Assays

Genes required for proliferation in Listeria monocytogenes are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 44

### Identification of Genes Required for Moraxella catarrhalis Proliferation, Operon Prediction, Inhibition of Proliferation and Cell-Based Assays

Genes required for proliferation in Moraxella catarrhalis are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 45

### Identification of Genes Required for Mycobacterium avium Proliferation, Operon Prediction, Inhibition of Proliferation and Cell-Based Assays

Genes required for proliferation in Mycobacterium avium are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 46

### Identification of Genes Required for Mycobacterium bovis Proliferation, Operon Prediction, Inhibition of Proliferation and Cell-Based Assays

Genes required for proliferation in Mycobacterium bovis are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 47

### Identification of Genes Required for Mycobacterium leprae Proliferation, Operon Prediction, Inhibition of Proliferation and Cell-Based Assays

Genes required for proliferation in Mycobacterium leprae are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 48

### Identification of Genes Required for Mycobacterium tuberculosis Proliferation, Operon Prediction, Inhibition of Proliferation and Cell-Based Assays

Genes required for proliferation in Mycobacterium tuberculosis are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 49

### Identification of Genes Required for Mycoplasma genitalium Proliferation, Operon Prediction, Inhibition of Proliferation and Cell-Based Assays

Genes required for proliferation in Mycoplasma genitalium are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 50

### Identification of Genes Required for Mycoplasma pneumoniae Proliferation, Operon Prediction, Inhibition of Proliferation and Cell-Based Assays

Genes required for proliferation in Mycoplasma pneumoniae are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 51

### Identification of Genes Required for Neisseria gonorrhoeae Proliferation, Operon Prediction, Inhibition of Proliferation and Cell-Based Assays

Genes required for proliferation in Neisseria gonorrhoeae are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 52

### Identification of Genes Required for Neisseria meningitidis Proliferation, Operon Prediction, Inhibition of Proliferation and Cell-Based Assays

Genes required for proliferation in Neisseria meningitidis are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 53

### Identification of Genes Required for Pasteurella multocida Proliferation, Operon Prediction, Inhibition of Proliferation and Cell-Based Assays

Genes required for proliferation in Pasteurella multocida are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 54

### Identification of Genes Required for Proteus mirabilis Proliferation, Operon Prediction, Inhibition of Proliferation and Cell-Based Assays

Genes required for proliferation in Proteus mirabilis are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 55

### Identification of Genes Required for Pseudomonas putida Proliferation, Operon Prediction, Inhibition of Proliferation and Cell-Based Assays

Genes required for proliferation in Pseudomonas putida are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 56

### Identification of Genes Required for Pseudomonas syringae Proliferation, Operon Prediction, Inhibition of Proliferation and Cell-Based Assays

Genes required for proliferation in Pseudomonas syringae are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 57

### Identification of Genes Required for Salmonella paratyphi Proliferation, Operon Prediction, Inhibition of Proliferation and Cell-Based Assays

Genes required for proliferation in Salmonella paratyphi are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 58

### Identification of Genes Required for Salmonella typhi Proliferation, Operon Prediction, Inhibition of Proliferation and Cell-Based Assays

Genes required for proliferation in Salmonella typhi are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 59

### Identification of Genes Required for Staphylococcus epidermidis Proliferation, Operon Prediction, Inhibition of Proliferation and Cell-Based Assays

Genes required for proliferation in Staphylococcus epidermidis are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 60

### Identification of Genes Required for Staphylococcus haemolyticus Proliferation, Operon Prediction, Inhibition of Proliferation and Cell-Based Assays

Genes required for proliferation in Staphylococcus haemolyticus are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 61

### Identification of Genes Required for Streptococcus mutans Proliferation, Operon Prediction, Inhibition of Proliferation and Cell-Based Assays

Genes required for proliferation in Streptococcus mutans are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 62

### Identification of Genes Required for Streptococcus pneumoniae Proliferation, Operon Prediction, Inhibition of Proliferation and Cell-Based Assays

Genes required for proliferation in Streptococcus pneumoniae are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 63

### Identification of Genes Required for Streptococcus pyogenes Proliferation, Operon Prediction, Inhibition of Proliferation and Cell-Based Assays

Genes required for proliferation in Streptococcus pyogenes are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 64

### Identification of Genes Required for Treponema pallidum Proliferation, Operon Prediction, Inhibition of Proliferation and Cell-Based Assays

Genes required for proliferation in Treponema pallidum are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 65

### Identification of Genes Required for Ureaplasma urealyticum Proliferation, Operon Prediction, Inhibition of Proliferation and Cell-Based Assays

Genes required for proliferation in Ureaplasma urealyticum are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 66

### Identification of Genes Required for Vibrio cholerae Proliferation, Operon Prediction, Inhibition of Proliferation and Cell-Based Assays

Genes required for proliferation in Vibrio cholerae are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 67

### Identification of Genes Required for Yersinia pestis Proliferation, Operon Prediction, Inhibition of Proliferation and Cell-Based Assays

Genes required for proliferation in Yersinia pestis are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 68

### Identification of Genes Required for Salmonella enterica Proliferation, Operon Prediction, Inhibition of Proliferation and Cell-Based Assays

Genes required for proliferation in Salmonella enterica are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 69

### Identification of Genes Required for Salmonella cholerasuis Proliferation, Operon Prediction, Inhibition of Proliferation and Cell-Based Assays

Genes required for proliferation in Salmonella cholerasuis are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 70

### Identification of Genes Required for Brucella abortus Proliferation, Operon Prediction, Inhibition of Proliferation and Cell-Based Assays

Genes required for proliferation in Brucella abortus are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 71

### Identification of Genes Required for Brucella melitensis Proliferation, Operon Prediction, Inhibition of Proliferation and Cell-Based Assays

Genes required for proliferation in Brucella melitensis are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 72

### Identification of Genes Required for Coxiella burnetii Proliferation, Operon Prediction, Inhibition of Proliferation and Cell-Based Assays

Genes required for proliferation in Coxiella burnetii are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 73

### Identification of Genes Required for Francisella tularensis Proliferation, Operon Prediction, Inhibition of Proliferation and Cell-Based Assays

Genes required for proliferation in Francisella tularensis are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 74

### Identification of Genes Required for Rickettsia rickettsii Proliferation, Operon Prediction, Inhibition of Proliferation and Cell-Based Assays

Genes required for proliferation in Rickettsia rickettsii are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

### Example 75

### Identification of Genes Required for Rochalimaea quintana Proliferation, Operon Prediction, Inhibition of Proliferation and Cell-Based Assays

Genes required for proliferation in Rochalimaea quintana are identified according to the methods described above. For example, promoters and vectors described herein can be used to identify proliferation required genes. Operons which comprise one or more of the above-identified, proliferation-required genes are predicted using the operon prediction methods described herein. An antisense nucleic acid that is complementary to at least a portion of a predicted proliferation-required operon is provided to the cell thereby inhibiting proliferation. Because the antisense nucleic acid can be produced at levels that range from lethal to sublethal, inhibition of proliferation can be either complete or partial. If proliferation of the cell is only partially inhibited, the cell becomes sensitized. Sensitized cells are contacted with compounds present in a library of compounds. A further reduction in the proliferation of the sensitized cell after being contacted with the compound indicates that the compound possesses the ability to inhibit cellular proliferation.

Use of Antisense Nucleic Acids as Therapeutics

In addition to using antisense nucleic acids that are complementary to at least a portion of a proliferation-required operon to enable screening of molecule libraries to identify compounds useful to identify antibiotics, these antisense nucleic acids can be used as therapeutic agents. Specifically, the antisense nucleic acids complementary to at least a portion of a proliferation-required operon that has been identified by using the operon prediction methods described herein, including antisense nucleic acids complementary to at least a portion of a proliferation-required operon comprising a nucleotide sequence selected from the group consisting of SEQ ID NOs: 1-194, can be provided to an individual to inhibit the translation of one or more bacterial target genes.

### Example 76

### Generation of Antisense Therapeutics from Predicted Operons

Antisense nucleic acids complementary to at least a portion of a proliferation-required operon that has been identified by using the operon prediction methods described herein, including antisense nucleic acids complementary to at least a portion of a proliferation-required operon comprising a nucleotide sequence selected from the group consisting of SEQ ID NOs: 1-194, can be used as antisense therapeutics for the treatment of bacterial infections or simply for inhibition of bacterial growth in vitro or in vivo. For example, the antisense therapeutics may be used to treat bacterial infections caused by Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species.

The therapy exploits the biological process in cells where genes are transcribed into messenger RNA (mRNA) that is then translated into proteins. Antisense RNA technology contemplates the use of antisense nucleic acids, including antisense oligonucleotides, complementary to at least a portion of a proliferation-required operon that comprises a target gene, that will bind to the operon or a transcript thereof thereby decreasing or inhibiting the expression of the target gene. For example, the antisense nucleic acid may inhibit the translation or transcription of the target nucleic acid. In one embodiment, antisense oligonucleotides can be used to treat and control a bacterial infection of a cell culture containing a population of desired cells contaminated with bacteria. In another embodiment, the antisense oligonucleotides can be used to treat an organism with a bacterial infection.

Antisense oligonucleotides can be synthesized that are complementary to portions of any of the operon sequences of the present invention using methods well known in the art. In a preferred embodiment, antisense oligonucleotides are synthesized using artificial means. Uhlmann & Peymann, Chemical Rev. 90:543-584 (1990) review antisense oligonucleotide technology in detail. Modified or unmodified antisense oligonucleotides can be used as therapeutic agents. Modified antisense oligonucleotides are preferred. Modification of the phosphate backbones of the antisense oligonucleotides can be achieved by substituting the internucleotide phosphate residues with methylphosphonates, phosphorothioates, phosphoramidates, and phosphate esters. Nonphosphate internucleotide analogs such as siloxane bridges, carbonate brides, thioester bridges, as well as many others known in the art may also be used. The preparation of certain antisense oligonucleotides with modified internucleotide linkages is described in U.S. Pat. No. 5,142,047, the disclosure of which is incorporated herein by reference in its entirety.

Modifications to the nucleoside units of the antisense oligonucleotides are also contemplated. These modifications can increase the half-life and increase cellular rates of uptake for the oligonucleotides in vivo. For example, α-anomeric nucleotide units and modified nucleotides such as 1,2-dideoxy-d-ribofuranose, 1,2-dideoxy-1-phenylribofuranose, and N⁴, N⁴-ethano-5-methyl-cytosine are contemplated for use in the present invention.

An additional form of modified antisense molecules is found in peptide nucleic acids. Peptide nucleic acids (PNA) have been developed to hybridize to single and double stranded nucleic acids. PNA are nucleic acid analogs in which the entire deoxyribose-phosphate backbone has been exchanged with a chemically different, but structurally homologous, polyamide (peptide) backbone containing 2-aminoethyl glycine units. Unlike DNA, which is highly negatively charged, the PNA backbone is neutral. Therefore, there is much less repulsive energy between complementary strands in a PNA-DNA hybrid than in the comparable DNA-DNA hybrid, and consequently they are much more stable. PNA can hybridize to DNA in either a Watson/Crick or Hoogsteen fashion (Demidov et al., Proc. Natl. Acad. Sci. U.S.A. 92:2637-2641, 1995; Egholm, Nature 365:566-568, 1993; Nielsen et al., Science 254:1497-1500, 1991; Dueholm et al., New J. Chem. 21:19-31, 1997).

Molecules called PNA “clamps” have been synthesized which have two identical PNA sequences joined by a flexible hairpin linker containing three 8-amino-3,6-dioxaoctanoic acid units. When a PNA clamp is mixed with a complementary homopurine or homopyrimidine DNA target sequence, a PNA-DNA-PNA triplex hybrid can form which has been shown to be extremely stable (Bentin et al., Biochemistry 35:8863-8869, 1996; Egholm et al., Nucleic Acids Res. 23:217-222, 1995; Griffith et al., J. Am. Chem. Soc. 117:831-832, 1995).

The sequence-specific and high affinity duplex and triplex binding of PNA have been extensively described (Nielsen et al., Science 254:1497-1500, 1991; Egholm et al., J. Am. Chem. Soc. 114:9677-9678, 1992; Egholm et al., Nature 365:566-568, 1993; Almarsson et al., Proc. Natl. Acad. Sci. U.S.A. 90:9542-9546, 1993; Demidov et al., Proc. Natl. Acad. Sci. U.S.A. 92:2637-2641, 1995). They have also been shown to be resistant to nuclease and protease digestion (Demidov et al., Biochem. Pharm. 48:1010-1313, 1994). PNA has been used to inhibit gene expression (Hanvey et al., Science 258:1481-1485,1992; Nielsen et al., Nucl. Acids. Res., 21:197-200, 1993; Nielsen et al., Gene 149:139-145, 1994; Good & Nielsen, Science, 95: 2073-2076, 1998; all of which are hereby incorporated by reference), to block restriction enzyme activity (Nielsen et al., supra., 1993), to act as an artificial transcription promoter (Mollegaard, Proc. Natl. Acad. Sci. U.S.A. 91:3892-3895, 1994) and as a pseudo restriction endonuclease (Demidov et al., Nucl. Acids. Res. 21:2103-2107, 1993). Recently, PNA has also been shown to have antiviral and antitumoral activity mediated through an antisense mechanism (Norton, Nature Biotechnol., 14:615-619, 1996; Hirschman et al., J. Investig. Med. 44:347-351, 1996). PNAs have been linked to various peptides in order to promote PNA entry into cells (Basu et al., Bioconj. Chem. 8:481-488, 1997; Pardridge et al., Proc. Natl. Acad. Sci. U.S.A. 92:5592-5596, 1995).

The antisense oligonucleotides contemplated by the present invention can be administered by direct application of oligonucleotides to a target using standard techniques well known in the art. The antisense oligonucleotides can be generated within the target using a plasmid, or a phage. Alternatively, the antisense nucleic acid may be expressed from a sequence in the chromosome of the target cell. For example, a promoter may be introduced into the chromosome of the target cell near the target gene such that the promoter directs the transcription of the antisense nucleic acid. Alternatively, a nucleic acid containing the antisense sequence operably linked to a promoter may be introduced into the chromosome of the target cell. It is further contemplated that the antisense oligonucleotides are incorporated in a ribozyme sequence to enable the antisense to specifically bind and cleave its target mRNA. For technical applications of ribozyme and antisense oligonucleotides see Rossi et al., Pharmacol. Ther. 50(2):245-254, (1991), which is hereby incorporated by reference. The present invention also contemplates using a retron to introduce an antisense oligonucleotide to a cell. Retron technology is exemplified by U.S. Pat. No. 5,405,775, which is hereby incorporated by reference. Antisense oligonucleotides can also be delivered using liposomes or by electroporation techniques which are well known in the art.

The antisense nucleic acids described above can also be used to design antibiotic compounds comprising nucleic acids which function by intracellular triple helix formation. Triple helix oligonucleotides are used to inhibit transcription from a genome. The antisense nucleic acids can be used to inhibit cell or microorganism gene expression in individuals infected with such microorganisms or containing such cells. Traditionally, homopurine sequences were considered the most useful for triple helix strategies. However, homopyrimidine sequences can also inhibit gene expression. Such homopyrimidine oligonucleotides bind to the major groove at homopurine:homopyrimidine sequences. Thus, both types of sequences based on sequences complementary to operons identified from Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigellaflexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species are contemplated for use as antibiotic compound templates.

The antisense nucleic acids, such as antisense oligonucleotides, which are complementary to at least a portion of a proliferation-required operon identified using the operon prediction methods described herein from Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigellaflexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species can be used to induce bacterial cell death or at least bacterial stasis by inhibiting target nucleic acid transcription or translation. Antisense oligonucleotides complementary to about 8 to 40 nucleotides of the proliferation-required operons, including the operons comprising a nucleotide sequence selected from the group consisting of SEQ ID NOs: 1-194, have sufficient complementarity to form a duplex with the proliferation operon comprising the one or more target genes under physiological conditions.

To kill bacterial cells or inhibit their growth, the antisense oligonucleotides are applied to the bacteria or to the target cells under conditions that facilitate their uptake. These conditions include sufficient incubation times of cells and oligonucleotides so that the antisense oligonucleotides are taken up by the cells. In one embodiment, an incubation period of 7-10 days is sufficient to kill bacteria in a sample. An optimum concentration of antisense oligonucleotides is selected for use.

The concentration of antisense oligonucleotides to be used can vary depending on the type of bacteria sought to be controlled, the nature of the antisense oligonucleotide to be used, and the relative toxicity of the antisense oligonucleotide to the desired cells in the treated culture. Antisense oligonucleotides can be introduced to cell samples at a number of different concentrations preferably between 1×10⁻¹⁰M to 1×10⁻⁴M. Once the minimum concentration that can adequately control gene expression is identified, the optimized dose is translated into a dosage suitable for use in vivo. For example, an inhibiting concentration in culture of 1×10⁻⁷ translates into a dose of approximately 0.6 mg/kg body weight. Levels of oligonucleotide approaching 100 mg/kg body weight or higher may be possible after testing the toxicity of the oligonucleotide in laboratory animals. It is additionally contemplated that cells from the subject are removed, treated with the antisense oligonucleotide, and reintroduced into the subject. This range is merely illustrative and one of skill in the art are able to determine the optimal concentration to be used in a given case.

After the bacterial cells have been killed or controlled in a desired culture, the desired cell population may be used for other purposes.

### Example 77

### Use of Antisense Oligonucleotides to Treat Infections

A subject suffering from an infection with Staphylococcus aureus is treated with the antisense oligonucleotide preparation described above wherein the antisense preparation comprises an antisense or antisense analog that is complementary to at least a portion of an operon comprising a nucleotide sequence selected from the group consisting of SEQ ID NOs: 1-194.

It will be appreciated that subject suffering from an infection with Staphylococcus aureus can be treated with the antisense oligonucleotide preparation described above wherein the antisense preparation comprises an antisense or antisense analog that is complementary to at least a portion of an operon comprising a nucleotide sequence selected from the group consisting of SEQ ID NOs: 1-194, wherein the portion does not include a nucleotide sequence selected from the group consisting of SEQ ID NOs: 201-550.

It will be appreciated that subject suffering from an infection with Acinetobacter baumannit, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsit, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species can be treated with the antisense oligonucleotide preparation described above wherein the antisense preparation comprises an antisense or antisense analog that is complementary to at least a portion of an operon comprising a nucleotide sequence selected from the group consisting of SEQ ID NOs: 1-194

It also will be appreciated that a subject suffering from an infection with Acinetobacter baumannit, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species which contains an operon which comprises a gene that is homologous to a gene selected from the group consisting of SEQ ID NOs: 201-550 or a gene that encodes a polypeptide that is homologous to a polypeptide having an amino acid sequence selected from the group consisting of SEQ ID NOs: 551-824 can be treated with an antisense preparation described above wherein the antisense preparation comprises an antisense or antisense analog that is complementary to at least a portion of the operon that comprises the proliferation-required gene.

The antisense oligonucleotide is provided in a pharmaceutically acceptable carrier at a concentration effective to inhibit the transcription or translation of the target nucleic acid. The present subject is treated with a concentration of antisense oligonucleotide sufficient to achieve a blood concentration of about 0.1-100 micromolar. The patient receives daily injections of antisense oligonucleotide to maintain this concentration for a period of 1 week. At the end of the week a blood sample is drawn and analyzed for the presence or absence of the organism using standard techniques well known in the art. There is no detectable evidence of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigella flexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis or any species falling within the genera of any of the above species and the treatment is terminated.

### Example 78

### Preparation and Use of Triple Helix Forming Oligonucleotides

The above-described antisense nucleic acids are scanned to identify 10-mer to 20-mer homopyrimidine or homopurine stretches that could be used in triple-helix based strategies for inhibiting gene expression. Following identification of candidate homopyrirmidine or homopurine stretches, their efficiency in inhibiting gene expression is assessed by introducing varying amounts of oligonucleotides containing the candidate sequences into a population of bacterial cells that normally express the target gene. The oligonucleotides may be prepared on an oligonucleotide synthesizer or they may be purchased commercially from a company specializing in custom oligonucleotide synthesis.

The oligonucleotides can be introduced into the cells using a variety of methods known to those skilled in the art, including but not limited to calcium phosphate precipitation, DEAE-Dextran, electroporation, liposome-mediated transfection or native uptake.

Treated cells are monitored for a reduction in proliferation using techniques such as monitoring growth levels as compared to untreated cells using optical density measurements. The oligonucleotides that are effective in inhibiting gene expression in cultured cells can then be introduced in vivo using the techniques well known in that art at a dosage level shown to be effective.

In some embodiments, the natural (beta) anomers of the oligonucleotide units can be replaced with alpha anomers to render the oligonucleotide more resistant to nucleases. Further, an intercalating agent such as ethidium bromide, or the like, can be attached to the 3′ end of the alpha oligonucleotide to stabilize the triple helix. For information on the generation of oligonucleotides suitable for triple helix formation see Griffin et al. (Science 245:967-971 (1989), the disclosure of which is incorporated herein by reference in its entirety).

It will be appreciated that no matter how detailed the foregoing appears in text, the invention can be practiced in many ways. As is also stated above, it should further be noted that the use of particular terminology when describing certain features or aspects of the present invention should not be taken to imply that the broadest reasonable meaning of such terminology is not intended, or that the terminology is being re-defined herein to be restricted to including any specific characteristics of the features or aspects of the invention with which that terminology is associated. Thus, although this invention has been described in terms of certain preferred embodiments, other embodiments which will be apparent to those of ordinary skill in the art in view of the disclosure herein are also within the scope of this invention. Accordingly, the scope of the invention is intended to be defined only by reference to the appended claims and any equivalents thereof. All documents cited herein are incorporated herein by reference in their entireties

1. A method for predicting operons, the method comprising:
identifying consecutive genes within at least a portion of a target prokaryotic organism's genome;
determining each gene's orientation in the genome relative to its flanking genes;
segregating the genes into a plurality of bins on the basis of their orientation such that consecutive genes in the same orientation are grouped into the same bin;
performing a composite operon prediction analysis comprising pairing each gene within a selected bin with its respective flanking genes and associating a confidence score with each gene pair, wherein the confidence score reflects the likelihood that a selected gene pair resides in the same operon using the criteria of orientation and the application of at least one operon prediction method;
determining operon boundaries by identifying gene pairs having confidence scores that fall below a selected threshold; and
associating genes contained between operon boundaries as putative operons.

2. The method of claim 1, wherein the target prokaryotic organism is selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigellaflexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis and any species falling within the genera of any of the above species.

3. The method of claim 1, wherein the target prokaryotic organism is Staphylococcus aureus.

4. The method of claim 1, wherein segregation of the genes into the plurality of bins further comprises identifying monocistronic operons by identifying genes where both the 5′ and 3′ flanking genes are oppositely oriented relative to the selected gene.

5. The method of claim 1, wherein segregation of the genes into the plurality of bins further comprises identifying genes having at least one 5′ or 3′ flanking gene in the same orientation.

6. The method of claim 1, wherein the results from the at least one operon prediction method are evaluated with respect to gene pair orientation and according to a pre-selected scoring criteria to determine the confidence score value associated with each gene pair.

7. The method of claim 1, wherein the at least one operon prediction method comprises an intergenic distance analysis.

8. The method of claim 1, wherein the at least one operon prediction method comprises a pairwise assessment of gene conservation.

9. The method of claim 1, wherein the at least one operon prediction method comprises a conserved gene cluster analysis.

10. The method of claim 9, wherein the conserved gene cluster analysis further comprises the steps of:
(a) identifying gene pairs contained in the bins for the target organism having an intergenic distance below a selected intergenic distance threshold;
(b) comparing the sequence and location of identified target organism gene pairs with homologous gene pairs from at least one comparison organism to identify conserved gene pairs between the target organism and the comparision organism;
(c) repeating steps (a)-(b) for each remaining comparision organism; and
(d) evaluating the conserved gene pairs between the target organism and each comparision organism with respect to one another to identify conserved gene clusters.

11. The method of claim 1, wherein the at least one operon prediction method comprises a transcriptional terminator analysis.

12. The method of claim 11, wherein the transcriptional terminator analysis further comprises:
extracting a portion of the sequence on both sides of a stop codon associated with each gene;
evaluating the portion of the sequence on both sides of the stop codon of each gene to identify prospective transcriptional terminators; and
associating a transcriptional terminator value with each gene on the basis of identified prospective transcriptional terminators.

13. The method of claim 1, wherein a numerical value is associated with the confidence score and reflects the likelihood of selected genes residing in the same operon, wherein the numerical value is selected from the group consisting of:
(a) a first numerical value which indicates that the genes of the gene pair are unlikely to be in the same operon if any of the following three criteria are met:
(i) the selected genes are in different orientations;
(ii) the intergenic distance between the selected genes is greater than approximately 300 bp; or
(iii) the intergenic distance between the selected genes is greater than 100 bp and there are no conserved gene clusters;
(b) a second numerical value which indicates that the selected genes might be in the same operon but the confidence is low if:
(i) the selected genes have an intergenic distance greater than approximately 60 bp,
(ii) the selected genes are conserved over approximately five or fewer comparison organisms, and
(iii) a predicted transcriptional terminator exists between the selected genes;
(c) a third numerical value which indicates that the selected genes are probably in the same operon if any of the following criteria are met:
(i) the selected genes are conserved in at least approximately 10 comparison organisms;
(ii) the intergenic distance between the selected genes is approximately less than or equal to 30 bp;
or if at least two of the following requirements are met:
(i) the intergenic distance between the selected genes is approximately less than or equal to 50 bp;
(ii) no predicted transcriptional terminators exist; or
(iii) the selected genes are conserved in approximately greater or equal to 5 comparison organisms but less than approximately 10 comparison organisms;
(d) a fourth numerical value which indicates that the selected genes are likely to reside in the same operon if the genes do not meet any of the above requirements.

14. A computer-based system for predicting operons within a target prokaryotic organism, the system comprising:
a database for storing information describing a plurality of genes relating to at least a portion of the target organism's genome;
a program which performs the operations of;
identifying consecutive genes from the plurality of genes stored in the database;
determining the orientation of each consecutive gene relative to its flanking genes;
segregating the consecutive genes into a plurality of bins on the basis of their orientation such that consecutive genes in the same orientation are grouped into the same bin;
performing a composite operon prediction analysis comprising pairing each consecutive gene within a selected bin with its respective flanking genes and associating a confidence score with each gene pair, wherein the confidence score reflects the likelihood that a selected gene pair resides in the same operon using the criteria of orientation and the application of at least one operon prediction method;
determining operon boundaries by identifying gene pairs having confidence scores that fall below a selected threshold; and
associating genes contained between operon boundaries as operons.

15. The system of claim 14, wherein the target prokaryotic organism is selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigellaflexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis and any species falling within the genera of any of the above species.

16. The system of claim 14, wherein the target prokaryotic organism is Staphylococcus aureus.

17. The system of claim 14, wherein the program operations of segregating the consecutive genes into the plurality of bins further comprises identifying monocistronic operons by identifying genes where both the 5′ and 3′ flanking genes are oppositely oriented relative to the selected gene.

18. The system of claim 14, wherein the program operations of segregating the consecutive genes into the plurality of bins further comprises identifying genes having at least one 5′ or 3′ flanking gene in the same orientation.

19. The system of claim 14, wherein the program operations further comprises evaluating the results from the at least one operon prediction method with respect to gene pair orientation and according to a pre-selected scoring criteria to determine the confidence score value associated with each gene pair.

20. The system of claim 14, wherein the program operations of application of the at least one operon prediction method comprises performing an intergenic distance analysis.

21. The system of claim 14, wherein the program operations of application of the at least one operon prediction method comprises performing a pairwise assessment of gene conservation.

22. The system of claim 14, wherein the database further stores information describing a plurality of genes relating to at least one other comparison organism and at least one operon prediction method performed by the program comprises an a conservation analysis wherein the program further performs the operations of:
determining an intergenic distance distribution across a selected number of gene pairs stored in the database for the target organism;
determining an intergenic distance distribution for genes stored in the database relating to the at least one other comparison organism;
comparing the intergenic distance distribution across the selected number of gene pairs with that of the comparison organism; and
associating a conservation value with each gene pair on the basis of identified similarities and differences in intergenic distance distribution between the target organism and the at least one other comparison organism.

23. The system of claim 14, wherein the database further stores information describing a plurality of genes relating to at least one other comparison organism and at least one operon prediction method performed by the program comprises a conserved gene cluster analysis wherein the program further performs the operations of:
(a) identifying target organism gene pairs having an intergenic distance below a selected intergenic distance threshold;
(b) comparing the sequence and location of identified target organism gene pairs with homologous gene pairs from at least one comparison organism to identify conserved gene pairs between the target organism and the comparision organism;
(c) repeating steps (a)-(b) for each remaining comparision organism; and
(d) evaluating the conserved gene pairs between the target organism and each comparision organism with respect to one another to identify conserved gene clusters.

24. The system of claim 14, wherein the at least one operon prediction method performed by the program comprises a transcriptional terminator analysis wherein the program further performs the operations of:
extracting a portion of the sequence on both sides of a stop codon associated with each gene from the database;
evaluating the portion of the sequence on both sides of the stop codon of each gene to identify prospective transcriptional terminators; and
associating a transcriptional terminator value with each gene pair on the basis of identified prospective transcriptional terminators.

25. A computer readable medium having stored thereon instructions which cause a general purpose computer to perform the steps of:
identifying consecutive genes within at least a portion of a target prokaryotic organism's genome;
determining each gene's orientation in the genome relative to its flanking genes;
segregating the genes into a plurality of bins on the basis of their orientation such that consecutive genes in the same orientation are grouped into the same bin;
performing a composite operon prediction analysis comprising pairing each gene within a selected bin with its respective flanking genes and associating a confidence score with each gene pair, wherein the confidence score reflects the likelihood that a selected gene pair resides in the same operon using the criteria of orientation and the application of at least one operon prediction method;
determining operon boundaries by identifying gene pairs having confidence scores that fall below a selected threshold; and
associating genes contained between operon boundaries as putative operons.

26. The computer readable medium of claim 25, wherein the steps performed by the computer operate on a target prokaryotic organism selected from the group consisting of Acinetobacter baumannii, Anaplasma marginale, Bacillus anthracis, Bacteroides fragilis, Bordetella pertussis, Borrelia burgdorferi, Brucella abortus, Brucella melitensis, Burkholderia cepacia, Burkholderia fungorum, Burkholderia mallei, Campylobacter jejuni, Chlamydia pneumoniae, Chlamydia trachomatis, Clostridium acetobutylicum, Clostridium botulinum, Clostridium difficile, Clostridium perfringens, Corynebacterium diptheriae, Coxiella burnetii, Enterobacter cloacae, Enterococcus faecalis, Enterococcus faecium, Escherichia coli, Francisella tularensis, Haemophilus influenzae, Helicobacter pylori, Klebsiella pneumoniae, Legionella pneumophila, Listeria monocytogenes, Moraxella catarrhalis, Mycobacterium avium, Mycobacterium bovis, Mycobacterium leprae, Mycobacterium tuberculosis, Mycoplasma genitalium, Mycoplasma pneumoniae, Neisseria gonorrhoeae, Neisseria meningitidis, Nocardia asteroides, Pasteurella haemolytica, Pasteurella multocida, Proteus mirabilis, Proteus vulgaris, Pseudomonas aeruginosa, Pseudomonas putida, Pseudomonas syringae, Rickettsia rickettsii, Rochalimaea quintana, Salmonella bongori, Salmonella cholerasuis, Salmonella enterica, Salmonella paratyphi, Salmonella typhi, Salmonella typhimurium, Shigella boydii, Shigella dysenteriae, Shigellaflexneri, Shigella sonnei, Staphylococcus aureus, Staphylococcus epidermidis, Staphylococcus haemolyticus, Streptococcus pneumoniae, Streptococcus mutans, Streptococcus pyogenes, Treponema pallidum, Ureaplasma urealyticum, Vibrio cholerae, Vibrio parahaemolyticus, Vibrio vulnificans, Yersinia enterocolitica, Yersinia pestis and any species falling within the genera of any of the above species.

27. The computer readable medium of claim 25, wherein the steps performed by the computer operate on a target prokaryotic organism that is Staphylococcus aureus.

28. The computer readable medium of claim 25, wherein segregation of the genes into the plurality of bins further comprises identifying putative polycistronic operons comprising a gene having at least one 5′ or 3′ flanking gene in the same orientation.

29. The computer readable medium of claim 25, wherein the results from the at least one operon prediction method are evaluated with respect to gene pair orientation and according to a pre-selected scoring criteria to determine the confidence score value associated with each gene pair.

30. The computer readable medium of claim 25, wherein the at least one operon prediction method comprises a pairwise assessment of gene conservation.

31. The computer readable medium of claim 25, wherein the at least one operon prediction method comprises a conserved gene cluster analysis.

32. The computer readable medium of claim 31, wherein the conserved gene cluster analysis further comprises the steps of:
(a) identifying gene pairs contained in the bins for the target organism having an intergenic distance below a selected intergenic distance threshold;
(b) comparing the sequence and location of identified target organism gene pairs with homologous gene pairs from at least one comparison organism to identify conserved gene pairs between the target organism and the comparision organism;
(c) repeating steps (a)-(b) for each remaining comparision organism; and
(d) evaluating the conserved gene pairs between the target organism and each comparision organism with respect to one another to identify conserved gene clusters.

33. The computer readable medium of claim 25, wherein the at least one operon prediction method comprises a transcriptional terminator analysis.

34. The computer readable medium of claim 33, wherein the transcriptional terminator analysis further comprises:
extracting a portion of the sequence on both sides of a stop codon associated with each gene;
evaluating the portion of the sequence on both sides of the stop codon of each gene to identify prospective transcriptional terminators; and
associating a transcriptional terminator value with each gene on the basis of identified prospective transcriptional terminators.

35. The computer readable medium of claim 25, wherein a numerical value is associated with the confidence score and reflects the likelihood of selected genes residing in the same operon, wherein the numerical value is selected from the group consisting of:
(a) a first numerical value which indicates that the genes of the gene pair are unlikely to be in the same operon if any of the following three criteria are met:
(i) the selected genes are in different orientations;
(ii) the intergenic distance between the selected genes is greater than approximately 300 bp; or
(iii) the intergenic distance between the selected genes is greater than 100 bp and there are no conserved gene clusters;
(b) a second numerical value which indicates that the selected genes might be in the same operon but the confidence is low if:
(i) the selected genes have an intergenic distance greater than approximately 60 bp,
(ii) the selected genes are conserved over approximately five or fewer comparison organisms, and
(iii) a predicted transcriptional terminator exists between the selected genes;
(c) a third numerical value which indicates that the selected genes are probably in the same operon if any of the following criteria are met:
(i) the selected genes are conserved in at least approximately 10 comparison organisms;
(ii) the intergenic distance between the selected genes is approximately less than or equal to 30 bp;
or if at least two of the following requirements are met:
(i) the intergenic distance between the selected genes is approximately less than or equal to 50 bp;
(ii) no predicted transcriptional terminators exist; or
(iii) the selected genes are conserved in approximately greater or equal to 5 comparison organisms but less than approximately 10 comparison organisms;
(d) a fourth numerical value which indicates that the selected genes are likely to reside in the same operon if the genes do not meet any of the above requirements.
